SEQUENCE LISTING (1140 sequences)

SEQ ID NO: 1 - protein, 8 aa, unidentified
  REGION 1..8: note = Description of Unknown: Protease cleavage site sequence
KRALGLPG

SEQ ID NO: 2 - protein, 40 aa, unidentified
  REGION 1..40: note = Description of Unknown: Protease cleavage site sequence
DEDEDEDEDEDEDEDERPLALWRSDRDRDRDRDRDRDRDR

SEQ ID NO: 3 - skipped

SEQ ID NO: 4 - protein, 5 aa, unidentified
  REGION 1..5: note = Description of Unknown: Protease cleavage site sequence
LEATA

SEQ ID NO: 5 - protein, 10 aa, unidentified
  REGION 1..10: note = Description of Unknown: Protease cleavage site sequence
GGAANLVRGG

SEQ ID NO: 6 - protein, 10 aa, unidentified
  REGION 1..10: note = Description of Unknown: Protease cleavage site sequence
SGRIGFLRTA

SEQ ID NO: 7 - protein, 6 aa, unidentified
  REGION 1..6: note = Description of Unknown: Protease cleavage site sequence
PLGLAG

SEQ ID NO: 8 - protein, 6 aa, unidentified
  REGION 1..6: note = Description of Unknown: Protease cleavage site sequence
  VARIANT 6: note = Any amino acid
PLGLAX

SEQ ID NO: 9 - protein, 6 aa, unidentified
  REGION 1..6: note = Description of Unknown: Protease cleavage site sequence
PLGCAG

SEQ ID NO: 10 - protein, 8 aa, unidentified
  REGION 1..8: note = Description of Unknown: Protease cleavage site sequence
ESPAYYTA

SEQ ID NO: 11 - protein, 6 aa, unidentified
  REGION 1..6: note = Description of Unknown: Protease cleavage site sequence
RLQLKL

SEQ ID NO: 12 - protein, 7 aa, unidentified
  REGION 1..7: note = Description of Unknown: Protease cleavage site sequence
RLQLKAC

SEQ ID NO: 13 - protein, 7 aa, unidentified
  REGION 1..7: note = Description of Unknown: Protease cleavage site sequence
  SITE 3: note = Citrulline (Cit)
  SITE 5: note = Homophenylalanine (Hof)
EPXGXYL

SEQ ID NO: 14 - protein, 5 aa, unidentified
  REGION 1..5: note = Description of Unknown: Protease cleavage site sequence
SGRSA

SEQ ID NO: 15 - protein, 4 aa, unidentified
  REGION 1..4: note = Description of Unknown: Protease cleavage site sequence
DAFK

SEQ ID NO: 16 - protein, 5 aa, unidentified
  REGION 1..5: note = Description of Unknown: Protease cleavage site sequence
GGGRR

SEQ ID NO: 17 - protein, 4 aa, unidentified
  REGION 1..4: note = Description of Unknown: Protease cleavage site sequence
GFLG

SEQ ID NO: 18 - protein, 4 aa, unidentified
  REGION 1..4: note = Description of Unknown: Protease cleavage site sequence
ALAL

SEQ ID NO: 19-20 - skipped (2 entries)

SEQ ID NO: 21 - protein, 5 aa, unidentified
  REGION 1..5: note = Description of Unknown: Protease cleavage site sequence
PICFF

SEQ ID NO: 22 - protein, 8 aa, unidentified
  REGION 1..8: note = Description of Unknown: Protease cleavage site sequence
GGPRGLPG

SEQ ID NO: 23 - protein, 6 aa, unidentified
  REGION 1..6: note = Description of Unknown: Protease cleavage site sequence
HSSKLQ

SEQ ID NO: 24 - protein, 7 aa, unidentified
  REGION 1..7: note = Description of Unknown: Protease cleavage site sequence
HSSKLQL

SEQ ID NO: 25 - protein, 9 aa, unidentified
  REGION 1..9: note = Description of Unknown: Protease cleavage site sequence
HSSKLQEDA

SEQ ID NO: 26 - protein, 10 aa, unidentified
  REGION 1..10: note = Description of Unknown: Protease cleavage site sequence
LVLASSSFGY

SEQ ID NO: 27 - protein, 10 aa, unidentified
  REGION 1..10: note = Description of Unknown: Protease cleavage site sequence
GVSQNYPIVG

SEQ ID NO: 28 - protein, 10 aa, unidentified
  REGION 1..10: note = Description of Unknown: Protease cleavage site sequence
GVVQASCRLA

SEQ ID NO: 29 - skipped

SEQ ID NO: 30 - protein, 6 aa, unidentified
  REGION 1..6: note = Description of Unknown: Protease cleavage site sequence
DPRSFL

SEQ ID NO: 31 - protein, 6 aa, unidentified
  REGION 1..6: note = Description of Unknown: Protease cleavage site sequence
PPRSFL

SEQ ID NO: 32 - protein, 4 aa, unidentified
  REGION 1..4: note = Description of Unknown: Protease cleavage site sequence
DEVD

SEQ ID NO: 33 - protein, 5 aa, unidentified
  REGION 1..5: note = Description of Unknown: Protease cleavage site sequence
DEVDP

SEQ ID NO: 34 - protein, 8 aa, unidentified
  REGION 1..8: note = Description of Unknown: Protease cleavage site sequence
KGSGDVEG

SEQ ID NO: 35 - protein, 6 aa, unidentified
  REGION 1..6: note = Description of Unknown: Protease cleavage site sequence
GWEHDG

SEQ ID NO: 36 - protein, 7 aa, unidentified
  REGION 1..7: note = Description of Unknown: Protease cleavage site sequence
EDDDDKA

SEQ ID NO: 37 - protein, 9 aa, unidentified
  REGION 1..9: note = Description of Unknown: Protease cleavage site sequence
KQEQNPGST

SEQ ID NO: 38 - protein, 6 aa, unidentified
  REGION 1..6: note = Description of Unknown: Protease cleavage site sequence
GKAFRR

SEQ ID NO: 39 - protein, 4 aa, unidentified
  REGION 1..4: note = Description of Unknown: Protease cleavage site sequence
DAFK

SEQ ID NO: 40 - protein, 4 aa, unidentified
  REGION 1..4: note = Description of Unknown: Protease cleavage site sequence
DVLK

SEQ ID NO: 41 - protein, 4 aa, unidentified
  REGION 1..4: note = Description of Unknown: Protease cleavage site sequence
DAFK

SEQ ID NO: 42 - protein, 7 aa, unidentified
  REGION 1..7: note = Description of Unknown: Protease cleavage site sequence
ALLLALL

SEQ ID NO: 43 - protein, 518 aa, synthetic construct
  REGION 1..518: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 44 - protein, 518 aa, synthetic construct
  REGION 1..518: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 45 - protein, 382 aa, synthetic construct
  REGION 1..382: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 46 - protein, 534 aa, synthetic construct
  REGION 1..534: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 47 - protein, 534 aa, synthetic construct
  REGION 1..534: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 48 - protein, 398 aa, synthetic construct
  REGION 1..398: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 49 - protein, 503 aa, synthetic construct
  REGION 1..503: note = Description of Artificial Sequence: Synthetic polypeptide
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 50 - protein, 14 aa, synthetic construct
  REGION 1..14: note = Description of Artificial Sequence: Synthetic peptide
GGGGGLDGNEEPGG

SEQ ID NO: 51 - protein, 19 aa, synthetic construct
  REGION 1..19: note = Description of Artificial Sequence: Synthetic peptide
GGGGSGGGGSGGVVGGGGT

SEQ ID NO: 52 - protein, 509 aa, synthetic construct
  REGION 1..509: note = Description of Artificial Sequence: Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 53 - protein, 19 aa, synthetic construct
  REGION 1..19: note = Description of Artificial Sequence: Synthetic peptide
GGGGKPLGLQARVVGGGGT

SEQ ID NO: 54 - protein, 143 aa, synthetic construct
  REGION 1..143: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGT

SEQ ID NO: 55 - protein, 127 aa, synthetic construct
  REGION 1..127: note = Description of Artificial Sequence: Synthetic polypeptide
EVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSS

SEQ ID NO: 56 - protein, 249 aa, synthetic construct
  REGION 1..249: note = Description of Artificial Sequence: Synthetic polypeptide
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 57 - protein, 111 aa, synthetic construct
  REGION 1..111: note = Description of Artificial Sequence: Synthetic polypeptide
EVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSS

SEQ ID NO: 58 - protein, 10 aa, unidentified
  REGION 1..10: note = Description of Unknown: Protease cleavage site sequence
KPLGLQARVV

SEQ ID NO: 59 - protein, 10 aa, unidentified
  REGION 1..10: note = Description of Unknown: Protease cleavage site sequence
PQASTGRSGG

SEQ ID NO: 60 - protein, 10 aa, unidentified
  REGION 1..10: note = Description of Unknown: Protease cleavage site sequence
PQGSTGRAAG

SEQ ID NO: 61 - protein, 10 aa, unidentified
  REGION 1..10: note = Description of Unknown: Protease cleavage site sequence
PPASSGRAGG

SEQ ID NO: 62 - protein, 9 aa, unidentified
  REGION 1..9: note = Description of Unknown: Protease cleavage site sequence
PIPVQGRAH

SEQ ID NO: 63 - protein, 111 aa, synthetic construct
  REGION 1..111: note = Description of Artificial Sequence: Synthetic polypeptide
EVQLVESGGGLVQPGGSLRLSCAASRFMISEYHMHWVRQAPGKGLEWVSDINPAGTTDYAESVKGRFTISRDNAKNTLYLQMNSLRAEDTAVYYCDSYGYRGQGTLVTVSS

SEQ ID NO: 64 - protein, 111 aa, synthetic construct
  REGION 1..111: note = Description of Artificial Sequence: Synthetic polypeptide
EVQLVESGGGLVQPGGSLRLSCAASRFMISEYHMHWVRQAPGKGLEWVSTINPAGTTDYAESVKGRFTISRDNAKNTLYLQMNSLRAEDTAVYYCDSYGYRGQGTLVTVSS

SEQ ID NO: 65 - protein, 111 aa, synthetic construct
  REGION 1..111: note = Description of Artificial Sequence: Synthetic polypeptide
EVQLVESGGGLVQPGGSLRLSCAASRFMISEYSMHWVRQAPGKGLEWVSTINPAKTTDYAESVKGRFTISRDNAKNTLYLQMNSLRAEDTAVYYCDSYGYRGQGTLVTVSS

SEQ ID NO: 66 - protein, 111 aa, synthetic construct
  REGION 1..111: note = Description of Artificial Sequence: Synthetic polypeptide
EVQLVESGGGLVQPGGSLRLSCAASRFMISPYSMHWVRQAPGKGLEWVSTINPAGTTDYAESVKGRFTISRDNAKNTLYLQMNSLRAEDTAVYYCDGYGYRGQGTLVTVSS

SEQ ID NO: 67 - protein, 111 aa, synthetic construct
  REGION 1..111: note = Description of Artificial Sequence: Synthetic polypeptide
EVQLVESGGGLVQPGGSLRLSCAASRFMISEYSMHWVRQAPGKGLEWVSTINPAGQTDYAESVKGRFTISRDNAKNTLYLQMNSLRAEDTAVYYCDGYGYRGQGTLVTVSS

SEQ ID NO: 68 - protein, 111 aa, synthetic construct
  REGION 1..111: note = Description of Artificial Sequence: Synthetic polypeptide
EVQLVESGGGLVQPGGSLRLSCAASRFMISEYSMHWVRQAPGKGLEWVSTINPAGTTDYAEYVKGRFTISRDNAKNTLYLQMNSLRAEDTAVYYCDGYGYRGQGTLVTVSS

SEQ ID NO: 69 - protein, 111 aa, synthetic construct
  REGION 1..111: note = Description of Artificial Sequence: Synthetic polypeptide
EVQLVESGGGLVQPGGSLRLSCAASRFMISEYHMHWVRQAPGKGLEWVSDINPAKTTDYAESVKGRFTISRDNAKNTLYLQMNSLRAEDTAVYYCDSYGYRGQGTLVTVSS

SEQ ID NO: 70 - protein, 111 aa, synthetic construct
  REGION 1..111: note = Description of Artificial Sequence: Synthetic polypeptide
EVQLVESGGGLVQPGGSLRLSCAASRFMISPYHMHWVRQAPGKGLEWVSDINPAGTTDYAESVKGRFTISRDNAKNTLYLQMNSLRAEDTAVYYCDSYGYRGQGTLVTVSS

SEQ ID NO: 71 - protein, 111 aa, synthetic construct
  REGION 1..111: note = Description of Artificial Sequence: Synthetic polypeptide
EVQLVESGGGLVQPGGSLRLSCAASRFMISEYHMHWVRQAPGKGLEWVSDINPAGQTDYAESVKGRFTISRDNAKNTLYLQMNSLRAEDTAVYYCDSYGYRGQGTLVTVSS

SEQ ID NO: 72 - protein, 111 aa, synthetic construct
  REGION 1..111: note = Description of Artificial Sequence: Synthetic polypeptide
EVQLVESGGGLVQPGGSLRLSCAASRFMISEYHMHWVRQAPGKGLEWVSDINPAGTTDYAEYVKGRFTISRDNAKNTLYLQMNSLRAEDTAVYYCDSYGYRGQGTLVTVSS

SEQ ID NO: 73 - protein, 111 aa, synthetic construct
  REGION 1..111: note = Description of Artificial Sequence: Synthetic polypeptide
EVQLVESGGGLVQPGGSLTLSCAASRFMISEYHMHWVRQAPGKGLEWVSDINPAGTTDYAESVKGRFTISRDNAKNTLYLQMNSLKPEDTAVYYCDSYGYRGQGTQVTVSS

SEQ ID NO: 74 - protein, 249 aa, synthetic construct
  REGION 1..249: note = Description of Artificial Sequence: Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 75 - protein, 249 aa, synthetic construct
  REGION 1..249: note = Description of Artificial Sequence: Synthetic polypeptide
EVQLVESGGGLVQPGGSLKLSCAASGFEFNKYAMNWVRQAPGKGLEWVARIRSKYNKYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSFGAVTSGNYPNWVQQKPGQAPRGLIGGTKFLAPGTPARFSGSLLGGKAALTLSGVQPEDEAEYYCVLWYDNRWVFGGGTKLTVL

SEQ ID NO: 76 - protein, 249 aa, synthetic construct
  REGION 1..249: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSHISYWAYWGQGTLVTVSSGGGGSGGGGSGGGGSQTVVTQEPSLTVSPGGTVTLTCGSSTGYVTSGNYPNWVQQKPGQAPRGLIGGTSFLAPGTPARFSGSLLGGKAALTLSGVQPEDEAEYYCVLWYSNRWIFGGGTKLTVL

SEQ ID NO: 77 - protein, 249 aa, synthetic construct
  REGION 1..249: note = Description of Artificial Sequence: Synthetic polypeptide
EVQLVESGGGLVQPGGSLKLSCAASGFMFNKYAMNWVRQAPGKGLEWVARIRSKSNNYATYYADSVKDRFTISRDDSKNTAYLQMNNLKTEDTAVYYCVRHGNFGNSYISYWATWGQGTLVTVSSGGGGSGGGGSGGGGSQTVVTQEPSLTVSPGGTVTLTCGSSFGAVTSGNYPNWVQQKPGQAPRGLIGGTKLLAPGTPARFSGSLLGGKAALTLSGVQPEDEAEYYCVLWYSNSWVFGGGTKLTVL

SEQ ID NO: 78 - protein, 249 aa, synthetic construct
  REGION 1..249: note = Description of Artificial Sequence: Synthetic polypeptide
EVQLVESGGGLVQPGGSLKLSCAASGFTFNTYAMNWVRQAPGKGLEWVARIRSKYNNYATYYKDSVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVVSGNYPNWVQQKPGQAPRGLIGGTEFLAPGTPARFSGSLLGGKAALTLSGVQPEDEAEYYCVLWYSNRWVFGGGTKLTVL

SEQ ID NO: 79 - protein, 249 aa, synthetic construct
  REGION 1..249: note = Description of Artificial Sequence: Synthetic polypeptide
EVQLVESGGGLVQPGGSLKLSCAASGFTYNKYAMNWVRQAPGKGLEWVARIRSKYNNYATYYADEVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSKGAVTSGNYPNWVQQKPGQAPRGLIGGTKELAPGTPARFSGSLLGGKAALTLSGVQPEDEAEYYCTLWYSNRWVFGGGTKLTVL

SEQ ID NO: 80 - protein, 249 aa, synthetic construct
  REGION 1..249: note = Description of Artificial Sequence: Synthetic polypeptide
EVQLVESGGGLVQPGGSLKLSCAASGNTFNKYAMNWVRQAPGKGLEWVARIRSKYNNYETYYADSVKDRFTISRDDSKNTAYLQMNNLKTEDTAVYYCVRHTNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTYFLAPGTPARFSGSLLGGKAALTLSGVQPEDEAEYYCVLWYSNRWVFGGGTKLTVL

SEQ ID NO: 81 - protein, 249 aa, synthetic construct
  REGION 1..249: note = Description of Artificial Sequence: Synthetic polypeptide
EVQLVESGGGLVQPGGSLKLSCAASGFTFNNYAMNWVRQAPGKGLEWVARIRSKYNNYATYYADAVKDRFTISRDDSKNTAYLQMNNLKTEDTAVYYCVRHGNFGNSQISYWAYWGQGTLVTVSSGGGGSGGGGSGGGGSQTVVTQEPSLTVSPGGTVTLTCGSSTGAVTDGNYPNWVQQKPGQAPRGLIGGIKFLAPGTPARFSGSLLGGKAALTLSGVQPEDEAEYYCVLWYSNRWVFGGGTKLTVL

SEQ ID NO: 82 - protein, 249 aa, synthetic construct
  REGION 1..249: note = Description of Artificial Sequence: Synthetic polypeptide
EVQLVESGGGLVQPGGSLKLSCAASGFTFNKYAVNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGESTGAVTSGNYPNWVQQKPGQAPRGLIGGTKILAPGTPARFSGSLLGGKAALTLSGVQPEDEAEYYCVLWYSNRWVFGGGTKLTVL

SEQ ID NO: 83 - protein, 249 aa, synthetic construct
  REGION 1..249: note = Description of Artificial Sequence: Synthetic polypeptide
EVQLVESGGGLVQPGGSLKLSCAASGFTFNKYPMNWVRQAPGKGLEWVARIRSKYNNYATYYADSVKDRFTISRDDSKNTAYLQMNNLKNEDTAVYYCVRHGNFNNSYISYWAYWGQGTLVTVSSGGGGSGGGGSGGGGSQTVVTQEPSLTVSPGGTVTLTCGSSTGAVTKGNYPNWVQQKPGQAPRGLIGGTKMLAPGTPARFSGSLLGGKAALTLSGVQPEDEAEYYCALWYSNRWVFGGGTKLTVL

SEQ ID NO: 84 - protein, 249 aa, synthetic construct
  REGION 1..249: note = Description of Artificial Sequence: Synthetic polypeptide
EVQLVESGGGLVQPGGSLKLSCAASGFTFNGYAMNWVRQAPGKGLEWVARIRSKYNNYATYYADEVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVVSGNYPNWVQQKPGQAPRGLIGGTEFLAPGTPARFSGSLLGGKAALTLSGVQPEDEAEYYCVLWYSNRWVFGGGTKLTVL

SEQ ID NO: 85 - protein, 249 aa, synthetic construct
  REGION 1..249: note = Description of Artificial Sequence: Synthetic polypeptide
EVQLVESGGGLVQPGGSLKLSCAASGNTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSSGGGGSGGGGSGGGGSQTVVTQEPSLTVSPGGTVTLTCGSSTGAVTHGNYPNWVQQKPGQAPRGLIGGTKVLAPGTPARFSGSLLGGKAALTLSGVQPEDEAEYYCVLWYSNRWVFGGGTKLTVL

SEQ ID NO: 86 - protein, 249 aa, synthetic construct
  REGION 1..249: note = Description of Artificial Sequence: Synthetic polypeptide
EVQLVESGGGLVQPGGSLKLSCAASGFTFNNYAMNWVRQAPGKGLEWVARIRSGYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSYTGAVTSGNYPNWVQQKPGQAPRGLIGGTKFNAPGTPARFSGSLLGGKAALTLSGVQPEDEAEYYCVLWYANRWVFGGGTKLTVL

SEQ ID NO: 87 - protein, 249 aa, synthetic construct
  REGION 1..249: note = Description of Artificial Sequence: Synthetic polypeptide
EVQLVESGGGLVQPGGSLKLSCAASGFEFNKYAMNWVRQAPGKGLEWVARIRSKYNNYETYYADSVKDRFTISRDDSKNTAYLQMNNLKTEDTAVYYCVRHGNFGNSLISYWAYWGQGTLVTVSSGGGGSGGGGSGGGGSQTVVTQEPSLTVSPGGTVTLTCGSSSGAVTSGNYPNWVQQKPGQAPRGLIGGTKFGAPGTPARFSGSLLGGKAALTLSGVQPEDEAEYYCVLWYSNRWVFGGGTKLTVL

SEQ ID NO: 88 - protein, 249 aa, synthetic construct
  REGION 1..249: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 89 - protein, 249 aa, synthetic construct
  REGION 1..249: note = Description of Artificial Sequence: Synthetic polypeptide
EVQLVESGGGLVQPGGSLKLSCAASGFTFNKYALNWVRQAPGKGLEWVARIRSKYNNYATEYADSVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVTSGNYPNWVQQKPGQAPRGLIGGTNFLAPGTPERFSGSLLGGKAALTLSGVQPEDEAEYYCVLWYSNRWAFGGGTKLTVL

SEQ ID NO: 90 - protein, 249 aa, synthetic construct
  REGION 1..249: note = Description of Artificial Sequence: Synthetic polypeptide
EVQLVESGGGLVQPGGSLKLSCAASGFTFNEYAMNWVRQAPGKGLEWVARIRSKYNNYATYYADDVKDRFTISRDDSKNTAYLQMNNLKTEDTAVYYCVRHGNFGNSGISYWAYWGQGTLVTVSSGGGGSGGGGSGGGGSQTVVTQEPSLTVSPGGTVTLTCGSSTGAVTVGNYPNWVQQKPGQAPRGLIGGTEFLAPGTPARFSGSLLGGKAALTLSGVQPEDEAEYYCVLWYSNRWVFGGGTKLTVL

SEQ ID NO: 91 - protein, 119 aa, synthetic construct
  REGION 1..119: note = Description of Artificial Sequence: Synthetic polypeptide
EVQLVESGGGLVQPGRSLTLSCAASTNIFSISPMGWYRQAPGKQRELVAAIHGFSTLYADSVKGRFTISRDNAKNSIYLQMNSLRPEDTALYYCNKVPWGDYHPRNVYWGQGTQVTVSS

SEQ ID NO: 92 - protein, 119 aa, synthetic construct
  REGION 1..119: note = Description of Artificial Sequence: Synthetic polypeptide
EVQLVESGGGLVQPGRSLTLSCAASTDIFSISPMGWYRQAPGKQRELVAAIHGGSTLYADSVKGRFTISRDNAKNSIYLQMNSLRPEDTALYYCNKVPWGDYHPRNVAWGQGTQVTVSS

SEQ ID NO: 93 - protein, 119 aa, synthetic construct
  REGION 1..119: note = Description of Artificial Sequence: Synthetic polypeptide
EVQLVESGGGLVQPGRSLTLSCAASTNDFSISPMGWYRQAPGKQRELVAAIHGGSTLYADSVKGRFTISRDNAKNSIYLQMNSLRPEDTALYYCNKVPWGDYHPRNVAWGQGTQVTVSS

SEQ ID NO: 94 - protein, 119 aa, synthetic construct
  REGION 1..119: note = Description of Artificial Sequence: Synthetic polypeptide
EVQLVESGGGLVQPGRSLTLSCAASTNIFSKSPMGWYRQAPGKQRELVAAIHGKSTLYADSVKGRFTISRDNAKNSIYLQMNSLRPEDTALYYCNKVPWGDYHPRNVVWGQGTQVTVSS

SEQ ID NO: 95 - protein, 119 aa, synthetic construct
  REGION 1..119: note = Description of Artificial Sequence: Synthetic polypeptide
EVQLVESGGGLVQPGRSLTLSCAASTNDFSISPMGWYRQAPGKQRELVAAIHGKSTLYADSVKGRFTISRDNAKNSIYLQMNSLRPEDTALYYCNKVPWGDYHPRNVKWGQGTQVTVSS

SEQ ID NO: 96 - protein, 119 aa, synthetic construct
  REGION 1..119: note = Description of Artificial Sequence: Synthetic polypeptide
EVQLVESGGGLVQPGRSLTLSCAASTNQFSISPMGWYRQAPGKQRELVAAIHGKSTLYADSVKGRFTISRDNAKNSIYLQMNSLRPEDTALYYCNKVPWGDYHPRNVVWGQGTQVTVSS

SEQ ID NO: 97 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAINGFSTLYADSVKGRFTISRDNAKNSIYLQMNSLRPEDTALYYCNKVPWGDYHPRNVHWGQGTQVTVSS

SEQ ID NO: 98 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPRNVYWGQGTQVTVSS

SEQ ID NO: 99 - protein, 119 aa, synthetic construct
  REGION 1..119: note = Description of Artificial Sequence: Synthetic polypeptide
EVQLVESGGGLVQPGRSLTLSCAASTNIFSTSPMGWYRQAPGKQRELVAAIHGFQTLYADSVKGRFTISRDNAKNSIYLQMNSLRPEDTALYYCNKVPWGDYHPRNVVWGQGTQVTVSS

SEQ ID NO: 100 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGFETLYADSVKGRFTISRDNAKNSIYLQMNSLRPEDTALYYCNKVPWGDYHPRNVLWGQGTQVTVSS

SEQ ID NO: 101 - protein, 119 aa, synthetic construct
  REGION 1..119: note = Description of Artificial Sequence: Synthetic polypeptide
EVQLVESGGGLVQPGRSLTLSCAASTNIFSESPMGWYRQAPGKQRELVAAIHGFTTLYADSVKGRFTISRDNAKNSIYLQMNSLRPEDTALYYCNKVPWGDYHPRNVTWGQGTQVTVSS

SEQ ID NO: 102 - protein, 119 aa, synthetic construct
  REGION 1..119: note = Description of Artificial Sequence: Synthetic polypeptide
EVQLVESGGGLVQPGRSLTLSCAASTNIFSDSPMGWYRQAPGKQRELVAAIHGFSTLYADSVKGRFTISRDNAKNSIYLQMNSLRPEDTALYYCNKVPWGDYHPRNVAWGQGTQVTVSS

SEQ ID NO: 103 - protein, 119 aa, synthetic construct
  REGION 1..119: note = Description of Artificial Sequence: Synthetic polypeptide
EVQLVESGGGLVQPGRSLTLSCAASTNIFSNSPMGWYRQAPGKQRELVAAIHGGSTLYADSVKGRFTISRDNAKNSIYLQMNSLRPEDTALYYCNKVPWGDYHPRNVHWGQGTQVTVSS

SEQ ID NO: 104 - protein, 119 aa, synthetic construct
  REGION 1..119: note = Description of Artificial Sequence: Synthetic polypeptide
EVQLVESGGGLVQPGRSLTLSCAASTNIFSNSPMGWYRQAPGKQRELVAAIHGRSTLYADSVKGRFTISRDNAKNSIYLQMNSLRPEDTALYYCNKVPWGDYHPRNVMWGQGTQVTVSS

SEQ ID NO: 105 - protein, 119 aa, synthetic construct
  REGION 1..119: note = Description of Artificial Sequence: Synthetic polypeptide
EVQLVESGGGLVQPGRSLTLSCAASTNIFSNSPMGWYRQAPGKQRELVAAIHGPSTLYADSVKGRFTISRDNAKNSIYLQMNSLRPEDTALYYCNKVPWGDYHPRNVTWGQGTQVTVSS

SEQ ID NO: 106 - protein, 119 aa, synthetic construct
  REGION 1..119: note = Description of Artificial Sequence: Synthetic polypeptide
EVQLVESGGGLVQPGRSLTLSCAASTNIFSNSPMGWYRQAPGKQRELVAAIHGDSTLYADSVKGRFTISRDNAKNSIYLQMNSLRPEDTALYYCNKVPWGDYHPRNVRWGQGTQVTVSS

SEQ ID NO: 107 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGDSTLYADSVKGRFTISRDNAKNSIYLQMNSLRPEDTALYYCNKVPWGDYHPRNVTWGQGTQVTVSS

SEQ ID NO: 108 - protein, 119 aa, synthetic construct
  REGION 1..119: note = Description of Artificial Sequence: Synthetic polypeptide
EVQLVESGGGLVQPGRSLTLSCAASTNIFSKSPMGWYRQAPGKQRELVAAIHGQSTLYADSVKGRFTISRDNAKNSIYLQMNSLRPEDTALYYCNKVPWGDYHPRNVTWGQGTQVTVSS

SEQ ID NO: 109 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAIHGHSTLYADSVKGRFTISRDNAKNSIYLQMNSLRPEDTALYYCNKVPWGDYHPRNVTWGQGTQVTVSS

SEQ ID NO: 110 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAIHGESTLYADSVKGRFTISRDNAKNSIYLQMNSLRPEDTALYYCNKVPWGDYHPRKVYWGQGTQVTVSS

SEQ ID NO: 111 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGNSTLYADSVKGRFTISRDNAKNSIYLQMNSLRPEDTALYYCNKVPWGIYHPRNVYWGQGTQVTVSS

SEQ ID NO: 112 - protein, 119 aa, synthetic construct
  REGION 1..119: note = Description of Artificial Sequence: Synthetic polypeptide
EVQLVESGGGLVQPGRSLTLSCAASTNIFSESPMGWYRQAPGKQRELVAAIHGNSTLYADSVKGRFTISRDNAKNSIYLQMNSLRPEDTALYYCNKVPWGTYHPRNVYWGQGTQVTVSS

SEQ ID NO: 113 - protein, 119 aa, synthetic construct
  REGION 1..119: note = Description of Artificial Sequence: Synthetic polypeptide
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGKYHPRNVYWGQGTQVTVSS

SEQ ID NO: 114 - protein, 119 aa, synthetic construct
  REGION 1..119: note = Description of Artificial Sequence: Synthetic polypeptide
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGDYHPRNVYWGQGTQVTVSS

SEQ ID NO: 115 - protein, 119 aa, synthetic construct
  REGION 1..119: note = Description of Artificial Sequence: Synthetic polypeptide
EVQLVESGGGLVQPGRSLTLSCAASTNIFSKSPMGWYRQAPGKQRELVAAIHGNSTLYADSVKGRFTISRDNAKNSIYLQMNSLRPEDTALYYCNKVPWGDYHPREVYWGQGTQVTVSS

SEQ ID NO: 116 - protein, 119 aa, synthetic construct
  REGION 1..119: note = Description of Artificial Sequence: Synthetic polypeptide
EVQLVESGGGLVQPGRSLTLSCAASTNIFSDSPMGWYRQAPGKQRELVAAIHGTSTLYADSVKGRFTISRDNAKNSIYLQMNSLRPEDTALYYCNKVPWGDYHPRNVYWGQGTQVTVSS

SEQ ID NO: 117 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGTSTLYADSVKGRFTISRDNAKNSIYLQMNSLRPEDTALYYCNKVPWGKYHPRNVYWGQGTQVTVSS

SEQ ID NO: 118 - protein, 119 aa, synthetic construct
  REGION 1..119: note = Description of Artificial Sequence: Synthetic polypeptide
EVQLVESGGGLVQPGRSLTLSCAASTNIFSHSPMGWYRQAPGKQRELVAAIHGTSTLYADSVKGRFTISRDNAKNSIYLQMNSLRPEDTALYYCNKVPWGRYHPRNVYWGQGTQVTVSS

SEQ ID NO: 119 - protein, 119 aa, synthetic construct
  REGION 1..119: note = Description of Artificial Sequence: Synthetic polypeptide
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 120 - protein, 119 aa, synthetic construct
  REGION 1..119: note = Description of Artificial Sequence: Synthetic polypeptide
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPRNVQWGQGTQVTVSS

SEQ ID NO: 121 - protein, 119 aa, synthetic construct
  REGION 1..119: note = Description of Artificial Sequence: Synthetic polypeptide
EVQLVESGGGLVQPGRSLTLSCAASTNIFSLSPMGWYRQAPGKQRELVAAIHGDSTLYADSVKGRFTISRDNAKNSIYLQMNSLRPEDTALYYCNKVPWGDYHPRNVYWGQGTQVTVSS

SEQ ID NO: 122 - protein, 119 aa, synthetic construct
  REGION 1..119: note = Description of Artificial Sequence: Synthetic polypeptide
EVQLVESGGGLVQPGRSLTLSCAASTNIFSISPGGWYRQAPGKQRELVAAIHGSSTLYADSVKGRFTISRDNAKNSIYLQMNSLRPEDTALYYCNKVPWGDYHPRNVYWGQGTQVTVSS

SEQ ID NO: 123 - protein, 119 aa, synthetic construct
  REGION 1..119: note = Description of Artificial Sequence: Synthetic polypeptide
EVQLVESGGGLVQPGRSLTLSCAASTNHFSISPMGWYRQAPGKQRELVAAIHGSSTLYADSVKGRFTISRDNAKNSIYLQMNSLRPEDTALYYCNKVPWGDYHPRVVYWGQGTQVTVSS

SEQ ID NO: 124 - protein, 119 aa, synthetic construct
  REGION 1..119: note = Description of Artificial Sequence: Synthetic polypeptide
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGDYHPRNVNWGQGTQVTVSS

SEQ ID NO: 125 - protein, 119 aa, synthetic construct
  REGION 1..119: note = Description of Artificial Sequence: Synthetic polypeptide
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGRYHPRNVYWGQGTQVTVSS

SEQ ID NO: 126 - protein, 119 aa, synthetic construct
  REGION 1..119: note = Description of Artificial Sequence: Synthetic polypeptide
EVQLVESGGGLVQPGRSLTLSCAASTNISSISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 127 - protein, 119 aa, synthetic construct
  REGION 1..119: note = Description of Artificial Sequence: Synthetic polypeptide
EVQLVESGGGLVQPGRSLTLSCAASTNIESISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 128 - protein, 119 aa, synthetic construct
  REGION 1..119: note = Description of Artificial Sequence: Synthetic polypeptide
EVQLVESGGGLVQPGRSLTLSCAASTNIFSISPYGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 129 - protein, 119 aa, synthetic construct
  REGION 1..119: note = Description of Artificial Sequence: Synthetic polypeptide
EVQLVESGGGLVQPGRSLTLSCAASTNIASISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 130 - protein, 119 aa, synthetic construct
  REGION 1..119: note = Description of Artificial Sequence: Synthetic polypeptide
EVQLVESGGGLVQPGRSLTLSCAASTNIASISPMGWYRQAPGKQRELVAAIHGKSTLYADSVKGRFTISRDNAKNSIYLQMNSLRPEDTALYYCNKVPWGDYHPGNVYWGQGTQVTVSS

SEQ ID NO: 131 - protein, 119 aa, synthetic construct
  REGION 1..119: note = Description of Artificial Sequence: Synthetic polypeptide
EVQLVESGGGLVQPGRSLTLSCAASTNIASISPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 132 - protein, 119 aa, synthetic construct
  REGION 1..119: note = Description of Artificial Sequence: Synthetic polypeptide
EVQLVESGGGLVQPGRSLTLSCAASTNITSISPMGWYRQAPGKQRELVAAIHGDSTLYADSVKGRFTISRDNAKNSIYLQMNSLRPEDTALYYCNKVPWGDYHPGNVYWGQGTQVTVSS

SEQ ID NO: 133 - protein, 119 aa, synthetic construct
  REGION 1..119: note = Description of Artificial Sequence: Synthetic polypeptide
EVQLVESGGGLVQPGRSLTLSCAASTNIMSISPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 134 - protein, 119 aa, synthetic construct
  REGION 1..119: note = Description of Artificial Sequence: Synthetic polypeptide
EVQLVESGGGLVQPGRSLTLSCAASTNITSISPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 135 - protein, 119 aa, synthetic construct
  REGION 1..119: note = Description of Artificial Sequence: Synthetic polypeptide
EVQLVESGGGLVQPGRSLTLSCAASTNIVSISPMGWYRQAPGKQRELVAAIHGHSTLYADSVKGRFTISRDNAKNSIYLQMNSLRPEDTALYYCNKVPWGDYHPGNVYWGQGTQVTVSS

SEQ ID NO: 136 - protein, 119 aa, synthetic construct
  REGION 1..119: note = Description of Artificial Sequence: Synthetic polypeptide
EVQLVESGGGLVQPGRSLTLSCAASTNIVSISPMGWYRQAPGKQRELVAAIHGKSTLYADSVKGRFTISRDNAKNSIYLQMNSLRPEDTALYYCNKVPWGDYHPGNVYWGQGTQVTVSS

SEQ ID NO: 137 - protein, 119 aa, synthetic construct
  REGION 1..119: note = Description of Artificial Sequence: Synthetic polypeptide
EVQLVESGGGLVQPGRSLTLSCAASTNVVSISPMGWYRQAPGKQRELVAAIHGKSTLYADSVKGRFTISRDNAKNSIYLQMNSLRPEDTALYYCNKVPWGDYHPNNVYWGQGTQVTVSS

SEQ ID NO: 138 - protein, 119 aa, synthetic construct
  REGION 1..119: note = Description of Artificial Sequence: Synthetic polypeptide
EVQLVESGGGLVQPGRSLTLSCAASTNIISISPMGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 139 - protein, 119 aa, synthetic construct
  REGION 1..119: note = Description of Artificial Sequence: Synthetic polypeptide
EVQLVESGGGLVQPGRSLTLSCAASTNIFSITPMGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 140 - protein, 119 aa, synthetic construct
  REGION 1..119: note = Description of Artificial Sequence: Synthetic polypeptide
EVQLVESGGGLVQPGRSLTLSCAASTNITSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 141 - protein, 119 aa, synthetic construct
  REGION 1..119: note = Description of Artificial Sequence: Synthetic polypeptide
EVQLVESGGGLVQPGRSLTLSCAASTNIQSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 142 - protein, 119 aa, synthetic construct
  REGION 1..119: note = Description of Artificial Sequence: Synthetic polypeptide
EVQLVESGGGLVQPGRSLTLSCAASTSDFSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 143 - protein, 119 aa, synthetic construct
  REGION 1..119: note = Description of Artificial Sequence: Synthetic polypeptide
EVQLVESGGGLVQPGRSLTLSCAASTNIDSISPMGWYRQAPGKQRELVAAIHGFQTLYADSVKGRFTISRDNAKNSIYLQMNSLRPEDTALYYCNKVPWGDYHPGNVYWGQGTQVTVSS

SEQ ID NO: 144 - protein, 119 aa, synthetic construct
  REGION 1..119: note = Description of Artificial Sequence: Synthetic polypeptide
EVQLVESGGGLVQPGRSLTLSCAASTNIMSISPMGWYRQAPGKQRELVAAIHGFSTVYADSVKGRFTISRDNAKNSIYLQMNSLRPEDTALYYCNKVPWGDYHPGNVYWGQGTQVTVSS

SEQ ID NO: 145 - protein, 119 aa, synthetic construct
  REGION 1..119: note = Description of Artificial Sequence: Synthetic polypeptide
EVQLVESGGGLVQPGRSLTLSCAASTNIESISPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 146 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAIHGFKTLYADSVKGRFTISRDNAKNSIYLQMNSLRPEDTARYYCNKVPWGDYHPGNVYWGQGTQVTVSS

SEQ ID NO: 147 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 148 - protein, 119 aa, synthetic construct
  REGION 1..119: note = Description of Artificial Sequence: Synthetic polypeptide
EVQLVESGGGLVQPGRSLTLSCAASTNIFSNSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 149 - protein, 119 aa, synthetic construct
  REGION 1..119: note = Description of Artificial Sequence: Synthetic polypeptide
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 150 - protein, 119 aa, synthetic construct
  REGION 1..119: note = Description of Artificial Sequence: Synthetic polypeptide
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPLNVYWGQGTQVTVSS

SEQ ID NO: 151 - protein, 119 aa, synthetic construct
  REGION 1..119: note = Description of Artificial Sequence: Synthetic polypeptide
EVQLVESGGGLVQPGRSLTLSCVASTNIFSTSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 152 - protein, 119 aa, synthetic construct
  REGION 1..119: note = Description of Artificial Sequence: Synthetic polypeptide
EVQLVESGGGLVQPGRSLTLSCAASTNIFSDSPMGWYRQAPGKQRELVAAIHGFSTFYADSVKGRFTISRDNAKNSIYLQMNSLRPEDTALYYCNKVPWGDYHPGNVYWGQGTQVTVSS

SEQ ID NO: 153 - protein, 119 aa, synthetic construct
  REGION 1..119: note = Description of Artificial Sequence: Synthetic polypeptide
EVQLVESGGGLVQPGRSLTLSCAASTNIFSQSPMGWYRQAPGKQRELVAAIHGDSTLYADSVKGRFTISRDNAKNSIYLQMNSLRPEDTALYYCNKVPWGDYHPGNVCWGQGTQVTVSS

SEQ ID NO: 154 - protein, 119 aa, synthetic construct
  REGION 1..119: note = Description of Artificial Sequence: Synthetic polypeptide
EVQLVESGGGLVQPGRSLTLSCAASTNIFSQSPMGWYRQAPGKQRELVAAIHGKSTLYADSVKGRFTISRDNAKNSIYLQMNSLRPEDTALYYCNKVPWGDYHPSNVYWGKGTQVTVSS

SEQ ID NO: 155 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGESTLYADSVKGRFTISRDNAKNSIYLQMNSLRPEDTALYYCNKVPWGRYHPGNVYWGQGTQVTVSS

SEQ ID NO: 156 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGISTLYADSVKGRFTISRDNAKNSIYLQMNSLRPEDTALYYCNKVPWGDYHPGNVYWGQGTQVTVSS

SEQ ID NO: 157 - protein, 119 aa, synthetic construct
  REGION 1..119: note = Description of Artificial Sequence: Synthetic polypeptide
EVQLVESGGGLVQPGRSLTLSCAASTNIFSRSPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 158 - protein, 119 aa, synthetic construct
  REGION 1..119: note = Description of Artificial Sequence: Synthetic polypeptide
EVQLVESGGGLVQPGRSLTLSCAASTNIFSGSPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 159 - protein, 119 aa, synthetic construct
  REGION 1..119: note = Description of Artificial Sequence: Synthetic polypeptide
EVQLVESGGGLVQPGRSLTLSCAASSNIFSISPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 160 - protein, 119 aa, synthetic construct
  REGION 1..119: note = Description of Artificial Sequence: Synthetic polypeptide
EVQLVESGGGLVQPGRSLTLSCAASTNIFSIYPMGWYRQAPGKQRELVAAIHGSSTLYADSVKGRFTISRDNAKNSIYLQMNSLRPEDTALYYCNKVPWGDYHPKNVYWGQGTQVTVSS

SEQ ID NO: 161 - protein, 119 aa, synthetic construct
  REGION 1..119: note = Description of Artificial Sequence: Synthetic polypeptide
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 162 - protein, 119 aa, synthetic construct
  REGION 1..119: note = Description of Artificial Sequence: Synthetic polypeptide
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPRNVYWGQGTQVTVSS

SEQ ID NO: 163 - protein, 119 aa, synthetic construct
  REGION 1..119: note = Description of Artificial Sequence: Synthetic polypeptide
EVQLVESGGGLVQPGRSLTLSCAASTNEFSISPMGWYRQAPGKQRELVAAIHGLSTLYADSVKGRFTISRDNAKNSIYLQMNSLRPEDTALYYCNKVPWGAYHPRNVYWGQGTQVTVSS

SEQ ID NO: 164 - protein, 119 aa, synthetic construct
  REGION 1..119: note = Description of Artificial Sequence: Synthetic polypeptide
EVQLVESGGGLVQPGRSLTLSCAASTNEFSISPMGWYRQAPGKQRELVAAIHGESTLYADSVKGRFTISRDNAKNSIYLQMNSLRPEDTALYYCNKVPWGDYHPGNVYWGQGTQVTVSS

SEQ ID NO: 165 - protein, 119 aa, synthetic construct
  REGION 1..119: note = Description of Artificial Sequence: Synthetic polypeptide
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVAWGQGTQVTVSS

SEQ ID NO: 166 - protein, 119 aa, synthetic construct
  REGION 1..119: note = Description of Artificial Sequence: Synthetic polypeptide
EVQLVESGGGLVQPGRSLTLSCAASTNIPSISPMGWYRQAPGKQRELVAAIHGASTLYADSVKGRFTISRDNAKNSIYLQMNSLRPEDTALYYCNKVPWGDYHPRNVAWGQGTQVTVSS

SEQ ID NO: 167 - protein, 119 aa, synthetic construct
  REGION 1..119: note = Description of Artificial Sequence: Synthetic polypeptide
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVYWGQGTQVTVSS

SEQ ID NO: 168 - protein, 119 aa, synthetic construct
  REGION 1..119: note = Description of Artificial Sequence: Synthetic polypeptide
EVQLVESGGGLVQPGRSLTLSCAASTNIPSISPMGWYRQAPGKQRELVAAIHGDSTLYADSVKGRFTISRDNAKNSIYLQMNSLRPEDTALYYCNKVPWGDYHPRNVYWGQGTQVTVSS

SEQ ID NO: 169 - protein, 119 aa, synthetic construct
  REGION 1..119: note = Description of Artificial Sequence: Synthetic polypeptide
EVQLVESGGGLVQPGRSLTLSCAASTNITSISPMGWYRQAPGKQRELVAAIHGVSTLYADSVKGRFTISRDNAKNSIYLQMNSLRPEDTALYYCNKVPWGDYHPRNVQWGQGTQVTVSS

SEQ ID NO: 170 - protein, 119 aa, synthetic construct
  REGION 1..119: note = Description of Artificial Sequence: Synthetic polypeptide
EVQLVESGGGLVQPGRSLTLSCAASTNIPSISPMGWYRQAPGKQRELVAAIHGQSTLYADSVKGRFTISRDNAKNSIYLQMNSLRPEDTALYYCNKVPWGDYHPRNVQWGQGTQVTVSS

SEQ ID NO: 171 - protein, 119 aa, synthetic construct
  REGION 1..119: note = Description of Artificial Sequence: Synthetic polypeptide
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRNVSWGQGTQVTVSS

SEQ ID NO: 172 - protein, 119 aa, synthetic construct
  REGION 1..119: note = Description of Artificial Sequence: Synthetic polypeptide
EVQLVESGGGLVQPGRSLTLSCAASSNIFSISPMGWYRQAPGKQRELVAAIHGESTLYADSVKGRFTISRDNAKNSIYLQMNSLRPEDTALYYCNKVPWGDYHPRNVTWGQGTQVTVSS

SEQ ID NO: 173 - protein, 119 aa, synthetic construct
  REGION 1..119: note = Description of Artificial Sequence: Synthetic polypeptide
EVQLVESGGGLVQPGRSLTLSCAASTNIDSISPMGWYRQAPGKQRELVAAIHGESTLYADSVKGRFTISRDNAKNSIYLQMNSLRPEDTALYYCNKVPWGDYHPRNVTWGQGTQVTVSS

SEQ ID NO: 174 - protein, 119 aa, synthetic construct
  REGION 1..119: note = Description of Artificial Sequence: Synthetic polypeptide
EVQLVESGGGLVQPGRSLTLSCAASTNIDSISPMGWYRQAPGKQRELVAAIHGSSTLYADSVKGRFTISRDNAKNSIYLQMNSLRPEDTALYYCNKVPWGDYHPRNVTWGQGTQVTVSS

SEQ ID NO: 175 - protein, 119 aa, synthetic construct
  REGION 1..119: note = Description of Artificial Sequence: Synthetic polypeptide
EVQLVESGGGLVQPGRSLTLSCAASTNIRSISPMGWYRQAPGKQRELVAAIHGSSTLYADSVKGRFTISRDNAKNSIYLQMNSLRPEDTALYYCNKVPWGDYHPRNVVWGQGTQVTVSS

SEQ ID NO: 176 - protein, 119 aa, synthetic construct
  REGION 1..119: note = Description of Artificial Sequence: Synthetic polypeptide
EVQLVESGGGLVQPGRSLTLSCAASTNITSISPMGWYRQAPGKQRELVAAISGFSTLYADSVKGRFTISRDNAKNSIYLQMNSLRPEDTALYYCNEVPWGDYHPRNVYWGQGTQVTVSS

SEQ ID NO: 177 - protein, 119 aa, synthetic construct
  REGION 1..119: note = Description of Artificial Sequence: Synthetic polypeptide
EVQLVESGGGLVQPGRSLTLSCAASTNITSISPMGWYRQAPGKQRELVAAIHGESTLYADSVKGRFTISRDNAKNSIYLQMNSLRPEDTALYYCNKVPWGDYHPRNVYWGQGTQVTVSS

SEQ ID NO: 178 - protein, 119 aa, synthetic construct
  REGION 1..119: note = Description of Artificial Sequence: Synthetic polypeptide
EVQLVESGGGLVQPGRSLTLSCAASTNITSVSPMGWYRQAPGKQRELVAAIHGPSTLYADSVKGRFTISRDNAKNSIYLQMNSLRPEDTALYYCNKVPWGDYHPTNVYWGQGTQVTVSS

SEQ ID NO: 179 - protein, 119 aa, synthetic construct
  REGION 1..119: note = Description of Artificial Sequence: Synthetic polypeptide
EVQLVESGGGLVQPGRSLTLSCAASTNIGSISPMGWYRQAPGKQRELVAAIHGQSTLYADSVKGRFTISRDNAKNSIYLQMNSLRPEDTALYYCNKVPWGDYHPQNVYWGQGTQVTVSS

SEQ ID NO: 180 - protein, 119 aa, synthetic construct
  REGION 1..119: note = Description of Artificial Sequence: Synthetic polypeptide
EVQLVESGGGLVQPGRSLTLSCAASTNIESISPMGWYRQAPGKQRELVAAIHGKSTLYADSVKGRFTISRDNAKNSIYLQMNSLRPEDTALYYCNKVPWGDYHPRRVYWGQGTQVTVSS

SEQ ID NO: 181-188 - skipped (8 entries)

SEQ ID NO: 189 - protein, 119 aa, synthetic construct
  REGION 1..119: note = Description of Artificial Sequence: Synthetic polypeptide
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRRVYWGQGTQVTVSS

SEQ ID NO: 190 - protein, 119 aa, synthetic construct
  REGION 1..119: note = Description of Artificial Sequence: Synthetic polypeptide
EVQLVESGGGLVQPGRSLTLSCAASTNIDSISPMGWYRQAPGKQRELVAAIHGNSTLYADSVKGRFTISRDNAKNSIYLQMNSLRPEDTALYYCNKVPWGDYHPRMVYWGQGTQVTVSS

SEQ ID NO: 191 - protein, 119 aa, synthetic construct
  REGION 1..119: note = Description of Artificial Sequence: Synthetic polypeptide
EVQLVESGGGLVQPGRSLTLSCAASTNIFMISPMGWYRQAPGKQRELVAAIHGDSTLYADSVKGRFTISRDNAKNSIYLQMNSLRPEDTALYYCNKVPWGRYHPRNVYWGQGTQVTVSS

SEQ ID NO: 192 - protein, 119 aa, synthetic construct
  REGION 1..119: note = Description of Artificial Sequence: Synthetic polypeptide
EVQLVESGGGLVQPGRSLTLSCAASTNIFRISPMGWYRQAPGKQRELVAAIHGDSTLYADSVKGRFTISRDNAKNSIYLQMNSLRPEDTALYYCNKVPWGRYHPRNVYWGQGTQVTVSS

SEQ ID NO: 193 - protein, 119 aa, synthetic construct
  REGION 1..119: note = Description of Artificial Sequence: Synthetic polypeptide
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGEYHPRNVYWGQGTQVTVSS

SEQ ID NO: 194 - protein, 119 aa, synthetic construct
  REGION 1..119: note = Description of Artificial Sequence: Synthetic polypeptide
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGKYHPRNVYWGQGTQVTVSS

SEQ ID NO: 195 - protein, 119 aa, synthetic construct
  REGION 1..119: note = Description of Artificial Sequence: Synthetic polypeptide
EVQLVESGGGLVQPGRSLTLSCAASTNIESISPMGWYRQAPGKQRELVAAIHGSSTLYADSVKGRFTISRDNAKNSIYLQMNSLRPEDTALYYCNKVPWGRYHPRNVYWGQGTQVTVSS

SEQ ID NO: 196 - protein, 119 aa, synthetic construct
  REGION 1..119: note = Description of Artificial Sequence: Synthetic polypeptide
EVQLVESGGGLVQPGRSLTLSCAASTNIESISPMGWYRQAPGKQRELVAAIHGNSTLYADSVKGRFTISRDNAKNSIYLQMNSLRPEDTALYYCNKVPWGRYHPRNVYWGQGTQVTVSS

SEQ ID NO: 197 - protein, 119 aa, synthetic construct
  REGION 1..119: note = Description of Artificial Sequence: Synthetic polypeptide
EVQLVESGGGLVQPGRSLTLSCAASTNISSISPMGWYRQAPGKQRELVAAIHGFSTLYADSVKGRFTISRDNAKNSIYLQMNSLRPEDTALYYCNKVPWGYYHPRNVYWGQGTQVTVSS

SEQ ID NO: 198 - protein, 119 aa, synthetic construct
  REGION 1..119: note = Description of Artificial Sequence: Synthetic polypeptide
EVQLVESGGGLVQPGRSLTLSCAASTNISSISPMGWYRQAPGKQRELVAAIHGHSTLYADSVKGRFTISRDNAKNSIYLQMNSLRPEDTALYYCNKVPWGRYHPRNVYWGQGTQVTVSS

SEQ ID NO: 199 - protein, 119 aa, synthetic construct
  REGION 1..119: note = Description of Artificial Sequence: Synthetic polypeptide
EVQLVESGGGLVQPGRSLTLSCAASTNISSISPMGWYRQAPGKQRELVAAIHGFSTVYADSVKGRFTISRDNAKNSIYLQMNSLRPEDTALYYCNKVPWGRYHPRNVYWGQGTQVTVSS

SEQ ID NO: 200 - protein, 119 aa, synthetic construct
  REGION 1..119: note = Description of Artificial Sequence: Synthetic polypeptide
EVQLVESGGGLVQPGRSLTLSCAASTNIFSIRPMGWYRQAPGKQRELVAAIHGFSTVYADSVKGRFTISRDNAKNSIYLQMNSLRPEDTALYYCNKVPWGDYHPRNVYWGQGTQVTVSS

SEQ ID NO: 201 - protein, 119 aa, synthetic construct
  REGION 1..119: note = Description of Artificial Sequence: Synthetic polypeptide
EVQLVESGGGLVQPGRSLTLSCAASTNIFSIYPMGWYRQAPGKQRELVAAIHGFSTYYADSVKGRFTISRDNAKNSIYLQMNSLRPEDTALYYCNKVPWGSYHPRNVYWGQGTQVTVSS

SEQ ID NO: 202 - protein, 119 aa, synthetic construct
  REGION 1..119: note = Description of Artificial Sequence: Synthetic polypeptide
EVQLVESGGGLVQPGRSLTLSCAASTNIFNISPMGWYRQAPGKQRELVAAIHGFSTYYADSVKGRFTISRDNAKNSIYLQMNSLRPEDTALYYCNKVPWGRYHPRNVYWGQGTQVTVSS

SEQ ID NO: 203 - protein, 119 aa, synthetic construct
  REGION 1..119: note = Description of Artificial Sequence: Synthetic polypeptide
EVQLVESGGGLVQPGRSLTLSCAASTNIFSSSPMGWYRQAPGKQRELVAAIHGFSTWYADSVKGRFTISRDNAKNSIYLQMNSLRPEDTALYYCNKVPWGRYHPRNVYWGQGTQVTVSS

SEQ ID NO: 204 - protein, 119 aa, synthetic construct
  REGION 1..119: note = Description of Artificial Sequence: Synthetic polypeptide
EVQLVESGGGLVQPGRSLTLSCAASTNISSISPMGWYRQAPGKQRELVAAIHGFDTLYADSVKGRFTISRDNAKNSIYLQMNSLRPEDTALYYCNKVPWGDYHPRNVYWGQGTQVTVSS

SEQ ID NO: 205 - protein, 119 aa, synthetic construct
  REGION 1..119: note = Description of Artificial Sequence: Synthetic polypeptide
EVQLVESGGGLVQPGRSLTLSCAASTNIFSINPMGWYRQAPGKQRELVAAIHGFDTLYADSVKGRFTISRDNAKNSIYLQMNSLRPEDTALYYCNKVPWGDYHPRNVSWGQGTQVTVSS

SEQ ID NO: 206 - protein, 119 aa, synthetic construct
  REGION 1..119: note = Description of Artificial Sequence: Synthetic polypeptide
EVQLVESGGGLVQPGRSLTLSCAASTNIFSITPMGWYRQAPGKQRELVAAIHGRSTLYADSVKGRFTISRDNAKNSIYLQMNSLRPEDTALYYCNKVPWGSYHPRNVYWGQGTQVTVSS

SEQ ID NO: 207 - protein, 119 aa, synthetic construct
  REGION 1..119: note = Description of Artificial Sequence: Synthetic polypeptide
EVQLVESGGGLVQPGRSLTLSCAASTNIFSITPMGWYRQAPGKQRELVAAIHGTSTLYADSVKGRFTISRDNAKNSIYLQMNSLRPEDTALYYCNKVPWGRYHPRNVYWGQGTQVTVSS

SEQ ID NO: 208 - protein, 119 aa, synthetic construct
  REGION 1..119: note = Description of Artificial Sequence: Synthetic polypeptide
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGRYHPRNVYWGQGTQVTVSS

SEQ ID NO: 209 - protein, 119 aa, synthetic construct
  REGION 1..119: note = Description of Artificial Sequence: Synthetic polypeptide
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGDYHPRDVYWGQGTQVTVSS

SEQ ID NO: 210 - protein, 119 aa, synthetic construct
  REGION 1..119: note = Description of Artificial Sequence: Synthetic polypeptide
EVQLVESGGGLVQPGRSLTLSCAASTNIFSTSPYGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 211 - protein, 119 aa, synthetic construct
  REGION 1..119: note = Description of Artificial Sequence: Synthetic polypeptide
EVQLVESGGGLVQPGRSLTLSCAASTNIFSTSPGGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 212 - protein, 119 aa, synthetic construct
  REGION 1..119: note = Description of Artificial Sequence: Synthetic polypeptide
EVQLVESGGGLVQPGRSLTLSCAASTNIFSITPYGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 213 - protein, 119 aa, synthetic construct
  REGION 1..119: note = Description of Artificial Sequence: Synthetic polypeptide
EVQLVESGGGLVQPGRSLTLSCAASTNIFSITPGGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 214 - protein, 119 aa, synthetic construct
  REGION 1..119: note = Description of Artificial Sequence: Synthetic polypeptide
QVQLVESGGGLVQPGESLRLSCAASTNIFSISPMGWYRQAPGKQRELVAAIHGFSTLYADSVKGRFTISRDNAKNTIYLQMNSLKPEDTAVYYCNKVPWGDYHPRNVYWGQGTQVTVSS

SEQ ID NO: 215 - protein, 110 aa, synthetic construct
  REGION 1..110: note = Description of Artificial Sequence: Synthetic polypeptide
QVQLVESGGGLVQPGGSLRLSCAASGRTFSVRGMAWYRQAGNNRALVATMNPDGFPNYADAVKGRFTISWDIAENTVYLQMNSLNSEDTTVYYCNSGPYWGQGTQVTVSS

SEQ ID NO: 216 - protein, 123 aa, synthetic construct
  REGION 1..123: note = Description of Artificial Sequence: Synthetic polypeptide
QVQLVESGGGLVQAGGSLRLSCAASGSIPSIEQMGWYRQAPGKQRELVAALTSGGRANYADSVKGRFTISGDNVRNMVYLQMNSLKPEDTAIYYCSAGRFKGDYAQRSGMDYWGKGTLVTVSS

SEQ ID NO: 217 - protein, 116 aa, synthetic construct
  REGION 1..116: note = Description of Artificial Sequence: Synthetic polypeptide
QVQLVESGGGLVQAGGSLRLSCAFSGTTYTFDLMSWYRQAPGKQRTVVASISSDGRTSYADSVRGRFTISGENGKNTVYLQMNSLKLEDTAVYYCLGQRSGVRAFWGQGTQVTVSS

SEQ ID NO: 218 - protein, 123 aa, synthetic construct
  REGION 1..123: note = Description of Artificial Sequence: Synthetic polypeptide
QVQLVESGGGLVQAGGSLRLSCVASGSTSNINNMRWYRQAPGKERELVAVITRGGYAIYLDAVKGRFTISRDNANNAIYLEMNSLKPEDTAVYVCNADRVEGTSGGPQLRDYFGQGTQVTVSS

SEQ ID NO: 219 - protein, 116 aa, synthetic construct
  REGION 1..116: note = Description of Artificial Sequence: Synthetic polypeptide
QVQLVESGGGLVQAGGSLRLSCAASGSTFGINAMGWYRQAPGKQRELVAVISRGGSTNYADSVKGRFTISRDNAENTVSLQMNTLKPEDTAVYFCNARTYTRHDYWGQGTQVTVSS

SEQ ID NO: 220 - protein, 122 aa, synthetic construct
  REGION 1..122: note = Description of Artificial Sequence: Synthetic polypeptide
QVRLVESGGGLVQAGGSLRLSCAASISAFRLMSVRWYRQDPSKQREWVATIDQLGRTNYADSVKGRFAISKDSTRNTVYLQMNMLRPEDTAVYYCNAGGGPLGSRWLRGRHWGQGTQVTVSS

SEQ ID NO: 221 - protein, 119 aa, synthetic construct
  REGION 1..119: note = Description of Artificial Sequence: Synthetic polypeptide
QVRLVESGGGLVQAGESLRLSCAASGRPFSINTMGWYRQAPGKQRELVASISSSGDFTYTDSVKGRFTISRDNAKNTVYLQMNSLKPEDTAVYYCNARRTYLPRRFGSWGQGTQVTVSS

SEQ ID NO: 222 - protein, 117 aa, synthetic construct
  REGION 1..117: note = Description of Artificial Sequence: Synthetic polypeptide
QVQPVESGGGLVQPGGSLRLSCVVSGSDFTEDAMAWYRQASGKERESVAFVSKDGKRILYLDSVRGRFTISRDIDKKTVYLQMDNLKPEDTGVYYCNSAPGAARNYWGQGTQVTVSS

SEQ ID NO: 223 - protein, 117 aa, synthetic construct
  REGION 1..117: note = Description of Artificial Sequence: Synthetic polypeptide
QVQPVESGGGLVQPGGSLRLSCVVSGSDFTEDAMAWYRQASGKERESVAFVSKDGKRILYLDSVRGRFTISRDIYKKTVYLQMDNLKPEDTGVYYCNSAPGAARNVWGQGTQVTVSS

SEQ ID NO: 224 - protein, 115 aa, synthetic construct [sequence omitted]

SEQ ID NO: 225 - protein, 124 aa, synthetic construct
  REGION 1..124: note = Description of Artificial Sequence: Synthetic polypeptide
QVQIVESGGGLVQAGGSLRLSCVASGLTYSIVAVGWYRQAPGKEREMVADISPVGNTNYADSVKGRFTISKENAKNTVYLQMNSLKPEDTAVYYCHIVRGWLDERPGPGPIVYWGQGTQVTVSS

SEQ ID NO: 226 - protein, 115 aa, synthetic construct
  REGION 1..115: note = Description of Artificial Sequence: Synthetic polypeptide
QVQLVESGGGLVQTGGSLRLSCAASGLTFGVYGMEWFRQAPGKQREWVASHTSTGYVYYRDSVKGRFTISRDNAKSTVYLQMNSLKPEDTAIYYCKANRGSYEYWGQGTQVTVSS

SEQ ID NO: 227 - protein, 114 aa, synthetic construct
  REGION 1..114: note = Description of Artificial Sequence: Synthetic polypeptide
QVQLVESGGGLVQAGGSLRLSCAASTTSSINSMSWYRQAQGKQREPVAVITDRGSTSYADSVKGRFTISRDNAKNTVYLQMNSLKPEDTAIYTCHVIADWRGYWGQGTQVTVSS

SEQ ID NO: 228 - protein, 120 aa, synthetic construct
  REGION 1..120: note = Description of Artificial Sequence: Synthetic polypeptide
QVQLVESGGGLVQAGGSLRLSCAASGRTLSRYAMGWFRQAPGKERQFVAAISRSGGTTRYSDSVKGRFTISRDNAANTFYLQMNNLRPDDTAVYYCNVRRRGWGRTLEYWGQGTQVTVSS

SEQ ID NO: 229 - protein, 117 aa, synthetic construct
  REGION 1..117: note = Description of Artificial Sequence: Synthetic polypeptide
QVQLGESGGGLVQAGGSLRLSCAASGSIFSPNAMIWHRQAPGKQREPVASINSSGSTNYGDSVKGRFTVSRDIVKNTMYLQMNSLKPEDTAVYYCSYSDFRRGTQYWGQGTQVTVSS

SEQ ID NO: 230 - protein, 119 aa, synthetic construct
  REGION 1..119: note = Description of Artificial Sequence: Synthetic polypeptide
QVQLVESGGGLVPSGGSLRLSCAASGATSAITNLGWYRRAPGQVREMVARISVREDKEDYEDSVKGRFTISRDNTQNLVYLQMNNLQPHDTAIYYCGAQRWGRGPGTTWGQGTQVTVSS

SEQ ID NO: 231 - protein, 116 aa, synthetic construct
  REGION 1..116: note = Description of Artificial Sequence: Synthetic polypeptide
QVQLVESGGGLVQAGGSLRLSCAASGSTFRIRVMRWYRQAPGTERDLVAVISGSSTYYADSVKGRFTISRDNAKNTLYLQMNNLKPEDTAVYYCNADDSGIARDYWGQGTQVTVSS

SEQ ID NO: 232 - protein, 118 aa, synthetic construct
  REGION 1..118: note = Description of Artificial Sequence: Synthetic polypeptide
QVQLVESGGGLVQAGESRRLSCAVSGDTSKFKAVGWYRQAPGAQRELLAWINNSGVGNTAESVKGRFTISRDNAKNTVYLQMNRLTPEDTDVYYCRFYRRFGINKNYWGQGTQVTVSS

SEQ ID NO: 233 - protein, 122 aa, synthetic construct
  REGION 1..122: note = Description of Artificial Sequence: Synthetic polypeptide
QVQLVESGGGLVQAGGSLRLSCAASGSTFGNKPMGWYRQAPGKQRELVAVISSDGGSTRYAALVKGRFTISRDNAKNTVYLQMESLVAEDTAVYYCNALRTYYLNDPVVFSWGQGTQVTVSS

SEQ ID NO: 234 - protein, 119 aa, synthetic construct
  REGION 1..119: note = Description of Artificial Sequence: Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLTPEDTAVYYCNTYIPLRGTLHDYWGQGTQVTVSS

SEQ ID NO: 235 - protein, 115 aa, synthetic construct
  REGION 1..115: note = Description of Artificial Sequence: Synthetic polypeptide
QVQLVESGGGLVQAGGSLRLSCVASGRTDRITTMGWYRQAPGKQRELVATISNRGTSNYANSVKGRFTISRDNAKNTVYLQMNSLKPEDTAVYYCNARKWGRNYWGQGTQVTVSS

SEQ ID NO: 236 - protein, 119 aa, synthetic construct
  REGION 1..119: note = Description of Artificial Sequence: Synthetic polypeptide
QVQLVESGGGLVQARGSLRLSCTASGRTIGINDMAWYRQAPGNQRELVATITKGGTTDYADSVDGRFTISRDNAKNTVYLQMNSLKPEDTAVYYCNTKRREWAKDFEYWGQGTQVTVSS

SEQ ID NO: 237 - protein, 114 aa, synthetic construct
  REGION 1..114: note = Description of Artificial Sequence: Synthetic polypeptide
QVQLVESGGGLVQAGGSLRLSCAASAIGSINSMSWYRQAPGKQREPVAVITDRGSTSYADSVKGRFTISRDNAKNTVYLQMNSLKPEDTAIYTCHVIADWRGYWGQGTQVTVSS

SEQ ID NO: 238 - protein, 119 aa, synthetic construct
  REGION 1..119: note = Description of Artificial Sequence: Synthetic polypeptide
QVQLVESGGGLVQAGGSLRLSCAASGSTSSINTMYWFRQAPGEERELVATINRGGSTNVRDSVKGRFSVSRDSAKNIVYLQMNRLKPEDTAVYYCNTYIPYGGTLHDFWGQGTQVTVSS

SEQ ID NO: 239 - protein, 114 aa, synthetic construct
  REGION 1..114: note = Description of Artificial Sequence: Synthetic polypeptide
QVQLVESGGGLVQAGGSLRLSCTTSTTFSINSMSWYRQAPGNQREPVAVITNRGTTSYADSVKGRFTISRDNARNTVYLQMDSLKPEDTAIYTCHVIADWRGYWGQGTQVTVSS

SEQ ID NO: 240 - protein, 116 aa, synthetic construct
  REGION 1..116: note = Description of Artificial Sequence: Synthetic polypeptide
QVQLVESGGGLVQAGGSLTLSCAASGSTFSIRAMRWYRQAPGTERDLVAVIYGSSTYYADAVKGRFTISRDNAKNTLYLQMNNLKPEDTAVYYCNADTIGTARDYWGQGTQVTVSS

SEQ ID NO: 241 - protein, 117 aa, synthetic construct
  REGION 1..117: note = Description of Artificial Sequence: Synthetic polypeptide
QVQLVESGGGLVQAGGSLRLSCVASGRTSTIDTMYWHRQAPGNERELVAYVTSRGTSNVADSVKGRFTISRDNAKNTAYLQMNSLKPEDTAVYYCSVRTTSYPVDFWGQGTQVTVSS

SEQ ID NO: 242 - protein, 119 aa, synthetic construct
  REGION 1..119: note = Description of Artificial Sequence: Synthetic polypeptide
QVQLVESGGGLVQAGGSLRLSCAASGSTSSINTMYWYRQAPGKERELVAFISSGGSTNVRDSVKGRFSVSRDSAKNIVYLQMNSLKPEDTAVYYCNTYIPYGGTLHDFWGQGTQVTVSS

SEQ ID NO: 243 - protein, 111 aa, synthetic construct
  REGION 1..111: note = Description of Artificial Sequence: Synthetic polypeptide
QVQLVESGGGLVQPGGSLRLSCAASGGDWSANFMYWYRQAPGKQRELVARISGRGVVDYVESVKGRFTISRDNAKNTVYLQMNSLKPEDTAVYYCAVASYWGQGTQVTVSS

SEQ ID NO: 244 - protein, 111 aa, synthetic construct
  REGION 1..111: note = Description of Artificial Sequence: Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 245 - protein, 111 aa, synthetic construct
  REGION 1..111: note = Description of Artificial Sequence: Synthetic polypeptide
EVQLVESGGGLVQPGGSLRLSCAASGGDWSANFMYWVRQAPGKGLEWVSRISGRGVVDYVESVKGRFTISRDNSKNTLYLQMNSLRAEDTAVYYCAVASYWGQGTLVTVSS

SEQ ID NO: 246 - protein, 119 aa, synthetic construct
  REGION 1..119: note = Description of Artificial Sequence: Synthetic polypeptide
EVQLVESGGGLVQAGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 247 - protein, 119 aa, synthetic construct
  REGION 1..119: note = Description of Artificial Sequence: Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 248 - protein, 119 aa, synthetic construct
  REGION 1..119: note = Description of Artificial Sequence: Synthetic polypeptide
EVQLVESGGGLVQPGGSLRLSCAASGSTSSINTMYWVRQAPGKGLEWVSFISSGGSTNVRDSVKGRFTISRDNSKNTLYLQMNSLRAEDTAVYYCNTYIPYGGTLHDFWGQGTLVTVSS

SEQ ID NO: 249 - protein, 116 aa, synthetic construct
  REGION 1..116: note = Description of Artificial Sequence: Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 250 - protein, 118 aa, synthetic construct
  REGION 1..118: note = Description of Artificial Sequence: Synthetic polypeptide
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSSGG

SEQ ID NO: 251 - protein, 118 aa, synthetic construct
  REGION 1..118: note = Description of Artificial Sequence: Synthetic polypeptide
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSSGG

SEQ ID NO: 252 - protein, 118 aa, synthetic construct
  REGION 1..118: note = Description of Artificial Sequence: Synthetic polypeptide
EVQLVESGGGLVQAGGSLRLSCVASGRTSTIDTMYWHRQAPGNERELVAYVTSRGTSNVADSVKGRFTISRDNSKNTLYLQMNSLRAEDTAVYYCSVRTTSYPVDFWGQGTLVTVSGG

SEQ ID NO: 253 - protein, 117 aa, synthetic construct
  REGION 1..117: note = Description of Artificial Sequence: Synthetic polypeptide
EVQLVESGGGLVQPGGSLRLSCAASGRTSTIDTMYWHRQAPGKERELVAYVTSRGTSNVADSVKGRFTISRDNSKNTLYLQMNSLRAEDTAVYYCSVRTTSYPVDFWGQGTLVTVSS

SEQ ID NO: 254 - protein, 117 aa, synthetic construct
  REGION 1..117: note = Description of Artificial Sequence: Synthetic polypeptide
EVQLVESGGGLVQPGGSLRLSCAASGRTSTIDTMYWVRQAPGKGLEWVSYVTSRGTSNVADSVKGRFTISRDNSKNTLYLQMNSLRAEDTAVYYCSVRTTSYPVDFWGQGTLVTVSS

SEQ ID NO: 255 - protein, 116 aa, synthetic construct
  REGION 1..116: note = Description of Artificial Sequence: Synthetic polypeptide
QVQLVESGGGVVQAGGSLTLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 256 - protein, 116 aa, synthetic construct; identical to SEQ ID NO: 249
  REGION 1..116: note = Description of Artificial Sequence: Synthetic polypeptide

SEQ ID NO: 257 - protein, 116 aa, synthetic construct
  REGION 1..116: note = Description of Artificial Sequence: Synthetic polypeptide
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSS

SEQ ID NO: 258 - protein, 116 aa, synthetic construct
  REGION 1..116: note = Description of Artificial Sequence: Synthetic polypeptide
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSS

SEQ ID NO: 259 - protein, 12 aa, synthetic construct
  REGION 1..12: note = Description of Artificial Sequence: Synthetic peptide
GGQDGNEEMGGG

SEQ ID NO: 260 - protein, 10 aa, synthetic construct
  REGION 1..10: note = Description of Artificial Sequence: Synthetic peptide
GGQDGNEEGG

SEQ ID NO: 261 - protein, 16 aa, synthetic construct
  REGION 1..16: note = Description of Artificial Sequence: Synthetic peptide
GGGGQDGNEEMGGGGG

SEQ ID NO: 262 - protein, 7 aa, synthetic construct
  REGION 1..7: note = Description of Artificial Sequence: Synthetic peptide
APFGSEM

SEQ ID NO: 263 - protein, 7 aa, synthetic construct
  REGION 1..7: note = Description of Artificial Sequence: Synthetic peptide
AWNGPYE

SEQ ID NO: 264 - protein, 10 aa, synthetic construct
  REGION 1..10: note = Description of Artificial Sequence: Synthetic peptide
AQDNGDTKTG

SEQ ID NO: 265 - protein, 8 aa, synthetic construct
  REGION 1..8: note = Description of Artificial Sequence: Synthetic peptide
AEAKETQG

SEQ ID NO: 266 - protein, 9 aa, synthetic construct
  REGION 1..9: note = Description of Artificial Sequence: Synthetic peptide
ATRREQVEG

SEQ ID NO: 267 - protein, 7 aa, synthetic construct
  REGION 1..7: note = Description of Artificial Sequence: Synthetic peptide
AQAPSQP

SEQ ID NO: 268 - protein, 10 aa, synthetic construct
  REGION 1..10: note = Description of Artificial Sequence: Synthetic peptide
ATRSRTRNDG

SEQ ID NO: 269 - protein, 10 aa, synthetic construct
  REGION 1..10: note = Description of Artificial Sequence: Synthetic peptide
ADVDAPDGLG

SEQ ID NO: 270 - protein, 9 aa, synthetic construct
  REGION 1..9: note = Description of Artificial Sequence: Synthetic peptide
AADISDPGG

SEQ ID NO: 271 - protein, 8 aa, synthetic construct
  REGION 1..8: note = Description of Artificial Sequence: Synthetic peptide
ALSVDPSG

SEQ ID NO: 272 - protein, 8 aa, synthetic construct
  REGION 1..8: note = Description of Artificial Sequence: Synthetic peptide
ARLSVDPG

SEQ ID NO: 273 - protein, 8 aa, synthetic construct
  REGION 1..8: note = Description of Artificial Sequence: Synthetic peptide
AVEAADRG

SEQ ID NO: 274 - protein, 12 aa, synthetic construct
  REGION 1..12: note = Description of Artificial Sequence: Synthetic peptide
GGPDGNEEMGGG

SEQ ID NO: 275 - protein, 12 aa, synthetic construct
  REGION 1..12: note = Description of Artificial Sequence: Synthetic peptide
GGFDGNEEMGGG

SEQ ID NO: 276 - protein, 12 aa, synthetic construct
  REGION 1..12: note = Description of Artificial Sequence: Synthetic peptide
GGGDGNEEMGGG

SEQ ID NO: 277 - protein, 12 aa, synthetic construct
  REGION 1..12: note = Description of Artificial Sequence: Synthetic peptide
GGEMDGEGQNGG

SEQ ID NO: 278 - protein, 14 aa, synthetic construct
  REGION 1..14: note = Description of Artificial Sequence: Synthetic peptide
GGGGGPDGNEEPGG

SEQ ID NO: 279 - protein, 14 aa, synthetic construct
  REGION 1..14: note = Description of Artificial Sequence: Synthetic peptide
GGGGSLDGNEEPGG

SEQ ID NO: 280 - protein, 14 aa, synthetic construct
  REGION 1..14: note = Description of Artificial Sequence: Synthetic peptide
GGGGALDGNEEPGG

SEQ ID NO: 281 - protein, 14 aa, synthetic construct
  REGION 1..14: note = Description of Artificial Sequence: Synthetic peptide
GGGGGLDGNEEPGG

SEQ ID NO: 282 - protein, 13 aa, synthetic construct
  REGION 1..13: note = Description of Artificial Sequence: Synthetic peptide
GGGALDGNEEPGG

SEQ ID NO: 283 - protein, 15 aa, synthetic construct
  REGION 1..15: note = Description of Artificial Sequence: Synthetic peptide
GGGGGPDGNEEPGGG

SEQ ID NO: 284 - protein, 14 aa, synthetic construct
  REGION 1..14: note = Description of Artificial Sequence: Synthetic peptide
GGSGALDGNEEPGG

SEQ ID NO: 285 - protein, 14 aa, synthetic construct
  REGION 1..14: note = Description of Artificial Sequence: Synthetic peptide
GGSGSLDGNEEPGG

SEQ ID NO: 286 - protein, 14 aa, synthetic construct
  REGION 1..14: note = Description of Artificial Sequence: Synthetic peptide
GGSGGPDGNEEPGG

SEQ ID NO: 287 - protein, 15 aa, synthetic construct
  REGION 1..15: note = Description of Artificial Sequence: Synthetic peptide
GGVRDGPDGNEEPGG

SEQ ID NO: 288 - protein, 16 aa, synthetic construct
  REGION 1..16: note = Description of Artificial Sequence: Synthetic peptide
GGSGGPDGNEEPGGGG

SEQ ID NO: 289 - protein, 14 aa, synthetic construct
  REGION 1..14: note = Description of Artificial Sequence: Synthetic peptide
GGGRGPDGNEEPGG

SEQ ID NO: 290 - protein, 14 aa, synthetic construct
  REGION 1..14: note = Description of Artificial Sequence: Synthetic peptide
GGSGGLDGNEEPGG

SEQ ID NO: 291 - protein, 14 aa, synthetic construct
  REGION 1..14: note = Description of Artificial Sequence: Synthetic peptide
GGGVGPDGNEEPGG

SEQ ID NO: 292 - protein, 14 aa, synthetic construct
  REGION 1..14: note = Description of Artificial Sequence: Synthetic peptide
GGGEGPDGNEEPGG

SEQ ID NO: 293 - protein, 14 aa, synthetic construct
  REGION 1..14: note = Description of Artificial Sequence: Synthetic peptide
GGGVALDGNEEPGG

SEQ ID NO: 294 - protein, 14 aa, synthetic construct
  REGION 1..14: note = Description of Artificial Sequence: Synthetic peptide
GGGRALDGNEEPGG

SEQ ID NO: 295 - protein, 14 aa, synthetic construct
  REGION 1..14: note = Description of Artificial Sequence: Synthetic peptide
GGYAGLDGNEEPGG

SEQ ID NO: 296 - protein, 14 aa, synthetic construct
  REGION 1..14: note = Description of Artificial Sequence: Synthetic peptide
GGAGGPDGNEEPGG

SEQ ID NO: 297 - protein, 14 aa, synthetic construct
  REGION 1..14: note = Description of Artificial Sequence: Synthetic peptide
GGRGGPDGNEEPGG

SEQ ID NO: 298 - protein, 15 aa, synthetic construct
  REGION 1..15: note = Description of Artificial Sequence: Synthetic peptide
GGGGPDGNEEPGGGG

SEQ ID NO: 299 - protein, 14 aa, synthetic construct
  REGION 1..14: note = Description of Artificial Sequence: Synthetic peptide
GGGEALDGNEEPGG

SEQ ID NO: 300 - protein, 14 aa, synthetic construct
  REGION 1..14: note = Description of Artificial Sequence: Synthetic peptide
GGDASLDGNEEPGG

SEQ ID NO: 301 - protein, 13 aa, synthetic construct
  REGION 1..13: note = Description of Artificial Sequence: Synthetic peptide
GGRDAPDGNEEGG

SEQ ID NO: 302 - protein, 10 aa, synthetic construct
  REGION 1..10: note = Description of Artificial Sequence: Synthetic peptide
SGGGGSGGVV

SEQ ID NO: 303 - protein, 16 aa, synthetic construct
  REGION 1..16: note = Description of Artificial Sequence: Synthetic peptide
SGGGGSGGGGSGGGGS

SEQ ID NO: 304 - protein, 17 aa, synthetic construct
  REGION 1..17: note = Description of Artificial Sequence: Synthetic peptide
SGGGGSGGGGSGGGGGS

SEQ ID NO: 305 - protein, 10 aa, synthetic construct
  REGION 1..10: note = Description of Artificial Sequence: Synthetic peptide
SGGGGSGGGS

SEQ ID NO: 306 - protein, 115 aa, synthetic construct
  REGION 1..115: note = Description of Artificial Sequence: Synthetic polypeptide
QVQLQESGGGLVQAGGSLRLSCAASGSIFSIASMGWYRQAPGKQRELVAVITSFSSTNYADSVKGRFTISRDNAKNTVYLQMNSLKPEDTGVYYCNARYFERTDWGQGTQVTVSS

SEQ ID NO: 307 - protein, 115 aa, synthetic construct
  REGION 1..115: note = Description of Artificial Sequence: Synthetic polypeptide
QVQLQESGGGLVQAGGSLRLSCAAPGSIFSIASMGWYRQAPGKQRELVAVITSFSSTNYADSVKGRFTISRDNAKNTVYLQMNSLKPEDTGVYYCNARYFERTDWGQGTQVTVSS

SEQ ID NO: 308 - protein, 115 aa, synthetic construct
  REGION 1..115: note = Description of Artificial Sequence: Synthetic polypeptide
QVQLQESGGGLVQAGGSLRLSCAASGSIFSIASMAWYRQAPGKQRELVAAITSFSSTNYADSVKGRFTISRDNAKNTVYLQMNSLKPEDTGVYYCNARYFERTDWGQGTQVTVSS

SEQ ID NO: 309 - protein, 115 aa, synthetic construct
  REGION 1..115: note = Description of Artificial Sequence: Synthetic polypeptide
QVQLQESGGGLVQAGGSLRLSCAASESIFSINVMAWHRQAPGKQRELVARITSGGSTNYADSVKGRFTISRDNAKNTVYLQMNSLKPEDTGVYYCGAYQGLYAYWGQGTQVTVSS

SEQ ID NO: 310 - protein, 115 aa, synthetic construct
  REGION 1..115: note = Description of Artificial Sequence: Synthetic polypeptide
QVQLQESGGGLVQAGGSLRLSCVASGSSFSITSMAWYRQAPGKQRDLVAAITSFGSTNYADSVKDRFTISRDNAKNTVYLQMNSLKPEDTAVYYCNGRVFDHVYWGQGTQVTVSS

SEQ ID NO: 311 - protein, 115 aa, synthetic construct
  REGION 1..115: note = Description of Artificial Sequence: Synthetic polypeptide
QVQLQESGGGLVQAGGSLKLSCAASSSIFSISSMSWYRQAPGKQRELVAAITTFDYTNYADSVKGRFTISRDNAKNMMYLQMNSLKPEDTAVYLCNARAFGRDYWGQGTQVTVSS

SEQ ID NO: 312 - protein, 115 aa, synthetic construct
  REGION 1..115: note = Description of Artificial Sequence: Synthetic polypeptide
QVQLQESGGGLVQAGGSLKLSCAASSSIFSISSMSWYRQAPGKQRELVAAITSFGSTNYADSVKGRFTISRDNAKNMMYLQMNSLKPEDTAVYRCNARTMGRDYWGQGTQVTVSS

SEQ ID NO: 313 - protein, 116 aa, synthetic construct
  REGION 1..116: note = Description of Artificial Sequence: Synthetic polypeptide
QVQLQESGGGLVQPGGSLRLSCAASGSTLNIKIMAWHRQAPGKQRELVATLTSGGNTNYADSVKGRFTISRDNAKNTVYLQMNSLQPEDTAVYYCGLWDGVGGAYWGRGTQVTVSS

SEQ ID NO: 314 - protein, 116 aa, synthetic construct
  REGION 1..116: note = Description of Artificial Sequence: Synthetic polypeptide
QVQLQESGGGLVQPGGSLRISCAASGSTLNIKIMAWHRQAPGKQRELVATLTSGGNTNYADSVKGRFTISRDNAKNTVYLQMNSLQPEDTAVYYCGLWDGVGGAYWGRGTQVTVSS

SEQ ID NO: 315 - protein, 116 aa, synthetic construct
  REGION 1..116: note = Description of Artificial Sequence: Synthetic polypeptide
QVQLQESGGGLVQAGGSLRLSCAASGSTFNIKTMAWHRQAPGNQRELVATLTSGGNTNYADSVKGRFTISRDNAKNTVYLQMNSLKPEDTAVYYCGLWNGVGGAYWGRGTQVTVSS

SEQ ID NO: 316 - protein, 115 aa, synthetic construct
  REGION 1..115: note = Description of Artificial Sequence: Synthetic polypeptide
QVQLQDGGGLVQPGGSLRLSCAASGSTFNIKLMAWHRQAPGNQRELVATLTSGGNTNYADSVKGRFTISRDNASNIVYLQMNSLKPEDTAVYYCGLWDGVGGAYWGRGTQVTVSS

SEQ ID NO: 317 - protein, 116 aa, synthetic construct
  REGION 1..116: note = Description of Artificial Sequence: Synthetic polypeptide
QVQLQESGGGLVQAGGSLRLSCAASGSTFNFKIMAWHRQAPGKQRELVASLTSEGLTNYRDSVKGRFTISRDNAKNTVYLQMNNLKPEDTAVYYCGLWDGVGGAYWGRGTQVTVSS

SEQ ID NO: 318 - protein, 114 aa, synthetic construct
  REGION 1..114: note = Description of Artificial Sequence: Synthetic polypeptide
QVQLQESGGGLVQPGGSLRLSCAASGFMFSSYSMSWYRQAPGKQRELVAAITTWGSTNYADSVKGRFTISRDNAKNTVWLQMNSLEPEDTAVYFCNARSWNNYWGQGTQVTVSS

SEQ ID NO: 319 - protein, 114 aa, synthetic construct
  REGION 1..114: note = Description of Artificial Sequence: Synthetic polypeptide
QVQLQESGGGLVQVGGSLRLSCAASGFMFSSYSMSWYRQAPGKQRELVAAITSYGSTNYADSVKGRFTISRDNAKNTVWLQMNSLKPEDTAVYFCNARSWNNYWGQGTQVTVSS

SEQ ID NO: 320 - protein, 114 aa, synthetic construct
  REGION 1..114: note = Description of Artificial Sequence: Synthetic polypeptide
QVQLQESGGGLVQPGGSLRLSCAASGFTFSSHSMSWYRQAPGKQRELVAAITTYGSTNYIDSVKGRFTISRDNTKNTVYLQMNSLKPEDTAVYFCNARSWNNYWGQGTQVTVSS

SEQ ID NO: 321 - protein, 117 aa, synthetic construct
  REGION 1..117: note = Description of Artificial Sequence: Synthetic polypeptide
QVQLQESGGGLVQAGGSLRLSCVASGSSFSHNTMGWYRQAPGKQRDLVARITTFGTTNYADSVKGRFTISRDNAKNTVYLQMNSLKPEDTAVYYCNGESFGRIWYNWGQGTQVTVSS

SEQ ID NO: 322 - protein, 116 aa, synthetic construct
  REGION 1..116: note = Description of Artificial Sequence: Synthetic polypeptide
QVQLQESGGGLVQAGASLRLTCTASGGRFSYATMGWSRQAPGKQREMVARITSSGFSTNYADSVKGRFTISRDNAKNAVYLQMDSLKPEDTAVYYCNAQHFGTDSWGQGTQVTVSS

SEQ ID NO: 323 - protein, 116 aa, synthetic construct
  REGION 1..116: note = Description of Artificial Sequence: Synthetic polypeptide
QVQLQESGGGLVQAGASLRLTCTASGSRFSYATMGWSRQAPGKQRELVARITSSGFSTNYADSVKGRFTISRDNAKNAVYLQMDSLKPEDTAVYYCNAQQFGTDSWGQGTQVTVSS

SEQ ID NO: 324 - protein, 117 aa, synthetic construct
  REGION 1..117: note = Description of Artificial Sequence: Synthetic polypeptide
QVQLQESGGGLVQAGGSLRLSCAASGSTFTSNVMGWHRQAPGKQRELVANMHSGGSTNYADSVKGRFTISRDNAKNIVYLQMNNLKIEDTAVYYCRWYGIQRAEGYWGQGTQVTVSS

SEQ ID NO: 325 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISVDGSTNYADSVKGRFTVSRDNAKNTVYLQMNSLQPEDTAVYYCYAYRWVGRDTYWGQGTQVTVSS

SEQ ID NO: 326 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISVDGSTNYADSVKGRFTISRDNAKNTVYLQMNSLQPEDTAVYYCYAYRWVGRDTYWGQGTQVTVSS

SEQ ID NO: 327 - protein, 117 aa, synthetic construct
  REGION 1..117: note = Description of Artificial Sequence: Synthetic polypeptide
QVQLQESGGGLVVSGGSLRLSCAASGSSVSFLSMAWYRQAPGKKRELVAGISVDGSTNYADSVKGRFTISRDNAKNTVYLQMNSLQPEDTAAYYCYAYRWVGRDTYWGQGTQVTVSS

SEQ ID NO: 328 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISVDGSTNYADSVKGRFTISRDNAENTVYLQMNSLQPEDTAVYYCYAYRWEGRDTYWGQGTQVTVSS

SEQ ID NO: 329 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISVDGSTNYADSVKGRFTISRDNAENTVYLQMNSLQPEDTAVYYCYAYRWEGRNTYWGQGTQVTVSS

SEQ ID NO: 330 - protein, 117 aa, synthetic construct
  REGION 1..117: note = Description of Artificial Sequence: Synthetic polypeptide
QVQLQESGGGLVQPGGSLRLSCAASGSSVSFLSMAWYRQAPGKKRELVAGISTDGSTNYVDSVKGRFTISRDNAKNTVYLQMNSLQPEDTAVYYCYAYRWVGRYTYWGQGTQVTVSS

SEQ ID NO: 331 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISTDGTTNYVDSVKDRFTISRDNAKNTVYLQMNSLQPEDTAAYYCYAYRWVGRDTYWGQGTQVTVSS

SEQ ID NO: 332 - protein, 117 aa, synthetic construct
  REGION 1..117: note = Description of Artificial Sequence: Synthetic polypeptide
QVQLQESGGGLVVAGGSLRLSCAASGSSVSFLSIAWYRQAPGKKRELVAGISTDGTTNYVDSVKDRFTISRDNAKNTVYLQMNSLQPEDTAAYYCYAYRWVGRDTYWGQGTQVTVSS

SEQ ID NO: 333 - protein, 117 aa, synthetic construct
  REGION 1..117: note = Description of Artificial Sequence: Synthetic polypeptide
QVQLQESGGGLVQAGGSLTLSCAASGSSVSFLSMAWYRQAPGKKRELVAGISTDGTTNYVDSVKDRFTISRDNAKNTVYLQMNSLQPEDTAAYYCYAYRWVGRDTYWGQGTQVTVSS

SEQ ID NO: 334 - protein, 117 aa, synthetic construct
  REGION 1..117: note = Description of Artificial Sequence: Synthetic polypeptide
QVQLQESGGGLVQAGGSLRLSCAASGSSVSFLSMAWYRQAPGKKRELVAGISTDGSTNYADSVKGRFTISEGNAKNTVDLQMNSLQPEDTAVYYCYAYRWVDRYTYWGQGTQVTVSS

SEQ ID NO: 335 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISTDGSTNYADSVKGRFTISEDNAKNTVDLQMNSLQPEDTAVYYCYAYRWIDRYTYWGQGTQVTVSS

SEQ ID NO: 336 - protein, 117 aa, synthetic construct
  REGION 1..117: note = Description of Artificial Sequence: Synthetic polypeptide
QVQLQESGGGLVQPGGSLRLSCAASGSSVSFLSMAWYRQAPGKKRELVAGISTDGSTNYADSVKGRFTISEDNAKNTVDLQMNSLQPEDTAVYYCYAYRWVDRYTYWGQGTQVTVSS

SEQ ID NO: 337 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISSDGSTNYVDSVKGRFTISRDNAKNIVFLQMNSLQPQDTAVYYCYAYRWVGRDTYWGQGTQVTVSS

SEQ ID NO: 338 - protein, 117 aa, synthetic construct
  REGION 1..117: note = Description of Artificial Sequence: Synthetic polypeptide
QVQLQESGGGLVVAGGSLRLSCAASGSSVSFLSMAWYRQAPGKKRELVAGISADGSTDYIDSVKGRFTISRDSANNTMYLQMNSLQPEDTAVYYCYAYRWTTRYTYWGQGTQVTVSS

SEQ ID NO: 339 - protein, 117 aa, synthetic construct
  REGION 1..117: note = Description of Artificial Sequence: Synthetic polypeptide
QVQLQESGGGLVQPGGSLRLSCAASGFTLDYYAIGWYRQAPGKKRELVAGISSDGSTHYVDSVKGRFAISRDNAENTVYLQMNDLQPDDTAVYYCYAYRWVGGYTYWGQGTQVTVSS

SEQ ID NO: 340 - protein, 119 aa, synthetic construct
  REGION 1..119: note = Description of Artificial Sequence: Synthetic polypeptide
QVQLQESGGGLVQAGGSLRLSCVASGSTSSINAMGWYRRAPGKQRELVAGISSDGSKNYADSVKGRFTISRDNAKNTVYLQMNSLKPEDTAVYYCYYFRTVAASSMQYWGQGTQVTVSS

SEQ ID NO: 341 - protein, 119 aa, synthetic construct
  REGION 1..119: note = Description of Artificial Sequence: Synthetic polypeptide
QVQLQESGGGLVQAGGSLRLSCVASGSTSSINAMGWYRRAPGKQRELVAGISSDGSKVYADSVKGRFTISRDNAKNTVYLQMNSLKPEDTAVFYCYYFRTVSGSSMRYWGQGTQVTVSS

SEQ ID NO: 342 - protein, 118 aa, synthetic construct
  REGION 1..118: note = Description of Artificial Sequence: Synthetic polypeptide
QVQLQESGGGLVQAGGSLRLSCAASGITSSVYSMGWYRQAPGKQRELVAGSSSDGSTHYVDSVRGRFTISRDNAKNTVYLQMSSLKPEDTAVYYCYANRGFAGAPSYWGQGTQVTVSS

SEQ ID NO: 343 - protein, 114 aa, synthetic construct
  REGION 1..114: note = Description of Artificial Sequence: Synthetic polypeptide
QVQLQESGGGLVQAGGSLRLSCAASGRTSMFNSMGWHRQAPGKQRELVAIIRSGGSSNYADTVKGRFTISRDNTKNTVYLQMNDLKPEDTAVYYCFYYFQSSYWGQGTQVTVSS

SEQ ID NO: 344 - protein, 114 aa, synthetic construct
  REGION 1..114: note = Description of Artificial Sequence: Synthetic polypeptide
QVQLQESGGGLVQAGGSLRLSCAASGRTSMVNSMGWHRQAPGKQRELVALITSGGSSNYADTVKGRFTISRDNTKNTVYLQMNDLKPEDTAVYYCFYYFQSSYWGQGTQVTVSS

SEQ ID NO: 345 - protein, 119 aa, synthetic construct
  REGION 1..119: note = Description of Artificial Sequence: Synthetic polypeptide
QVQLQESGGGLVQAGGSLRLSCTASGSIFSIAVMGWYRQVPGKRREWVATIFDGSYTNYADSVKGRFTISRDNARNKVYLQMNNLKPEDTAVYYCQTHWTQGSVPKESWGQGTQVTVSS

SEQ ID NO: 346 - protein, 115 aa, synthetic construct
  REGION 1..115: note = Description of Artificial Sequence: Synthetic polypeptide
QVQLQESGGGLVQAGGSLRLSCVASSGIFSDMSMVWYRQAPGKQRELVASITTFGSTNYADPVKGRFTISRDNAKNTVYLQMNSLKPEDTAVYYCSGRSYSSDYWGRGTQVTVSS

SEQ ID NO: 347 - protein, 115 aa, synthetic construct
  REGION 1..115: note = Description of Artificial Sequence: Synthetic polypeptide
QVQLQESGGGLVQAGGSLRLSCVASGSISSIIVMGWSRQAPGKQRESVATITRDGTRNYADSLKGRFTISRDNAKNTSYLQINSLKPEDTAVYSCYARYGDINYWGKGTQVTVSS

SEQ ID NO: 348 - protein, 115 aa, synthetic construct
  REGION 1..115: note = Description of Artificial Sequence: Synthetic polypeptide
QVQLQESGGGLVQAGGSLRLSCVASGSISSIIVMGWSRQAPGKQRESLATISRGGTRTYADSVKGRFTISRDNAKNTSYLQMNSLKPEDTAVYSCYARYGDINYWGKGTQVTVSS

SEQ ID NO: 349 - protein, 114 aa, synthetic construct
  REGION 1..114: note = Description of Artificial Sequence: Synthetic polypeptide
QVQLQESGGGLVQAGGSLRLSCVASGSIFTTNSMGWHRQGPGKQRELVALIGSAGSTKYADSVKGRFTISRDNAKNTVSLQMDSLKPEDTAVYYCFYYDSRSYWGQGTQVTVSS

SEQ ID NO: 350 - protein, 114 aa, synthetic construct
  REGION 1..114: note = Description of Artificial Sequence: Synthetic polypeptide
QVQLQESGGGMVQPGGSLRLSCAASGSREISTMGWHRQAPGKQRELAARITSGGITKYADSVKGRFTISRDNAKKTVYLQMNSLKSEDTAVYYCFAYDNINAYWGQGTQVTVSS

SEQ ID NO: 351 - protein, 114 aa, synthetic construct
  REGION 1..114: note = Description of Artificial Sequence: Synthetic polypeptide
QVQLQESGGGWVQAGGSLRLSCAASGSREISTMGWHRQAPGKQRELAARITSGGITKYADSVKGRFTISRDNAKKTVYLQMNSLKSEDTAVYYCFAYDNINAYWGQGTQVTVSS

SEQ ID NO: 352 - protein, 114 aa, synthetic construct
  REGION 1..114: note = Description of Artificial Sequence: Synthetic polypeptide
QVQLQESGGGWVQAGGSLRLSCTASGSREISTMGWHRQAPGKQRELAARITSGGITKYADSVKGRFTISRDNAKKTVYLQMDSLKSEDTAVYYCFAYDNINAYWGQGTQVTVSS

SEQ ID NO: 353 - protein, 114 aa, synthetic construct
  REGION 1..114: note = Description of Artificial Sequence: Synthetic polypeptide
QVQLQESGGGSVQAGRSLGLSCAASGSREISTMGWHRQAPGKQRELAARITSGGITKYADSVKGRFTISRDNAKKTVYLQMNSLKSEDTAVYYCFAYDNINAYWGQGTQVTVSS

SEQ ID NO: 354 - protein, 115 aa, synthetic construct
  REGION 1..115: note = Description of Artificial Sequence: Synthetic polypeptide
QVQLQESGGGLVQAGGSLRLSCTASGSIFRGAAMYWHRQAPGKQRELVAAITTSGNTSYADSVKGRFTISRDNAKNTMYLQIISLKPEDTAVYYCAFWIAGKAYWGQGTQVTVSS

SEQ ID NO: 355 - protein, 115 aa, synthetic construct
  REGION 1..115: note = Description of Artificial Sequence: Synthetic polypeptide
QVQLQESGGGLVQPGGSLRLSCAASGSISSFNFMSWHRQAPGKERELAGVITRGGATNYADSVKGRFTISRDNVKNTVYLQMNGLKPEDTAVYYCHGRSQLGSTWGQGTQVTVSS

SEQ ID NO: 356 - protein, 116 aa, synthetic construct
  REGION 1..116: note = Description of Artificial Sequence: Synthetic polypeptide
QVQLQESGGGLVQAGGSLRLSCLASGTIFTASTMGWHRQPPGKQRELVASIAGDGRTNYAESTEGRFTISRDDAKNTMYLQMNSLKPEDTAVYYCYAYYLDTYAYWGQGTQVTVSS

SEQ ID NO: 357 - protein, 115 aa, synthetic construct
  REGION 1..115: note = Description of Artificial Sequence: Synthetic polypeptide
EVQLVESGGGLVQPGGSLTLSCAASGSIFSIASMGWYRQAPGKQRELVAVITSFSSTNYADSVKGRFTISRDNAKNSVYLQMNSLRAEDTAVYYCNARYFERTDWGQGTLVTVSS

SEQ ID NO: 358 - protein, 114 aa, synthetic construct
  REGION 1..114: note = Description of Artificial Sequence: Synthetic polypeptide
EVQLVESGGGLVQPGGSLTLSCAASGRTSMFNSMGWHRQAPGKQRELVAIIRSGGSSNYADTVKGRFTISRDNAKNSVYLQMNSLRAEDTAVYYCFYYFQSSYWGQGTLVTVSS

SEQ ID NO: 359 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISVDGSTNYADSVKGRFTISRDNAKNSVYLQMNSLRAEDTAVYYCYAYRWVGRDTYWGQGTLVTVSS

SEQ ID NO: 360 - protein, 115 aa, synthetic construct
  REGION 1..115: note = Description of Artificial Sequence: Synthetic polypeptide
EVQLVESGGGLVQPGGSLTLSCTASGSIFRGAAMYWHRQAPGKQRELVAAITTSGNTSYADSVKGRFTISRDNAKNSMYLQMNSLRAEDTAVYYCAFWIAGKAYWGQGTLVTVSS

SEQ ID NO: 361 - protein, 116 aa, synthetic construct
  REGION 1..116: note = Description of Artificial Sequence: Synthetic polypeptide
EVQLVESGGGLVQPGGSLTLSCAASGSTFNIKTMAWHRQAPGNQRELVATLTSGGNTNYADSVKGRFTISRDNAKNSVYLQMNSLRAEDTAVYYCGLWNGVGGAYWGQGTLVTVSS

SEQ ID NO: 362 - protein, 116 aa, synthetic construct
  REGION 1..116: note = Description of Artificial Sequence: Synthetic polypeptide
EVQLVESGGGLVQPGGSLTLSCAASGSTLNIKIMAWHRQAPGKQRELVATLTSGGNTNYADSVKGRFTISRDNAKNSVYLQMNSLRAEDTAVYYCGLWDGVGGAYWGQGTLVTVSS

SEQ ID NO: 363 - protein, 115 aa, synthetic construct
  REGION 1..115: note = Description of Artificial Sequence: Synthetic polypeptide
EVQLVESGGGLVQPGGSLTLSCAASSSIFSISSMSWYRQAPGKQRELVAAITTFDYTNYADSVKGRFTISRDNAKNSMYLQMNSLRAEDTAVYYCNARAFGRDYWGQGTLVTVSS

SEQ ID NO: 364 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKNYADSVKGRFTISRDNAKNSVYLQMNSLRAEDTAVYYCYYFRTVAASSMQYWGQGTLVTVSS

SEQ ID NO: 365 - protein, 114 aa, synthetic construct
  REGION 1..114: note = Description of Artificial Sequence: Synthetic polypeptide
EVQLVESGGGLVQPGGSLTLSCAASGFMFSSYSMSWYRQAPGKQRELVAAITSYGSTNYADSVKGRFTISRDNAKNSVYLQMNSLRAEDTAVYYCNARSWNNYWGQGTLVTVSS

SEQ ID NO: 366 - protein, 114 aa, synthetic construct
  REGION 1..114: note = Description of Artificial Sequence: Synthetic polypeptide
EVQLVESGGGLVQPGGSLTLSCAASGSVSMFNSMGWHRQPPGKQRELVAIITSGGSSNYADTVKGRFTISRDNAKNSVYLQMNSLRAEDTAVYYCFYYFQSSYWGQGTLVTVSS

SEQ ID NO: 367 - protein, 116 aa, synthetic construct
  REGION 1..116: note = Description of Artificial Sequence: Synthetic polypeptide
EVQLVESGGGLVQPGGSLTLSCTASGGRFSYATMGWSRQAPGKQREMVARITSSGFSTNYADSVKGRFTISRDNAKNSVYLQMNSLRAEDTAVYYCNAQHFGTDSWGQGTLVTVSS

SEQ ID NO: 368 - protein, 115 aa, synthetic construct
  REGION 1..115: note = Description of Artificial Sequence: Synthetic polypeptide
EVQLVESGGGLVQPGGSLTLSCAASGSISSFNFMSWHRQAPGKERELAGVITRGGATNYADSVKGRFTISRDNAKNSVYLQMNSLRAEDTAVYYCHGRSQLGSTWGQGTLVTVSS

SEQ ID NO: 369 - protein, 115 aa, synthetic construct
  REGION 1..115: note = Description of Artificial Sequence: Synthetic polypeptide
EVQLVESGGGLVQPGGSLTLSCAASESIFSINVMAWHRQAPGKQRELVARITSGGSTNYADSVKGRFTISRDNAKNSVYLQMNSLRAEDTAVYYCGAYQGLYAYWGQGTLVTVSS

SEQ ID NO: 370 - protein, 114 aa, synthetic construct
  REGION 1..114: note = Description of Artificial Sequence: Synthetic polypeptide
EVQLVESGGGLVQPGGSLTLSCAASGSREISTMGWHRQAPGKQRELAARITSGGITKYADSVKGRFTISRDNAKNSVYLQMNSLRAEDTAVYYCFAYDNINAYWGQGTLVTVSS

SEQ ID NO: 371 - protein, 119 aa, synthetic construct
  REGION 1..119: note = Description of Artificial Sequence: Synthetic polypeptide
EVQLVESGGGLVQPGGSLTLSCTASGSIFSIAVMGWYRQVPGKRREWVATIFDGSYTNYADSVKGRFTISRDNAKNSVYLQMNSLRAEDTAVYYCQTHWTQGSVPKESWGQGTLVTVSS

SEQ ID NO: 372 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYADSVKGRFTISRDNAKNSVYLQMNSLRAEDTAVYYCYYFRTVSGSSMRYWGQGTLVTVSS

SEQ ID NO: 373 - protein, 115 aa, synthetic construct
  REGION 1..115: note = Description of Artificial Sequence: Synthetic polypeptide
EVQLVESGGGLVQPGGSLTLSCVASSGIFSDMSMVWYRQAPGKQRELVASITTFGSTNYADPVKGRFTISRDNAKNSVYLQMNSLRAEDTAVYYCSGRSYSSDYWGQGTLVTVSS

SEQ ID NO: 374 - protein, 114 aa, synthetic construct
  REGION 1..114: note = Description of Artificial Sequence: Synthetic polypeptide
EVQLVESGGGLVQPGGSLTLSCAASGFMFSSYSMSWYRQAPGKQRELVAAITTWGSTNYADSVKGRFTISRDNAKNSVYLQMNSLRAEDTAVYYCNARSWNNYWGQGTLVTVSS

SEQ ID NO: 375 - protein, 116 aa, synthetic construct
  REGION 1..116: note = Description of Artificial Sequence: Synthetic polypeptide
EVQLVESGGGLVQPGGSLTLSCTASGSRFSYATMGWSRQAPGKQRELVARITSSGFSTNYADSVKGRFTISRDNAKNSVYLQMNSLRAEDTAVYYCNAQQFGTDSWGQGTLVTVSS

SEQ ID NO: 376 - protein, 117 aa, synthetic construct
  REGION 1..117: note = Description of Artificial Sequence: Synthetic polypeptide
EVQLVESGGGLVQPGGSLTLSCAASGSTFTSNVMGWHRQAPGKQRELVANMHSGGSTNYADSVKGRFTISRDNAKNSVYLQMNSLRAEDTAVYYCRWYGIQRAEGYWGQGTLVTVSS

SEQ ID NO: 377 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISTDGSTNYVDSVKGRFTISRDNAKNSVYLQMNSLRAEDTAVYYCYAYRWVGRYTYWGQGTLVTVSS

SEQ ID NO: 378 - protein, 115 aa, synthetic construct
  REGION 1..115: note = Description of Artificial Sequence: Synthetic polypeptide
EVQLVESGGGLVQPGGSLTLSCVASGSISSIIVMGWSRQAPGKQRESVATITRDGTRNYADSLKGRFTISRDNAKNSSYLQMNSLRAEDTAVYYCYARYGDINYWGQGTLVTVSS

SEQ ID NO: 379 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTDYIDSVKGRFTISRDNAKNSVYLQMNSLRAEDTAVYYCYAYRWTTRYTYWGQGTLVTVSS

SEQ ID NO: 380 - protein, 116 aa, synthetic construct
  REGION 1..116: note = Description of Artificial Sequence: Synthetic polypeptide
EVQLVESGGGLVQPGGSLTLSCLASGTIFTASTMGWHRQPPGKQRELVASIAGDGRTNYAESTEGRFTISRDNAKNSMYLQMNSLRAEDTAVYYCYAYYLDTYAYWGQGTLVTVSS

SEQ ID NO: 381 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISVDGSTNYADSVKGRFTISRDNAKNSVYLQMNSLRAEDTAVYYCYAYRWEGRNTYWGQGTLVTVSS

SEQ ID NO: 382 - protein, 117 aa, synthetic construct
  REGION 1..117: note = Description of Artificial Sequence: Synthetic polypeptide
EVQLVESGGGLVQPGGSLTLSCVASGSSFSHNTMGWYRQAPGKQRDLVARITTFGTTNYADSVKGRFTISRDNAKNSVYLQMNSLRAEDTAVYYCNGESFGRIWYNWGQGTLVTVSS

SEQ ID NO: 383 - protein, 115 aa, synthetic construct
  REGION 1..115: note = Description of Artificial Sequence: Synthetic polypeptide
EVQLVESGGGLVQPGGSLTLSCVASGSISSIIVMGWSRQAPGKQRESLATISRGGTRTYADSVKGRFTISRDNAKNSSYLQMNSLRAEDTAVYYCYARYGDINYWGQGTLVTVSS

SEQ ID NO: 384 - protein, 115 aa, synthetic construct
  REGION 1..115: note = Description of Artificial Sequence: Synthetic polypeptide
EVQLVESGGGLVQPGGSLTLSCVASGSSFSITSMAWYRQAPGKQRDLVAAITSFGSTNYADSVKDRFTISRDNAKNSVYLQMNSLRAEDTAVYYCNGRVFDHVYWGQGTLVTVSS

SEQ ID NO: 385 - protein, 114 aa, synthetic construct
  REGION 1..114: note = Description of Artificial Sequence: Synthetic polypeptide
EVQLVESGGGLVQPGGSLTLSCAASGRTSMVNSMGWHRQAPGKQRELVALITSGGSSNYADTVKGRFTISRDNAKNSVYLQMNSLRAEDTAVYYCFYYFQSSYWGQGTLVTVSS

SEQ ID NO: 386 - protein, 116 aa, synthetic construct
  REGION 1..116: note = Description of Artificial Sequence: Synthetic polypeptide
EVQLVESGGGLVQPGGSLTLSCAASGSTFNFKIMAWHRQAPGKQRELVASLTSEGLTNYRDSVKGRFTISRDNAKNSVYLQMNSLRAEDTAVYYCGLWDGVGGAYWGQGTLVTVSS

SEQ ID NO: 387 - protein, 117 aa, synthetic construct
  REGION 1..117: note = Description of Artificial Sequence: Synthetic polypeptide
EVQLVESGGGLVQPGGSLTLSCAASGFTLDYYAIGWYRQAPGKKRELVAGISSDGSTHYVDSVKGRFTISRDNAKNSVYLQMNSLRAEDTAVYYCYAYRWVGGYTYWGQGTLVTVSS

SEQ ID NO: 388 - protein, 114 aa, synthetic construct
  REGION 1..114: note = Description of Artificial Sequence: Synthetic polypeptide
EVQLVESGGGLVQPGGSLTLSCVASGSIFTTNSMGWHRQGPGKQRELVALIGSAGSTKYADSVKGRFTISRDNAKNSVYLQMNSLRAEDTAVYYCFYYDSRSYWGQGTLVTVSS

SEQ ID NO: 389 - protein, 118 aa, synthetic construct
  REGION 1..118: note = Description of Artificial Sequence: Synthetic polypeptide
EVQLVESGGGLVQPGGSLTLSCAASGITSSVYSMGWYRQAPGKQRELVAGSSSDGSTHYVDSVRGRFTISRDNAKNSVYLQMNSLRAEDTAVYYCYANRGFAGAPSYWGQGTLVTVSS

SEQ ID NO: 390 - protein, 115 aa, synthetic construct
  REGION 1..115: note = Description of Artificial Sequence: Synthetic polypeptide
EVQLVESGGGLVQPGGSLTLSCAASSSIFSISSMSWYRQAPGKQRELVAAITSFGSTNYADSVKGRFTISRDNAKNSMYLQMNSLRAEDTAVYYCNARTMGRDYWGQGTLVTVSS

SEQ ID NO: 391 - protein, 119 aa, synthetic construct
  REGION 1..119: note = Description of Artificial Sequence: Synthetic polypeptide
EVQLVESGGGLVQPGGSLTLSCVASGFTSSINAMGWYRRAPGKQRELVAGISSDGSFVYADSVKGRFTISRDNAKNSVYLQMNSLRAEDTAVYYCYYFRHVSGSSMRYWGQGTLVTVSS

SEQ ID NO: 392 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVSGSSSRYWGQGTLVTVSS

SEQ ID NO: 393 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYEDSVKGRFTISRDNAKNSVYLQMNSLRAEDTAVYYCYYFRTVSGSSMRYWGQGTLVTVSS

SEQ ID NO: 394 - protein, 119 aa, synthetic construct
  REGION 1..119: note = Description of Artificial Sequence: Synthetic polypeptide
EVQLVESGGGLVQPGGSLTLSCVASGSPSSINAMGWYRRAPGKQRELSAGISSDGSKVYADSVKGRFTISRDNAKNSVYLQMNSLRAEDTAVYYCYYFRTVRGSSMSYWGQGTLVTVSS

SEQ ID NO: 395 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELAAGISSDGSSVYADSVKGRFTISRDNAKNSVYLQMNSLRAEDTAVYYCYYFRTVSGSSKRYWGQGTLVTVSS

SEQ ID NO: 396 - protein, 119 aa, synthetic construct
  REGION 1..119: note = Description of Artificial Sequence: Synthetic polypeptide
EVQLVESGGGLVQPGGSLTLSCVASGSISSINAMGWYRRAPGKQRELVAGISSDGSKVYADSVKGRFTISRDNAKNSVYLQMNSLRAEDTAVYYCYYFRMVSGSSMRYWGQGTLVTVSS

SEQ ID NO: 397 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKLYADSVKGRFTISRDNAKNSVYLQMNSLRAEDTAVYYCYYFRTVQGSSMRYWGQGTLVTVSS

SEQ ID NO: 398 - protein, 119 aa, synthetic construct
  REGION 1..119: note = Description of Artificial Sequence: Synthetic polypeptide
EVQLVESGGGLVQPGGSLTLSCVASGSTSSINAYGWYRRAPGKQRELVAGISSDGSKVYADSVKGRFTISRDNAKNSVYLQMNSLRAEDTAVYYCYYFRTVYGSSMRYWGQGTLVTVSS

SEQ ID NO: 399 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVYIDSVKGRFTISRDNAKNSVYLQMNSLRAEDTAVYYCYYFRTVSGSSYRYWGQGTLVTVSS

SEQ ID NO: 400 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYSDSVKGRFTISRDNAKNSVYLQMNSLRAEDTAVYYCYYFRTVLGSSMRYWGQGTLVTVSS

SEQ ID NO: 401 - protein, 119 aa, synthetic construct
  REGION 1..119: note = Description of Artificial Sequence: Synthetic polypeptide
EVQLVESGGGLVQPGGSLTLSCVASGSTSIINAMGWYRRAPGKQRELAAGISSDGSKVIADSVKGRFTISRDNAKNSVYLQMNSLRAEDTAVYYCYYFRRVSGSSMRYWGQGTLVTVSS

SEQ ID NO: 402 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKIYADSVKGRFTISRDNAKNSVYLQMNSLRAEDTAVYYCYYFRTVSGSSMRYWGQGTLVTVSS

SEQ ID NO: 403 - protein, 119 aa, synthetic construct
  REGION 1..119: note = Description of Artificial Sequence: Synthetic polypeptide
EVQLVESGGGLVQPGGSLTLSCVASGKTSSINAMAWYRRAPGKQRELVAGISSDGSKVYTDSVKGRFTISRDNAKNSVYLQMNSLRAEDTAVYYCYYFRTVSGSSARYWGQGTLVTVSS

SEQ ID NO: 404 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSLVYADSVKGRFTISRDNAKNSVYLQMNSLRAEDTAVYYCYYFRIVRGSSMRYWGQGTLVTVSS

SEQ ID NO: 405 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYYRTVSGSSMRYWGQGTLVTVSS

SEQ ID NO: 406 - protein, 119 aa, synthetic construct
  REGION 1..119: note = Description of Artificial Sequence: Synthetic polypeptide
EVQLVESGGGLVQPGGSLTLSCVASGSGSSINAMGWYRRAPGKQRELVAGISSDGSKVYSDSVKGRFTISRDNAKNSVYLQMNSLRAEDTAVYYCYYFRHVSGSSMRYWGQGTLVTVSS

SEQ ID NO: 407 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVYVDSVKGRFTISRDNAKNSVYLQMNSLRAEDTAVYYCYYFRFVSGSSMRYWGQGTLVTVSS

SEQ ID NO: 408 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYVDSVKGRFTISRDNAKNSVYLQMNSLRAEDTAVYYCYYFRTVSGSSMRYWGQGTLVTVSS

SEQ ID NO: 409 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRTKSGSSMRYWGQGTLVTVSS

SEQ ID NO: 410 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRTVYGSSMRYWGQGTLVTVSS

SEQ ID NO: 411 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYRDSVKGRFTISRDNAKNSVYLQMNSLRAEDTAVYYCYYFRTVSGSSMGYWGQGTLVTVSS

SEQ ID NO: 412 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYSDSVKGRFTISRDNAKNSVYLQMNSLRAEDTAVYYCYYFRTVSGSSMRSWGQGTLVTVSS

SEQ ID NO: 413 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDNSKVYADSVKGRFTISRDNAKNSVYLQMNSLRAEDTAVYYCYYFRTVGGSSMRYWGQGTLVTVSS

SEQ ID NO: 414 - protein, 119 aa, synthetic construct
  REGION 1..119: note = Description of Artificial Sequence: Synthetic polypeptide
EVQLVESGGGLVQPGGSLTLSCVASGNTSSINAMAWYRRAPGKQRELVAGISSDGSKVYADSVKGRFTISRDNAKNSVYLQMNSLRAEDTAVYYCYYFRTVSGSSMRYWGQGTLVTVSS

SEQ ID NO: 415 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVSGSHMRYWGQGTLVTVSS

SEQ ID NO: 416 - protein, 119 aa, synthetic construct
  REGION 1..119: note = Description of Artificial Sequence: Synthetic polypeptide
EVQLVESGGGLVQPGGSLTLSCVASGSTSIINAMGWYRRAPGKQRELVAGISSDGSKVYEDSVKGRFTISRDNAKNSVYLQMNSLRAEDTAVYYCYYFRAVSGSSMRYWGQGTLVTVSS

SEQ ID NO: 417 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVSGSSMRYWGQGTLVTVSS

SEQ ID NO: 418 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELPAGISSDGSKVYAVSVKGRFTISRDNAKNSVYLQMNSLRAEDTAVYYCYYFRTVSGSPMRYWGQGTLVTVSS

SEQ ID NO: 419 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGVSSDGSKVYADSVKGRFTISRDNAKNSVYLQMNSLRAEDTAVYYCYYFRTVSGSSMSYWGQGTLVTVSS

SEQ ID NO: 420 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVYEDSVKGRFTISRDNAKNSVYLQMNSLRAEDTAVYYCYYFRTVSGSSMRYWGQGTLVTVSS

SEQ ID NO: 421 - protein, 119 aa, synthetic construct
  REGION 1..119: note = Description of Artificial Sequence: Synthetic polypeptide
EVQLVESGGGLVQPGGSLTLSCVASGITSSINAMGWYRRAPGKQRELVAGISSDGSKVYAGSVKGRFTISRDNAKNSVYLQMNSLRAEDTAVYYCYYFRTVRGSSMRYWGQGTLVTVSS

SEQ ID NO: 422 - protein, 119 aa, synthetic construct
  REGION 1..119: note = Description of Artificial Sequence: Synthetic polypeptide
EVQLVESGGGLVQPGGSLTLSCVASGSTSDINAMGWYRRAPGKQRELVAGISSDKSKVYADSVKGRFTISRDNAKNSVYLQMNSLRAEDTAVYYCYYFRTVRGSSMRYWGQGTLVTVSS

SEQ ID NO: 423 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSNGSKVYADSVKGRFTISRDNAKNSVYLQMNSLRAEDTAVYYCYYFRQVSGSSMRYWGQGTLVTVSS

SEQ ID NO: 424 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVLADSVKGRFTISRDNAKNSVYLQMNSLRAEDTAVYYCYYFRIVSGSSMGYWGQGTLVTVSS

SEQ ID NO: 425 - protein, 119 aa, synthetic construct
  REGION 1..119: note = Description of Artificial Sequence: Synthetic polypeptide
EVQLVESGGGLVQPGGSLTLSCVASGSTSSKNAMGWYRRAPGKQRELVAGISSDGSKVYADSVKGRFTISRDNAKNSVYLQMNSLRAEDTAVYYCYYFRTVSGASMRYWGQGTLVTVSS

SEQ ID NO: 426 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDNSKVYADSVKGRFTISRDNAKNSVYLQMNSLRAEDTAVYYCYYFRTVHGSSMRYWGQGTLVTVSS

SEQ ID NO: 427 - protein, 119 aa, synthetic construct
  REGION 1..119: note = Description of Artificial Sequence: Synthetic polypeptide
EVQLVESGGGLVQPGGSLTLSCVASGLTSSINAMGWYRRAPGKQRELVAGISSDGSKVYADSVKGRFTISRDNAKNSVYLQMNSLRAEDTAVYYCYYFRMVSGSSMRYWGQGTLVTVSS

SEQ ID NO: 428 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYTDSVKGRFTISRDNAKNSVYLQMNSLRAEDTAVYYCYYFRTISGSSMRYWGQGTLVTVSS

SEQ ID NO: 429 - protein, 119 aa, synthetic construct
  REGION 1..119: note = Description of Artificial Sequence: Synthetic polypeptide
EVQLVESGGGLVQPGGSLTLSCVASGSTSSNNAMAWYRRAPGKQRELVAGISSDGSKVYTDSVKGRFTISRDNAKNSVYLQMNSLRAEDTAVYYCYYFRTRSGSSMRYWGQGTLVTVSS

SEQ ID NO: 430 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDNSKVYADSVKGRFTISRDNAKNSVYLQMNSLRAEDTAVYYCYYFRTVSGHSMRYWGQGTLVTVSS

SEQ ID NO: 431 - protein, 119 aa, synthetic construct
  REGION 1..119: note = Description of Artificial Sequence: Synthetic polypeptide
EVQLVESGGGLVQPGGSLTLSCVASGSTSHINAMGWYRRAPGKQRELVAGISSDGSRVYADSVKGRFTISRDNAKNSVYLQMNSLRAEDTAVYYCYYFRTVSGGSMRYWGQGTLVTVSS

SEQ ID NO: 432 - protein, 119 aa, synthetic construct
  REGION 1..119: note = Description of Artificial Sequence: Synthetic polypeptide
EVQLVESGGGLVQPGGSLTLSCVASGQTSSINAMGWYRRAPGKQRELVAGISSDGSQVYADSVKGRFTISRDNAKNSVYLQMNSLRAEDTAVYYCYYFRTKSGSSMRYWGQGTLVTVSS

SEQ ID NO: 433 - protein, 119 aa, synthetic construct
  REGION 1..119: note = Description of Artificial Sequence: Synthetic polypeptide
EVQLVESGGGLVQPGGSLTLSCVASGSTSRINGMGWYRRAPGKQRELPAGISSDGSKAYADSVKGRFTISRDNAKNSVYLQMNSLRAEDTAVYYCYYFRTASGTSMRYWGQGTLVTVSS

SEQ ID NO: 434 - protein, 119 aa, synthetic construct
  REGION 1..119: note = Description of Artificial Sequence: Synthetic polypeptide
EVQLVESGGGLVQPGGSLTLSCVASGSTSVINAMAWYRRAPGKQRELAAGISSDGSKVYAKSAKGRFTISRDNAKNSVYLQMNSLRAEDTAVYYCYYFNTVSGSSMRYWGQGTLVTVSS

SEQ ID NO: 435 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYNDSVKGRFTISRDNAKNSVYLQMNSLRAEDTAVYYCYYFRTVRGSSQRYWGQGTLVTVSS

SEQ ID NO: 436 - protein, 119 aa, synthetic construct
  REGION 1..119: note = Description of Artificial Sequence: Synthetic polypeptide
EVQLVESGGGLVQPGGSLTLSCVASGKTSSINAMGWYRRAPGKQRELVAGISSDGSKVIADSVKGRFTISRDNAKNSVYLQMNSLRAEDTAVYYCYYFRTVLGSSMRYWGQGTLVTVSS

SEQ ID NO: 437 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYTDSVKGRFTISRDNAKNSVYLQMNSLRAEDTAVYYCYYFRTRSGSSMRYWGQGTLVTVSS

SEQ ID NO: 438 - protein, 119 aa, synthetic construct
  REGION 1..119: note = Description of Artificial Sequence: Synthetic polypeptide
EVQLVESGGGLVQPGGSLTLSCVASGSVSSINAMGWYRRAPGKQRELVAGISSDGSKVYIDSVKGRFTISRDNAKNSVYLQMNSLRAEDTAVYYCYYFRTVSGLSMRYWGQGTLVTVSS

SEQ ID NO: 439 - protein, 119 aa, synthetic construct
  REGION 1..119: note = Description of Artificial Sequence: Synthetic polypeptide
EVQLVESGGGLVQPGGSLTLSCVASGNTSSINAMGWYRRAPGKQRELVAGISSDGSKVYYDSVKGRFTISRDNAKNSVYLQMNSLRAEDTAVYYCYYFRTVRGSSQRYWGQGTLVTVSS

SEQ ID NO: 440 - protein, 119 aa, synthetic construct
  REGION 1..119: note = Description of Artificial Sequence: Synthetic polypeptide
EVQLVESGGGLVQPGGSLTLSCVASGSTSSTNAMGWYRRAPGKQRELVAGISSDGSKVYVDSVKGRFTISRDNAKNSVYLQMNSLRAEDTAVYYCYYFRTVSGSSMVYWGQGTLVTVSS

SEQ ID NO: 441 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYGDSVKGRFTISRDNAKNSVYLQMNSLRAEDTAVYYCYYFRTVSRSSMRYWGQGTLVTVSS

SEQ ID NO: 442 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELAAGISSDQSKVYADSAKGRFTISRDNAKNSVYLQMNSLRAEDTAVYYCYYFRTVSGSSMSYWGQGTLVTVSS

SEQ ID NO: 443 - protein, 119 aa, synthetic construct
  REGION 1..119: note = Description of Artificial Sequence: Synthetic polypeptide
EVQLVESGGGLVQPGGSLTLSCVASGGTSSINAMGWYRRAPGKQRELVAGISSDGSKVYSDSVKGRFTISRDNAKNSVYLQMNSLRAEDTAVYYCYYFRTVSGSSARYWGQGTLVTVSS

SEQ ID NO: 444 - protein, 119 aa, synthetic construct
  REGION 1..119: note = Description of Artificial Sequence: Synthetic polypeptide
EVQLVESGGGLVQPGGSLTLSCVASGSTRSINAMGWYRRAPGKQRELVAGISSDGSKVYADSVKGRFTISRDNAKNSVYLQMNSLRAEDTAVYYCYYFHTVSGSSMRYWGQGTLVTVSS

SEQ ID NO: 445 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVIADSVKGRFTISRDNAKNSVYLQMNSLRAEDTAVYYCYYFRTVLGSSMRYWGQGTLVTVSS

SEQ ID NO: 446 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVDADSVKGRFTISRDNAKNSVYLQMNSLRAEDTAVYYCYYFRTVSGSSMRYWGQGTLVTVSS

SEQ ID NO: 447 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYKDSVKGRFTISRDNAKNSVYLQMNSLRAEDTAVYYCYYFRNVSGSSMRYWGQGTLVTVSS

SEQ ID NO: 448 - protein, 119 aa, synthetic construct
  REGION 1..119: note = Description of Artificial Sequence: Synthetic polypeptide
EVQLVESGGGLVQPGGSLTLSCVASGNTSSINAMGWYRRAPGKQRELVAGISSNGSKVYADSVKGRFTISRDNAKNSVYLQMNSLRAEDTAVYYCYYFRTVTGSSMRYWGQGTLVTVSS

SEQ ID NO: 449 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGISSDGSKVYKDSVKGRFTISRDNAKNSVYLQMNSLRAEDTAVYYCYYFRTVSGSSMRYWGQGTLVTVSS

SEQ ID NO: 450 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVYADSVKGRFTISRDNAKNSVYLQMNSLRAEDTAVYYCYYFRTVKGSSMRYWGQGTLVTVSS

SEQ ID NO: 451 - protein, 119 aa, synthetic construct
  REGION 1..119: note = Description of Artificial Sequence: Synthetic polypeptide
EVQLVESGGGLVQPGGSLTLSCVASGLTSSINAMGWYRRAPGKQRELVAGISSDGSKVYQDSVKGRFTISRDNAKNSVYLQMNSLRAEDTAVYYCYYFRTNSGSSMRYWGQGTLVTVSS

SEQ ID NO: 452 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYAESVKGRFTISRDNAKNSVYLQMNSLRAEDTAVYYCYYFRTVSGASMRYWGQGTLVTVSS

SEQ ID NO: 453 - protein, 119 aa, synthetic construct
  REGION 1..119: note = Description of Artificial Sequence: Synthetic polypeptide
EVQLVESGGGLVQPGGSLTLSCVASGSTSSTNAMGWYRRAPGKQRELVAGISSDGSKVLADSVKGRFTISRDNAKNSVYLQMNSLRAEDTAVYYCYYFRTVNLSSMRYWGQGTLVTVSS

SEQ ID NO: 454 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKYYADSVKGRFTISRDNAKNSVYLQMNSLRAEDTAVYYCYYFRTVTGSSMRYWGQGTLVTVSS

SEQ ID NO: 455 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVYAVSVKGRFTISRDNAKNSVYLQMNSLRAEDTAVYYCYYFRKVSGSSARYWGQGTLVTVSS

SEQ ID NO: 456 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVVADSVKGRFTISRDNAKNSVYLQMNSLRAEDTAVYYCYYFRTYSGSSMRYWGQGTLVTVSS

SEQ ID NO: 457 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYADSVKGRFTISRDNAKNSVYLQMNSLRAEDTAVYYCYYFRTVSKSSMRYWGQGTLVTVSS

SEQ ID NO: 458 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFKTVSGSSMRYWGQGTLVTVSS

SEQ ID NO: 459 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELAAGISSDNSKVYADSVKGRFTISRDNAKNSVYLQMNSLRAEDTAVYYCYYFRTRSGSSMRYWGQGTLVTVSS

SEQ ID NO: 460 - protein, 119 aa, synthetic construct
  REGION 1..119: note = Description of Artificial Sequence: Synthetic polypeptide
EVQLVESGGGLVQPGGSLTLSCVASGSKSSINAMGWYRRAPGKQRELAAGISSDGSKVYAQSVKGRFTISRDNAKNSVYLQMNSLRAEDTAVYYCYYFRTSSGSSMRYWGQGTLVTVSS

SEQ ID NO: 461 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVYVDSVKGRFTISRDNAKNSVYLQMNSLRAEDTAVYYCYYFRFLSGSSMRYWGQGTLVTVSS

SEQ ID NO: 462 - protein, 119 aa, synthetic construct
  REGION 1..119: note = Description of Artificial Sequence: Synthetic polypeptide
EVQLVESGGGLVQPGGSLTLSCVASGSTSSINAFGWYRRAPGKQRELVAGISSDGSKVYSDSVKGRFTISRDNAKNSVYLQMNSLRAEDTAVYYCYYFRTVSGSSMRYWGQGTLVTVSS

SEQ ID NO: 463 - protein, 119 aa, synthetic construct
  REGION 1..119: note = Description of Artificial Sequence: Synthetic polypeptide
EVQLVESGGGLVQPGGSLTLSCVASGSTFSINAMGWYRRAPGKQRELVAGISSDGSKVLADSVKGRFTISRDNAKNSVYLQMNSLRAEDTAVYYCYYFRLVSGSSMRYWGQGTLVTVSS

SEQ ID NO: 464 - protein, 119 aa, synthetic construct
  REGION 1..119: note = Description of Artificial Sequence: Synthetic polypeptide
EVQLVESGGGLVQPGGSLTLSCVASGSTRSINAMGWYRRAPGKQRELVAGISSDGSKVYNDSVKGRFTISRDNAKNSVYLQMNSLRAEDTAVYYCYYFRTVSGSSMRFWGQGTLVTVSS

SEQ ID NO: 465 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVYNDSVKGRFTISRDNAKNSVYLQMNSLRAEDTAVYYCYYFRTQSGSSMRYWGQGTLVTVSS

SEQ ID NO: 466 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYVDSVKGRFTISRDNAKNSVYLQMNSLRAEDTAVYYCYYFRTVSGSSMPYWGQGTLVTVSS

SEQ ID NO: 467 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVVADSVKGRFTISRDNAKNSVYLQMNSLRAEDTAVYYCYYFRTLSGSSMRYWGQGTLVTVSS

SEQ ID NO: 468 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVYGDSVKGRFTISRDNAKNSVYLQMNSLRAEDTAVYYCYYFRTVSGSAMRYWGQGTLVTVSS

SEQ ID NO: 469 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYTDSVKGRFTISRDNAKNSVYLQMNSLRAEDTAVYYCYYFRTTSGSSMRYWGQGTLVTVSS

SEQ ID NO: 470 - protein, 119 aa, synthetic construct
  REGION 1..119: note = Description of Artificial Sequence: Synthetic polypeptide
EVQLVESGGGLVQPGGSLTLSCVASGRTSSINAMGWYRRAPGKQRELVAGISSDGSKVYNDSVKGRFTISRDNAKNSVYLQMNSLRAEDTAVYYCYYFRTVSGTSMRYWGQGTLVTVSS

SEQ ID NO: 471 - protein, 119 aa, synthetic construct
  REGION 1..119: note = Description of Artificial Sequence: Synthetic polypeptide
EVQLVESGGGLVQPGGSLTLSCVASGSTSSRNAMGWYRRAPGKQRELVAGISSDGSKVTADSVKGRFTISRDNAKNSVYLQMNSLRAEDTAVYYCYYFRTRSGSSMRYWGQGTLVTVSS

SEQ ID NO: 472 - protein, 119 aa, synthetic construct
  REGION 1..119: note = Description of Artificial Sequence: Synthetic polypeptide
EVQLVESGGGLVQPGGSLTLSCVASGSTKSINAMGWYRRAPGKQRELVAGISSDGSKVYRDSVKGRFTISRDNAKNSVYLQMNSLRAEDTAVYYCYYFRTSSGSSMRYWGQGTLVTVSS

SEQ ID NO: 473 - protein, 119 aa, synthetic construct
  REGION 1..119: note = Description of Artificial Sequence: Synthetic polypeptide
EVQLVESGGGLVQPGGSLTLSCVASGSTSSRNAMGWYRRAPGKQRELVAGISSNGSKVYSDSVKGRFTISRDNAKNSVYLQMNSLRAEDTAVYYCYYFRTVSGSSMSYWGQGTLVTVSS

SEQ ID NO: 474 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVYSDSVKGRFTISRDNAKNSVYLQMNSLRAEDTAVYYCYYFRPVSGSSMRYWGQGTLVTVSS

SEQ ID NO: 475 - protein, 119 aa, synthetic construct
  REGION 1..119: note = Description of Artificial Sequence: Synthetic polypeptide
EVQLVESGGGLVQPGGSLTLSCVASGSTSLINAMGWYRRAPGKQRELVAGISSDGSKVYADSVKGRFTISRDNAKNSVYLQMNSLRAEDTAVYYCYYFRHVSGSSMRYWGQGTLVTVSS

SEQ ID NO: 476 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTKSGSSMRYWGQGTLVTVSS

SEQ ID NO: 477 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSLVYADSVKGRFTISRDNAKNSVYLQMNSLRAEDTAVYYCYYFTTVSGSSMRYWGQGTLVTVSS

SEQ ID NO: 478 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGTKVYADSVKGRFTISRDNAKNSVYLQMNSLRAEDTAVYYCYYFHTVSGSSMRYWGQGTLVTVSS

SEQ ID NO: 479 - protein, 119 aa, synthetic construct
  REGION 1..119: note = Description of Artificial Sequence: Synthetic polypeptide
EVQLVESGGGLVQPGGSLTLSCVASGSTSSINAFGWYRRAPGKQRELVAGISSDGSKVYADSVKGRFTISRDNAKNSVYLQMNSLRAEDTAVYYCYYFRTVRGSSMRYWGQGTLVTVSS

SEQ ID NO: 480 - protein, 119 aa, synthetic construct
  REGION 1..119: note = Description of Artificial Sequence: Synthetic polypeptide
EVQLVESGGGLVQPGGSLTLSCVASGSTSSRNAMGWYRRAPGKQRELVAGISSDGSKLYLDSVKGRFTISRDNAKNSVYLQMNSLRAEDTAVYYCYYFRTVLGSSMRYWGQGTLVTVSS

SEQ ID NO: 481 - protein, 119 aa, synthetic construct
  REGION 1..119: note = Description of Artificial Sequence: Synthetic polypeptide
EVQLVESGGGLVQPGGSLTLSCVASGNTSSINAMGWYRRAPGKQRELVAGISSDGSRVYADSVKGRFTISRDNAKNSVYLQMNSLRAEDTAVYYCYYFRTVSGSSMRSWGQGTLVTVSS

SEQ ID NO: 482 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVYNDSVKGRFTISRDNAKNSVYLQMNSLRAEDTAVYYCYYFRTVRGSSMRYWGQGTLVTVSS

SEQ ID NO: 483 - protein, 119 aa, synthetic construct
  REGION 1..119: note = Description of Artificial Sequence: Synthetic polypeptide
EVQLVESGGGLVQPGGSLTLSCVASGSTASINAMGWYRRAPGKQRELVAGISSDGSKVYADSVKGRFTISRDNAKNSVYLQMNSLRAEDTAVYYCYYFRYVSGSSMRYWGQGTLVTVSS

SEQ ID NO: 484 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGSKVYVDSVKGRFTISRDNAKNSVYLQMNSLRAEDTAVYYCYYFRTVYGSSMRYWGQGTLVTVSS

SEQ ID NO: 485 - protein, 119 aa, synthetic construct
  REGION 1..119: note = Description of Artificial Sequence: Synthetic polypeptide
EVQLVESGGGLVQPGGSLTLSCVASGSTSSRNAMGWYRRAPGKQRELVAGISSDGSKLYADSVKGRFTISRDNAKNSVYLQMNSLRAEDTAVYYCYYFRTVLGSSMRYWGQGTLVTVSS

SEQ ID NO: 486 - protein, 119 aa, synthetic construct
  REGION 1..119: note = Description of Artificial Sequence: Synthetic polypeptide
EVQLVESGGGLVQPGGSLTLSCVASGSTNSINAMGWYRRAPGKQRELVAGISSDGSKVYKDSVKGRFTISRDNAKNSVYLQMNSLRAEDTAVYYCYYYRTVSGSSMRYWGQGTLVTVSS

SEQ ID NO: 487 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRSVSGSSMRYWGQGTLVTVSS

SEQ ID NO: 488 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGISSDGSKVYQDSVKGRFTISRDNAKNSVYLQMNSLRAEDTAVYYCYYFRRVSGSSMRYWGQGTLVTVSS

SEQ ID NO: 489 - protein, 119 aa, synthetic construct
  REGION 1..119: note = Description of Artificial Sequence: Synthetic polypeptide
EVQLVESGGGLVQPGGSLTLSCVPSGSTSNINAMGWYRRAPGKQRELPAGISSDGTKIYADSAKVPFTITRDNAKNSVYLQMNSLRAEDTAVYYCYYFRTVSGTSMRYWGQGTLVTVSS

SEQ ID NO: 490 - protein, 119 aa, synthetic construct
  REGION 1..119: note = Description of Artificial Sequence: Synthetic polypeptide
EVQLVESGGGLVQPGGSLTLSCVASGSTSKINAMGWYRRAPGKQRELVAGISSDRSKVYADSVKGRFTISRDNAKNSVYLQMNSLRAEDTAVYYCYYFRTVAGSSMRYWGQGTLVTVSS

SEQ ID NO: 491 - protein, 119 aa, synthetic construct
  REGION 1..119: note = Description of Artificial Sequence: Synthetic polypeptide
EVQLVESGGGLVQPGGSLTLSCVASGSTSSINALGWYRRAPGKQRELVAGISSDGSLVYADSVKGRFTISRDNAKNSVYLQMNSLRAEDTAVYYCYYFRIVSGSSMRYWGQGTLVTVSS

SEQ ID NO: 492 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRTASGSSMRYWGQGTLVTVSS

SEQ ID NO: 493 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYSDSVKGRFTISRDNAKNSVYLQMNSLRAEDTAVYYCYYFRTVSGSSSRYWGQGTLVTVSS

SEQ ID NO: 494 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGTKVYRDSVKGRFTISRDNAKNSVYLQMNSLRAEDTAVYYCYYFRTVQGSSMRYWGQGTLVTVSS

SEQ ID NO: 495 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELAAGISSDGSKVYNDSVKGRFTISRDNAKNSVYLQMNSLRAEDTAVYYCYYFRTVRGSSMRYWGQGTLVTVSS

SEQ ID NO: 496 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYADSVKGRFTISRDNAKNSVYLQMNSLRAEDTAVYYCYYFRTKSGSSMRYWGQGTLVTVSS

SEQ ID NO: 497 - protein, 119 aa, synthetic construct
  REGION 1..119: note = Description of Artificial Sequence: Synthetic polypeptide
EVQLVESGGGLVQPGGSLTLSCVASGTTSSINAMGWYRRAPGKQRELVAGISSDGSKVYADSVKGRFTISRDNAKNSVYLQMNSLRAEDTAVYYCYYFRTVWGSSMRYWGQGTLVTVSS

SEQ ID NO: 498 - protein, 119 aa, synthetic construct
  REGION 1..119: note = Description of Artificial Sequence: Synthetic polypeptide
EVQLVESGGGLVQPGGSLTLSCVASGKTSSINAMGWYRRAPGKQRELVAGISSDGSKVYTDSVKGRFTISRDNAKNSVYLQMNSLRAEDTAVYYCYYFRTRSGSSMRYWGQGTLVTVSS

SEQ ID NO: 499 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPFKQGELPAGISPDGTKAYADSAKVRFTISRDNAKNSVYLQMNSLRAEDTAVYYCYYFHTVCGTSMGYWGQGTLVTVSS

SEQ ID NO: 500 - protein, 119 aa, synthetic construct
  REGION 1..119: note = Description of Artificial Sequence: Synthetic polypeptide
EVQLVESGGGLVQPGGSLTLSCVASGSTSAINAMGWYRRAPGKQRELVAGISSDGSKVYVDSVKGRFTISRDNAKNSVYLQMNSLRAEDTAVYYCYYFRTVSGSSQRYWGQGTLVTVSS

SEQ ID NO: 501 - protein, 119 aa, synthetic construct
  REGION 1..119: note = Description of Artificial Sequence: Synthetic polypeptide
EVQLVESGGGLVQPGGSLTLSCVASGSPSSINAYGWYRRAPGKQRELVAGISSDGSKVYSDSVKGRFTISRDNAKNSVYLQMNSLRAEDTAVYYCYYFRTVSGSSMSYWGQGTLVTVSS

SEQ ID NO: 502 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYASSVKGRFTISRDNAKNSVYLQMNSLRAEDTAVYYCYYFRTVRGSSMRYWGQGTLVTVSS

SEQ ID NO: 503 - protein, 119 aa, synthetic construct
  REGION 1..119: note = Description of Artificial Sequence: Synthetic polypeptide
EVQLVESGGGLVQPGGSLTLSCVASGSRSSINAMGWYRRAPGKQRELVAGISADGSKVYADSVKGRFTISRDNAKNSVYLQMNSLRAEDTAVYYCYYFRTQSGSSMRYWGQGTLVTVSS

SEQ ID NO: 504 - protein, 119 aa, synthetic construct
  REGION 1..119: note = Description of Artificial Sequence: Synthetic polypeptide
EVQLVESGGGLVQPGGSLTLSCVASGSVSSINAMGWYRRAPGKQRELVAGISSDGSKVYASSAKGRFTISRDNAKNSVYLQMNSLRAEDTAVYYCYYFRTLSGSSMRYWGQGTLVTVSS

SEQ ID NO: 505 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFHTVSGSSMRYWGQGTLVTVSS

SEQ ID NO: 506 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSSVYADSVKGRFTISRDNAKNSVYLQMNSLRAEDTAVYYCYYFRRVSGSSMRYWGQGTLVTVSS

SEQ ID NO: 507 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSKVYSDSVKGRFTISRDNAKNSVYLQMNSLRAEDTAVYYCYYFRLVSGSSMRYWGQGTLVTVSS

SEQ ID NO: 508 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGSKVYAGSVKGRFTISRDNAKNSVYLQMNSLRAEDTAVYYCYYFRTVSGSYMRYWGQGTLVTVSS

SEQ ID NO: 509 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELAAGISSDNSKVYADSVKGRFTISRDNAKNSVYLQMNSLRAEDTAVYYCYYFRTVGGSSMRYWGQGTLVTVSS

SEQ ID NO: 510 - protein, 119 aa, synthetic construct
  REGION 1..119: note = Description of Artificial Sequence: Synthetic polypeptide
EVQLVESGGGLVQPGGSLTLSCVASGSTSSINAYGWYRRAPGKQRELVAGISSDGSAVYADSVKGRFTISRDNAKNSVYLQMNSLRAEDTAVYYCYYFRTHSGSSMRYWGQGTLVTVSS

SEQ ID NO: 511 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGSSVYADSVKGRFTISRDNAKNSVYLQMNSLRAEDTAVYYCYYFRTVSTSSMRYWGQGTLVTVSS

SEQ ID NO: 512 - protein, 119 aa, synthetic construct
  REGION 1..119: note = Description of Artificial Sequence: Synthetic polypeptide
EVQLVESGGGLVQPGGSLTLSCVASGSKSSINAMGWYRRAPGKQRELPAGISSNGTKVYADSAKVRFTISRDNAKNSVYLQMNSLRAEDTAVYYCYYFRTVLGTSMRYWGQGTLVTVSS

SEQ ID NO: 513 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKLYADSVKGRFTISRDNAKNSVYLQMNSLRAEDTAVYYCYYFRTVSGSSMRYWGQGTLVTVSS

SEQ ID NO: 514 - protein, 119 aa, synthetic construct
  REGION 1..119: note = Description of Artificial Sequence: Synthetic polypeptide
EVQLVESGGGLVQPGGSLTLSCVASGSVSSINAMGWYRRAPGKQRELVAGISSDGSKVYKDSVKGRFTISRDNAKNSVYLQMNSLRAEDTAVYYCYYFRTVSGSSMGYWGQGTLVTVSS

SEQ ID NO: 515 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSLVYADSVKGRFTISRDNAKNSVYLQMNSLRAEDTAVYYCYYFRTVSGSSMRAWGQGTLVTVSS

SEQ ID NO: 516 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSLVYADSVKGRFTISRDNAKNSVYLQMNSLRAEDTAVYYCYYFRILSGSSMRYWGQGTLVTVSS

SEQ ID NO: 517 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVQGSSMRYWGQGTLVTVSS

SEQ ID NO: 518 - protein, 119 aa, synthetic construct
  REGION 1..119: note = Description of Artificial Sequence: Synthetic polypeptide
EVQLVESGGGLVQPGGSLTLSCVASGSTSYINAMGWYRRAPGKQRELVAGISSDGSKVYADSVKGRFTISRDNAKNSVYLQMNSLRAEDTAVYYCYYFRTVSGQSMGYWGQGTLVTVSS

SEQ ID NO: 519 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGSTVYADSVKGRFTISRDNAKNSVYLQMNSLRAEDTAVYYCYYFRTVSGHSMRYWGQGTLVTVSS

SEQ ID NO: 520 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELAAGISKDGSKVYADSAKGRFTISRDNAKNSVYLQMNSLRAEDTAVYYCYYFRTVSGSSSRYWGQGTLVTVSS

SEQ ID NO: 521 - protein, 119 aa, synthetic construct
  REGION 1..119: note = Description of Artificial Sequence: Synthetic polypeptide
EVQLVESGGGLVQPGGSLTLSCVASGSPSSINAYGWYRRAPGKQRELVAGISSDGSKVYSDSVKGRFTISRDNAKNSVYLQMNSLRAEDTAVYYCYYFRTVSGSSYSYWGQGTLVTVSS

SEQ ID NO: 522 - protein, 119 aa, synthetic construct
  REGION 1..119: note = Description of Artificial Sequence: Synthetic polypeptide
EVQLVESGGGLVQPGGSLTLSCVASGSPSSINAYGWYRRAPGKQRELVAGISSDGSKVYSDSVKGRFTISRDNAKNSVYLQMNSLRAEDTAVYYCYYFRTVSGSSQSYWGQGTLVTVSS

SEQ ID NO: 523 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTDYIDSVKGRFTISRDNAKNSVYLQMNSLRAEDTAVYYCYAYRWTRRYTYWGQGTLVTVSS

SEQ ID NO: 524 - protein, 117 aa, synthetic construct
  REGION 1..117: note = Description of Artificial Sequence: Synthetic polypeptide
EVQLVESGGGLVQPGGSLTLSCAASGSSVSFLSLAWYRQAPGKKRELVAGISADGSTAYIDSVKGRFTISRDNAKNSVYLQMNSLRAEDTAVYYCYAYRWRTRYTYWGQGTLVTVSS

SEQ ID NO: 525 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISRDGSTDYIDSVKGRFTISRDNAKNSVYLQMNSLRAEDTAVYYCYAYRWTTRRTYWGQGTLVTVSS

SEQ ID NO: 526 - protein, 117 aa, synthetic construct
  REGION 1..117: note = Description of Artificial Sequence: Synthetic polypeptide
EVQLVESGGGLVQPGGSLTLSCAASGSQVSFLSMAWYRQAPGKKRELVAGISRDGSTDYIDSVKGRFTISRDNAKNSVYLQMNSLRAEDTAVYYCYAYRWTTRYIYWGQGTLVTVSS

SEQ ID NO: 527 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISEAGSTDYIDSVKGRFTISRDNAKNSVYLQMNSLRAEDTAVYYCYAYRWRTRYTYWGQGTLVTVSS

SEQ ID NO: 528 - protein, 117 aa, synthetic construct
  REGION 1..117: note = Description of Artificial Sequence: Synthetic polypeptide
EVQLVESGGGLVQPGGSLTLRCAASGSKVSFLSMAWYRQAPGKKRELVAGISADGSTDYVDSVKGRFTISRDNAKNSVYLQMNSLRAEDTAVYYCYAYRWTRRYTYWGQGTLVTVSS

SEQ ID NO: 529 - protein, 117 aa, synthetic construct
  REGION 1..117: note = Description of Artificial Sequence: Synthetic polypeptide
EVQLVESGGGLVQPGGSLTLSCAASGSSVGFLSMAWYRQAPGKKRELVAGISADGSTDYIRSVKGRFTISRDNAKNSVYLQMNSLRAEDTAVYYCYAYRWRTRYTYWGQGTLVTVSS

SEQ ID NO: 530 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSVDYIDSVKGRFTISRDNAKNSVYLQMNSLRAEDTAVYYCYAYRWTTRYIYWGQGTLVTVSS

SEQ ID NO: 531 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTLYIDSVKGRFTISRDNAKNSVYLQMNSLRAEDTAVYYCYAYRWTTRRTYWGQGTLVTVSS

SEQ ID NO: 532 - protein, 117 aa, synthetic construct
  REGION 1..117: note = Description of Artificial Sequence: Synthetic polypeptide
EVQLVESGGGLVQPGGSLTLSCAASGSSVSFLSLAWYRQAPGKKRELVAGISTDGSTDYIDSVKGRFTISRDNAKNSVYLQMNSLRAEDTAVYYCYAYRWRTRYTYWGQGTLVTVSS

SEQ ID NO: 533 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISGDGSTDYIDSVKGRFTISRDNAKNSVYLQMNSLRAEDTAVYYCYAYRWTTRYTYWGQGTLVTVSS

SEQ ID NO: 534 - protein, 117 aa, synthetic construct
  REGION 1..117: note = Description of Artificial Sequence: Synthetic polypeptide
EVQLVESGGGLVQPGGSLTLSCAASGSSVQFLSMAWYRQAPGKKRELVAGISADGSTDYINSVKGRFTISRDNAKNSVYLQMNSLRAEDTAVYYCYAYRWRTRYTYWGQGTLVTVSS

SEQ ID NO: 535 - protein, 117 aa, synthetic construct
  REGION 1..117: note = Description of Artificial Sequence: Synthetic polypeptide
EVQLVESGGGLVQPGGSLTLSCAASGSNVSFLSMAWYRQAPGKKRELVAGISARGSTDYIDSVKGRFTISRDNAKNSVYLQMNSLRAEDTAVYYCYAYHWTTRYTYWGQGTLVTVSS

SEQ ID NO: 536 - protein, 117 aa, synthetic construct
  REGION 1..117: note = Description of Artificial Sequence: Synthetic polypeptide
EVQLVESGGGLVQPGGSLTLSCVASGSSVKFLSMAWYRQAPGKKRELVAGISADGSTTYIDSVKGRFTISRDNAKNSVYLQMNSLRAEDTAVYYCYAYRWTTRRTYWGQGTLVTVSS

SEQ ID NO: 537 - protein, 117 aa, synthetic construct
  REGION 1..117: note = Description of Artificial Sequence: Synthetic polypeptide
EVQLVESGGGLVQPGGSLTLSCAASGKSVSFLSMAWYRQAPGKKRELVAGISKDGSTDYIDSVKGRFTISRDNAKNSVYLQMNSLRAEDTAVYYCYAYRWTTRYTYWGQGTLVTVSS

SEQ ID NO: 538 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTTYIDSVKGRFTISRDNAKNSVYLQMNSLRAEDTAVYYCYAYRWTTRYTYWGQGTLVTVSS

SEQ ID NO: 539 - protein, 117 aa, synthetic construct
  REGION 1..117: note = Description of Artificial Sequence: Synthetic polypeptide
EVQLVESGGGLVQPGGSLTLSCAASGSHVSFLSMAWYRQAPGKKRELVAGISANGSTDYIDSVKGRFTISRDNAKNSVYLQMNSLRAEDTAVYYCYAYRWTTRYAYWGQGTLVTVSS

SEQ ID NO: 540 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISRDGSTDYIDSVKGRFTISRDNAKNSVYLQMNSLRAEDTAVYYCYAYRWVTRYTYWGQGTLVTVSS

SEQ ID NO: 541 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSADYIDSVKGRFTISRDNAKNSVYLQMNSLRAEDTAVYYCYAYRWVTRYTYWGQGTLVTVSS

SEQ ID NO: 542 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISAHGSTDYIDSVKGRFTISRDNAKNSVYLQMNSLRAEDTAVYYCYAYRWSTRYTYWGQGTLVTVSS

SEQ ID NO: 543 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTIYIDSVKGRFTISRDNAKNSVYLQMNSLRAEDTAVYYCYAYRWRTRYTYWGQGTLVTVSS

SEQ ID NO: 544 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISRDGSTVYIDSVKGRFTISRDNAKNSVYLQMNSLRAEDTAVYYCYAYRWTTRGTYWGQGTLVTVSS

SEQ ID NO: 545 - protein, 117 aa, synthetic construct
  REGION 1..117: note = Description of Artificial Sequence: Synthetic polypeptide
EVQLVESGGGLVQPGGSLTLSCAASGSHVSFLSMAWYRQAPGKKRELVAGISADGPTDYIDSVKGRFTISRDNAKNSVYLQMNSLRAEDTAVYYCYAYRWDTRYTYWGQGTLVTVSS

SEQ ID NO: 546 - protein, 117 aa, synthetic construct
  REGION 1..117: note = Description of Artificial Sequence: Synthetic polypeptide
EVQLVESGGGLVQPGGSLTLSCVASGTSVSFLSMAWYRQAPGKKRELVAGISADGSTTYIDSVKGRFTISRDNAKNSVYLQMNSLRAEDTAVYYCYAYRWTTRRTYWGQGTLVTVSS

SEQ ID NO: 547 - protein, 117 aa, synthetic construct
  REGION 1..117: note = Description of Artificial Sequence: Synthetic polypeptide
EVQLVESGGGLVQPGGSLTLSCAASGTSVSFLSIAWYRQAPGKKRELVAGISADGSTDYIASVKGRFTISRDNAKNSVYLQMNSLRAEDTAVYYCYAYRWRTRYTYWGQGTLVTVSS

SEQ ID NO: 548 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISLDGSTDYIDSVKGRFTISRDNAKNSVYLQMNSLRAEDTAVYYCYAYRWTGRYTYWGQGTLVTVSS

SEQ ID NO: 549 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTIYIDSVKGRFTISRDNAKNSVYLQMNSLRAEDTAVYYCYAYRWRTRYTYWGQGTLVTVSS

SEQ ID NO: 550 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISAHGSTDYIDSVKGRFTISRDNAKNSVYLQMNSLRAEDTAVYYCYAYRWRTRYTYWGQGTLVTVSS

SEQ ID NO: 551 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISRDGSTDYIDSVKGRFTISRDNAKNSVYLQMNSLRAEDTAVYYCYAYRWITRYTYWGQGTLVTVSS

SEQ ID NO: 552 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWITRYTYWGQGTLVTVSS

SEQ ID NO: 553 - protein, 117 aa, synthetic construct
  REGION 1..117: note = Description of Artificial Sequence: Synthetic polypeptide
EVQLVESGGGLVQPGGSLTLSCAASGSSVVFLSMAWYRQAPGKKRELVAGISADGSMDYIDSVKGRFTISRDNAKNSVYLQMNSLRAEDTAVYYCYAYRWRTRYTYWGQGTLVTVSS

SEQ ID NO: 554 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTDYIDSVKGRFTISRDNAKNSVYLQMNSLRAEDTAVYYCYAYRWTRRYTYWGQGTLVTVSS

SEQ ID NO: 555 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTDYIDSVKGRFTISRDNAKNSVYLQMNSLRAEDTAVYYCYAYSWTTRYTYWGQGTLVTVSS

SEQ ID NO: 556 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISANGSTDYIDSVKGRFTISRDNAKNSVYLQMNSLRAEDTAVYYCYAYRWTNRYTYWGQGTLVTVSS

SEQ ID NO: 557 - protein, 117 aa, synthetic construct
  REGION 1..117: note = Description of Artificial Sequence: Synthetic polypeptide
EVQLVESGGGLVQPGGSLTLSCAASGSSVSRLSMAWYRQAPGKKRELVAGISANGSTTYIDSVKGRFTISRDNAKNSVYLQMNSLRAEDTAVYYCYAYRWTTRYRYWGQGTLVTVSS

SEQ ID NO: 558 - protein, 117 aa, synthetic construct
  REGION 1..117: note = Description of Artificial Sequence: Synthetic polypeptide
EVQLVESGGGLVQPGGSLTLSCAASGSSKSFLSMAWYRQAPGKKRELVAGISADGSTSYIDSVKGRFTISRDNAKNSVYLQMNSLRAEDTAVYYCYAYRWTTRYTYWGQGTLVTVSS

SEQ ID NO: 559 - protein, 117 aa, synthetic construct
  REGION 1..117: note = Description of Artificial Sequence: Synthetic polypeptide
EVQLVESGGGLVQPGGSLTLSCAASGSSVSRLSMAWYRQAPGKKRELVAGISADGSRDYIDSVKGRFTISRDNAKNSVYLQMNSLRAEDTAVYYCYAYRWTTRYKYWGQGTLVTVSS

SEQ ID NO: 560 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTMYIDSVKGRFTISRDNAKNSVYLQMNSLRAEDTAVYYCYAYRWHTRYTYWGQGTLVTVSS

SEQ ID NO: 561 - protein, 117 aa, synthetic construct
  REGION 1..117: note = Description of Artificial Sequence: Synthetic polypeptide
EVQLVESGGGLVQPGGSLTLSCAASGSGVRFLSMAWYRQAPGKKRELVAGISPDGSTDYIDSVKGRFTISRDNAKNSVYLQMNSLRAEDTAVYYCYAYRWTRRYTYWGQGTLVTVSS

SEQ ID NO: 562 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISGDGSTDYIDSVKGRFTISRDNAKNSVYLQMNSLRAEDTAVYYCYAYRWMTRYTYWGQGTLVTVSS

SEQ ID NO: 563 - protein, 117 aa, synthetic construct
  REGION 1..117: note = Description of Artificial Sequence: Synthetic polypeptide
EVQLVESGGGLVQPGGSLTLSCAASGSSVHFLSMAWYRQAPGKKRELVAGISRDGSTDYIDSVKGRFTISRDNAKNSVYLQMNSLRAEDTAVYYCYAYRWTTRYRYWGQGTLVTVSS

SEQ ID NO: 564 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWSTRYTYWGQGTLVTVSS

SEQ ID NO: 565 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISRDGSTDYIDSVKGRFTISRDNAKNSVYLQMNSLRAEDTAVYYCYAYRWTTRYTFWGQGTLVTVSS

SEQ ID NO: 566 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTDYIDSVKGRFTISRDNAKNSVYLQMNSLRAEDTAVYYCYAYRWRTRYTYWGQGTLVTVSS

SEQ ID NO: 567 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISTDGSTDYIDSVKGRFTISRDNAKNSVYLQMNSLRAEDTAVYYCYAYRWTTRRTYWGQGTLVTVSS

SEQ ID NO: 568 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTSYIDSVKGRFTISRDNAKNSVYLQMNSLRAEDTAVYYCYAYRWATRYTYWGQGTLVTVSS

SEQ ID NO: 569 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTLYIDSVKGRFTISRDNAKNSVYLQMNSLRAEDTAVYYCYAYRWHTRYTYWGQGTLVTVSS

SEQ ID NO: 570 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWGTRYTYWGQGTLVTVSS

SEQ ID NO: 571 - protein, 117 aa, synthetic construct
  REGION 1..117: note = Description of Artificial Sequence: Synthetic polypeptide
EVQLVESGGGLVQPGGSLTLSCAASYSSVSRLSMAWYRQAPGKKRELVAGISADGSTVYIDSVKGRFTISRDNAKNSVYLQMNSLRAEDTAVYYCYAYRWTTRNTYWGQGTLVTVSS

SEQ ID NO: 572 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISTDGSTDYIDSVKGRFTISRDNAKNSVYLQMNSLRAEDTAVYYCYAYRWRTRYTYWGQGTLVTVSS

SEQ ID NO: 573 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTLYIDSVKGRFTISRDNAKNSVYLQMNSLRAEDTAVYYCYAYRWTTRYAYWGQGTLVTVSS

SEQ ID NO: 574 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGRTDYIDSVKGRFTISRDNAKNSVYLQMNSLRAEDTAVYYCYAYRWRTRYTYWGQGTLVTVSS

SEQ ID NO: 575 - protein, 117 aa, synthetic construct
  REGION 1..117: note = Description of Artificial Sequence: Synthetic polypeptide
EVQLVESGGGLVQPGGSLTLSCVASGTSVSFLSMAWYRQAPGKKRELVAGISADGSTIYIDSVKGRFTISRDNAKNSVYLQMNSLRAEDTAVYYCYAYRWTTRRTYWGQGTLVTVSS

SEQ ID NO: 576 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTLYIDSVKGRFTISRDNAKNSVYLQMNSLRAEDTAVYYCYAYRWTTRRTYWGQGTLVTVSS

SEQ ID NO: 577 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISRDGSTDYIDSVKGRFTISRDNAKNSVYLQMNSLRAEDTAVYYCYAYRWTSRYTYWGQGTLVTVSS

SEQ ID NO: 578 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISKDGSTDYIDSVKGRFTISRDNAKNSVYLQMNSLRAEDTAVYYCYAYRWTTRVTYWGQGTLVTVSS

SEQ ID NO: 579 - protein, 117 aa, synthetic construct
  REGION 1..117: note = Description of Artificial Sequence: Synthetic polypeptide
EVQLVESGGGLVQPGGSLTLSCAASGSSVSVLSMAWYRQAPGKKRELVAGISADGSTDYIGSVKGRFTISRDNAKNSVYLQMNSLRAEDTAVYYCYAYRWTTRTTYWGQGTLVTVSS

SEQ ID NO: 580 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISVDGSTDYIDSVKGRFTISRDNAKNSVYLQMNSLRAEDTAVYYCYAYRWRTRYTYWGQGTLVTVSS

SEQ ID NO: 581 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTGYIDSVKGRFTISRDNAKNSVYLQMNSLRAEDTAVYYCYAYRWATRYTYWGQGTLVTVSS

SEQ ID NO: 582 - protein, 117 aa, synthetic construct
  REGION 1..117: note = Description of Artificial Sequence: Synthetic polypeptide
EVQLVESGGGLVQPGGSLTLSCVASGSSVKFLSMAWYRQAPGKKRELVAGISGDGSTTYIDSVKGRFTISRDNAKNSVYLQMNSLRAEDTAVYYCYAYRWTTRRTYWGQGTLVTVSS

SEQ ID NO: 583 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISTDGSTDYIDSVKGRFTISRDNAKNSVYLQMNSLRAEDTAVYYCYALRWTTRYTYWGQGTLVTVSS

SEQ ID NO: 584 - protein, 117 aa, synthetic construct
  REGION 1..117: note = Description of Artificial Sequence: Synthetic polypeptide
EVQLVESGGGLVQPGGSLTLSCAASGSSVSQLSMAWYRQAPGKKRELVAGISADGSTDYFDSVKGRFTISRDNAKNSVYLQMNSLRAEDTAVYYCYAYRWTTRGTYWGQGTLVTVSS

SEQ ID NO: 585 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTSYIDSVKGRFTISRDNAKNSVYLQMNSLRAEDTAVYYCYAYRWRTRYTYWGQGTLVTVSS

SEQ ID NO: 586 - protein, 117 aa, synthetic construct
  REGION 1..117: note = Description of Artificial Sequence: Synthetic polypeptide
EVQLVESGGGLVQPGGSLTLSCAASKSSVSFLSMAWYRQAPGKKRELVAGISADGSTDYIDSVKGRFTISRDNAKNSVYLQMNSLRAEDTAVYYCYAYRWTTRATYWGQGTLVTVSS

SEQ ID NO: 587 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTAYIDSVKGRFTISRDNAKNSVYLQMNSLRAEDTAVYYCYAYRWTTRRTYWGQGTLVTVSS

SEQ ID NO: 588 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTVYIDSVKGRFTISRDNAKNSVYLQMNSLRAEDTAVYYCYAYRWPTRYTYWGQGTLVTVSS

SEQ ID NO: 589 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISQDGSTDYIDSVKGRFTISRDNAKNSVYLQMNSLRAEDTAVYYCYAYRWRTRYTYWGQGTLVTVSS

SEQ ID NO: 590 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISNDGSTDYIDSVKGRFTISRDNAKNSVYLQMNSLRAEDTAVYYCYAYRWKTRYTYWGQGTLVTVSS

SEQ ID NO: 591 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISARGSTDYIDSVKGRFTISRDNAKNSVYLQMNSLRAEDTAVYYCYAYRWSTRYTYWGQGTLVTVSS

SEQ ID NO: 592 - protein, 117 aa, synthetic construct
  REGION 1..117: note = Description of Artificial Sequence: Synthetic polypeptide
EVQLVESGGGLVQPGGSLTLSCAASGSSVSFLSLAWYRQAPGKKRELVAGISADGSTDYIDSVKGRFTISRDNAKNSVYLQMNSLRAEDTAVYYCYAYRWKTRRTYWGQGTLVTVSS

SEQ ID NO: 593 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWTTRRTYWGQGTLVTVSS

SEQ ID NO: 594 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISADGSTLYIDSVKGRFTISRDNAKNSVYLQMNSLRAEDTAVYYCYAYRWTTRYRYWGQGTLVTVSS

SEQ ID NO: 595 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTNYIDSVKGRFTISRDNAKNSVYLQMNSLRAEDTAVYYCYAYRWRTRYTYWGQGTLVTVSS

SEQ ID NO: 596 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWTTRYKYWGQGTLVTVSS

SEQ ID NO: 597 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISADGSTTYIDSVKGRFTISRDNAKNSVYLQMNSLRAEDTAVYYCYAYRWKTRYTYWGQGTLVTVSS

SEQ ID NO: 598 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTDYIGSVKGRFTISRDNAKNSVYLQMNSLRAEDTAVYYCYAYRWTTRVTYWGQGTLVTVSS

SEQ ID NO: 599 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISRDGSTDYIDSVKGRFTISRDNAKNSVYLQMNSLRAEDTAVYYCYAYRWTTRFTYWGQGTLVTVSS

SEQ ID NO: 600 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTTYIDSVKGRFTISRDNAKNSVYLQMNSLRAEDTAVYYCYAYRWTTRFTYWGQGTLVTVSS

SEQ ID NO: 601 - protein, 117 aa, synthetic construct
  REGION 1..117: note = Description of Artificial Sequence: Synthetic polypeptide
EVQLVESGGGLVQPGGSLTLSCAASGSSVLFLSMAWYRQAPGKKRELVAGVSSDGSTDYIDSVKGRFTISRDNAKNSVYLQMNSLRAEDTAVYYCYAYRWRTRYTYWGQGTLVTVSS

SEQ ID NO: 602 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGHTDYIDSVKGRFTISRDNAKNSVYLQMNSLRAEDTAVYYCYAYRWTTRYTHWGQGTLVTVSS

SEQ ID NO: 603 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTDYFDSVKGRFTISRDNAKNSVYLQMNSLRAEDTAVYYCYAYRWTRRYTYWGQGTLVTVSS

SEQ ID NO: 604 - protein, 117 aa, synthetic construct
  REGION 1..117: note = Description of Artificial Sequence: Synthetic polypeptide
EVQLVESGGGLVQPGGSLTLSCAASGSSVGFLSMAWYRQAPGKKRELVAGISADGSTVYIDSVKGRFTISRDNAKNSVYLQMNSLRAEDTAVYYCYAYRWTTRYTYWGQGTLVTVSS

SEQ ID NO: 605 - protein, 117 aa, synthetic construct
  REGION 1..117: note = Description of Artificial Sequence: Synthetic polypeptide
EVQLVESGGGLVQPGGSLTLSCAASGSSVSFMSMAWYRQAPGKKRELVAGISADGSTDYIASVKGRFTISRDNAKNSVYLQMNSLRAEDTAVYYCYAYRWTTRSTYWGQGTLVTVSS

SEQ ID NO: 606 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTDYISSVKGRFTISRDNAKNSVYLQMNSLRAEDTAVYYCYAYSWTTRYTYWGQGTLVTVSS

SEQ ID NO: 607 - protein, 117 aa, synthetic construct
  REGION 1..117: note = Description of Artificial Sequence: Synthetic polypeptide
EVQLVESGGGLVQPGGSLTLSCAASGSSVTFLSMAWYRQAPGKKRELVAGISADGSTVYIDSVKGRFTISRDNAKNSVYLQMNSLRAEDTAVYYCYAYRWTTRGTYWGQGTLVTVSS

SEQ ID NO: 608 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWKTRYTYWGQGTLVTVSS

SEQ ID NO: 609 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISADGSTTYIDSVKGRFTISRDNAKNSVYLQMNSLRAEDTAVYYCYAYRWTTRFTYWGQGTLVTVSS

SEQ ID NO: 610 - protein, 117 aa, synthetic construct
  REGION 1..117: note = Description of Artificial Sequence: Synthetic polypeptide
EVQLVESGGGLVQPGGSLTLSCAASGSKVSFMSMAWYRQAPGKKRELVAGISVDGSTDYIDSVKGRFTISRDNAKNSVYLQMNSLRAEDTAVYYCYAYRWRTRYTYWGQGTLVTVSS

SEQ ID NO: 611 - protein, 117 aa, synthetic construct
  REGION 1..117: note = Description of Artificial Sequence: Synthetic polypeptide
EVQLVESGGGLVQPGGSLTLSCAASGSSVSNLSMAWYRQAPGKKRELVAGISADGSTAYIDSVKGRFTISRDNAKNSVYLQMNSLRAEDTAVYYCYAYRWTTRRTYWGQGTLVTVSS

SEQ ID NO: 612 - protein, 117 aa, synthetic construct
  REGION 1..117: note = Description of Artificial Sequence: Synthetic polypeptide
EVQLVESGGGLVQPGGSLTLSCAASNSSVSKLSMAWYRQAPGKKRELVAGISADGSTAYIDSVKGRFTISRDNAKNSVYLQMNSLRAEDTAVYYCYAYRWSTRYTYWGQGTLVTVSS

SEQ ID NO: 613 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISADGSKDYIDSVKGRFTISRDNAKNSVYLQMNSLRAEDTAVYYCYAYRWTTRLTYWGQGTLVTVSS

SEQ ID NO: 614 - protein, 117 aa, synthetic construct
  REGION 1..117: note = Description of Artificial Sequence: Synthetic polypeptide
EVQLVESGGGLVQPGGSLTLSCVASGSQVSFLSMAWYRQAPGKKRELVAGISADGSTDYFDSVKGRFTISRDNAKNSVYLQMNSLRAEDTAVYYCYAYRWTRRYTYWGQGTLVTVSS

SEQ ID NO: 615 - protein, 117 aa, synthetic construct
  REGION 1..117: note = Description of Artificial Sequence: Synthetic polypeptide
EVQLVESGGGLVQPGGSLTLSCAASGSKVSFMSMAWYRQAPGKKRELVAGISADGSTDYIDSVKGRFTISRDNAKNSVYLQMNSLRAEDTAVYYCYAYRWTTRLTYWGQGTLVTVSS

SEQ ID NO: 616 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTVYIDSVKGRFTISRDNAKNSVYLQMNSLRAEDTAVYYCYAYRWTRRYTYWGQGTLVTVSS

SEQ ID NO: 617 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWTTRRTYWGQGTLVTVSS

SEQ ID NO: 618 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISARGSTDYIDSVKGRFTISRDNAKNSVYLQMNSLRAEDTAVYYCYAYQWTTRYTYWGQGTLVTVSS

SEQ ID NO: 619 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISATGSTDYIDSVKGRFTISRDNAKNSVYLQMNSLRAEDTAVYYCYAYRWTRRYTYWGQGTLVTVSS

SEQ ID NO: 620 - protein, 117 aa, synthetic construct
  REGION 1..117: note = Description of Artificial Sequence: Synthetic polypeptide
EVQLVESGGGLVQPGGSLTLSCAASGSSVSFLSIAWYRQAPGKKRELVAGISKDGSTDYIDSVKGRFTISRDNAKNSVYLQMNSLRAEDTAVYYCYAYRWTTRMTYWGQGTLVTVSS

SEQ ID NO: 621 - protein, 117 aa, synthetic construct
  REGION 1..117: note = Description of Artificial Sequence: Synthetic polypeptide
EVQLVESGGGLVQPGGSLTLSCAASGSSSSFLSMAWYRQAPGKKRELVAGISADGSTVYIDSVKGRFTISRDNAKNSVYLQMNSLRAEDTAVYYCYAYRWTTRRTYWGQGTLVTVSS

SEQ ID NO: 622 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISPDGSTDYIDSVKGRFTISRDNAKNSVYLQMNSLRAEDTAVYYCYAYRWTTRYRYWGQGTLVTVSS

SEQ ID NO: 623 - protein, 117 aa, synthetic construct
  REGION 1..117: note = Description of Artificial Sequence: Synthetic polypeptide
EVQLVESGGGLVQPGGSLTLSCAASGSSVNFLSMAWYRQAPGKKRELVAGISADGSTHYIDSVKGRFTISRDNAKNSVYLQMNSLRAEDTAVYYCYAYRWLTRYTYWGQGTLVTVSS

SEQ ID NO: 624 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTDYILSVKGRFTISRDNAKNSVYLQMNSLRAEDTAVYYCYAYEWTTRYTYWGQGTLVTVSS

SEQ ID NO: 625 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISADGSTDYIHSVKGRFTISRDNAKNSVYLQMNSLRAEDTAVYYCYAYRWRTRYTYWGQGTLVTVSS

SEQ ID NO: 626 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISVDGSTDYIDSVKGRFTISRDNAKNSVYLQMNSLRAEDTAVYYCYAYRWRTRYTYWGQGTLVTVSS

SEQ ID NO: 627 - protein, 117 aa, synthetic construct
  REGION 1..117: note = Description of Artificial Sequence: Synthetic polypeptide
EVQLVESGGGLVQPGGSLTLSCAASGSSVSFLSVAWYRQAPGKKRELVAGISRDGSTDYIDSVKGRFTISRDNAKNSVYLQMNSLRAEDTAVYYCYAYRWRTRYTYWGQGTLVTVSS

SEQ ID NO: 628 - protein, 117 aa, synthetic construct
  REGION 1..117: note = Description of Artificial Sequence: Synthetic polypeptide
EVQLVESGGGLVQPGGSLTLSCAASGSQVSFLSMAWYRQAPGKKRELVAGISADGSTVYIDSVKGRFTISRDNAKNSVYLQMNSLRAEDTAVYYCYAYRWSTRYTYWGQGTLVTVSS

SEQ ID NO: 629 - protein, 117 aa, synthetic construct
  REGION 1..117: note = Description of Artificial Sequence: Synthetic polypeptide
EVQLVESGGGLVQPGGSLTLSCAASGTSVSFLSMAWYRQAPGKKRELVAGISADGSTDYIRSVKGRFTISRDNAKNSVYLQMNSLRAEDTAVYYCYAYRWTTRLTYWGQGTLVTVSS

SEQ ID NO: 630 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTMYIDSVKGRFTISRDNAKNSVYLQMNSLRAEDTAVYYCYAYRWTTRLTYWGQGTLVTVSS

SEQ ID NO: 631 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISTDGSTDYIDSVKGRFTISRDNAKNSVYLQMNSLRAEDTAVYYCYAYKWTTRYTYWGQGTLVTVSS

SEQ ID NO: 632 - protein, 117 aa, synthetic construct
  REGION 1..117: note = Description of Artificial Sequence: Synthetic polypeptide
EVQLVESGGGLVQPGGSLTLSCAASGSSVSFLSSAWYRQAPGKKRELVAGISADGSTLYIDSVKGRFTISRDNAKNSVYLQMNSLRAEDTAVYYCYAYRWTTRSTYWGQGTLVTVSS

SEQ ID NO: 633 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTDYIDSVKGRFTISRDNAKNSVYLQMNSLRAEDTAVYYCYAYRWRTRYTYWGQGTLVTVSS

SEQ ID NO: 634 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISATGSTDYIDSVKGRFTISRDNAKNSVYLQMNSLRAEDTAVYYCYAYRWSTRYTYWGQGTLVTVSS

SEQ ID NO: 635 - protein, 117 aa, synthetic construct
  REGION 1..117: note = Description of Artificial Sequence: Synthetic polypeptide
EVQLVESGGGLVQPGGSLTLSCAASGSTVSFLSMAWYRQAPGKKRELVAGISHDGSTDYIDSVKGRFTISRDNAKNSVYLQMNSLRAEDTAVYYCYAYRWTTRYTYWGQGTLVTVSS

SEQ ID NO: 636 - protein, 117 aa, synthetic construct
  REGION 1..117: note = Description of Artificial Sequence: Synthetic polypeptide
EVQLVESGGGLVQPGGSLTLSCAASGSSVQFLSMAWYRQAPGKKRELVAGISYDGSTDYIDSVKGRFTISRDNAKNSVYLQMNSLRAEDTAVYYCYAYRWRTRYTYWGQGTLVTVSS

SEQ ID NO: 637 - protein, 117 aa, synthetic construct
  REGION 1..117: note = Description of Artificial Sequence: Synthetic polypeptide
EVQLVESGGGLVQPGGSLTLSCAASRSSVSFLSMAWYRQAPGKKRELVAGISTDGSTDYIDSVKGRFTISRDNAKNSVYLQMNSLRAEDTAVYYCYAYRWLTRYTYWGQGTLVTVSS

SEQ ID NO: 638 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISADGSTAYIDSVKGRFTISRDNAKNSVYLQMNSLRAEDTAVYYCYAYRWRTRYTYWGQGTLVTVSS

SEQ ID NO: 639 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTDYIESVKGRFTISRDNAKNSVYLQMNSLRAEDTAVYYCYAYRWTTRYTYWGQGTLVTVSS

SEQ ID NO: 640 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISIDGSTDYIKSVKGRFTISRDNAKNSVYLQMNSLRAEDTAVYYCYAYRWTTRYRYWGQGTLVTVSS

SEQ ID NO: 641 - protein, 117 aa, synthetic construct
  REGION 1..117: note = Description of Artificial Sequence: Synthetic polypeptide
EVQLVESGGGLVQPGGSLTLSCAASGSKVSFLSMAWYRQAPGKKRELVAGISADGSKDYIDSVKGRFTISRDNAKNSVYLQMNSLRAEDTAVYYCYAYRWTTRYTYWGQGTLVTVSS

SEQ ID NO: 642 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWPTRYTYWGQGTLVTVSS

SEQ ID NO: 643 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISRDGSTDYIDSVKGRFTISRDNAKNSVYLQMNSLRAEDTAVYYCYAYRWTTRHTYWGQGTLVTVSS

SEQ ID NO: 644 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTDYIHSVKGRFTISRDNAKNSVYLQMNSLRAEDTAVYYCYAYRWTRRYTYWGQGTLVTVSS

SEQ ID NO: 645 - protein, 117 aa, synthetic construct
  REGION 1..117: note = Description of Artificial Sequence: Synthetic polypeptide
EVQLVESGGGLVQPGGSLTLSCAASGSSVSILSMAWYRQAPGKKRELVAGISADGSTIYIDSVKGRFTISRDNAKNSVYLQMNSLRAEDTAVYYCYAYRWHTRYTYWGQGTLVTVSS

SEQ ID NO: 646 - protein, 117 aa, synthetic construct
  REGION 1..117: note = Description of Artificial Sequence: Synthetic polypeptide
EVQLVESGGGLVQPGGSLTLSCAASGSSVSFLSVAWYRQAPGKKRELVAGISANGSTDYIDSVKGRFTISRDNAKNSVYLQMNSLRAEDTAVYYCYAYRWTNRYTYWGQGTLVTVSS

SEQ ID NO: 647 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISTDGSTDYIDSVKGRFTISRDNAKNSVYLQMNSLRAEDTAVYYCYAYRWTTRYRYWGQGTLVTVSS

SEQ ID NO: 648 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISYDGSTDYIDSVKGRFTISRDNAKNSVYLQMNSLRAEDTAVYYCYAYRWTTRRTYWGQGTLVTVSS

SEQ ID NO: 649 - protein, 117 aa, synthetic construct
  REGION 1..117: note = Description of Artificial Sequence: Synthetic polypeptide
EVQLVESGGGLVQPGGSLTLSCAASGHSVSFLSMAWYRQAPGKKRELVAGISADGSTDYIASVKGRFTISRDNAKNSVYLQMNSLRAEDTAVYYCYAYRWSTRYTYWGQGTLVTVSS

SEQ ID NO: 650 - protein, 117 aa, synthetic construct
  REGION 1..117: note = Description of Artificial Sequence: Synthetic polypeptide
EVQLVESGGGLVQPGGSLTLSCAASGSSVRFLSMAWYRQAPGKKRELVAGISADGSTDYIGSVKGRFTISRDNAKNSVYLQMNSLRAEDTAVYYCYAYRWSTRYTYWGQGTLVTVSS

SEQ ID NO: 651 - protein, 117 aa, synthetic construct
  REGION 1..117: note = Description of Artificial Sequence: Synthetic polypeptide
EVQLVESGGGLVQPGGSLTLSCAASGSSVSFLSMAWYRQAPGKKRELVAGISANGSTDYYDSVKGRFTISRDNAKNSVYLQMNSLRAEDTAVYYCYAYRWRTRYTYWGQGTLVTVSS

SEQ ID NO: 652 - protein, 117 aa, synthetic construct
  REGION 1..117: note = Description of Artificial Sequence: Synthetic polypeptide
EVQLVESGGGLVQPGGSLTLSCAASGSRVSFLSMAWYRQAPGKKRELVAGISADGSTSYIDSVKGRFTISRDNAKNSVYLQMNSLRAEDTAVYYCYAYRWTTRYTYWGQGTLVTVSS

SEQ ID NO: 653 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGVSADGSTDYIDSVKGRFTISRDNAKNSVYLQMNSLRAEDTAVYYCYAYEWTTRYTYWGQGTLVTVSS

SEQ ID NO: 654 - protein, 117 aa, synthetic construct
  REGION 1..117: note = Description of Artificial Sequence: Synthetic polypeptide
EVQLVESGGGLVQPGGSLTLSCAASGSSVSRLSMAWYRQAPGKKRELVAGISARGSTDYIDSVKGRFTISRDNAKNSVYLQMNSLRAEDTAVYYCYAYRWTTRSTYWGQGTLVTVSS

SEQ ID NO: 655 - protein, 117 aa, synthetic construct
  REGION 1..117: note = Description of Artificial Sequence: Synthetic polypeptide
EVQLVESGGGLVQPGGSLTLSCAASGRSVSFLSMAWYRQAPGKKRELVAGISADGSTIYIDSVKGRFTISRDNAKNSVYLQMNSLRAEDTAVYYCYAYRWTTRYTYWGQGTLVTVSS

SEQ ID NO: 656 - protein, 117 aa, synthetic construct
  REGION 1..117: note = Description of Artificial Sequence: Synthetic polypeptide
EVQLVESGGGLVQPGGSLTLSCAASGRSVSFLSMAWYRQAPGKKRELVAGISANGSTDYIDSVKGRFTISRDNAKNSVYLQMNSLRAEDTAVYYCYAYRWSTRYTYWGQGTLVTVSS

SEQ ID NO: 657 - protein, 117 aa, synthetic construct
  REGION 1..117: note = Description of Artificial Sequence: Synthetic polypeptide
EVQLVESGGGLVQPGGSLTLSCAASGSSVKFLSMAWYRQAPGKKRELVAGISADGSTDYVDSVKGRFTISRDNAKNSVYLQMNSLRAEDTAVYYCYAYRWSTRYTYWGQGTLVTVSS

SEQ ID NO: 658 - protein, 117 aa, synthetic construct
  REGION 1..117: note = Description of Artificial Sequence: Synthetic polypeptide
EVQLVESGGGLVQPGGSLTLSCAASGSSVSKLSMAWYRQAPGKKRELVAGISADGSTDYRDSVKGRFTISRDNAKNSVYLQMNSLRAEDTAVYYCYAYRWTYRYTYWGQGTLVTVSS

SEQ ID NO: 659 - protein, 117 aa, synthetic construct
  REGION 1..117: note = Description of Artificial Sequence: Synthetic polypeptide
EVQLVESGGGLVQPGGSLTLSCAASGSSVSRLSMAWYRQAPGKKRELVAGISVDGSTDYIDSVKGRFTISRDNAKNSVYLQMNSLRAEDTAVYYCYAYRWTTRLTYWGQGTLVTVSS

SEQ ID NO: 660 - protein, 117 aa, synthetic construct
  REGION 1..117: note = Description of Artificial Sequence: Synthetic polypeptide
EVQLVESGGGLVQPGGSLTLSCAASGSSVKFLSLAWYRQAPGKKRELVAGISADGSTDYILSVKGRFTISRDNAKNSVYLQMNSLRAEDTAVYYCYAYEWTTRYTYWGQGTLVTVSS

SEQ ID NO: 661 - protein, 117 aa, synthetic construct
  REGION 1..117: note = Description of Artificial Sequence: Synthetic polypeptide
EVQLVESGGGLVQPGGSLTLSCAASGSSVSRLSLAWYRQAPGKKRELVAGISVDGSTDYIDSVKGRFTISRDNAKNSVYLQMNSLRAEDTAVYYCYAYRWTTRLTYWGQGTLVTVSS

SEQ ID NO: 662 - protein, 119 aa, synthetic construct
  REGION 1..119: note = Description of Artificial Sequence: Synthetic polypeptide
EVQLVESGGGLVQPGGSLTLSCVASGTSSSINAMGWYRRAPGKQRELVAGISSDGSKVFNESVKGRFTISRDNAKNSVYLQMNSLRAEDTAVYYCYYFRPAAGSPMRYWGQGTLVTVSS

SEQ ID NO: 663 - protein, 119 aa, synthetic construct
  REGION 1..119: note = Description of Artificial Sequence: Synthetic polypeptide
EVQLVESGGGLVQPGGSLTLSCVASGTTSSINAIGWYRRAPGKQRELVAGISSDGSEVYTDSVKGRFTISRDNAKNSVYLQMNSLRAEDTAVYYCYYFRTVDGSPLRYWGQGTLVTVSS

SEQ ID NO: 664 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDDSNVYYESVKGRFTISRDNAKNSVYLQMNSLRAEDTAVYYCYYFRTVSGSSKRYWGQGTLVTVSS

SEQ ID NO: 665 - protein, 119 aa, synthetic construct
  REGION 1..119: note = Description of Artificial Sequence: Synthetic polypeptide
EVQLVESGGGLVQPGGSLTLSCVASGQTYRVNAFGWYRRAPGKQRELVAGISSDGSKVYADSVKGRFTISRDNAKNSVYLQMNSLRAEDTAVYYCYYFSAGSGTEMSYWGQGTLVTVSS

SEQ ID NO: 666 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDESTLYVDSVKGRFTISRDNAKNSVYLQMNSLRAEDTAVYYCYYFGSLSGSSTTYWGQGTLVTVSS

SEQ ID NO: 667 - protein, 119 aa, synthetic construct
  REGION 1..119: note = Description of Artificial Sequence: Synthetic polypeptide
EVQLVESGGGLVQPGGSLTLSCVASGSASLTNATGWYRRAPGKQRELVAGISSDDSKVYSDSVKGRFTISRDNAKNSVYLQMNSLRAEDTAVYYCYYFGSVSGSWTRYWGQGTLVTVSS

SEQ ID NO: 668 - protein, 119 aa, synthetic construct
  REGION 1..119: note = Description of Artificial Sequence: Synthetic polypeptide
EVQLVESGGGLVQPGGSLTLSCVASGYPSLNNAMGWYRRAPGKQRELVAGISSDGSQVYGASVKGRFTISRDNAKNSVYLQMNSLRAEDTAVYYCYYFRLVSGSSMSYWGQGTLVTVSS

SEQ ID NO: 669 - protein, 119 aa, synthetic construct
  REGION 1..119: note = Description of Artificial Sequence: Synthetic polypeptide
EVQLVESGGGLVQPGGSLTLSCVASGSSSTINAIGWYRRAPGKQRELVAGISSDGSKVYADSVKGRFTISRDNAKNSVYLQMNSLRAEDTAVYYCYYFRTGSGTSKSYWGQGTLVTVSS

SEQ ID NO: 670 - protein, 119 aa, synthetic construct
  REGION 1..119: note = Description of Artificial Sequence: Synthetic polypeptide
EVQLVESGGGLVQPGGSLTLSCVASGSTSYINAMGWYRRAPGKQRELVAGISSDGSNMYADSVKGRFTISRDNAKNSVYLQMNSLRAEDTAVYYCYYFSNMSGTTRRYWGQGTLVTVSS

SEQ ID NO: 671 - protein, 119 aa, synthetic construct
  REGION 1..119: note = Description of Artificial Sequence: Synthetic polypeptide
EVQLVESGGGLVQPGGSLTLSCVASGSTSSVNALGWYRRAPGKQRELVAGISSDGSKVYTDSVKGRFTISRDNAKNSVYLQMNSLRAEDTAVYYCYYFRTVPGSAMGYWGQGTLVTVSS

SEQ ID NO: 672 - protein, 119 aa, synthetic construct
  REGION 1..119: note = Description of Artificial Sequence: Synthetic polypeptide
EVQLVESGGGLVQPGGSLTLSCVASGSTSLSNAVGWYRRAPGKQRELVAGISSDGSKVSAESVKGRFTISRDNAKNSVYLQMNSLRAEDTAVYYCYYFRAESGSSMGYWGQGTLVTVSS

SEQ ID NO: 673 - protein, 119 aa, synthetic construct
  REGION 1..119: note = Description of Artificial Sequence: Synthetic polypeptide
EVQLVESGGGLVQPGGSLTLSCVASGSTSSTNAIGWYRRAPGKQRELVAGISSDGSKVYDDSVKGRFTISRDNAKNSVYLQMNSLRAEDTAVYYCYYFRTLYGSSRSYWGQGTLVTVSS

SEQ ID NO: 674 - protein, 119 aa, synthetic construct
  REGION 1..119: note = Description of Artificial Sequence: Synthetic polypeptide
EVQLVESGGGLVQPGGSLTLSCVASGLTSTINAMGWYRRAPGKQRELVAGISSDGSKVYDDSVKGRFTISRDNAKNSVYLQMNSLRAEDTAVYYCYYFSPFSGSDTGYWGQGTLVTVSS

SEQ ID NO: 675 - protein, 119 aa, synthetic construct
  REGION 1..119: note = Description of Artificial Sequence: Synthetic polypeptide
EVQLVESGGGLVQPGGSLTLSCVASGVSPSKNAIGWYRRAPGKQRELVAGISSDGSAVYVGSVKGRFTISRDNAKNSVYLQMNSLRAEDTAVYYCYYFSTFSGSSISYWGQGTLVTVSS

SEQ ID NO: 676 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGSYVYSESVKGRFTISRDNAKNSVYLQMNSLRAEDTAVYYCYYFRTLAGSEMRYWGQGTLVTVSS

SEQ ID NO: 677 - protein, 119 aa, synthetic construct
  REGION 1..119: note = Description of Artificial Sequence: Synthetic polypeptide
EVQLVESGGGLVQPGGSLTLSCVASGSTTMNNAMAWYRRAPGKQRELVAGISSDSSHVYADSVKGRFTISRDNAKNSVYLQMNSLRAEDTAVYYCYYFRTVSGSGVRYWGQGTLVTVSS

SEQ ID NO: 678 - protein, 119 aa, synthetic construct
  REGION 1..119: note = Description of Artificial Sequence: Synthetic polypeptide
EVQLVESGGGLVQPGGSLTLSCVASGSTSKINAIGWYRRAPGKQRELVAGISSDSSIVYTDSVKGRFTISRDNAKNSVYLQMNSLRAEDTAVYYCYYFRPGAGHSNSYWGQGTLVTVSS

SEQ ID NO: 679 - protein, 119 aa, synthetic construct
  REGION 1..119: note = Description of Artificial Sequence: Synthetic polypeptide
EVQLVESGGGLVQPGGSLTLSCVASGQTTALNAMGWYRRAPGKQRELVAGISSDGSEVNTDSVKGRFTISRDNAKNSVYLQMNSLRAEDTAVYYCYYFRRASGTAMSYWGQGTLVTVSS

SEQ ID NO: 680 - protein, 119 aa, synthetic construct
  REGION 1..119: note = Description of Artificial Sequence: Synthetic polypeptide
EVQLVESGGGLVQPGGSLTLSCVASGATSSINAIGWYRRAPGKQRELVAGISSDGSKLSSDSVKGRFTISRDNAKNSVYLQMNSLRAEDTAVYYCYYFTSASGTDLSYWGQGTLVTVSS

SEQ ID NO: 681 - protein, 119 aa, synthetic construct
  REGION 1..119: note = Description of Artificial Sequence: Synthetic polypeptide
EVQLVESGGGLVQPGGSLTLSCVASGSTSTINAMGWYRRAPGKQRELVAGISSDNSKVYADSVKGRFTISRDNAKNSVYLQMNSLRAEDTAVYYCYYFRSANGSSKRYWGQGTLVTVSS

SEQ ID NO: 682 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDGSRVYFDSVKGRFTISRDNAKNSVYLQMNSLRAEDTAVYYCYYFKTIAGAGMRYWGQGTLVTVSS

SEQ ID NO: 683 - protein, 119 aa, synthetic construct
  REGION 1..119: note = Description of Artificial Sequence: Synthetic polypeptide
EVQLVESGGGLVQPGGSLTLSCVASGSTSLVNAMGWYRRAPGKQRELVAGISSDGSLVYAESVKGRFTISRDNAKNSVYLQMNSLRAEDTAVYYCYYFRYGSGSSLSYWGQGTLVTVSS

SEQ ID NO: 684 - protein, 119 aa, synthetic construct
  REGION 1..119: note = Description of Artificial Sequence: Synthetic polypeptide
EVQLVESGGGLVQPGGSLTLSCVASGSTSLNNAIGWYRRAPGKQRELVAGISSDGSVVYVDSVKGRFTISRDNAKNSVYLQMNSLRAEDTAVYYCYYFRTVPGASMKYWGQGTLVTVSS

SEQ ID NO: 685 - protein, 119 aa, synthetic construct
  REGION 1..119: note = Description of Artificial Sequence: Synthetic polypeptide
EVQLVESGGGLVQPGGSLTLSCVASGSTSPVNAMAWYRRAPGKQRELVAGISSDGSKVYVDSVKGRFTISRDNAKNSVYLQMNSLRAEDTAVYYCYYFRTVDGSAISYWGQGTLVTVSS

SEQ ID NO: 686 - protein, 119 aa, synthetic construct
  REGION 1..119: note = Description of Artificial Sequence: Synthetic polypeptide
EVQLVESGGGLVQPGGSLTLSCVASGTTSSMNAIGWYRRAPGKQRELVAGISSDGSKLYDESVKGRFTISRDNAKNSVYLQMNSLRAEDTAVYYCYYFRTVKGSGGSYWGQGTLVTVSS

SEQ ID NO: 687 - protein, 119 aa, synthetic construct
  REGION 1..119: note = Description of Artificial Sequence: Synthetic polypeptide
EVQLVESGGGLVQPGGSLTLSCVASGETSSINAMAWYRRAPGKQRELVAGISSDYSKLYADSVKGRFTISRDNAKNSVYLQMNSLRAEDTAVYYCYYFRTVSGSSRGYWGQGTLVTVSS

SEQ ID NO: 688 - protein, 119 aa, synthetic construct
  REGION 1..119: note = Description of Artificial Sequence: Synthetic polypeptide
EVQLVESGGGLVQPGGSLTLSCVASGSTSTINAIGWYRRAPGKQRELVAGISSDSSKVYTESVKGRFTISRDNAKNSVYLQMNSLRAEDTAVYYCYYFRPGPGSQMAYWGQGTLVTVSS

SEQ ID NO: 689 - protein, 119 aa, synthetic construct
  REGION 1..119: note = Description of Artificial Sequence: Synthetic polypeptide
EVQLVESGGGLVQPGGSLTLSCVASGSTYSMNAMGWYRRAPGKQRELVAGISSDGSQVYVDSVKGRFTISRDNAKNSVYLQMNSLRAEDTAVYYCYYFRTVAGSASGYWGQGTLVTVSS

SEQ ID NO: 690 - protein, 119 aa, synthetic construct; identical to SEQ ID NO: 521
  REGION 1..119: note = Description of Artificial Sequence: Synthetic polypeptide

SEQ ID NO: 691 - protein, 119 aa, synthetic construct
  REGION 1..119: note = Description of Artificial Sequence: Synthetic polypeptide
EVQLVESGGGLVQPGGSLTLSCVASGSTSTINAIGWYRRAPGKQRELVAGISSDGSKVYVDSVKGRFTISRDNAKNSVYLQMNSLRAEDTAVYYCYYFINLKGSSMAYWGQGTLVTVSS

SEQ ID NO: 692 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDGSKVYADSVKGRFTISRDNAKNSVYLQMNSLRAEDTAVYYCYYFRMVTGSYGGYWGQGTLVTVSS

SEQ ID NO: 693 - protein, 119 aa, synthetic construct
  REGION 1..119: note = Description of Artificial Sequence: Synthetic polypeptide
EVQLVESGGGLVQPGGSLTLSCVASGSISSINAMGWYRRAPGKQRELVAGISSDGSSVYADSVKGRFTISRDNAKNSVYLQMNSLRAEDTAVYYCYYFKSSYGLPMRYWGQGTLVTVSS

SEQ ID NO: 694 - protein, 119 aa, synthetic construct
  REGION 1..119: note = Description of Artificial Sequence: Synthetic polypeptide
EVQLVESGGGLVQPGGSLTLSCVASGSTQVNNAMAWYRRAPGKQRELVAGISSDGSQVYYGSVKGRFTISRDNAKNSVYLQMNSLRAEDTAVYYCYYFKTVSGQSLRYWGQGTLVTVSS

SEQ ID NO: 695 - protein, 119 aa, synthetic construct
  REGION 1..119: note = Description of Artificial Sequence: Synthetic polypeptide
EVQLVESGGGLVQPGGSLTLSCVASGSTASFNAMAWYRRAPGKQRELVAGISSDGSKVYTDSVKGRFTISRDNAKNSVYLQMNSLRAEDTAVYYCYYFRTVTGRAARYWGQGTLVTVSS

SEQ ID NO: 696 - protein, 119 aa, synthetic construct
  REGION 1..119: note = Description of Artificial Sequence: Synthetic polypeptide
EVQLVESGGGLVQPGGSLTLSCVASGSPLSINAIGWYRRAPGKQRELVAGISSDGSKVSADSVKGRFTISRDNAKNSVYLQMNSLRAEDTAVYYCYYFGPAIGASRTYWGQGTLVTVSS

SEQ ID NO: 697 - protein, 119 aa, synthetic construct
  REGION 1..119: note = Description of Artificial Sequence: Synthetic polypeptide
EVQLVESGGGLVQPGGSLTLSCVASGSTTFINAIGWYRRAPGKQRELVAGISSDGSKVYEDSVKGRFTISRDNAKNSVYLQMNSLRAEDTAVYYCYYFRTVSGAPKSYWGQGTLVTVSS

SEQ ID NO: 698 - protein, 119 aa, synthetic construct
  REGION 1..119: note = Description of Artificial Sequence: Synthetic polypeptide
EVQLVESGGGLVQPGGSLTLSCVASGSTSSINAIGWYRRAPGKQRELVAGISSDRSKVYADSVKGRFTISRDNAKNSVYLQMNSLRAEDTAVYYCYYFHTVSGSSMSYWGQGTLVTVSS

SEQ ID NO: 699 - protein, 119 aa, synthetic construct
  REGION 1..119: note = Description of Artificial Sequence: Synthetic polypeptide
EVQLVESGGGLVQPGGSLTLSCVASGETDTINAVGWYRRAPGKQRELVAGISSDGSKVYAESVKGRFTISRDNAKNSVYLQMNSLRAEDTAVYYCYYFRRLEGYSNRYWGQGTLVTVSS

SEQ ID NO: 700 - protein, 119 aa, synthetic construct
  REGION 1..119: note = Description of Artificial Sequence: Synthetic polypeptide
EVQLVESGGGLVQPGGSLTLSCVASGSTSPINAIGWYRRAPGKQRELVAGISSDGSVVTTESVKGRFTISRDNAKNSVYLQMNSLRAEDTAVYYCYYFRTGSGSSMGYWGQGTLVTVSS

SEQ ID NO: 701 - protein, 119 aa, synthetic construct
  REGION 1..119: note = Description of Artificial Sequence: Synthetic polypeptide
EVQLVESGGGLVQPGGSLTLSCVASGSITSSNAMGWYRRAPGKQRELVAGISSDGSHVHQESVKGRFTISRDNAKNSVYLQMNSLRAEDTAVYYCYYFTTVTGSSMSYWGQGTLVTVSS

SEQ ID NO: 702 - protein, 117 aa, synthetic construct
  REGION 1..117: note = Description of Artificial Sequence: Synthetic polypeptide
EVQLVESGGGLVQPGGSLTLSCAASRYSVSNLSMAWYRQAPGKKRELVAGISADGSTVYVESVKGRFTISRDNAKNSVYLQMNSLRAEDTAVYYCYAYYWTERRPYWGQGTLVTVSS

SEQ ID NO: 703 - protein, 117 aa, synthetic construct
  REGION 1..117: note = Description of Artificial Sequence: Synthetic polypeptide
EVQLVESGGGLVQPGDSLTLSCAASMSTVSVLSMAWYRQAPGKKRELVAGISSDGSTVYIDSVKGRFTISRDNAKNSVYLQMNSLRAEDTAIYYCYAYSWDDAHPYWGQGTLVTVSS

SEQ ID NO: 704 - protein, 117 aa, synthetic construct
  REGION 1..117: note = Description of Artificial Sequence: Synthetic polypeptide
EVQLVESGGGLVQPGGSLTLSCAASDSYVSLLSMAWYRQAPGKKRELVAGISVDGSTHYVASVKGRFTISRDNAKNSVYLQMNSLRAEDTAVYYCYAYRWMTRLTYWGQGTLVTVSS

SEQ ID NO: 705 - protein, 117 aa, synthetic construct
  REGION 1..117: note = Description of Artificial Sequence: Synthetic polypeptide
EVQLVESGGGLVQPGGSLTLSCAASDSAVSVLSIAWYRQAPGKKRELVAGISTDGSKHYIDSVKGRFTISRDNAKNSVYLQMNSLRAEDTAVYYCYAYDWADAQPYWGQGTLVTVSS

SEQ ID NO: 706 - protein, 117 aa, synthetic construct
  REGION 1..117: note = Description of Artificial Sequence: Synthetic polypeptide
EVQLVESGGGLVQPGGSLTLSCAASHSSVTSLSLAWYRQAPGKKRELVAGISYDGSKYYAESVKGRFTISRDNAKNSVYLQMNSLRAEDTAVYYCYAYSWTDRLPYWGQGTLVTVSS

SEQ ID NO: 707 - protein, 117 aa, synthetic construct
  REGION 1..117: note = Description of Artificial Sequence: Synthetic polypeptide
EVQLVESGGGLVQPGGSLTLSCAASDSVVKFLSMAWYRQAPGKKRELVAGISANGSRTYMESVKGRFTISRDNAKNSVYLQMNSLRAEDTAVYYCYAYRWATRLPYWGQGTLVTVSS

SEQ ID NO: 708 - protein, 117 aa, synthetic construct
  REGION 1..117: note = Description of Artificial Sequence: Synthetic polypeptide
EVQLVESGGGLVQPGGSLTLSCAASDPSVWNLSMAWYRQAPGKKRELVAGISPDGSTDYVDSVKGRFTISRDNAKNSVYLQMNSLRAEDTAVYYCYAYKWSNRLPYWGQGTLVTVSS

SEQ ID NO: 709 - protein, 117 aa, synthetic construct
  REGION 1..117: note = Description of Artificial Sequence: Synthetic polypeptide
EVQLVESGGGLVQPGGSLTLSCAASGTSVMLLSLAWYRQAPGKKRELVAGISPNGSAVYTESVKGRFTISRDNAKNSVYLQMNSLRAEDTAVYYCYAYGWKTRQPYWGQGTLVTVSS

SEQ ID NO: 710 - protein, 117 aa, synthetic construct
  REGION 1..117: note = Description of Artificial Sequence: Synthetic polypeptide
EVQLVESGGGLVQPGGSLTLSCAASSSPVSNLSLAWYRQAPGKKRELVAGISPDGSTAYMESVKGRFTISRDNAKNSVYLQMNSLRAEDTAVYYCYAYRWPNRRGYWGQGTLVTVSS

SEQ ID NO: 711 - protein, 117 aa, synthetic construct
  REGION 1..117: note = Description of Artificial Sequence: Synthetic polypeptide
EVQLVESGGGLVQPGGSLTLSCAASWRSVLLLSVAWYRQAPGKKRELVAGISNDGSTDYIDSVKGRFTISRDNAKNSVYLQMNSLRAEDTAVYYCYAYDWTTRQRYWGQGTLVTVSS

SEQ ID NO: 712 - protein, 117 aa, synthetic construct
  REGION 1..117: note = Description of Artificial Sequence: Synthetic polypeptide
EVQLVESGGGLVQPGGSLTLSCAASSSSVQYLSMAWYRQAPGKKRELVAGISTDGSAVYFDSVKGRFTISRDNAKNSVYLQMNSLRAEDTAVYYCYAYNWSYAQPYWGQGTLVTVSS

SEQ ID NO: 713 - protein, 117 aa, synthetic construct
  REGION 1..117: note = Description of Artificial Sequence: Synthetic polypeptide
EVQLVESGGGLVQPGGSLTLSCAASGTSVSLLSLAWYRQAPGKKRELVAGISTGGSTHYIESVKGRFTISRDNAKNSVYLQMNSLRAEDTAVYYCYAYNWTDSLQYWGQGTLVTVSS

SEQ ID NO: 714 - protein, 117 aa, synthetic construct
  REGION 1..117: note = Description of Artificial Sequence: Synthetic polypeptide
EVQLVESGGGLVQPGGSLTLSCAASLSSVSNLSIAWYRQAPGKKRELVAGISTDGSTVYIDSVKGRFTISRDNAKNSVYLQMNSLRAEDTAVYYCYAYSWTTSLPYWGQGTLVTVSS

SEQ ID NO: 715 - protein, 117 aa, synthetic construct
  REGION 1..117: note = Description of Artificial Sequence: Synthetic polypeptide
EVQLVESGGGLVQPGGSLTLSCAASMYSVSFLSMAWYRQAPGKKRELVAGISNEGSTYYMDSVKGRFTISRDNAKNSVYLQMNSLRAEDTAVYYCYAYKWRSRSTYWGQGTLVTVSS

SEQ ID NO: 716 - protein, 117 aa, synthetic construct
  REGION 1..117: note = Description of Artificial Sequence: Synthetic polypeptide
EVQLVESGGGLVQPGGSLTLSCAASKSSVSHLSLAWYRQAPGKKRELVAGISADGSHVYTNSVKGRFTISRDNAKNSVYLQMNSLRAEDTAVYYCYAYSQTTRDPYWGQGTLVTVSS

SEQ ID NO: 717 - protein, 117 aa, synthetic construct
  REGION 1..117: note = Description of Artificial Sequence: Synthetic polypeptide
EVQLVESGGGLVQPGGSLTLSCAASYTSVLDLSIAWYRQAPGKKRELVAGISDDGSRYYTDSVKGRFTISRDNAKNSVYLQMNSLRAEDTAVYYCYAYRWTARDTYWGQGTLVTVSS

SEQ ID NO: 718 - protein, 117 aa, synthetic construct
  REGION 1..117: note = Description of Artificial Sequence: Synthetic polypeptide
EVQLVESGGGLVQPGGSLTLSCAASMSDVSFLSMAWYRQAPGKKRELVAGISAEGSTLYMESVKGRFTISRDNAKNSVYLQMNSLRAEDTAVYYCYAYRWTSRLSYWGQGTLVTVSS

SEQ ID NO: 719 - protein, 117 aa, synthetic construct
  REGION 1..117: note = Description of Artificial Sequence: Synthetic polypeptide
EVQLVESGGGLVQPGGSLTLSCAASESSVSFLSSAWYRQAPGKKRELVAGISTDGSTVYIDSVKGRFTISRDNAKNSVYLQMNSLRAEDTAVYYCYAYSWTTRSRYWGQGTLVTVSS

SEQ ID NO: 720 - protein, 117 aa, synthetic construct
  REGION 1..117: note = Description of Artificial Sequence: Synthetic polypeptide
EVQLVESGGGLVQPGGSLTLSCAASGDSVSLLSMAWYRQAPGKKRELVAGISANGSTSYIDSVKGRFTISRDNAKNSVYLQMNSLRAEDTAVYYCYAYNWTSRYRYWGQGTLVTVSS

SEQ ID NO: 721 - protein, 117 aa, synthetic construct
  REGION 1..117: note = Description of Artificial Sequence: Synthetic polypeptide
EVQLVESGGGLVQPGGSLTLSCAASGSDVWYLSLAWYRQAPGKKRELVAGISDDGSRHYIESVKGRFTISRDNAKNSVYLQMNSLRAEDTAVYYCYAYSWKTRFPYWGQGTLVTVSS

SEQ ID NO: 722 - protein, 117 aa, synthetic construct
  REGION 1..117: note = Description of Artificial Sequence: Synthetic polypeptide
EVQLVESGGGLVQPGGSLTLSCAASKSAVAFLSIAWYRQAPGKKRELVAGISPDGSTVYIESVKGRFTISRDNAKNSVYLQMNSLRAEDTAVYYCYAYSWTTRYPYWGQGTLVTVSS

SEQ ID NO: 723 - protein, 117 aa, synthetic construct
  REGION 1..117: note = Description of Artificial Sequence: Synthetic polypeptide
EVQLVESGGGLVQPGGSLTLSCAASFSAVAYLSMAWYRQAPGKKRELVAGISDDGSTVYVDSVKGRFTISRDNAKNSVYLQMNSLRAEDTAVYYCYAYEWTNALPYWGQGTLVTVSS

SEQ ID NO: 724 - protein, 117 aa, synthetic construct
  REGION 1..117: note = Description of Artificial Sequence: Synthetic polypeptide
EVQLVESGGGLVQPGGSLTLSCAASVYSVYDLSTAWYRQAPGKKRELVAGISDDGSTVYFDSVKGRFTISRDNAKNSVYLQMNSLRAEDTAVYYCYAYSWITRSPYWGQGTLVTVSS

SEQ ID NO: 725 - protein, 117 aa, synthetic construct
  REGION 1..117: note = Description of Artificial Sequence: Synthetic polypeptide
EVQLVESGGGLVQPGGSLTLSCAASGDSVSFLSMAWYRQAPGKKRELVAGISDEGSTVYIGSVKGRFTISRDNAKNSVYLQMNSLRAEDTAVYYCYAYSWTTRRQYWGQGTLVTVSS

SEQ ID NO: 726 - protein, 117 aa, synthetic construct
  REGION 1..117: note = Description of Artificial Sequence: Synthetic polypeptide
EVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSS

SEQ ID NO: 727 - protein, 117 aa, synthetic construct
  REGION 1..117: note = Description of Artificial Sequence: Synthetic polypeptide
EVQLVESGGGLVQPGGSLTLSCAASADSVSFLSIAWYRQAPGKKRELVAGISDDGSKHYFDSVKGRFTISRDNAKNSVYLQMNSLRAEDTAVYYCYAYRWEESRQYWGQGTLVTVSS

SEQ ID NO: 728 - protein, 117 aa, synthetic construct
  REGION 1..117: note = Description of Artificial Sequence: Synthetic polypeptide
EVQLVESGGGLVQPGGSLTLSCAASASSVTLLSIAWYRQAPGKKRELVAGISTDGSTDYLHSVKGRFTISRDNAKNSVYLQMNSLRAEDTAVYYCYAYTWTTRLPYWGQGTLVTVTS

SEQ ID NO: 729 - protein, 117 aa, synthetic construct; identical to SEQ ID NO: 727
  REGION 1..117: note = Description of Artificial Sequence: Synthetic polypeptide

SEQ ID NO: 730 - protein, 117 aa, synthetic construct
  REGION 1..117: note = Description of Artificial Sequence: Synthetic polypeptide
EVQLVESGGGLVQPGGSLTLSCAASGTSVWLLSMAWYRQAPGKKRELVAGISYDGSTVYVESVKGRFTISRDNAKNSVYLQMNSLRAEDTAVYYCYAYSWTTRQPYWGQGTLVTVSS

SEQ ID NO: 731 - protein, 117 aa, synthetic construct
  REGION 1..117: note = Description of Artificial Sequence: Synthetic polypeptide
EVQLVESGGGLVQPGGSLTLSCAASGSSVSILSIAWYRQAPGKKRELVAGISDDGSTVYIDSVKGRFTISRDNAKNSVYLQMNSLRAEDTAVYYCYAYVWGTRLPYWGQGTLVTVSS

SEQ ID NO: 732 - protein, 117 aa, synthetic construct
  REGION 1..117: note = Description of Artificial Sequence: Synthetic polypeptide
EVQLVESGGGLVQPGGSLTLSCAASGTAVSNLSIAWYRQAPGKKRELVAGISDDGSTVYVDSVKGRFTISRDNAKNSVYLQMNSLRAEDTAVYYCYAYEWTNALPYWGQGTLVTVSS

SEQ ID NO: 733 - protein, 117 aa, synthetic construct
  REGION 1..117: note = Description of Artificial Sequence: Synthetic polypeptide
EVQLVESGGGLVQPGGSLTLSCAASGSAVSMLSLAWYRQAPGKKRELVAGISDDGSQVYIDSVKGRFTISRDNAKNSVYLQMNSLRAEDTAVYYCYAYRWEDALTYWGQGTLVTVSS

SEQ ID NO: 734 - protein, 117 aa, synthetic construct
  REGION 1..117: note = Description of Artificial Sequence: Synthetic polypeptide
EVQLVESGGGLVQPGGSLTLSCAASGMTVFFLSMAWYRQAPGKKRELVAGISVDGSTVYSDSVKGRFTISRDNAKNSVYLQMNSLRAEDTAVYYCYAYSWTTRYPYWGQGTLVTVSS

SEQ ID NO: 735 - protein, 117 aa, synthetic construct
  REGION 1..117: note = Description of Artificial Sequence: Synthetic polypeptide
EVQLVESGGGLVQPGGSLTLSCAASQYSVTFLSVAWYRQAPGKKRELVAGISDDGSNVYIDSVKGRFTISRDNAKNSVYLQMNSLRAEDTAVYYCYAYSWIDSLRYWGQGTLVTVSS

SEQ ID NO: 736 - protein, 117 aa, synthetic construct
  REGION 1..117: note = Description of Artificial Sequence: Synthetic polypeptide
EVQLVESGGGLVQPGGSLTLSCAASGETVSFLSLAWYRQAPGKKRELVAGISTDGSTVYFVSVKGRFTISRDNAKNSVYLQMNSLRAEDTAVYYCYAYSWTTPRAYWGQGTLVTVSS

SEQ ID NO: 737 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTRDGNVDYAESVKGRFTISRDNAKNTVYLQMNSLKPEDTAVYYCNADLRTAVDLIRANYWGQGTQVTVSS

SEQ ID NO: 738 - protein, 125 aa, synthetic construct
  REGION 1..125: note = Description of Artificial Sequence: Synthetic polypeptide
QVQLQESGGGLVQAGDSLRLSCVVSGRTDSWYVMGWFRQAPGKDREFVAGVSWSYGNTYYADSVKGRFTASRDNAKNTAYLQMNSLNAEDTAVYYCAARVSREVIPTRWDLYNYWGQGTQVTVSS

SEQ ID NO: 739 - protein, 114 aa, synthetic construct
  REGION 1..114: note = Description of Artificial Sequence: Synthetic polypeptide
QVQLQESGGGSVQPGGSLRVSCVVSRTIISINAMTWYHQAPGKRRELVAIITSGGETNYADSVKGRFTISRDNAKNTAYLQMNNLKPEDTGVYYCNVVPPLGSWGQGTQVTVSS

SEQ ID NO: 740 - protein, 127 aa, synthetic construct
  REGION 1..127: note = Description of Artificial Sequence: Synthetic polypeptide
QVQLQESGGGRVQAGGSLRLSCSASARTLRLYAVGWFRQAPGKEREFVAGIGRSERTYYTDSVKGRFTLSRDNAKNTVFLEMNDLEPEDTAVYFCALTFQTTDMVDVPTTQHEYDYWGRGTQVTVSS

SEQ ID NO: 741 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTHDGNTDYADSVKGRFTISRDNVKNTVYLQMNSLKPEDTAVYYCNADLRTAVDLIRANYWGQGTQVTVSS

SEQ ID NO: 742 - protein, 121 aa, synthetic construct
  REGION 1..121: note = Description of Artificial Sequence: Synthetic polypeptide
QVQLQESGGGLVQPGGSLRLSCAASGSIAYIYTMDWYRQAPGKQRELVATSTRDGNTDYADSVKGRFTISRDNAKDTVYLQMNSLKPEDTAVYYCNADLRTAVDLIRANYWGQGTQVTVSS

SEQ ID NO: 743 - protein, 119 aa, synthetic construct
  REGION 1..119: note = Description of Artificial Sequence: Synthetic polypeptide
QVQLQESGGGLVQAGGSLTLSCAASGRYQMAWFRQAPEKEREFVGTISSGDSTWYTNSVKGRFAISRDSARNTVYLQMNDLKPEDTAIYYCAAALYYRDSRRAADYPYWGQGTQVTVSS

SEQ ID NO: 744 - protein, 127 aa, synthetic construct
  REGION 1..127: note = Description of Artificial Sequence: Synthetic polypeptide
QVQLQESGGGRVQAGESLRLSCSTSTRTLKLYAVGWFRQAPGKERDFVAGIGRSERIYYIDSVKGRFTLSRDNAKNTVFLEMNDLEPEDTAVYFCAATFQTSDNVGVPTVQHEYDYWGQGTQVTVSS

SEQ ID NO: 745 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTHDGNTDYADSVKGRFTISRDNAKNTVTLQMNSLKPEDTAVYYCNADLRTAVDLIRANYWGQGTQVTVSS

SEQ ID NO: 746 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATFTRDGNTDYADSVKGRFTISRDNAKNTVYLQMNSLKPEDTAVYYCNTDLRTAVDLIRANYWGQGTQVTVSS

SEQ ID NO: 747 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTHDGNTDYADSLKGRFTISRDNAKNTVYLQMNSLKPEDTAVYYCNADLRTAVDLIRANYWGQGTQVTVSS

SEQ ID NO: 748 - protein, 119 aa, synthetic construct
  REGION 1..119: note = Description of Artificial Sequence: Synthetic polypeptide
QVQLQESGGGSVQAGGSLRLSCAASGRYQMAWFRQAPEKEREFVGTISSGDSTWYTNSLKGRFAISRDSARDTVYLQMNDLKPEDTAVYYCAAALYYRDSRRAADYPYWGQGTQVTVSS

SEQ ID NO: 749 - protein, 114 aa, synthetic construct
  REGION 1..114: note = Description of Artificial Sequence: Synthetic polypeptide
QVQLQESGGGLVQTGGSLRLSCAVSGSIVTINAMTWYRQAPGKRRELVAIITSGGETNYADSVKGRFTISRDNAKNTAHLQMNSLNPEDTGVYYCNVVPPLGSWGQGTQVTVSS

SEQ ID NO: 750 - protein, 114 aa, synthetic construct
  REGION 1..114: note = Description of Artificial Sequence: Synthetic polypeptide
QVQLQESGGGLVQTGGSLRLSCAVSRSIVSIKSMTWYRQAPGKRRELVALITSGGETNYSDSVKGRFTISRDNAKNTVYLQMNSLKPEDTGVYYCNVVPPLGSWGQGTQVTVSS

SEQ ID NO: 751 - protein, 125 aa, synthetic construct
  REGION 1..125: note = Description of Artificial Sequence: Synthetic polypeptide
QVQLQESGGGLVETGGSLRLSCAGSGSTFRHHAMAWFRQTPGKEREFVSAINDHGDRTKYLDSVRGRFTISRDNTDNMVYLQMTDLRPEDTANYSCAAGPLVDYLETTPLVYTYWGHGTQVTVSS

SEQ ID NO: 752 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTHDGNTDYADSVKGRFTISRDNAKNTVYLQMNSLKPEDTAVYYCNADLRTAVDLIRANYWGQGTQVTVSS

SEQ ID NO: 753 - protein, 121 aa, synthetic construct
  REGION 1..121: note = Description of Artificial Sequence: Synthetic polypeptide
QVQLQESGGGSVQAGGSLTLSCAASGSIAYIYTMDWYRQAPGKQRELVATSTHDGNTDYADSVKGRFTISRDNAKNTVYLQMNSLKPEDTAVYYCNADLRTAVDLIRANYWGQGTQVTVSS

SEQ ID NO: 754 - protein, 113 aa, synthetic construct
  REGION 1..113: note = Description of Artificial Sequence: Synthetic polypeptide
QVQLQESGGGLVQPGGSLRLSCTASVSIFSVNAVDWYRQSPGKERELVAIMTSDGSTNYGDSVKGRFTISRDNAKNTVYLQMNNLKPEDTAVYYCNTVPPRYWGQGTQVTVSS

SEQ ID NO: 755 - protein, 121 aa, synthetic construct
  REGION 1..121: note = Description of Artificial Sequence: Synthetic polypeptide
QVQLQESGGGLVQPGGSLRLSCAASGSIAYIYTMDWYRQAPGKQRELVATSTRDGNIDYADSVKGRFTISRDSAKNTVYLQMSSLKPEDTAVYYCNADLRTAVDLIRANYWGQGTQVTVSS

SEQ ID NO: 756 - protein, 119 aa, synthetic construct
  REGION 1..119: note = Description of Artificial Sequence: Synthetic polypeptide
QVQLQESGGGLVQAGGSLTLSCAASGRYQLAWFRQAPEKVREFVGTISSGDSTWYTNSVKGRFAISRDSARNTVYLQMNDLKPEDTAVYYCAAALYYRDSRRAADYPYWGQGTQVTVSS

SEQ ID NO: 757 - protein, 119 aa, synthetic construct
  REGION 1..119: note = Description of Artificial Sequence: Synthetic polypeptide
QVQLQESGGGSVQAGGSLRLSCAASGRYHMAWFRQAPEKEREFVGTISSGDSTWYTNSVKGRFAISRDSARNTAYLQMNDLKPEDTAVYYCAAALYYGDSRRAADYPYWGQGTQVTVSS

SEQ ID NO: 758 - protein, 121 aa, synthetic construct
  REGION 1..121: note = Description of Artificial Sequence: Synthetic polypeptide
QVQLQESGGGSVQAGGSLRLSCAASGSIAYIYTMDWYRQAPGKQRELVATSTHDGNTDYTDSVKGRFTISRDNAKNTVYLQMNSLKPEDTAVYYCNADLRTAVDLIRANYWGQGTQVTVSS

SEQ ID NO: 759 - protein, 121 aa, synthetic construct
  REGION 1..121: note = Description of Artificial Sequence: Synthetic polypeptide
QVQLQESGGGLVQAGGSLRLSCAASGSIAYIYTMDWYRQTPGKQRELVATSTRDANTDYAGSVKGRFTISRDNAKDTVYLQMNSLKPEDTAVYYCHADLRTAVDLIRANYWGQGTQVTVSS

SEQ ID NO: 760 - protein, 119 aa, synthetic construct
  REGION 1..119: note = Description of Artificial Sequence: Synthetic polypeptide
QVQLQESGGGLVQAGGSLRLSCAASGRYNMAWFRQAPEKEREFVGTITSADSTWYTNSVKGRFAITQDSARNTVYLQMNDLKPEDTAVYYCAAALYYGDSRRAADYPYWGQGTQVTVSS

SEQ ID NO: 761 - protein, 119 aa, synthetic construct
  REGION 1..119: note = Description of Artificial Sequence: Synthetic polypeptide
QVQLQESGGGLVQPGGSLRLSCAASGRYQMAWFRQAPEKEREFVGTISSGDSTWYTNSVKGRFAISRDSARTTVYLQMNDLKPEDTAVYYCAAALYYRDSWRAADYPYWGQGTQVTVSS

SEQ ID NO: 762 - protein, 119 aa, synthetic construct
  REGION 1..119: note = Description of Artificial Sequence: Synthetic polypeptide
QVQLQESGGGLVQPGESLRLSCAATGRYHLAWFRQAPEKEREFVGTITSADSTWYTNSVKGRFAITRDSARNTVYLQMNDLKPEDTAVYYCAAALYYGDSRRAADYPYWGQGTQVTVSS

SEQ ID NO: 763 - protein, 124 aa, synthetic construct
  REGION 1..124: note = Description of Artificial Sequence: Synthetic polypeptide
QVQLQESGGGLVQAGGSLKLSCADSGRSFSNYIMGWFRQAPGKEREFVAGLGWSPGNTYYADSVKGRFTISRDNAKNMVYLQMNSLNPEDTAVYYCAARRGDVIYTTPWNYVYWGQGTQVTVSS

SEQ ID NO: 764 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTHDGNTDYADSVKGRFTISRDNAKNTVYLQMNSLKPEDTAVYYCNADLRTPVDLIRANYWGQGTQVTVSS

SEQ ID NO: 765 - protein, 119 aa, synthetic construct
  REGION 1..119: note = Description of Artificial Sequence: Synthetic polypeptide
QVQLQESGGGSVQAGGSLRLSCAAPGRYQMAWFRQAPEKEREFVGTISSGDSTWYTNSVKGRFAISRDSARNTVYLQMNDLKSEDTAVYYCAAALYYRDSRRAIDYPYWGQGTQVTVSS

SEQ ID NO: 766 - protein, 122 aa, synthetic construct
  REGION 1..122: note = Description of Artificial Sequence: Synthetic polypeptide
QVQLQESGGGSVQAGGSLRLSCAASGLTFSSYAMAWFRQAPGKQRELVARITSGGTTDYADSVKGRFTISRDNAKNTMYLQMNSLKPEDTAVYYCAADLTYRNLLLKLPHYWGQGTQVTVSS

SEQ ID NO: 767 - protein, 121 aa, synthetic construct
  REGION 1..121: note = Description of Artificial Sequence: Synthetic polypeptide
QVQLQESGGGLVQAGGSLRLSCAASGNIAYIYTMDWYRQAPGKQRELVATSTHDGSTDYADSVKGRFTISRDNAKNTVYLQMNSLKPEDTAVYYCNADLRTPVDLIRANYWGQGTQVTVSS

SEQ ID NO: 768 - protein, 121 aa, synthetic construct
  REGION 1..121: note = Description of Artificial Sequence: Synthetic polypeptide
QVQLQESGGGLVQPGGSLRLSCAASGSIAYIYTMDWYRQAPGKQRELVATSTWDGNTDYADSVKGRFTISRDNAKNTVYLQMNSLKPEDTAVYYCNADLRTAVDLIRANYWGQGTQVTVSS

SEQ ID NO: 769 - protein, 121 aa, synthetic construct
  REGION 1..121: note = Description of Artificial Sequence: Synthetic polypeptide
QVQLQESGGGLVQAGGSLRLSCAASGSIAYIYTMDWYRQAPGKQRELVATSTHDGNTDYADSVKGRFTISRDNAKNTVYLQMSSLKPDDTAVYYCNADLRTAVDLIRANYWGQGTQVTVSS

SEQ ID NO: 770 - protein, 123 aa, synthetic construct
  REGION 1..123: note = Description of Artificial Sequence: Synthetic polypeptide
QVQLQESGGGLVQAGESLSLSCAASGNDFVITDMHWYRQAPGKQREWVATITRFATTNYADSVKGRFTISRDNAKNTWYLQMNSLKPDDTAVYYCKAIGLRGVPDVNRQFEVWGQGTQVTVSS

SEQ ID NO: 771 - protein, 121 aa, synthetic construct
  REGION 1..121: note = Description of Artificial Sequence: Synthetic polypeptide
QVQLQESGGGLVQAGGSLRLSCAASGAIAYIYGMGWYRQAPGNQRELVAAISSGGSTDYADSVKGRFTISRDNAKNTVYLQMSSLKPEDTAVYYCNADVRTSRNLVRSDYWGQGTQVTVSS

SEQ ID NO: 772 - protein, 119 aa, synthetic construct
  REGION 1..119: note = Description of Artificial Sequence: Synthetic polypeptide
QVQLQESGGGLVQAGGSLRLSCAASGRYHTAWFRQAPEKEREFVGTISSGDSTWYTNSVKGRFAISRDSARNTVYLQMNDLKPEDTAVYYCAAALYYGDSRRAGDYPYWGQGTQVTVSS

SEQ ID NO: 773 - protein, 121 aa, synthetic construct
  REGION 1..121: note = Description of Artificial Sequence: Synthetic polypeptide
QVQLQESGGGLVQAGGSLRLSCAASGNIAYIYTMNWYRQAPGKQRELVATSTHAGNTDYADSVKGRFTISRDNAKNTVYLQMNSLKPEDTAVYYCNVDLRTAVDLIRANYWGQGTQVTVSS

SEQ ID NO: 774 - protein, 121 aa, synthetic construct
  REGION 1..121: note = Description of Artificial Sequence: Synthetic polypeptide
QVQLQESGGGLVQPGGSLRLSCAASGNIAYIYTMGWYRQAPGKQRELVATSTHDGNSDYADSVKGRFTISRDNAKNTVYLQMNTLKPDDTAVYYCNADLRTPVDRIRGNFWGQGTQVTVSS

SEQ ID NO: 775 - protein, 121 aa, synthetic construct
  REGION 1..121: note = Description of Artificial Sequence: Synthetic polypeptide
EVQLLESGGGLVQPGGSLTLSCAASGSIAYIYTMDWYRQAPGKQRELVATSTRDGNVDYADSVKGRFTISRDNSKNTLYLQMNSLRAEDTAVYYCNADLRTAVDLIRANYWGLGTQVTVSS

SEQ ID NO: 776 - protein, 125 aa, synthetic construct
  REGION 1..125: note = Description of Artificial Sequence: Synthetic polypeptide
EVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSS

SEQ ID NO: 777 - protein, 127 aa, synthetic construct
  REGION 1..127: note = Description of Artificial Sequence: Synthetic polypeptide
EVQLLESGGGLVQPGGSLTLSCAASARTLRLYAVGWFRQAPGKEREFVAGIGRSERTYYTDSVKGRFTISRDNSKNTLYLQMNSLRAEDTAVYYCALTFQTTDMVDVPTTQHEYDYWGLGTQVTVSS

SEQ ID NO: 778 - protein, 114 aa, synthetic construct
  REGION 1..114: note = Description of Artificial Sequence: Synthetic polypeptide
EVQLLESGGGLVQPGGSLTLSCAASGSIVTINAMTWYRQAPGKRRELVAIITSGGETNYADSVKGRFTISRDNSKNTLYLQMNSLRAEDTAVYYCNVVPPLGSWGLGTQVTVSS

SEQ ID NO: 779 - protein, 119 aa, synthetic construct
  REGION 1..119: note = Description of Artificial Sequence: Synthetic polypeptide
EVQLLESGGGLVQPGGSLTLSCAASGRYHMAWFRQAPGKEREFVGTISSGDSTWYTNSVKGRFTISRDNSKNTLYLQMNSLRAEDTAVYYCAAALYYGDSRRAADYPYWGLGTQVTVSS

SEQ ID NO: 780 - protein, 125 aa, synthetic construct
  REGION 1..125: note = Description of Artificial Sequence: Synthetic polypeptide
EVQLLESGGGLVQPGGSLTLSCAASGSTFRHHAMAWFRQTPGKEREFVSAINDHGDRTKYLDSVKGRFTISRDNSKNTLYLQMNSLRAEDTAVYYCAAGPLVDYLETTPLVYTYWGLGTQVTVSS

SEQ ID NO: 781 - protein, 124 aa, synthetic construct
  REGION 1..124: note = Description of Artificial Sequence: Synthetic polypeptide
EVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSS

SEQ ID NO: 782 - protein, 122 aa, synthetic construct
  REGION 1..122: note = Description of Artificial Sequence: Synthetic polypeptide
EVQLLESGGGLVQPGGSLTLSCAASGLTFSSYAMAWFRQAPGKQRELVARITSGGTTDYADSVKGRFTISRDNSKNTLYLQMNSLRAEDTAVYYCAADLTYRNLLLKLPHYWGLGTQVTVSS

SEQ ID NO: 783 - protein, 113 aa, synthetic construct
  REGION 1..113: note = Description of Artificial Sequence: Synthetic polypeptide
EVQLLESGGGLVQPGGSLTLSCAASVSIFSVNAVDWYRQSPGKERELVAIMTSDGSTNYDDSVKGRFTISRDNSKNTLYLQMNSLRAEDTAVYYCNTVPPRYWGLGTQVTVSS

SEQ ID NO: 784 - protein, 123 aa, synthetic construct
  REGION 1..123: note = Description of Artificial Sequence: Synthetic polypeptide
EVQLLESGGGLVQPGGSLTLSCAASGNDFVITDMHWYRQAPGKQREWVATITRFATTNYADSVKGRFTISRDNSKNTLYLQMNSLRAEDTAVYYCKAIGLRGVPDVNRQFEVWGLGTQVTVSS

SEQ ID NO: 785 - protein, 121 aa, synthetic construct
  REGION 1..121: note = Description of Artificial Sequence: Synthetic polypeptide
EVQLLESGGGLVQPGGSLTLSCAASGAIAYIYGMGWYRQAPGKQRELVAAISSGGSTDYADSVKGRFTISRDNSKNTLYLQMNSLRAEDTAVYYCNADVRTSRNLVRSDYWGLGTQVTVSS

SEQ ID NO: 786 - protein, 532 aa, synthetic construct
  REGION 1..532: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 787 - protein, 532 aa, synthetic construct
  REGION 1..532: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 788 - protein, 532 aa, synthetic construct
  REGION 1..532: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 789 - protein, 531 aa, synthetic construct
  REGION 1..531: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPPASSGRAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 790 - protein, 531 aa, synthetic construct
  REGION 1..531: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPIPVQGRAHGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 791 - protein, 532 aa, synthetic construct
  REGION 1..532: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 792 - protein, 503 aa, synthetic construct; identical to SEQ ID NO: 49
  REGION 1..503: note = Description of Artificial Sequence: Synthetic polypeptide

SEQ ID NO: 793 - protein, 513 aa, synthetic construct
  REGION 1..513: note = Description of Artificial Sequence: Synthetic polypeptide
EVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 794 - protein, 249 aa, synthetic construct; identical to SEQ ID NO: 56
  REGION 1..249: note = Description of Artificial Sequence: Synthetic polypeptide

SEQ ID NO: 795 - protein, 5 aa, Homo sapiens
APGKG

SEQ ID NO: 796 - protein, 143 aa, synthetic construct; identical to SEQ ID NO: 54
  REGION 1..143: note = Description of Artificial Sequence: Synthetic polypeptide

SEQ ID NO: 797 - protein, 143 aa, synthetic construct
  REGION 1..143: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGT

SEQ ID NO: 798 - protein, 143 aa, synthetic construct
  REGION 1..143: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGT

SEQ ID NO: 799 - protein, 142 aa, synthetic construct
  REGION 1..142: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPPASSGRAGGGGGT

SEQ ID NO: 800 - protein, 142 aa, synthetic construct
  REGION 1..142: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPIPVQGRAHGGGGT

SEQ ID NO: 801 - protein, 6 aa, Homo sapiens
QDGNEE

SEQ ID NO: 802 - protein, 9 aa, Homo sapiens
QDGNEEMGG

SEQ ID NO: 803 - protein, 9 aa, Homo sapiens
QDGNEEMGG

SEQ ID NO: 804 - protein, 119 aa, synthetic construct
  REGION 1..119: note = Description of Artificial Sequence: Synthetic polypeptide
QVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSS

SEQ ID NO: 805 - protein, 119 aa, synthetic construct
  REGION 1..119: note = Description of Artificial Sequence: Synthetic polypeptide
QVQLQESGGGLVQAGGSLRLSCAASGFIFRAASMGWFRQSPGNERELVATVSSGDFTNYADSVKGRFTISRDNAKNTVYLQMNSLKPEDTAVYFCGATFVRSDGHHTIYGQGTQVTVSS

SEQ ID NO: 806 - protein, 119 aa, synthetic construct
  REGION 1..119: note = Description of Artificial Sequence: Synthetic polypeptide
QVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSS

SEQ ID NO: 807 - protein, 119 aa, synthetic construct
  REGION 1..119: note = Description of Artificial Sequence: Synthetic polypeptide
QVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSS

SEQ ID NO: 808 - protein, 119 aa, synthetic construct
  REGION 1..119: note = Description of Artificial Sequence: Synthetic polypeptide
QVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSS

SEQ ID NO: 809 - protein, 124 aa, synthetic construct
  REGION 1..124: note = Description of Artificial Sequence: Synthetic polypeptide
QVQLQESGGGLVHTGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 810 - protein, 124 aa, synthetic construct
  REGION 1..124: note = Description of Artificial Sequence: Synthetic polypeptide
QVQLQESGGGLVQAGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 811 - protein, 124 aa, synthetic construct
  REGION 1..124: note = Description of Artificial Sequence: Synthetic polypeptide
QVQLQESGGGLVHTGGSLRLSCAFSGDTFLRYAMGWFRQAPGKEREFVAAITWNGGNTDYADSLKGRFTISRDNTKNTVYLQMNSLRPEDTAVYYCAADLTFGLASSHYQYDYWGQGTQVTVSS

SEQ ID NO: 812 - protein, 124 aa, synthetic construct
  REGION 1..124: note = Description of Artificial Sequence: Synthetic polypeptide
QVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 813 - protein, 112 aa, synthetic construct
  REGION 1..112: note = Description of Artificial Sequence: Synthetic polypeptide
QVQLQESGGGSVLAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSS

SEQ ID NO: 814 - protein, 112 aa, synthetic construct
  REGION 1..112: note = Description of Artificial Sequence: Synthetic polypeptide
QVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSS

SEQ ID NO: 815 - protein, 112 aa, synthetic construct
  REGION 1..112: note = Description of Artificial Sequence: Synthetic polypeptide
QVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSS

SEQ ID NO: 816 - protein, 112 aa, synthetic construct
  REGION 1..112: note = Description of Artificial Sequence: Synthetic polypeptide
QVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSS

SEQ ID NO: 817 - protein, 119 aa, synthetic construct
  REGION 1..119: note = Description of Artificial Sequence: Synthetic polypeptide
QVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSS

SEQ ID NO: 818 - protein, 119 aa, synthetic construct
  REGION 1..119: note = Description of Artificial Sequence: Synthetic polypeptide
  VARIANT 44: note = Any amino acid
QVQLQESGGGLVQPGGSLRLSCAASGNVFRAATMAWYRQAPEKXREMVATIASGGTTNYADFVKGRFTISRDNAKNTVYLQMNTLKPEDTAVYYCNAGYLTSLGPKNYWGQGTQVTVSS

SEQ ID NO: 819 - protein, 119 aa, synthetic construct
  REGION 1..119: note = Description of Artificial Sequence: Synthetic polypeptide
QVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSS

SEQ ID NO: 820 - protein, 111 aa, synthetic construct
  REGION 1..111: note = Description of Artificial Sequence: Synthetic polypeptide
QVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSS

SEQ ID NO: 821 - protein, 111 aa, synthetic construct
  REGION 1..111: note = Description of Artificial Sequence: Synthetic polypeptide
QVQLQESGGGLVQAGGSLRLSCAASGFAFGNHWMYWYRQAPGRGRELVASISSGGSTNYVDSVKGRFTISRDNARNTVYLQMYSLRPEDTAVYYCGTSDNWGQGTQVTVSS

SEQ ID NO: 822 - protein, 111 aa, synthetic construct
  REGION 1..111: note = Description of Artificial Sequence: Synthetic polypeptide
QVQLQESGGGLVQAGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSS

SEQ ID NO: 823 - protein, 119 aa, synthetic construct
  REGION 1..119: note = Description of Artificial Sequence: Synthetic polypeptide
QVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSS

SEQ ID NO: 824 - protein, 119 aa, synthetic construct
  REGION 1..119: note = Description of Artificial Sequence: Synthetic polypeptide
QVQLQESGGGLVQAGGSLRLSCAASGFIFRAASMGWYRQSPGNERELVATINSGGFTNYADSVKGRFTISRDNAKNTGYLQMNSLKPEDTAVYFCAATFLRSDGQPPIWGQGTQVTVSS

SEQ ID NO: 825 - protein, 122 aa, synthetic construct
  REGION 1..122: note = Description of Artificial Sequence: Synthetic polypeptide
QVQLQESGGGLVQAGGSLRLSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSS

SEQ ID NO: 826 - protein, 122 aa, synthetic construct
  REGION 1..122: note = Description of Artificial Sequence: Synthetic polypeptide
QVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSS

SEQ ID NO: 827 - protein, 122 aa, synthetic construct
  REGION 1..122: note = Description of Artificial Sequence: Synthetic polypeptide
QVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSS

SEQ ID NO: 828 - protein, 122 aa, synthetic construct
  REGION 1..122: note = Description of Artificial Sequence: Synthetic polypeptide
QVQLQESGGGLVQTGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSS

SEQ ID NO: 829 - protein, 119 aa, synthetic construct
  REGION 1..119: note = Description of Artificial Sequence: Synthetic polypeptide
QVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSS

SEQ ID NO: 830 - protein, 120 aa, synthetic construct
  REGION 1..120: note = Description of Artificial Sequence: Synthetic polypeptide
QVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSS

SEQ ID NO: 831 - protein, 121 aa, synthetic construct
  REGION 1..121: note = Description of Artificial Sequence: Synthetic polypeptide
QVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSS

SEQ ID NO: 832 - protein, 120 aa, synthetic construct
  REGION 1..120: note = Description of Artificial Sequence: Synthetic polypeptide
QVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSS

SEQ ID NO: 833 - protein, 121 aa, synthetic construct
  REGION 1..121: note = Description of Artificial Sequence: Synthetic polypeptide
QVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSS

SEQ ID NO: 834 - protein, 116 aa, synthetic construct
  REGION 1..116: note = Description of Artificial Sequence: Synthetic polypeptide
QVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSS

SEQ ID NO: 835 - protein, 120 aa, synthetic construct
  REGION 1..120: note = Description of Artificial Sequence: Synthetic polypeptide
QVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSS

SEQ ID NO: 836 - protein, 119 aa, synthetic construct
  REGION 1..119: note = Description of Artificial Sequence: Synthetic polypeptide
QVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSS

SEQ ID NO: 837 - protein, 124 aa, synthetic construct
  REGION 1..124: note = Description of Artificial Sequence: Synthetic polypeptide
QVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSS

SEQ ID NO: 838 - protein, 121 aa, synthetic construct
  REGION 1..121: note = Description of Artificial Sequence: Synthetic polypeptide
QVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSS

SEQ ID NO: 839 - protein, 127 aa, synthetic construct
  REGION 1..127: note = Description of Artificial Sequence: Synthetic polypeptide
QVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSS

SEQ ID NO: 840 - protein, 119 aa, synthetic construct
  REGION 1..119: note = Description of Artificial Sequence: Synthetic polypeptide
QVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSS

SEQ ID NO: 841 - protein, 122 aa, synthetic construct
  REGION 1..122: note = Description of Artificial Sequence: Synthetic polypeptide
QVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSS

SEQ ID NO: 842 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSSHHHHHH

SEQ ID NO: 843 - protein, 395 aa, synthetic construct
  REGION 1..395: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSSHHHHHH

SEQ ID NO: 844 - protein, 392 aa, synthetic construct
  REGION 1..392: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSSHHHHHH

SEQ ID NO: 845 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSSHHHHHH

SEQ ID NO: 846 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSSHHHHHH

SEQ ID NO: 847 - protein, 398 aa, synthetic construct
  REGION 1..398: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSSHHHHHH

SEQ ID NO: 848 - protein, 382 aa, synthetic construct
  REGION 1..382: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSSHHHHHH

SEQ ID NO: 849 - protein, 395 aa, synthetic construct
  REGION 1..395: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSSHHHHHH

SEQ ID NO: 850 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSSHHHHHH

SEQ ID NO: 851 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSSHHHHHH

SEQ ID NO: 852 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSSHHHHHH

SEQ ID NO: 853 - protein, 383 aa, synthetic construct
  REGION 1..383: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSSHHHHHH

SEQ ID NO: 854 - protein, 393 aa, synthetic construct
  REGION 1..393: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSSHHHHHH

SEQ ID NO: 855 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSSHHHHHH

SEQ ID NO: 856 - protein, 393 aa, synthetic construct
  REGION 1..393: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSSHHHHHH

SEQ ID NO: 857 - protein, 393 aa, synthetic construct
  REGION 1..393: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSSHHHHHH

SEQ ID NO: 858 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSSHHHHHH

SEQ ID NO: 859 - protein, 383 aa, synthetic construct
  REGION 1..383: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSSHHHHHH

SEQ ID NO: 860 - protein, 391 aa, synthetic construct
  REGION 1..391: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSSHHHHHH

SEQ ID NO: 861 - protein, 392 aa, synthetic construct
  REGION 1..392: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSSHHHHHH

SEQ ID NO: 862 - protein, 391 aa, synthetic construct
  REGION 1..391: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSSHHHHHH

SEQ ID NO: 863 - protein, 392 aa, synthetic construct
  REGION 1..392: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSSHHHHHH

SEQ ID NO: 864 - protein, 387 aa, synthetic construct
  REGION 1..387: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSSHHHHHH

SEQ ID NO: 865 - protein, 391 aa, synthetic construct
  REGION 1..391: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSSHHHHHH

SEQ ID NO: 866 - protein, 383 aa, synthetic construct
  REGION 1..383: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSSHHHHHH

SEQ ID NO: 867 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSSHHHHHH

SEQ ID NO: 868 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSSHHHHHH

SEQ ID NO: 869 - protein, 387 aa, synthetic construct
  REGION 1..387: note = Description of Artificial Sequence: Synthetic polypeptide
GGGSQVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 870 - protein, 392 aa, synthetic construct
  REGION 1..392: note = Description of Artificial Sequence: Synthetic polypeptide
GGGSQVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 871 - protein, 389 aa, synthetic construct
  REGION 1..389: note = Description of Artificial Sequence: Synthetic polypeptide
GGGSQVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 872 - protein, 387 aa, synthetic construct
  REGION 1..387: note = Description of Artificial Sequence: Synthetic polypeptide
GGGSQVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 873 - protein, 387 aa, synthetic construct
  REGION 1..387: note = Description of Artificial Sequence: Synthetic polypeptide
GGGSQVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 874 - protein, 395 aa, synthetic construct
  REGION 1..395: note = Description of Artificial Sequence: Synthetic polypeptide
GGGSQVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 875 - protein, 379 aa, synthetic construct
  REGION 1..379: note = Description of Artificial Sequence: Synthetic polypeptide
GGGSQVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 876 - protein, 392 aa, synthetic construct
  REGION 1..392: note = Description of Artificial Sequence: Synthetic polypeptide
GGGSQVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 877 - protein, 387 aa, synthetic construct
  REGION 1..387: note = Description of Artificial Sequence: Synthetic polypeptide
GGGSQVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 878 - protein, 387 aa, synthetic construct
  REGION 1..387: note = Description of Artificial Sequence: Synthetic polypeptide
GGGSQVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 879 - protein, 387 aa, synthetic construct
  REGION 1..387: note = Description of Artificial Sequence: Synthetic polypeptide
GGGSQVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 880 - protein, 380 aa, synthetic construct
  REGION 1..380: note = Description of Artificial Sequence: Synthetic polypeptide
GGGSQVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 881 - protein, 390 aa, synthetic construct
  REGION 1..390: note = Description of Artificial Sequence: Synthetic polypeptide
GGGSQVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 882 - protein, 387 aa, synthetic construct
  REGION 1..387: note = Description of Artificial Sequence: Synthetic polypeptide
GGGSQVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 883 - protein, 390 aa, synthetic construct
  REGION 1..390: note = Description of Artificial Sequence: Synthetic polypeptide
GGGSQVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 884 - protein, 390 aa, synthetic construct
  REGION 1..390: note = Description of Artificial Sequence: Synthetic polypeptide
GGGSQVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 885 - protein, 387 aa, synthetic construct
  REGION 1..387: note = Description of Artificial Sequence: Synthetic polypeptide
GGGSQVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 886 - protein, 380 aa, synthetic construct
  REGION 1..380: note = Description of Artificial Sequence: Synthetic polypeptide
GGGSQVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 887 - protein, 388 aa, synthetic construct
  REGION 1..388: note = Description of Artificial Sequence: Synthetic polypeptide
GGGSQVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 888 - protein, 389 aa, synthetic construct
  REGION 1..389: note = Description of Artificial Sequence: Synthetic polypeptide
GGGSQVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 889 - protein, 388 aa, synthetic construct
  REGION 1..388: note = Description of Artificial Sequence: Synthetic polypeptide
GGGSQVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 890 - protein, 389 aa, synthetic construct
  REGION 1..389: note = Description of Artificial Sequence: Synthetic polypeptide
GGGSQVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 891 - protein, 384 aa, synthetic construct
  REGION 1..384: note = Description of Artificial Sequence: Synthetic polypeptide
GGGSQVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 892 - protein, 388 aa, synthetic construct
  REGION 1..388: note = Description of Artificial Sequence: Synthetic polypeptide
GGGSQVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 893 - protein, 380 aa, synthetic construct
  REGION 1..380: note = Description of Artificial Sequence: Synthetic polypeptide
GGGSQVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 894 - protein, 387 aa, synthetic construct
  REGION 1..387: note = Description of Artificial Sequence: Synthetic polypeptide
GGGSQVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 895 - protein, 387 aa, synthetic construct
  REGION 1..387: note = Description of Artificial Sequence: Synthetic polypeptide
GGGSQVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 896 - protein, 111 aa, synthetic construct
  REGION 1..111: note = Description of Artificial Sequence: Synthetic polypeptide
EVQLVESGGGLVQPGGSLTLSCAASGFAFGNHWMYWYRQAPGRGRELVASISSGGSTNYVDSVKGRFTISRDNAKNTLYLQMNSLRAEDTAVYYCGTSDNWGQGTLVTVSS

SEQ ID NO: 897 - protein, 119 aa, synthetic construct
  REGION 1..119: note = Description of Artificial Sequence: Synthetic polypeptide
EVQLLESGGGLVQPGGSLTLSCAASGFIFRAASMDWYRQFPGNERESIATISSGGFTNYADSVKGRFTISRDNSKNTLYLQMNSLRAEDTAVYYCGATFLRSDGHHTINGQGTLVTVSS

SEQ ID NO: 898 - protein, 119 aa, synthetic construct
  REGION 1..119: note = Description of Artificial Sequence: Synthetic polypeptide
EVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSS

SEQ ID NO: 899 - protein, 382 aa, synthetic construct
  REGION 1..382: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGFAFGNHWMYWYRQAPGRGRELVASISSGGSTNYVDSVKGRFTISRDNAKNTLYLQMNSLRAEDTAVYYCGTSDNWGQGTLVTVSSHHHHHH

SEQ ID NO: 900 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMDWYRQFPGNERESIATISSGGFTNYADSVKGRFTISRDNSKNTLYLQMNSLRAEDTAVYYCGATFLRSDGHHTINGQGTLVTVSSHHHHHH

SEQ ID NO: 901 - protein, 390 aa, synthetic construct
  REGION 1..390: note = Description of Artificial Sequence: Synthetic polypeptide
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 902 - protein, 12 aa, synthetic construct
  REGION 1..12: note = Description of Artificial Sequence: Synthetic peptide
QDGNEEMGGITQ

SEQ ID NO: 903 - protein, 6 aa, Homo sapiens
LVQPGN

SEQ ID NO: 904 - protein, 6 aa, Homo sapiens
DSVKGR

SEQ ID NO: 905 - protein, 6 aa, Homo sapiens
SLRPED

SEQ ID NO: 906 - protein, 507 aa, synthetic construct
  REGION 1..507: note = Description of Artificial Sequence: Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 907 - protein, 383 aa, synthetic construct
  REGION 1..383: note = Description of Artificial Sequence: Synthetic polypeptide
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 908 - protein, 526 aa, synthetic construct
  REGION 1..526: note = Description of Artificial Sequence: Synthetic polypeptide
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 909 - protein, 10 aa, unidentified
  REGION 1..10: note = Description of Unknown: Protease cleavage site sequence
PQGSTARSAG

SEQ ID NO: 910 - protein, 114 aa, synthetic construct
  REGION 1..114: note = Description of Artificial Sequence: Synthetic polypeptide
QVQLQESGGGLVQAGGSLRLSCAASGSVSMFNSMGWHRQPPGKQRELVAIITSGGSSNYADTVKGRFTISRDNTKNTVYLQMNDLKPEDTAVYYCFYYFQSSYWGQGTQVTVSS

SEQ ID NO: 911 - protein, 119 aa, synthetic construct
  REGION 1..119: note = Description of Artificial Sequence: Synthetic polypeptide
EVQLVESGGGLVQPGGSLTLSCVASGKTSSINAMGWYRRAPGKQRELVAGISSDGSKVYADSVKGRFTISRDNAKNSVYLQMNSLRAEDTAVYYCYYFRTVSGVSMRYWGQGTLVTVSS

SEQ ID NO: 912 - protein, 119 aa, synthetic construct
  REGION 1..119: note = Description of Artificial Sequence: Synthetic polypeptide
EVQLVESGGGLVQPGGSLTLSCVASGSTSSINAVGWYRRAPGKQRELVAGISSDGSKVYRDSVKGRFTISRDNAKNSVYLQMNSLRAEDTAVYYCYYFRTVQGSSMRYWGQGTLVTVSS

SEQ ID NO: 913 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGVSSDGSKVYADSVKGRFTISRDNAKNSVYLQMNSLRAEDTAVYYCYYFRTVSGSSARYWGQGTLVTVSS

SEQ ID NO: 914 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELPAGISRDGSKVYADSVKGRFTISRDNAKNSVYLQMNSLRAEDTAVYYCYYFRYVSGSSMRYWGQGTLVTVSS

SEQ ID NO: 915 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELAAGISSDGSKLYADSVKGRFTISRDNAKNSVYLQMNSLRAEDTAVYYCYYFRTVSGSSMRYWGQGTLVTVSS

SEQ ID NO: 916 - protein, 119 aa, synthetic construct
  REGION 1..119: note = Description of Artificial Sequence: Synthetic polypeptide
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRRVSGSSMRYWGQGTLVTVSS

SEQ ID NO: 917 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELAAGISSDGSKVYADSVKGRFTISRDNAKNSVYLQMNSLRAEDTAVYYCYYFHTVSGSSMRYWGQGTLVTVSS

SEQ ID NO: 918 - protein, 119 aa, synthetic construct
  REGION 1..119: note = Description of Artificial Sequence: Synthetic polypeptide
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRQVSGSSMRYWGQGTLVTVSS

SEQ ID NO: 919 - protein, 119 aa, synthetic construct
  REGION 1..119: note = Description of Artificial Sequence: Synthetic polypeptide
EVQLVESGGGLVQPGGSLTLSCVASGSTSSINAMGWYRRAPGKQRELVAGISSDTSKVYADSVKGRFTISRDNAKNSVYLQMNSLRAEDTAVYYCYYFRTVSGSYMRYWGQGTLVTVSS

SEQ ID NO: 920 - protein, 119 aa, synthetic construct
  REGION 1..119: note = Description of Artificial Sequence: Synthetic polypeptide
EVQLVESGGGLVQPGGSLTLSCVASGSTSTINAMGWYRRAPGKQRELVAGISSDGSKVYADSVKGRFTISRDNAKNSVYLQMNSLRAEDTAVYYCYYFRTASGSSMRYWGQGTLVTVSS

SEQ ID NO: 921 - protein, 5 aa, Homo sapiens
QDGNE

SEQ ID NO: 922 - protein, 4 aa, Homo sapiens
DGNE

SEQ ID NO: 923 - protein, 5 aa, Homo sapiens
DGNEE

SEQ ID NO: 924 - protein, 8 aa, unidentified
  REGION 1..8: note = Description of Unknown: protease cleavage site
GPLGIAGQ

SEQ ID NO: 925 - protein, 8 aa, unidentified
  REGION 1..8: note = Description of Unknown: protease cleavage site
AVRWLLTA

SEQ ID NO: 926 - protein, 6 aa, unidentified
  REGION 1..6: note = Description of Unknown: protease cleavage site
RRRRRR

SEQ ID NO: 927 - protein, 6 aa, synthetic construct
  REGION 1..6: note = Description of Artificial Sequence: Synthetic 6xHis tag
HHHHHH

SEQ ID NO: 928 - protein, 9 aa, synthetic construct
  REGION 1..9: note = Description of Artificial Sequence: Synthetic peptide
GGGGSGGGS

SEQ ID NO: 929 - protein, 18 aa, synthetic construct
  REGION 1..18: note = Description of Artificial Sequence: Synthetic peptide
  VARIANT 5..14: note = Any amino acid
GGGGXXXXXXXXXXGGGG

SEQ ID NO: 930 - protein, 6 aa, synthetic construct
  REGION 1..6: note = Description of Artificial Sequence: Synthetic peptide
LVQPGN

SEQ ID NO: 931 - protein, 5 aa, synthetic construct
  REGION 1..5: note = Description of Artificial Sequence: Synthetic peptide
APGKG

SEQ ID NO: 932 - protein, 6 aa, synthetic construct
  REGION 1..6: note = Description of Artificial Sequence: Synthetic peptide
DSVKGR

SEQ ID NO: 933 - protein, 6 aa, synthetic construct
  REGION 1..6: note = Description of Artificial Sequence: Synthetic peptide
SLRPED

SEQ ID NO: 934 - protein, 8 aa, synthetic construct
  REGION 1..8: note = Description of Artificial Sequence: Synthetic peptide
  VARIANT 4: note = Any amino acid
CFCXLCFR

SEQ ID NO: 935 - protein, 5 aa, synthetic construct
  REGION 1..5: note = Description of Artificial Sequence: Synthetic peptide
STQDM

SEQ ID NO: 936 - protein, 6 aa, synthetic construct
  REGION 1..6: note = Description of Artificial Sequence: Synthetic peptide
DSVKGR

SEQ ID NO: 937 - protein, 6 aa, synthetic construct
  REGION 1..6: note = Description of Artificial Sequence: Synthetic peptide
SLRPED

SEQ ID NO: 938 - protein, 6 aa, synthetic construct
  REGION 1..6: note = Description of Artificial Sequence: Synthetic peptide
LVQPGN

SEQ ID NO: 939 - protein, 5 aa, synthetic construct
  REGION 1..5: note = Description of Artificial Sequence: Synthetic peptide
KQHSN

SEQ ID NO: 940 - protein, 6 aa, synthetic construct
  REGION 1..6: note = Description of Artificial Sequence: Synthetic peptide
DSVKGR

SEQ ID NO: 941 - protein, 6 aa, synthetic construct
  REGION 1..6: note = Description of Artificial Sequence: Synthetic peptide
SLRPED

SEQ ID NO: 942 - protein, 6 aa, synthetic construct
  REGION 1..6: note = Description of Artificial Sequence: Synthetic peptide
IVLYEW

SEQ ID NO: 943 - protein, 4 aa, synthetic construct
  REGION 1..4: note = Description of Artificial Sequence: Synthetic peptide
APLV

SEQ ID NO: 944 - protein, 6 aa, synthetic construct
  REGION 1..6: note = Description of Artificial Sequence: Synthetic peptide
SSMIQM

SEQ ID NO: 945 - protein, 6 aa, synthetic construct
  REGION 1..6: note = Description of Artificial Sequence: Synthetic peptide
SLRPED

SEQ ID NO: 946 - protein, 6 aa, synthetic construct
  REGION 1..6: note = Description of Artificial Sequence: Synthetic peptide
CFMCYW

SEQ ID NO: 947 - protein, 8 aa, synthetic construct
  REGION 1..8: note = Description of Artificial Sequence: Synthetic peptide
DMWGANCS

SEQ ID NO: 948 - protein, 6 aa, synthetic construct
  REGION 1..6: note = Description of Artificial Sequence: Synthetic peptide
DSVKGR

SEQ ID NO: 949 - protein, 6 aa, synthetic construct
  REGION 1..6: note = Description of Artificial Sequence: Synthetic peptide
SLRPED

SEQ ID NO: 950 - protein, 7 aa, synthetic construct
  REGION 1..7: note = Description of Artificial Sequence: Synthetic peptide
FYLAWAA

SEQ ID NO: 951 - protein, 8 aa, synthetic construct
  REGION 1..8: note = Description of Artificial Sequence: Synthetic peptide
INVGSPAY

SEQ ID NO: 952 - protein, 6 aa, synthetic construct
  REGION 1..6: note = Description of Artificial Sequence: Synthetic peptide
DSVKGR

SEQ ID NO: 953 - protein, 6 aa, synthetic construct
  REGION 1..6: note = Description of Artificial Sequence: Synthetic peptide
SLHPED

SEQ ID NO: 954 - protein, 8 aa, synthetic construct
  REGION 1..8: note = Description of Artificial Sequence: Synthetic peptide
GLSNRSGC

SEQ ID NO: 955 - protein, 8 aa, synthetic construct
  REGION 1..8: note = Description of Artificial Sequence: Synthetic peptide
CSVGISRQ

SEQ ID NO: 956 - protein, 7 aa, synthetic construct
  REGION 1..7: note = Description of Artificial Sequence: Synthetic peptide
LLLIFKF

SEQ ID NO: 957 - protein, 4 aa, synthetic construct
  REGION 1..4: note = Description of Artificial Sequence: Synthetic peptide
LMRI

SEQ ID NO: 958 - protein, 7 aa, synthetic construct
  REGION 1..7: note = Description of Artificial Sequence: Synthetic peptide
FFNSEQF

SEQ ID NO: 959 - protein, 5 aa, synthetic construct
  REGION 1..5: note = Description of Artificial Sequence: Synthetic peptide
APGKG

SEQ ID NO: 960 - protein, 9 aa, synthetic construct
  REGION 1..9: note = Description of Artificial Sequence: Synthetic peptide
FLILALTKP

SEQ ID NO: 961 - protein, 4 aa, synthetic construct
  REGION 1..4: note = Description of Artificial Sequence: Synthetic peptide
QLMI

SEQ ID NO: 962 - protein, 6 aa, synthetic construct
  REGION 1..6: note = Description of Artificial Sequence: Synthetic peptide
LVQPGN

SEQ ID NO: 963 - protein, 8 aa, synthetic construct
  REGION 1..8: note = Description of Artificial Sequence: Synthetic peptide
LRGGLHRV

SEQ ID NO: 964 - protein, 6 aa, synthetic construct
  REGION 1..6: note = Description of Artificial Sequence: Synthetic peptide
SKWACG

SEQ ID NO: 965 - protein, 8 aa, synthetic construct
  REGION 1..8: note = Description of Artificial Sequence: Synthetic peptide
VYMFNLVD

SEQ ID NO: 966 - protein, 6 aa, synthetic construct
  REGION 1..6: note = Description of Artificial Sequence: Synthetic peptide
QLETPC

SEQ ID NO: 967 - protein, 7 aa, synthetic construct
  REGION 1..7: note = Description of Artificial Sequence: Synthetic peptide
NYRWISL

SEQ ID NO: 968 - protein, 6 aa, synthetic construct
  REGION 1..6: note = Description of Artificial Sequence: Synthetic peptide
SLRPED

SEQ ID NO: 969 - protein, 6 aa, synthetic construct
  REGION 1..6: note = Description of Artificial Sequence: Synthetic peptide
LVQPGN

SEQ ID NO: 970 - protein, 5 aa, synthetic construct
  REGION 1..5: note = Description of Artificial Sequence: Synthetic peptide
APGKG

SEQ ID NO: 971 - protein, 9 aa, synthetic construct
  REGION 1..9: note = Description of Artificial Sequence: Synthetic peptide
SVLFGVAYL

SEQ ID NO: 972 - protein, 6 aa, synthetic construct
  REGION 1..6: note = Description of Artificial Sequence: Synthetic peptide
SLRPED

SEQ ID NO: 973 - protein, 8 aa, synthetic construct
  REGION 1..8: note = Description of Artificial Sequence: Synthetic peptide
FGPYNLVS

SEQ ID NO: 974 - protein, 7 aa, synthetic construct
  REGION 1..7: note = Description of Artificial Sequence: Synthetic peptide
KLSTIWM

SEQ ID NO: 975 - protein, 8 aa, synthetic construct
  REGION 1..8: note = Description of Artificial Sequence: Synthetic peptide
MRRPCVIS

SEQ ID NO: 976 - protein, 7 aa, synthetic construct
  REGION 1..7: note = Description of Artificial Sequence: Synthetic peptide
VPSYRVA

SEQ ID NO: 977 - protein, 7 aa, synthetic construct
  REGION 1..7: note = Description of Artificial Sequence: Synthetic peptide
DSGLTLR

SEQ ID NO: 978 - protein, 6 aa, synthetic construct
  REGION 1..6: note = Description of Artificial Sequence: Synthetic peptide
LLWHWD

SEQ ID NO: 979 - protein, 8 aa, synthetic construct
  REGION 1..8: note = Description of Artificial Sequence: Synthetic peptide
VTEMNAMS

SEQ ID NO: 980 - protein, 7 aa, synthetic construct
  REGION 1..7: note = Description of Artificial Sequence: Synthetic peptide
  VARIANT 2: note = Any amino acid
GXTDGGT

SEQ ID NO: 981 - protein, 5 aa, synthetic construct
  REGION 1..5: note = Description of Artificial Sequence: Synthetic peptide
  VARIANT 4: note = Any amino acid
FLIXF

SEQ ID NO: 982 - protein, 6 aa, synthetic construct
  REGION 1..6: note = Description of Artificial Sequence: Synthetic peptide
DSVKGR

SEQ ID NO: 983 - protein, 6 aa, synthetic construct
  REGION 1..6: note = Description of Artificial Sequence: Synthetic peptide
LKRTVY

SEQ ID NO: 984 - protein, 8 aa, synthetic construct
  REGION 1..8: note = Description of Artificial Sequence: Synthetic peptide
SICIVSFY

SEQ ID NO: 985 - protein, 5 aa, synthetic construct
  REGION 1..5: note = Description of Artificial Sequence: Synthetic peptide
TNTAS

SEQ ID NO: 986 - protein, 9 aa, synthetic construct
  REGION 1..9: note = Description of Artificial Sequence: Synthetic peptide
RGLIDNDYQ

SEQ ID NO: 987 - protein, 5 aa, synthetic construct
  REGION 1..5: note = Description of Artificial Sequence: Synthetic peptide
RQYSV

SEQ ID NO: 988 - protein, 6 aa, synthetic construct
  REGION 1..6: note = Description of Artificial Sequence: Synthetic peptide
  VARIANT 5..6: note = Any amino acid
LSRPXX

SEQ ID NO: 989 - protein, 7 aa, synthetic construct
  REGION 1..7: note = Description of Artificial Sequence: Synthetic peptide
CPPAIMP

SEQ ID NO: 990 - protein, 6 aa, synthetic construct
  REGION 1..6: note = Description of Artificial Sequence: Synthetic peptide
DSVKGL

SEQ ID NO: 991 - protein, 6 aa, synthetic construct
  REGION 1..6: note = Description of Artificial Sequence: Synthetic peptide
SLRPED

SEQ ID NO: 992 - protein, 8 aa, synthetic construct
  REGION 1..8: note = Description of Artificial Sequence: Synthetic peptide
  VARIANT 7: note = Any amino acid
FALQCPXL

SEQ ID NO: 993 - protein, 4 aa, synthetic construct
  REGION 1..4: note = Description of Artificial Sequence: Synthetic peptide
IVGN

SEQ ID NO: 994 - protein, 7 aa, synthetic construct
  REGION 1..7: note = Description of Artificial Sequence: Synthetic peptide
LINLITE

SEQ ID NO: 995 - protein, 6 aa, synthetic construct
  REGION 1..6: note = Description of Artificial Sequence: Synthetic peptide
SLCPED

SEQ ID NO: 996 - protein, 6 aa, synthetic construct
  REGION 1..6: note = Description of Artificial Sequence: Synthetic peptide
MSMSLF

SEQ ID NO: 997 - protein, 4 aa, synthetic construct
  REGION 1..4: note = Description of Artificial Sequence: Synthetic peptide
NVTL

SEQ ID NO: 998 - protein, 6 aa, synthetic construct
  REGION 1..6: note = Description of Artificial Sequence: Synthetic peptide
SDNILY

SEQ ID NO: 999 - protein, 6 aa, synthetic construct
  REGION 1..6: note = Description of Artificial Sequence: Synthetic peptide
SLRPED

SEQ ID NO: 1000 - protein, 6 aa, synthetic construct
  REGION 1..6: note = Description of Artificial Sequence: Synthetic peptide
IDVFIC

SEQ ID NO: 1001 - protein, 7 aa, synthetic construct
  REGION 1..7: note = Description of Artificial Sequence: Synthetic peptide
ISLPVMS

SEQ ID NO: 1002 - protein, 8 aa, synthetic construct
  REGION 1..8: note = Description of Artificial Sequence: Synthetic peptide
TPRTPTRP

SEQ ID NO: 1003 - protein, 6 aa, synthetic construct
  REGION 1..6: note = Description of Artificial Sequence: Synthetic peptide
SLRPED

SEQ ID NO: 1004 - protein, 6 aa, synthetic construct
  REGION 1..6: note = Description of Artificial Sequence: Synthetic peptide
LFQPGN

SEQ ID NO: 1005 - protein, 7 aa, synthetic construct
  REGION 1..7: note = Description of Artificial Sequence: Synthetic peptide
LYEPRAE

SEQ ID NO: 1006 - protein, 7 aa, synthetic construct
  REGION 1..7: note = Description of Artificial Sequence: Synthetic peptide
FLCFDLQ

SEQ ID NO: 1007 - protein, 5 aa, synthetic construct
  REGION 1..5: note = Description of Artificial Sequence: Synthetic peptide
RISAM

SEQ ID NO: 1008 - protein, 8 aa, synthetic construct
  REGION 1..8: note = Description of Artificial Sequence: Synthetic peptide
  VARIANT 8: note = Any amino acid
YGNNCVLX

SEQ ID NO: 1009 - protein, 6 aa, synthetic construct
  REGION 1..6: note = Description of Artificial Sequence: Synthetic peptide
VARAYD

SEQ ID NO: 1010 - protein, 7 aa, synthetic construct
  REGION 1..7: note = Description of Artificial Sequence: Synthetic peptide
YRNHNRN

SEQ ID NO: 1011 - protein, 6 aa, synthetic construct
  REGION 1..6: note = Description of Artificial Sequence: Synthetic peptide
LVQPGN

SEQ ID NO: 1012 - protein, 5 aa, synthetic construct
  REGION 1..5: note = Description of Artificial Sequence: Synthetic peptide
APGKG

SEQ ID NO: 1013 - protein, 5 aa, synthetic construct
  REGION 1..5: note = Description of Artificial Sequence: Synthetic peptide
VACGR

SEQ ID NO: 1014 - protein, 5 aa, synthetic construct
  REGION 1..5: note = Description of Artificial Sequence: Synthetic peptide
NTTAR

SEQ ID NO: 1015 - protein, 8 aa, synthetic construct
  REGION 1..8: note = Description of Artificial Sequence: Synthetic peptide
MLTGFPFG

SEQ ID NO: 1016 - protein, 4 aa, synthetic construct
  REGION 1..4: note = Description of Artificial Sequence: Synthetic peptide
EIWW

SEQ ID NO: 1017 - protein, 6 aa, synthetic construct
  REGION 1..6: note = Description of Artificial Sequence: Synthetic peptide
DSVKGR

SEQ ID NO: 1018 - protein, 6 aa, synthetic construct
  REGION 1..6: note = Description of Artificial Sequence: Synthetic peptide
SLRPED

SEQ ID NO: 1019 - protein, 6 aa, synthetic construct
  REGION 1..6: note = Description of Artificial Sequence: Synthetic peptide
LVQPGN

SEQ ID NO: 1020 - protein, 5 aa, synthetic construct
  REGION 1..5: note = Description of Artificial Sequence: Synthetic peptide
YKEWD

SEQ ID NO: 1021 - protein, 6 aa, synthetic construct
  REGION 1..6: note = Description of Artificial Sequence: Synthetic peptide
DSVKGR

SEQ ID NO: 1022 - protein, 6 aa, synthetic construct
  REGION 1..6: note = Description of Artificial Sequence: Synthetic peptide
SLRPED

SEQ ID NO: 1023 - protein, 6 aa, synthetic construct
  REGION 1..6: note = Description of Artificial Sequence: Synthetic peptide
LVQPGN

SEQ ID NO: 1024 - protein, 5 aa, synthetic construct
  REGION 1..5: note = Description of Artificial Sequence: Synthetic peptide
APGKG

SEQ ID NO: 1025 - protein, 6 aa, synthetic construct
  REGION 1..6: note = Description of Artificial Sequence: Synthetic peptide
DSDKGR

SEQ ID NO: 1026 - protein, 6 aa, synthetic construct
  REGION 1..6: note = Description of Artificial Sequence: Synthetic peptide
SLPPQD

SEQ ID NO: 1027 - protein, 6 aa, synthetic construct
  REGION 1..6: note = Description of Artificial Sequence: Synthetic peptide
LVQPGN

SEQ ID NO: 1028 - protein, 6 aa, synthetic construct
  REGION 1..6: note = Description of Artificial Sequence: Synthetic peptide
DSVKGR

SEQ ID NO: 1029 - protein, 6 aa, synthetic construct
  REGION 1..6: note = Description of Artificial Sequence: Synthetic peptide
SLRPED

SEQ ID NO: 1030 - protein, 7 aa, synthetic construct
  REGION 1..7: note = Description of Artificial Sequence: Synthetic peptide
GLINSIA

SEQ ID NO: 1031 - protein, 6 aa, synthetic construct
  REGION 1..6: note = Description of Artificial Sequence: Synthetic peptide
RGSEGG

SEQ ID NO: 1032 - protein, 6 aa, synthetic construct
  REGION 1..6: note = Description of Artificial Sequence: Synthetic peptide
DSVKGR

SEQ ID NO: 1033 - protein, 6 aa, synthetic construct
  REGION 1..6: note = Description of Artificial Sequence: Synthetic peptide
SLRPED

SEQ ID NO: 1034 - protein, 6 aa, synthetic construct
  REGION 1..6: note = Description of Artificial Sequence: Synthetic peptide
LVQPGN

SEQ ID NO: 1035 - protein, 5 aa, synthetic construct
  REGION 1..5: note = Description of Artificial Sequence: Synthetic peptide
APGKG

SEQ ID NO: 1036 - protein, 6 aa, synthetic construct
  REGION 1..6: note = Description of Artificial Sequence: Synthetic peptide
DSVKGR

SEQ ID NO: 1037 - protein, 6 aa, synthetic construct
  REGION 1..6: note = Description of Artificial Sequence: Synthetic peptide
SLRPED

SEQ ID NO: 1038 - protein, 6 aa, synthetic construct
  REGION 1..6: note = Description of Artificial Sequence: Synthetic peptide
LIQPGN

SEQ ID NO: 1039 - protein, 6 aa, synthetic construct
  REGION 1..6: note = Description of Artificial Sequence: Synthetic peptide
  VARIANT 1: note = Any amino acid
XLHGGS

SEQ ID NO: 1040 - protein, 6 aa, synthetic construct
  REGION 1..6: note = Description of Artificial Sequence: Synthetic peptide
DSVKGR

SEQ ID NO: 1041 - protein, 6 aa, synthetic construct
  REGION 1..6: note = Description of Artificial Sequence: Synthetic peptide
SLRPED

SEQ ID NO: 1042 - protein, 6 aa, synthetic construct
  REGION 1..6: note = Description of Artificial Sequence: Synthetic peptide
LVQPGN

SEQ ID NO: 1043 - protein, 5 aa, synthetic construct
  REGION 1..5: note = Description of Artificial Sequence: Synthetic peptide
LTNTS

SEQ ID NO: 1044 - protein, 6 aa, synthetic construct
  REGION 1..6: note = Description of Artificial Sequence: Synthetic peptide
SLRPED

SEQ ID NO: 1045 - protein, 6 aa, synthetic construct
  REGION 1..6: note = Description of Artificial Sequence: Synthetic peptide
LVQPGN

SEQ ID NO: 1046 - protein, 5 aa, synthetic construct
  REGION 1..5: note = Description of Artificial Sequence: Synthetic peptide
APGKG

SEQ ID NO: 1047 - protein, 7 aa, synthetic construct
  REGION 1..7: note = Description of Artificial Sequence: Synthetic peptide
GAYFTGR

SEQ ID NO: 1048 - protein, 6 aa, synthetic construct
  REGION 1..6: note = Description of Artificial Sequence: Synthetic peptide
SLRPED

SEQ ID NO: 1049 - protein, 6 aa, synthetic construct
  REGION 1..6: note = Description of Artificial Sequence: Synthetic peptide
LVQPGN

SEQ ID NO: 1050 - protein, 7 aa, synthetic construct
  REGION 1..7: note = Description of Artificial Sequence: Synthetic peptide
KQQSHIM

SEQ ID NO: 1051 - protein, 6 aa, synthetic construct
  REGION 1..6: note = Description of Artificial Sequence: Synthetic peptide
DSVKGR

SEQ ID NO: 1052 - protein, 6 aa, synthetic construct
  REGION 1..6: note = Description of Artificial Sequence: Synthetic peptide
SLRPED

SEQ ID NO: 1053 - protein, 6 aa, synthetic construct
  REGION 1..6: note = Description of Artificial Sequence: Synthetic peptide
LVQPGN

SEQ ID NO: 1054 - protein, 6 aa, synthetic construct
  REGION 1..6: note = Description of Artificial Sequence: Synthetic peptide
  VARIANT 3: note = Any amino acid
LPXMKR

SEQ ID NO: 1055 - protein, 6 aa, synthetic construct
  REGION 1..6: note = Description of Artificial Sequence: Synthetic peptide
DSVKGR

SEQ ID NO: 1056 - protein, 6 aa, synthetic construct
  REGION 1..6: note = Description of Artificial Sequence: Synthetic peptide
SLRPED

SEQ ID NO: 1057 - protein, 6 aa, synthetic construct
  REGION 1..6: note = Description of Artificial Sequence: Synthetic peptide
LVQPGN

SEQ ID NO: 1058 - protein, 5 aa, synthetic construct
  REGION 1..5: note = Description of Artificial Sequence: Synthetic peptide
VGHPK

SEQ ID NO: 1059 - protein, 6 aa, synthetic construct
  REGION 1..6: note = Description of Artificial Sequence: Synthetic peptide
DSVKGR

SEQ ID NO: 1060 - protein, 6 aa, synthetic construct
  REGION 1..6: note = Description of Artificial Sequence: Synthetic peptide
SLRPED

SEQ ID NO: 1061 - protein, 6 aa, synthetic construct
  REGION 1..6: note = Description of Artificial Sequence: Synthetic peptide
LVQPGN

SEQ ID NO: 1062 - protein, 5 aa, synthetic construct
  REGION 1..5: note = Description of Artificial Sequence: Synthetic peptide
APGKG

SEQ ID NO: 1063 - protein, 6 aa, synthetic construct
  REGION 1..6: note = Description of Artificial Sequence: Synthetic peptide
DSVKGR

SEQ ID NO: 1064 - protein, 6 aa, synthetic construct
  REGION 1..6: note = Description of Artificial Sequence: Synthetic peptide
SLRPED

SEQ ID NO: 1065 - protein, 6 aa, synthetic construct
  REGION 1..6: note = Description of Artificial Sequence: Synthetic peptide
LVQPGN

SEQ ID NO: 1066 - protein, 5 aa, synthetic construct
  REGION 1..5: note = Description of Artificial Sequence: Synthetic peptide
TVTTP

SEQ ID NO: 1067 - protein, 6 aa, synthetic construct
  REGION 1..6: note = Description of Artificial Sequence: Synthetic peptide
DSVKGR

SEQ ID NO: 1068 - protein, 6 aa, synthetic construct
  REGION 1..6: note = Description of Artificial Sequence: Synthetic peptide
SLRPED

SEQ ID NO: 1069 - protein, 6 aa, synthetic construct
  REGION 1..6: note = Description of Artificial Sequence: Synthetic peptide
LVQPGN

SEQ ID NO: 1070 - protein, 6 aa, synthetic construct
  REGION 1..6: note = Description of Artificial Sequence: Synthetic peptide
SNSLNS

SEQ ID NO: 1071 - protein, 6 aa, synthetic construct
  REGION 1..6: note = Description of Artificial Sequence: Synthetic peptide
DSVKGR

SEQ ID NO: 1072 - protein, 6 aa, synthetic construct
  REGION 1..6: note = Description of Artificial Sequence: Synthetic peptide
SLRPED

SEQ ID NO: 1073 - protein, 6 aa, synthetic construct
  REGION 1..6: note = Description of Artificial Sequence: Synthetic peptide
LVQPGN

SEQ ID NO: 1074 - protein, 5 aa, synthetic construct
  REGION 1..5: note = Description of Artificial Sequence: Synthetic peptide
TSSRT

SEQ ID NO: 1075 - protein, 6 aa, synthetic construct
  REGION 1..6: note = Description of Artificial Sequence: Synthetic peptide
DSVKGR

SEQ ID NO: 1076 - protein, 7 aa, synthetic construct
  REGION 1..7: note = Description of Artificial Sequence: Synthetic peptide
FPNKAIN

SEQ ID NO: 1077 - protein, 6 aa, synthetic construct
  REGION 1..6: note = Description of Artificial Sequence: Synthetic peptide
LVQPGN

SEQ ID NO: 1078 - protein, 6 aa, synthetic construct
  REGION 1..6: note = Description of Artificial Sequence: Synthetic peptide
SNSLNS

SEQ ID NO: 1079 - protein, 6 aa, synthetic construct
  REGION 1..6: note = Description of Artificial Sequence: Synthetic peptide
DSVKGR

SEQ ID NO: 1080 - protein, 6 aa, synthetic construct
  REGION 1..6: note = Description of Artificial Sequence: Synthetic peptide
SLRPED

SEQ ID NO: 1081 - protein, 6 aa, synthetic construct
  REGION 1..6: note = Description of Artificial Sequence: Synthetic peptide
LVQPGN

SEQ ID NO: 1082 - protein, 5 aa, synthetic construct
  REGION 1..5: note = Description of Artificial Sequence: Synthetic peptide
VANLY

SEQ ID NO: 1083 - protein, 6 aa, synthetic construct
  REGION 1..6: note = Description of Artificial Sequence: Synthetic peptide
DSVKGR

SEQ ID NO: 1084 - protein, 6 aa, synthetic construct
  REGION 1..6: note = Description of Artificial Sequence: Synthetic peptide
SLRPED

SEQ ID NO: 1085 - protein, 6 aa, synthetic construct
  REGION 1..6: note = Description of Artificial Sequence: Synthetic peptide
LVQPGN

SEQ ID NO: 1086 - protein, 5 aa, synthetic construct
  REGION 1..5: note = Description of Artificial Sequence: Synthetic peptide
SEAKT

SEQ ID NO: 1087 - protein, 6 aa, synthetic construct
  REGION 1..6: note = Description of Artificial Sequence: Synthetic peptide
DSVKGR

SEQ ID NO: 1088 - protein, 6 aa, synthetic construct
  REGION 1..6: note = Description of Artificial Sequence: Synthetic peptide
SLRPED

SEQ ID NO: 1089 - protein, 6 aa, synthetic construct
  REGION 1..6: note = Description of Artificial Sequence: Synthetic peptide
LVQPGN

SEQ ID NO: 1090 - protein, 5 aa, synthetic construct
  REGION 1..5: note = Description of Artificial Sequence: Synthetic peptide
APGEG

SEQ ID NO: 1091 - protein, 6 aa, synthetic construct
  REGION 1..6: note = Description of Artificial Sequence: Synthetic peptide
DSVKGR

SEQ ID NO: 1092 - protein, 6 aa, synthetic construct
  REGION 1..6: note = Description of Artificial Sequence: Synthetic peptide
SLRPED

SEQ ID NO: 1093 - protein, 6 aa, synthetic construct
  REGION 1..6: note = Description of Artificial Sequence: Synthetic peptide
LVQPGN

SEQ ID NO: 1094 - protein, 7 aa, synthetic construct
  REGION 1..7: note = Description of Artificial Sequence: Synthetic peptide
NMIRYPK

SEQ ID NO: 1095 - protein, 6 aa, synthetic construct
  REGION 1..6: note = Description of Artificial Sequence: Synthetic peptide
DSVKGR

SEQ ID NO: 1096 - protein, 6 aa, synthetic construct
  REGION 1..6: note = Description of Artificial Sequence: Synthetic peptide
SLRPED

SEQ ID NO: 1097 - protein, 6 aa, synthetic construct
  REGION 1..6: note = Description of Artificial Sequence: Synthetic peptide
LVQPGN

SEQ ID NO: 1098 - protein, 5 aa, synthetic construct
  REGION 1..5: note = Description of Artificial Sequence: Synthetic peptide
APGKG

SEQ ID NO: 1099 - protein, 6 aa, synthetic construct
  REGION 1..6: note = Description of Artificial Sequence: Synthetic peptide
DSVKGR

SEQ ID NO: 1100 - protein, 6 aa, synthetic construct
  REGION 1..6: note = Description of Artificial Sequence: Synthetic peptide
SLRPED

SEQ ID NO: 1101 - protein, 7 aa, synthetic construct
  REGION 1..7: note = Description of Artificial Sequence: Synthetic peptide
VAVLRGE

SEQ ID NO: 1102 - protein, 5 aa, synthetic construct
  REGION 1..5: note = Description of Artificial Sequence: Synthetic peptide
RALNL

SEQ ID NO: 1103 - protein, 6 aa, synthetic construct
  REGION 1..6: note = Description of Artificial Sequence: Synthetic peptide
DSVKGR

SEQ ID NO: 1104 - protein, 6 aa, synthetic construct
  REGION 1..6: note = Description of Artificial Sequence: Synthetic peptide
SLRPED

SEQ ID NO: 1105 - protein, 7 aa, synthetic construct
  REGION 1..7: note = Description of Artificial Sequence: Synthetic peptide
AAADNGV

SEQ ID NO: 1106 - protein, 5 aa, synthetic construct
  REGION 1..5: note = Description of Artificial Sequence: Synthetic peptide
APGKG

SEQ ID NO: 1107 - protein, 6 aa, synthetic construct
  REGION 1..6: note = Description of Artificial Sequence: Synthetic peptide
DSVKGR

SEQ ID NO: 1108 - protein, 6 aa, synthetic construct
  REGION 1..6: note = Description of Artificial Sequence: Synthetic peptide
SLRPED

SEQ ID NO: 1109 - protein, 6 aa, synthetic construct
  REGION 1..6: note = Description of Artificial Sequence: Synthetic peptide
LVQPGN

SEQ ID NO: 1110 - protein, 5 aa, synthetic construct
  REGION 1..5: note = Description of Artificial Sequence: Synthetic peptide
TSRNG

SEQ ID NO: 1111 - protein, 6 aa, synthetic construct
  REGION 1..6: note = Description of Artificial Sequence: Synthetic peptide
DSVKGR

SEQ ID NO: 1112 - protein, 6 aa, synthetic construct
  REGION 1..6: note = Description of Artificial Sequence: Synthetic peptide
SLRPED

SEQ ID NO: 1113 - protein, 6 aa, synthetic construct
  REGION 1..6: note = Description of Artificial Sequence: Synthetic peptide
LVQPGN

SEQ ID NO: 1114 - protein, 5 aa, synthetic construct
  REGION 1..5: note = Description of Artificial Sequence: Synthetic peptide
TNLSY

SEQ ID NO: 1115 - protein, 6 aa, synthetic construct
  REGION 1..6: note = Description of Artificial Sequence: Synthetic peptide
DSVKGR

SEQ ID NO: 1116 - protein, 6 aa, synthetic construct
  REGION 1..6: note = Description of Artificial Sequence: Synthetic peptide
SLRPED

SEQ ID NO: 1117 - protein, 6 aa, synthetic construct
  REGION 1..6: note = Description of Artificial Sequence: Synthetic peptide
LVQPGN

SEQ ID NO: 1118 - protein, 7 aa, synthetic construct
  REGION 1..7: note = Description of Artificial Sequence: Synthetic peptide
VKKSQNS

SEQ ID NO: 1119 - protein, 6 aa, synthetic construct
  REGION 1..6: note = Description of Artificial Sequence: Synthetic peptide
DSVRGR

SEQ ID NO: 1120 - protein, 6 aa, synthetic construct
  REGION 1..6: note = Description of Artificial Sequence: Synthetic peptide
SLRPED

SEQ ID NO: 1121 - protein, 6 aa, synthetic construct
  REGION 1..6: note = Description of Artificial Sequence: Synthetic peptide
LVQPGN

SEQ ID NO: 1122 - protein, 6 aa, synthetic construct
  REGION 1..6: note = Description of Artificial Sequence: Synthetic peptide
ADDRTH

SEQ ID NO: 1123 - protein, 6 aa, synthetic construct
  REGION 1..6: note = Description of Artificial Sequence: Synthetic peptide
YSVKGR

SEQ ID NO: 1124 - protein, 6 aa, synthetic construct
  REGION 1..6: note = Description of Artificial Sequence: Synthetic peptide
SLRPED

SEQ ID NO: 1125 - protein, 6 aa, synthetic construct
  REGION 1..6: note = Description of Artificial Sequence: Synthetic peptide
LVQPGN

SEQ ID NO: 1126 - protein, 5 aa, synthetic construct
  REGION 1..5: note = Description of Artificial Sequence: Synthetic peptide
APGKG

SEQ ID NO: 1127 - protein, 6 aa, synthetic construct
  REGION 1..6: note = Description of Artificial Sequence: Synthetic peptide
DSVKGR

SEQ ID NO: 1128 - protein, 6 aa, synthetic construct
  REGION 1..6: note = Description of Artificial Sequence: Synthetic peptide
SLRPED

SEQ ID NO: 1129 - protein, 6 aa, synthetic construct
  REGION 1..6: note = Description of Artificial Sequence: Synthetic peptide
LVQPGN

SEQ ID NO: 1130 - protein, 5 aa, synthetic construct
  REGION 1..5: note = Description of Artificial Sequence: Synthetic peptide
  VARIANT 4: note = Any amino acid
APNXV

SEQ ID NO: 1131 - protein, 5 aa, synthetic construct
  REGION 1..5: note = Description of Artificial Sequence: Synthetic peptide
  VARIANT 4: note = Any amino acid
SADXG

SEQ ID NO: 1132 - protein, 6 aa, synthetic construct
  REGION 1..6: note = Description of Artificial Sequence: Synthetic peptide
  VARIANT 2: note = Any amino acid
SXNPQD

SEQ ID NO: 1133 - protein, 6 aa, synthetic construct
  REGION 1..6: note = Description of Artificial Sequence: Synthetic peptide
  VARIANT 6: note = Any amino acid
PGGEGX

SEQ ID NO: 1134 - protein, 5 aa, synthetic construct
  REGION 1..5: note = Description of Artificial Sequence: Synthetic peptide
  VARIANT 1: note = Any amino acid
XSISP

SEQ ID NO: 1135 - protein, 6 aa, synthetic construct
  REGION 1..6: note = Description of Artificial Sequence: Synthetic peptide
DSVKGR

SEQ ID NO: 1136 - protein, 6 aa, synthetic construct
  REGION 1..6: note = Description of Artificial Sequence: Synthetic peptide
SLPPED

SEQ ID NO: 1137 - protein, 6 aa, synthetic construct
  REGION 1..6: note = Description of Artificial Sequence: Synthetic peptide
LVQPGN

SEQ ID NO: 1138 - protein, 5 aa, synthetic construct
  REGION 1..5: note = Description of Artificial Sequence: Synthetic peptide
APGKG

SEQ ID NO: 1139 - protein, 6 aa, synthetic construct
  REGION 1..6: note = Description of Artificial Sequence: Synthetic peptide
DSVKGR

SEQ ID NO: 1140 - protein, 6 aa, synthetic construct
  REGION 1..6: note = Description of Artificial Sequence: Synthetic peptide
SLRPED

(19) United States
(12) Patent Application Publication
(10) Pub. No.: US 2026/0347483 A1
(43) Pub. Date: Nov. 5, 2026

(54) BINDING MOIETY FOR CONDITIONAL ACTIVATION OF IMMUNOGLOBULIN MOLECULES

(71) Applicant: Harpoon Therapeutics, Inc., Rahway, NJ (US)

(72) Inventors: Shuoyen Jack LIN, San Bruno, CA (US); Richard J. AUSTIN, San Francisco, CA (US); Bryan D. LEMON, Mountian View, CA (US); Kathryn KWANT, San Bruno, CA (US); Sony S. ROCHA, San Francisco, CA (US); Holger WESCHE, San Francisco, CA (US)

(73) Assignee: Harpoon Therapeutics, Inc., Rahway, NJ (US)

(21) Appl. No.: 19/259,785

(22) Filed: Jul. 3, 2025

Related U.S. Application Data
(63) Continuation of application No. 17/055,096, filed on May 14, 2019, now Pat. No. 12,415,860.
(60) Provisional application No. 62/671,349, filed on May 14, 2018.
(60) Provisional application No. 62/671,344, filed on May 14, 2018.
(60) Provisional application No. 62/756,453, filed on Nov. 6, 2018.
(60) Provisional application No. 62/756,429, filed on Nov. 6, 2018.

(51) Int. Cl. C07K 16/28; A61K 39/00; C07K 16/30

(52) U.S. Cl. CPC C07K 16/2863; C07K 16/2809; C07K 16/2878; C07K 16/3069; A61K 2039/505; C07K 2317/24; C07K 2317/31; C07K 2317/33; C07K 2317/34; C07K 2317/565; C07K 2317/569; C07K 2317/60; C07K 2317/73; C07K 2317/92

(57) ABSTRACT

Disclosed herein are binding moieties that comprise non-CDR loops for masking the binding of a binding molecule to its target and CDRs for binding bulk serum proteins. Conditionally active target binding proteins that contain the binding moieties are also provided. Pharmaceutical compositions comprising the binding proteins disclosed herein and methods of using such formulations are further provided.

### CROSS-REFERENCE

This application is the U.S. National Stage Application of International Application No. PCT/US2019/032307, filed May 14, 2019, and claims the benefit of U.S. Provisional Application Nos. 62/671,344, filed May 14, 2018; 62/671,349, filed May 14, 2018; 62/756,429, filed Nov. 6, 2018; and 62/756,453 filed Nov. 6, 2018, all of which are incorporated by reference herein in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### SEQUENCE LISTING

The instant application contains a Sequence Listing which has been submitted electronically in XML file format and is hereby incorporated by reference in its entirety. Said XML copy, created on May 30, 2025, is named 25985-US-CNT_SL.xml and is 1,568,035 bytes in size.

### BACKGROUND OF THE INVENTION

T cell engagers transiently tether T cells to tumor cells and mediate T cell-directed tumor killing. T cell engagers, such as blinatumomab (BLINCYTO®), have demonstrated clinical activity in several hematological malignancies. Adoption of T cell engagers in solid tumors is limited by the scarcity of tumor antigens with sufficient differential expression between tumor and normal tissue. T cell engagers that are preferentially active in the tumor microenvironment may enable the safe targeting of more solid tumor antigens.

There is a need to extend the half-life of a therapeutic, diagnostic, or imaging molecule in circulation and also improve its ability to reach its target within an intended location (e.g., a tumor cell) without non-specific binding.

### SUMMARY OF THE INVENTION

One embodiment provides a binding moiety comprising a non-CDR loop and a cleavable linker, wherein the moiety is capable of masking the binding of binding molecule to its target, wherein the binding molecule comprises an immunoglobulin molecule or a non-immunoglobulin molecule. In some embodiments, the moiety is a natural peptide, a synthetic peptide, an engineered scaffold, or an engineered bulk serum protein. In some embodiments, the engineered scaffold comprises a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold. In some embodiments, the moiety is capable of binding to a bulk serum protein. In some embodiments, the non-CDR loop is from a variable domain, a constant domain, a C1-set domain, a C2-set domain, an I-domain, or any combinations thereof. In some embodiments, the moiety further comprises complementarity determining regions (CDRs). In some embodiments, the moiety is capable of binding to the bulk serum protein. In some embodiments, the bulk serum protein is a half-life extending protein. In some embodiments, the bulk serum protein is albumin, transferrin, IgG1, IgG2, IgG4, IgG3, IgA monomer, Factor XIII, Fibrinogen, IgE, or pentameric IgM. In some embodiments, the bulk serum protein is albumin, transferrin, Factor XIII, or Fibrinogen. In some embodiments, the CDRs within the binding moiety provide binding site specific for the bulk serum protein. In some embodiments, the binding moiety is capable of masking the binding of the target antigen binding domain (such as an immunoglobulin molecule) or a non-immunoglobulin binding molecule to its target via specific intermolecular interactions between the binding moiety and the target antigen binding domain or the non-immunoglobulin binding moiety. In some embodiments, the non-CDR loop within the binding moiety provides a binding site specific for binding of the binding moiety to the target antigen binding domain (such as an immunoglobulin molecule) or the non-immunoglobulin binding molecule.

In some embodiments, the binding moiety comprises a binding site specific for an immunoglobulin light chain. In some embodiments, the immunoglobulin light chain is an Igκ free light chain. In some embodiments, the CDRs provide the binding site specific for the bulk serum protein or the immunoglobulin light chain. In some embodiments, the immunoglobulin molecule is a target antigen binding domain. In some embodiments, the moiety is bound to the target antigen binding domain. In some embodiments, the moiety is covalently linked to the target antigen binding domain. In some embodiments, the moiety is capable of masking the binding of the target antigen binding domain to its target via specific intermolecular interactions between the binding moiety and the target antigen binding domain. In some embodiments, the non-CDR loop provides a binding site specific for binding of the moiety to the target antigen binding domain. In some embodiments, upon cleavage of the cleavable linker, the binding moiety is separated from the target antigen binding domain and the target antigen binding domain binds to its target. In some embodiments, the target antigen domain binds to a tumor antigen. In some embodiments, the tumor antigen comprises EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1. In some embodiments, the target antigen domain binds to an immune checkpoint protein. In some embodiments, the immune checkpoint protein is CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the target antigen binding domain binds to a T-cell. In some embodiments, the target antigen binding domain binds to CD3. In some embodiments, the cleavable linker comprises a cleavage site. In some embodiments, the cleavage site is recognized by a protease. In some embodiments, the protease cleavage site is recognized by a serine protease, a cysteine protease, an aspartate protease, a threonine protease, a glutamic acid protease, a metalloproteinase, a gelatinase, or a asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAM9, an ADAM10, an ADAM12, an urokinase plasminogen activator (uPA), an enterokinase, a prostate-specific target (PSA, hK3), an interleukin-1p converting enzyme, a thrombin, a FAP (FAP-α), a dipeptidyl peptidase, a type II transmembrane seine protease (TTSP), a neutrophil elastase, a cathepsin G, a proteinase 3, a neutrophil serine protease 4, a mast cell chymase, and a mast cell tryptase.

One embodiment provides a conditionally active binding protein comprising a binding moiety (M) which comprises a non-CDR loop, a cleavable linker (L), a first target antigen binding domain (T1), and a second target antigen binding domain (T2), wherein the first target antigen binding domain (T1) comprises an immunoglobulin molecule, wherein the non-CDR loop is capable of binding to the first target antigen binding domain, and wherein the binding moiety is capable of masking the binding of the first target antigen binding domain to its target. In some embodiments, the binding moiety is capable of binding to a half-life extending protein. In some embodiments, the binding moiety is a natural peptide, a synthetic peptide, an engineered scaffold, or an engineered serum bulk protein. In some embodiments, the engineered scaffold comprises a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold. In some embodiments, the non-CDR loop is from a variable domain, a constant domain, a C1-set domain, a C2-set domain, an I-domain, or any combinations thereof. In some embodiments, the binding moiety further comprises complementarity determining regions (CDRs). In some embodiments, the binding moiety comprises a binding site specific for a bulk serum protein. In some embodiments, the bulk serum protein is albumin, transferrin, IgG1, IgG2, IgG4, IgG3, IgA monomer, Factor XIII, Fibrinogen, IgE, or pentameric IgM. In some embodiments, the binding moiety further comprises a binding site specific for an immunoglobulin light chain. In some embodiments, the immunoglobulin light chain is an Igκ free light chain. T In some embodiments, the CDRs provide the binding site specific for the bulk serum protein or the immunoglobulin light chain, or any combinations thereof. In some embodiments, the binding moiety is capable of masking the binding of the first target antigen binding domain to its target via specific intermolecular interactions between the binding moiety and the first target antigen binding domain. In some embodiments, the non-CDR loop provides a binding site specific for binding of the binding moiety to the first target antigen binding domain. In some embodiments, the first or the second target antigen binding domain binds to a tumor antigen). In some embodiments, the tumor antigen comprises at least one of: EpCAM (exemplary protein sequence comprises UniProtkB ID No. P16422), EGFR (exemplary protein sequence comprises UniProtkB ID No. P00533), HER-2 (exemplary protein sequence comprises UniProtkB ID No. P04626), HER-3 (exemplary protein sequence comprises UniProtkB ID No. P21860), c-Met (exemplary protein sequence comprises UniProtkB ID No. P08581), FoIR (exemplary protein sequence comprises UniProtkB ID No. P15238), PSMA (exemplary protein sequence comprises UniProtkB ID No. Q04609), CD38 (exemplary protein sequence comprises UniProtkB ID No. P28907), BCMA (exemplary protein sequence comprises UniProtkB ID No. Q02223), and CEA (exemplary protein sequence comprises UniProtkB ID No. P06731. 5T4 (exemplary protein sequence comprises UniProtkB ID No. Q13641), AFP (exemplary protein sequence comprises comprises UniProtkB ID No. P02771), B7-H3 (exemplary protein sequence comprises UniProtkB ID No. Q5ZPR3), CDH-6 (exemplary protein sequence comprises UniProtkB ID No. P97326), CAIX (exemplary protein sequence comprises UniProtkB ID No. Q16790), CD117 (exemplary protein sequence comprises UniProtkB ID No. P10721), CD123 (exemplary protein sequence comprises UniProtkB ID No. P26951), CD138 (exemplary protein sequence comprises UniProtkB ID No. P18827), CD166 (exemplary protein sequence comprises UniProtkB ID No. Q13740), CD19 (exemplary protein sequence comprises UniProtkB ID No. P15931), CD20 (exemplary protein sequence comprises UniProtkB ID No. P11836), CD205 (exemplary protein sequence comprises UniProtkB ID No. 060449), CD22 (exemplary protein sequence comprises UniProtkB ID No. P20273), CD30 (exemplary protein sequence comprises UniProtkB ID No. P28908), CD33 (exemplary protein sequence comprises UniProtkB ID No. P20138), CD352 (exemplary protein sequence comprises UniProtkB ID No. Q96DU3), CD37 (exemplary protein sequence comprises UniProtkB ID No. P11049), CD44 (exemplary protein sequence comprises UniProtkB ID No. P16070), CD52 (exemplary protein sequence comprises UniProtkB ID No. P31358), CD56 (exemplary protein sequence comprises UniProtkB ID No. P13591), CD70 (exemplary protein sequence comprises UniProtkB ID No. P32970), CD71 (exemplary protein sequence comprises UniProtkB ID No. P02786), CD74 (exemplary protein sequence comprises UniProtkB ID No. P04233), CD79b (exemplary protein sequence comprises UniProtkB ID No. P40259), DLL3 (exemplary protein sequence comprises UniProtkB ID No. Q9NYJ7), EphA2 (exemplary protein sequence comprises UniProtkB ID No. P29317), FAP (exemplary protein sequence comprises UniProtkB ID No. Q12884), FGFR2 (exemplary protein sequence comprises UniProtkB ID No. P21802), FGFR3 (exemplary protein sequence comprises UniProtkB ID No. P22607), GPC3 (exemplary protein sequence comprises UniProtkB ID No. P51654), gpA33 (exemplary protein sequence comprises UniProtkB ID No. Q99795), FLT-3 (exemplary protein sequence comprises UniProtkB ID No. P36888), gpNMB (exemplary protein sequence comprises UniProtkB ID No. Q14956), HPV-16 E6 (exemplary protein sequence comprises UniProtkB ID No. P03126), HPV-16 E7 (exemplary protein sequence comprises UniProtkB ID No. P03129), ITGA2 (exemplary protein sequence comprises UniProtkB ID No. P17301), ITGA3 (exemplary protein sequence comprises UniProtkB ID No. P26006), SLC39A6 (exemplary protein sequence comprises UniProtkB ID No. Q13433), MAGE (exemplary protein sequence comprises UniProtkB ID No. Q9HC15), mesothelin (exemplary protein sequence comprises UniProtkB ID No. Q13421), Muc (exemplary protein sequence comprises UniProtkB ID No. P15941), Muc16 (exemplary protein sequence comprises UniProtkB ID No. Q8WX17), NaPi2b (exemplary protein sequence comprises UniProtkB ID No. 095436), Nectin-4 (exemplary protein sequence comprises UniProtkB ID No. Q96918), CDH-3 (exemplary protein sequence comprises UniProtkB ID No. Q8WX17), CDH-17 (exemplary protein sequence comprises UniProtkB ID No. E5RJT3), EPHB2 (exemplary protein sequence comprises UniProtkB ID No. P29323), ITGAV (exemplary protein sequence comprises UniProtkB ID No. P06756), ITGB6 (exemplary protein sequence comprises UniProtkB ID No. P18564), NY-ESO-1 (exemplary protein sequence comprises UniProtkB ID No. P78358), PRLR (exemplary protein sequence comprises UniProtkB ID No. P16471), PSCA (exemplary protein sequence comprises UniProtkB ID No. 043653), PTK7 (exemplary protein sequence comprises UniProtkB ID No. Q13308), ROR1 (exemplary protein sequence comprises UniProtkB ID No. Q01973), SLC44A4 (exemplary protein sequence comprises UniProtkB ID No. Q53GD3), SLITRK5 (exemplary protein sequence comprises UniProtkB ID No. Q8IW52), SLITRK6 (exemplary protein sequence comprises UniProtkB ID No. Q9HY7), STEAP1 (exemplary protein sequence comprises UniProtkB ID No. Q9UHE8), TIM1 (exemplary protein sequence comprises UniProtkB ID No. Q96D42), Trop2 (exemplary protein sequence comprises UniProtkB ID No. P09758), or WT1 (exemplary protein sequence comprises UniProtkB ID No. P19544), or any combinations thereof. In some embodiments, the first or the second target antigen binding domain binds to an immune checkpoint protein. In some embodiments, the immune checkpoint protein is at least one of CD27 (exemplary protein sequence comprises UniProtkB ID No. P26842), CD137 (exemplary protein sequence comprises UniProtkB ID No. Q07011), 2B4 (exemplary protein sequence comprises UniProtkB ID No. Q9bZW8), TIGIT (exemplary protein sequence comprises UniProtkB ID No. Q495A1), CD155 (exemplary protein sequence comprises UniProtkB ID No. P15151), ICOS (exemplary protein sequence comprises UniProtkB ID No. Q9Y6W8), HVEM (exemplary protein sequence comprises UniProtkB ID No. 043557), CD40L (exemplary protein sequence comprises UniProtkB ID No. P29965), LIGHT (exemplary protein sequence comprises UniProtkB ID No. 043557), OX40 (exemplary protein sequence comprises UniProtkB ID No. ), DNAM-1 (exemplary protein sequence comprises UniProtkB ID No. Q15762), PD-L1 (exemplary protein sequence comprises UniProtkB ID No. Q9ZQ7), PD1 (exemplary protein sequence comprises UniProtkB ID No. Q15116), PD-L2 (exemplary protein sequence comprises UniProtkB ID No. Q9BQ51), CTLA-4 (exemplary protein sequence comprises UniProtkB ID No. P16410), CD8 (exemplary protein sequence comprises UniProtkB ID No. P10966, P01732), CD40 (exemplary protein sequence comprises UniProtkB ID No. P25942), CEACAM1 (exemplary protein sequence comprises UniProtkB ID No. P13688), CD48 (exemplary protein sequence comprises UniProtkB ID No. P09326), CD70 (exemplary protein sequence comprises UniProtkB ID No. P32970), AA2AR (exemplary protein sequence comprises UniProtkB ID No. P29274), CD39 (exemplary protein sequence comprises UniProtkB ID No. P49961), CD73 (exemplary protein sequence comprises UniProtkB ID No. P21589), B7-H3 (exemplary protein sequence comprises UniProtkB ID No. Q5ZPR3), B7-H4 (exemplary protein sequence comprises UniProtkB ID No. Q7Z7D3), BTLA (exemplary protein sequence comprises UniProtkB ID No. Q76A9), IDO1 (exemplary protein sequence comprises UniProtkB ID No. P14902), IDO2 (exemplary protein sequence comprises UniProtkB ID No. Q6ZQW0), TDO (exemplary protein sequence comprises UniProtkB ID No. P48755), KIR (exemplary protein sequence comprises UniProtkB ID No. Q99706), LAG-3 (exemplary protein sequence comprises UniProtkB ID No. P18627), TIM-3 (also known as HAVCR2, exemplary protein sequence comprises UniProtkB ID No. Q8TDQ0), or VISTA (exemplary protein sequence comprises UniProtkB ID No. Q9D659). In some embodiments, the first or the second target antigen binding domain binds to an immune cell.

In some embodiments, the first or the second target antigen binding domain binds to a T-cell. In some embodiments, the first or the second target antigen binding domain binds to CD3. In some embodiments, the binding moiety (M), the cleavable linker (L), the first target antigen binding domain (T1), and the second target antigen binding domain (T2) are in one of the following configurations: M:L:T1:T2, and T2:T1:L:M. In some embodiments, the binding moiety comprises an albumin binding domain (anti-Alb), the first target antigen binding domain (T1) comprises a CD3 binding domain (e.g., an anti-CD3 scFV), and a ProTriTAC molecule has the following orientation: anti-Alb: anti-CD3: T2. In some embodiments, the binding moiety comprises an albumin binding domain (anti-Alb), the second target antigen binding domain (T2) comprises a CD3 binding domain (e.g., an anti-CD3 scFV), and a ProTriTAC molecule has the following orientation: anti-Alb: T1: anti-CD3. The T1 domain, in certain examples, is a tumor antigen binding domain, such as, but not limited to, an anti-EGFR domain, an anti-MSLN domain, an anti-BCMA domain, an anti-EpCAM domain, an anti-PSMA domain, or an anti-DLL3 domain.

In some embodiments, the cleavable linker comprises a cleavage site. In some embodiments, the cleavage site is recognized by a protease. In some embodiments, the protease cleavage site is recognized by a serine protease, a cysteine protease, an aspartate protease, a threonine protease, a glutamic acid protease, a metalloproteinase, a gelatinase, or an asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAM9, an ADAM10, an ADAM12, an urokinase plasminogen activator (uPA), an enterokinase, a prostate-specific target (PSA, hK3), an interleukin-iβ converting enzyme, a thrombin, a FAP (FAP-α), a dipeptidyl peptidase, a type II transmembrane serine protease (TTSP), a neutrophil elastase, a cathepsin G, a proteinase 3, a neutrophil serine protease 4, a mast cell chymase, and a mast cell tryptase. In some embodiments, the conditionally active protein further comprises a half-life extension domain bound to the binding moiety, wherein the half-life extension domain provides the binding protein with a safety switch, and wherein upon cleavage of the linker the binding protein is activated by separation of the binding moiety and the half-life extension domain from the first target antigen binding domain, and the binding protein is thereby separated from the safety switch. In some embodiments, the cleavage of the linker is in a tumor microenvironment.

One embodiment provides a conditionally active binding protein, comprising a binding moiety linked to a target antigen binding domain via a non-CDR loop within the binding moiety, wherein the binding moiety is further linked to a half-life extension domain and comprises a cleavable linker, wherein the target antigen binding domain comprises an immunoglobulin molecule, wherein the binding protein has an extended half-life prior to its activation by cleavage of the linker, and wherein upon activation the binding moiety and the half-life extension domain are separated from the target antigen binding domain, and wherein the binding protein, in its activated state, does not have an extended half-life. In some embodiments, the cleavage of the linker is in a tumor microenvironment.

In some embodiments, the non-CDR loop comprises a CC′ loop of at least one of: a camelid VHH domain, a human VH domain, a humanized VH domain, or a single domain antibody. In some embodiments, the binding moiety comprises a binding site specific for a CD3e domain, and wherein the binding site for the CD3e domain comprises at least one of the following motifs: QDGNE (SEQ ID NO: 921), QDGNEE (SEQ ID NO: 801), DGNE (SEQ ID NO: 922), and DGNEE (SEQ ID NO: 923).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 illustrates a variable domain of an exemplary immunoglobulin domain, comprising complementarity determining regions (CDR1, CDR2, and CDR3), and non-CDR loops connecting the beta strand (AB, CC′, C″ D, EF, and DE).

FIGS. 2A-2B provide exemplary arrangements of various domains of a conditionally active binding protein of this disclosure. FIG. 2A: Version 1. FIG. 2B: Version 2.

FIG. 3 shows an exemplary conditionally active target binding protein of this disclosure.

FIGS. 4A-4C show activation and possible mode of action of trispecific molecules (ProTriTAC). FIG. 4A shows ProTriTAC molecules in circulation, in tumor environment, and in circulation. FIG. 4B shows an exemplary sequence (SEQ ID NO: 929) for the protease cleavable site in a linker tethered to an anti-albumin binding moiety and FIG. 4C shows an SDS-PAGE gel showing the ProTriTAC in its activatable (prodrug) and activated (active drug) states.

FIGS. 5A-5B illustrate a process for making and purifying molecules described herein. FIG. 5A shows a schematic flowchart for manufacturing a ProTriTAC molecule and FIG. 5B shows an SDS-PAGE gel showing three purified ProTriTAC molecules.

FIGS. 6A-6B show analytical size exclusion chromatograms on a ProTriTAC molecule exposed to different stress conditions in graph form in FIG. 6A with the corresponding data in FIG. 6B. FIG. 6B provides the data for FIG. 6A.

FIG. 7 shows protease-dependent, anti-tumor activity of exemplary ProTriTAC molecules in HCT116 Colorectal Tumor Xenograft Model in NSG Mice.

FIGS. 8A-8D show various designs of exemplary ProTriTAC and control molecules. FIG. 8A: Control #1; FIG. 8B: Control #2; FIG. 8C: ProTriTAC; and FIG. 8D: Activated ProTriTAC.

FIG. 9 shows pharmacokinetic profiles for exemplary ProTriTAC and control molecules.

FIG. 10 shows conversion and half-life of exemplary ProTriTAC molecule.

FIG. 11 shows plasma clearance of an exemplary ProTriTAC molecule and its converted active drug format.

FIG. 12 shows CD3 binding potential of an exemplary ProTriTAC molecule, its converted active drug format, and a control non-cleavable ProTriTAC molecule.

FIG. 13 shows human primary T cell binding potential of an exemplary ProTriTAC molecule, its converted active drug format, and a control non-cleavable ProTriTAC molecule.

FIG. 14 shows T cell killing potential of an exemplary ProTriTAC molecule, its converted active drug format, and a control non-cleavable ProTriTAC molecule.

FIG. 15 shows the schematic structure of an exemplary trispecific molecule containing a binding moiety as described herein (also referred to herein as ProTriTAC or activatable ProTriTAC).

FIGS. 16A-16E shows exemplary schematic structures for Prodrug molecules combining functional masking and half-life extension. FIG. 16A shows a ProDrug molecule comprising an anti-albumin moiety which includes a masking moiety, and a cleavable linker connecting the anti-albumin moiety and the drug. FIG. 16B shows a ProDrug molecule comprising an anti-albumin moiety comprising two peptide motifs linked by linker, one of which includes a masking moiety, and a cleavable linker connecting the albumin binding moiety to a drug. FIG. 16C shows a ProDrug molecule comprising a modified albumin (containing a masking moiety) linked to a drug by a cleavable linker. FIG. 16D shows a ProDrug molecule comprising a modified albumin (containing a masking moiety and a protease cleavage site) linked to a drug. FIG. 16E shows an activated ProDrug. In each schematic structure (FIGS. 16A-16D) the drug molecule is functionally masked by the anti-albumin moiety or the modified albumin from binding its target or from being activated at an undesired site or from binding at non-target sites and thereby creating a drug sink.

FIGS. 17A-17E show exemplary schematic structures for ProTriTAC molecules combining functional masking and half-life extension. FIG. 17A shows a ProTriTAC molecule comprising an anti-albumin moiety which includes a masking moiety, and a cleavable linker connecting the anti-albumin moiety and a T cell engager molecule. FIG. 17B shows a ProTriTAC molecule comprising an anti-albumin moiety comprising two peptide motifs linked by linker, one of which includes a masking moiety, and a cleavable linker connecting the albumin binding moiety to a T cell engager molecule. FIG. 17C shows a ProTriTAC molecule comprising a modified albumin (containing a masking moiety) linked to a T cell engager by a cleavable linker. FIG. 17D shows a ProTriTAC molecule comprising a modified albumin (containing a masking moiety and a protease cleavage site) linked to a T cell engager. FIG. 17E shows an activated ProTriTAC. In each schematic structure (FIGS. 17A-17D) a target binding interface within the ProTriTAC molecule is functionally masked by the anti-albumin moiety or the modified albumin from binding its target or from being activated at an undesired site or from binding at non-target sites and thereby creating a sink.

FIG. 18 shows anti-tumor activity of exemplary ProTriTAC molecules and TriTAC molecules of this disclosure.

FIG. 19 shows pharmacokinetic profile of exemplary ProTriTAC molecules and TriTAC molecules of this disclosure.

FIGS. 20A-20F show admix xenograft individual tumor volumes following administering exemplary ProTriTAC molecules or TriTAC molecules of this disclosure. FIG. 20A illustrates results from a GFP TriTAC. FIG. 20B. illustrates results from EGFR ProTriTAC (NCLV). FIG. 20C illustrates results from EGFR ProTriTAC (L001). FIG. 20D illustrates results from EGFR ProTriTAC (L041). FIG. 20E illustrates results from EGFR ProTriTAC (L040). FIG. 20F illustrates results from EGFR ProTriTAC (L045).

FIGS. 21A-21C shows cytokine levels (IFN-gamma (FIG. 21A), IL-6 (FIG. 21B), and IL-10; FIG. 21C) following administering exemplary ProTriTAC molecules or TriTAC molecules of this disclosure.

FIGS. 22A-22E show body weight percent change in mice, following administering exemplary ProTriTAC molecules and TriTAC molecules of this disclosure. FIG. 22A illustrates results of 30 μg/kg; FIG. 22B illustrates results of 100 μg/kg; FIG. 22C illustrates results of 300 μg/kg; FIG. 22D illustrates results of 1000 μg/kg; and FIG. 22E provides fold protection at the various concentrations.

FIGS. 23A-23C show body weight percent change in mice, following administering varying concentrations of exemplary ProTriTAC molecules of this disclosure, containing non-cleavable or cleavable linkers. FIG. 23A illustrates results of 300 μg/kg; FIG. 23B illustrates results of 1000 μg/kg; FIG. 23C provides fold protection at the various concentrations.

FIGS. 24A-24C show serum concentrations of aspartate aminotransferase (AST), in mice, following administering varying concentrations of a ProTriTAC molecule containing a non-cleavable linker (ProTriTAC (NCLV)) (FIG. 24C), a TriTAC molecule (FIG. 24A), or a ProtriTAC molecule (FIG. 24B) containing a cleavable linker.

FIGS. 25A-25C show serum concentrations of alanine aminotransferase (ALT), in mice, following administering varying concentrations of a ProTriTAC molecule containing a non-cleavable linker (ProTriTAC (NCLV)) (FIG. 25C), a TriTAC molecule (FIG. 25A), or a ProtriTAC molecule (FIG. 25B) containing a cleavable linker.

FIGS. 26A-26B show show serum concentrations of ALT (right panel; FIG. 26B) or AST (left panel; FIB. 26A), in cynomolgus monkeys, following administering varying concentrations of an EGFR ProTriTAC molecule, or an EGFR ProTriTAC (NCLV) molecule.

FIGS. 27A-27D show tumor volume in mice following administration of a GFP TriTAC molecule, an EGFR TriTAC molecule, or an EGFR ProTriTAC molecule, in varying concentrations. FIG. 27A shows GFP TriTAC (at 300 μg/kg) and an EGFR TriTAC (at 10 μg/kg). FIG. 27B shows the EGFR TriTAC (at 30 μg/kg and at 100 μg/kg). FIG. 27C shows the EGFR TriTAC (at 300 μg/kg) and an EGFR ProTriTAC (at 30 μg/kg and 100 μg/kg). FIG. 27D shows the EGFR ProTriTAC (at 300 μg/kg and 1000 μg/kg)

FIG. 28 shows serum concentrations of ALT and AST, in mice, following administration of varying concentrations of a GFP TriTAC, an EGFR TriTAC, and an EGFR ProTriTAC molecule.

FIG. 29 shows grafting of a CD3R epitope into the CC′ loop of a binding moiety of this disclosure. HuCD3e: SEQ ID NO: 902; CC10: SEQ ID NO: 260; CC12: SEQ ID NO: 259; and CC16: SEQ ID NO: 261. WT disclosed as SEQ ID NO: 795.

FIG. 30 shows separation of a binding moiety of this disclosure, from a ProTriTAC molecule that contained the binding moiety, upon tumor associated protease activation by matriptase.

FIG. 31 shows CD3 binding of ProTriTAC molecules, with or without activation, containing an exemplary binding moiety of this disclosure.

FIG. 32 shows cell killing potential of a ProTriTAC molecule, with or without activation, containing an exemplary binding moiety of this disclosure.

FIG. 33 shows the soft library mutagenesis approach carried out to explore the non-CDR loops within an exemplary binding moiety of this disclosure.

FIG. 34 illustrates the results of the soft library mutagenesis approach carried out to explore the non-CDR loops within an exemplary binding moiety of this disclosure, after panning against HSA (human serum albumin or also referred to herein as albumin). Figure discloses “Naïve Library” sequences as SEQ ID NOS 930-1018, respectively, in order of appearance and discloses “After panning with HSA” sequences as SEQ ID NOS 1019-1140, respectively, in order of appearance.

FIG. 35 illustrates that a binding moiety of this disclosure is able to expand the therapeutic window of a molecule containing it (e.g., a ProTriTAC molecule) by both steric masking and specific masking.

FIG. 36 provides results from a representative T cell dependency cellular cytotoxicity assay with NCI-H508 cells using exemplary fusion proteins of this disclosure containing an anti-EpCAM domain as described herein and an anti-CD3 domain.

FIG. 37 provides results from a representative T cell dependency cellular cytotoxicity assay using exemplary fusion proteins of this disclosure containing an anti-EpCAM domain as described herein and an anti-CD3 domain.

FIG. 38 provides results from a representative T cell dependency cellular cytotoxicity assay using exemplary fusion proteins of this disclosure containing an anti-EpCAM domain as described herein and an anti-CD3 domain.

FIG. 39 provides results from a representative T cell dependency cellular cytotoxicity assay using exemplary fusion proteins of this disclosure containing an anti-EpCAM domain as described herein and an anti-CD3 domain.

FIG. 40 provides results from a representative T cell dependency cellular cytotoxicity assay using exemplary fusion proteins of this disclosure containing a humanized anti-EpCAM domain as described herein and an anti-CD3 domain.

FIGS. 41A-41C show body weight percent change in mice, following administering exemplary EpCAM ProTriTAC molecules and EpCAM TriTAC molecules of this disclosure.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Provided herein in certain embodiments are ProTriTAC molecules (also referred to herein as protrisecific molecules) that are T cell engager prodrugs designed to be conditionally active in a tumor microenvironment. In some cases, this enables targeting of a wider selection of tumor antigens (e.g., solid tumor antigens). The ProTriTAC molecules, in some examples, combine the desirable attributes of several prodrug approaches, including, but not limited to: combination of steric and specific masking, wherein the steric masking is, in some cases, is through albumin that is recognized by an anti-albumin domain in a ProTriTAC molecule, and the specific masking, in some cases, is through specific intermolecular interactions between an anti-albumin domain (in some examples) and a target antigen binding domain of the ProTriTAC molecule (such as, an anti-CD3 scFv domain, in some examples); additional safety imparted by half-life differential of prodrug versus an active drug, derived by activation of the conditionally activated ProTriTAC molecule; ability to plug-and-play with different tumor target binders.

### CERTAIN DEFINITIONS

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

A “single chain Fv” or “scFv”, as used herein, refers to a binding protein in which the variable domains of the heavy chain and of the light chain of a traditional two chain antibody are joined to form one chain. Typically, a linker peptide is inserted between the two chains to allow for proper folding and creation of an active binding site.

A “cleavage site for a protease,” or “protease cleavage site”, as meant herein, is an amino acid sequence that can be cleaved by a protease, such as, for example, a matrix metalloproteinase or a furin. Examples of such sites include Gly-Pro-Leu-Gly-Ile-Ala-Gly-Gln (SEQ ID NO: 924) or Ala-Val-Arg-Trp-Leu-Leu-Thr-Ala (SEQ ID NO: 925), which can be cleaved by metalloproteinases, and Arg-Arg-Arg-Arg-Arg-Arg (SEQ ID NO: 926), which is cleaved by a furin. In therapeutic applications, the protease cleavage site can be cleaved by a protease that is produced by target cells, for example cancer cells or infected cells, or pathogens.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2}the time required for 50% completion of the process. The units of these two constants are time⁻¹ and time, respectively. A first-order rate constant and the half-time of the reaction are simply related (k×t_{1/2=0.693}) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

A “therapeutic agent,” as used herein, includes a “binding molecule.”

The term “binding molecule,” as used herein is any molecule, or portion or fragment thereof, that can bind to a target molecule, cell, complex and/or tissue, and which includes proteins, nucleic acids, carbohydrates, lipids, low molecular weight compounds, and fragments thereof, each having the ability to bind to one or more of a soluble protein, a cell surface protein, a cell surface receptor protein, an intracellular protein, a carbohydrate, a nucleic acid, a hormone, or a low molecular weight compound (small molecule drug), or a fragment thereof. The binding molecule, in some instances, is a protein belonging to the immunoglobulin superfamily, or a non-immunoglobulin binding molecule. The “binding molecule” does do not include a cytokine.

The term “proteins belonging to immunoglobulin superfamily,” or “immunoglobulin molecules,” as used herein, include proteins that comprise an immunoglobulin fold, such as antibodies and target antigen binding fragments thereof, antigen receptors, antigen presenting molecules, receptors on natural killer cells, antigen receptor accessory molecules, receptors on leukocytes, IgSF cellular adhesion molecules, growth factor receptors, and receptor tyrosine kinases/phosphatases.

The term “antibodies” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, bi-specific antibodies, multi-specific antibodies, and fusion proteins comprising an antigen-binding (also referred to herein as antigen binding) portion of an antibody and a non-antibody protein. The antibodies, in some examples, are detectably labeled, e.g., with a radioisotope, an enzyme that generates a detectable product, a fluorescent protein, and the like. The antibodies, in some cases, are further conjugated to other moieties, such as members of specific binding pairs, e.g., biotin (member of biotin-avidin specific binding pair), and the like. The antibodies, in some cases, are bound to a solid support, including, but not limited to, polystyrene plates or beads, and the like. Also encompassed by the term are Fab′, Fv, F(ab′)2, and or other antigen binding fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody, in some instances, is monovalent or bivalent. An antibody, in some instances, is an Ig monomer, which is a “Y-shaped” molecule that consists of four polypeptide chains: two heavy chains and two light chains connected by disulfide bonds.

The term “non-immunoglobulin binding molecules,” as used herein, include, but is not limited to examples such as a growth factor, a hormone, a signaling protein, an inflammatory mediator, ligand, a receptor, or a fragment thereof, a native hormone or a variant thereof being able to bind to its natural receptor; a nucleic acid or polynucleotide sequence being able to bind to complementary sequence or a soluble cell surface or intracellular nucleic acid/polynucleotide binding proteins, a carbohydrate binding moiety being able to bind to other carbohydrate binding moieties, cell surface or intracellular proteins, a low molecular weight compound (drug) that binds to a soluble or cell surface or intracellular target protein. The non-immunoglobulin binding molecules, in some cases, include coagulation factors, plasma proteins, fusion proteins, and imaging agents. The non-immunoglobulin binding molecules do not include a cytokine.

A “cytokine,” as meant herein, refers to intercellular signaling molecules, and active fragments and portions thereof, which are involved in the regulation of mammalian somatic cells. A number of families of cytokines, for example, interleukins, interferons, and transforming growth factors are included.

As used herein, “non-CDR loops” within immunoglobulin (Ig) molecules are regions of a polypeptide other than the complementarity determining regions (CDRs) of an antibody. These regions may be derived from an antibody or an antibody fragment. These regions may also be synthetically or artificially derived, such as through mutagenesis or polypeptide synthesis.

In an Ig, Ig-like, or beta-sandwich scaffold that has 9 beta-strands (e.g., a VH, a VL, a camelid VHH, a sdAb), the non-CDR loops can refer to the AB, CC′, C″D, EF loops or loops connecting beta-strands proximal to the C-terminus. In an Ig, Ig-like, or beta-sandwich scaffold that has 7 beta-strands (e.g., a CH, a CL, an adnectin, a Fn-III), the non-CDR loops can refer to the AB, CD, and EF loops or loops connecting beta-strands proximal the C-terminus. In other Ig-like or beta-sandwich scaffolds, the non-CDR loops are the loops connecting beta-strands proximal to the C-terminus or topologically equivalent residues using the framework established in the Halaby 1999 publication (Prot Eng Des Sel 12:563-571).

In a non-beta-sandwich scaffold (e.g., a DARPin, an affimer, an affibody), the “non-CDR loops” refer to an area that is (1) amenable for sequence randomization to allow engineered specificities to a second antigen, and (2) distal to the primary specificity determining region(s) typically used on the scaffold to allow simultaneous engagement of the scaffold to both antigens without steric interference. For this purpose, the primary specificity determining region(s) can be defined using the framework established in the Skrlec 2015 publication (Trends in Biotechnol, 33:408-418). An excerpt of the framework is listed below.

| Scaffold | Primary specificity determining region(s) |
|---|---|
| Affibody | 13 residues in two helices |
| Affimer | 12-36 residues |
| Anticalin | Four loops (up to 24 aa) |
| Avimer | 11 residues |
| Scaffold | Primary specificity determining region(s) |
| Centyrin | 13 residues |
| DARPin | 7 residues in each n-repeat, or |
| | 8 residues in each n-repeat |
| Fynomer | 6 residues in the RT- and n-Src-loop |
| Kunitz domain | 1-2 loops |

“Target antigen binding domain”, as used herein, refers to a region which targets a specific antigen. A target antigen binding domain comprises, for example an sdAb, an scFv, a variable heavy chain antibody (VHH), a variable heavy (VH) or a variable light domain (VL), a full length antibody, or any other peptide that has a binding affinity towards a specific antigen. The target antigen binding domain does do not include a cytokine.

“TriTAC,” as used herein refers to a trispecific binding protein that is not conditionally activated.

### Binding Moiety, Cleavable Linker and Conditionally Active Binding Proteins

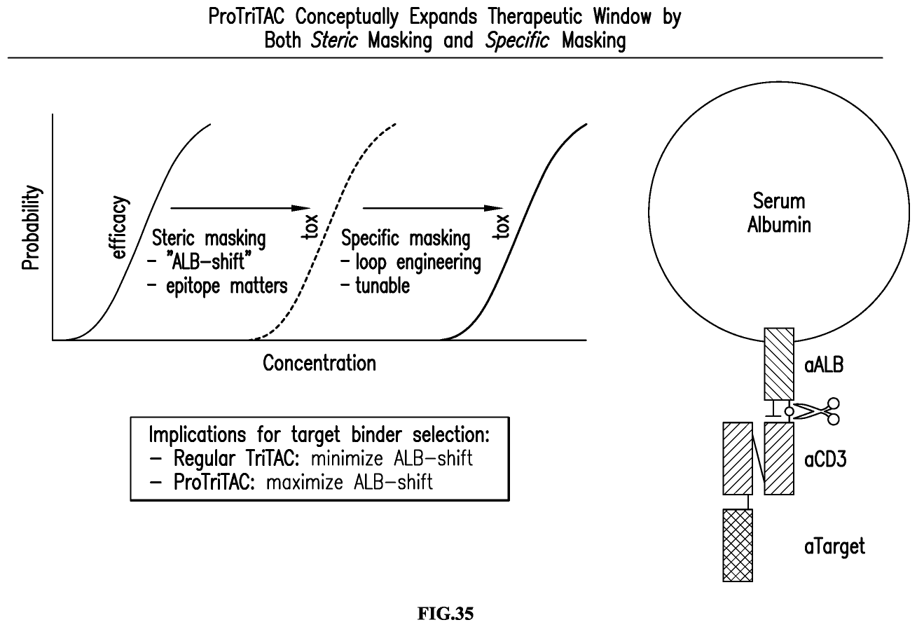

This disclosure provides, in some embodiments, binding moieties that are capable of masking the interaction of binding molecules with their targets. In some embodiments, a binding moiety of this disclosure comprises a masking moiety and a cleavable linker, such as a protease cleavable linker. In some embodiments, a binding moiety of this disclosure comprises a masking moiety (e.g., a modified non-CDR loop sequence) and a non-cleavable linker. As illustrated in FIG. 35, the binding moiety is capable of synergistically expanding a therapeutic window of a molecule that comprises the moiety, by both steric masking and specific masking. In some examples, the binding molecule is a protein belonging to an immunoglobulin superfamily, such as a target antigen binding domain comprising an immunoglobulin fold. In some embodiments, the binding molecule is a non-immunoglobulin protein. In some embodiments, the binding moiety combines both steric masking (for example, via binding to a bulky serum albumin) and specific masking (for example, via non-CDR loops binding to the CDRs of an anti-CD3 scFv domain). In some cases, modifying the non-CDR loops within the binding moiety does not affect albumin binding. The protease cleavable linker, in some cases, enables activation of a prodrug molecule comprising the binding moiety (such as a ProTriTAC molecule comprising a binding moiety as described herein, a CD3 binding domain, and an albumin binding domain), in a single proteolytic event, thereby allowing more efficient conversion of the prodrug molecule in tumor microenvironment. Further, tumor-associated proteolytic activation, in some cases, reveals active T cell engager (such as a ProTriTAC molecule comprising a binding moiety as described herein, a CD3 binding domain, and an albumin binding domain) with minimal off-tumor activity after activation. The present disclosure, in some embodiments, provides a half-life extended T cell engager format (ProTriTAC) comprising a binding moiety as described herein, which in some cases represents a new and improved approach to engineer conditionally active T cell engagers.

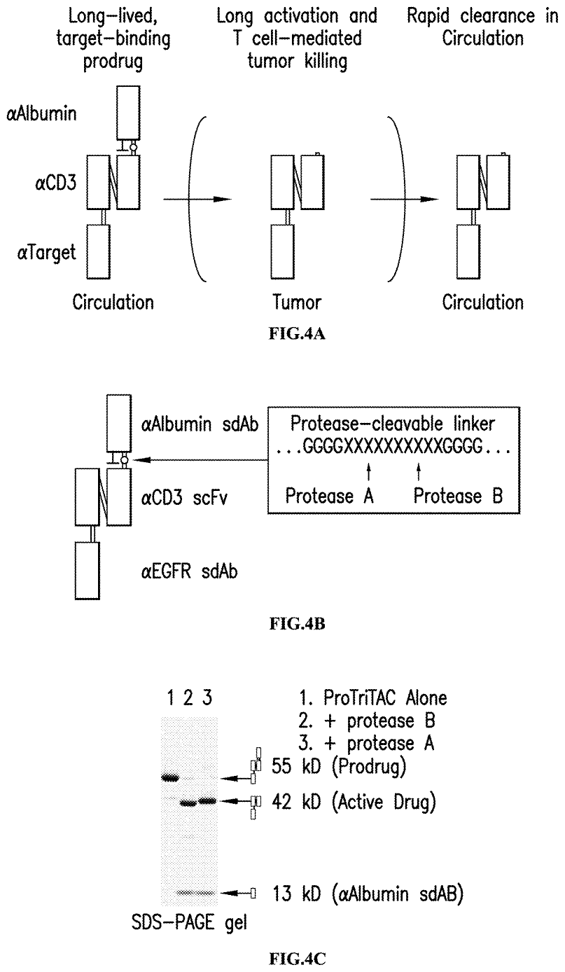

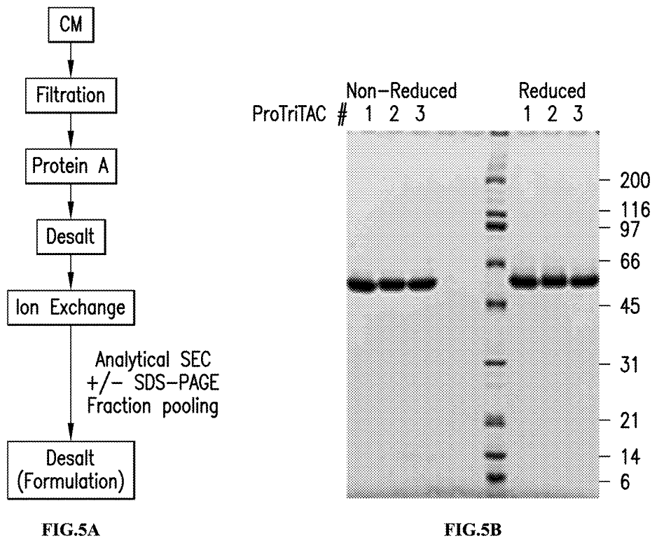

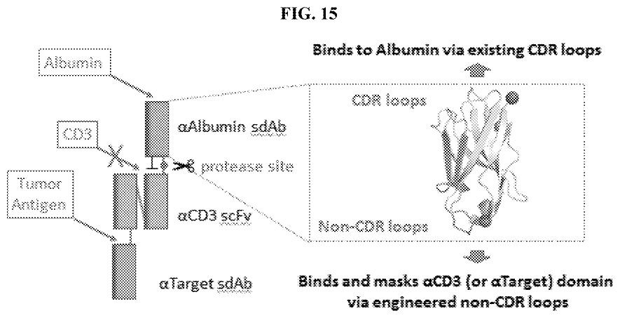

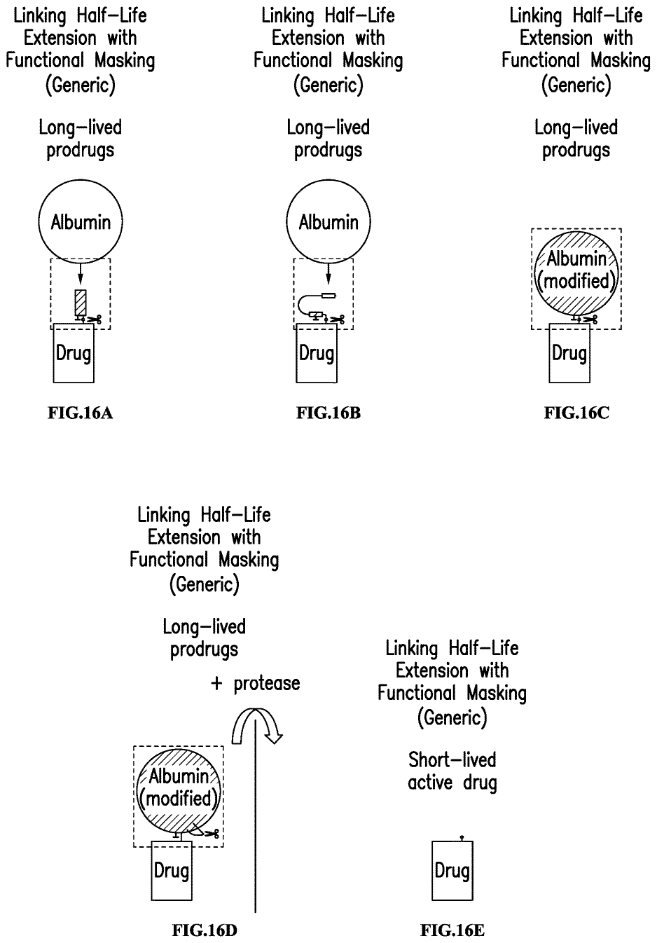

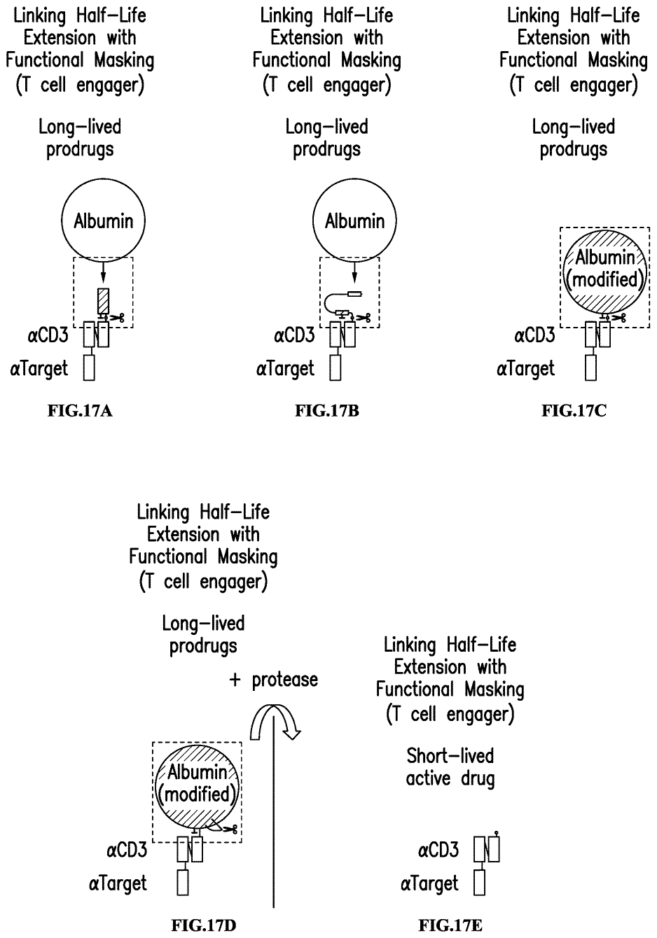

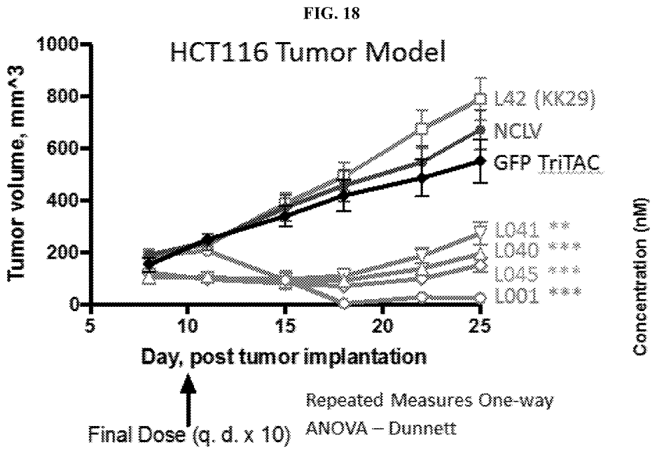

FIG. 4B provides a schematic for an exemplary ProTriTAC molecule comprising an exemplary binding moiety as described herein (the αalbumin sdAb) and a gel showing the ProTriTAC before and after activation by cleaving of the protease cleavable linker, and FIG. 4A shows a possible mode of action of the same. FIG. 15 shows the schematic structure of an exemplary trispecific molecule containing a binding moiety as described herein (also referred to herein as ProTriTAC or activatable ProTriTAC), with engineered non-CDR loops. The exemplary trispecific molecule contains an anti-albumin domain comprising a cleavable linker (such a linker comprising a protease cleavable site, also referred to herein as a substrate linker) and a masking domain; an anti-CD3 binding domain; and an anti-target domain (specific for a tumor antigen) which in some cases is a non-immunoglobulin molecule. In some cases, non-CDR loops in the anti-albumin domain is capable of binding and masking the anti-target domain. In some cases, non-CDR loops in the anti-albumin domain is capable of binding and masking the anti-CD3 domain. The binding moiety, in some embodiments, comprises a CDR loop specific for binding albumin.

Provided herein, in a first embodiment, is a binding moiety that masks the binding of a target antigen binding domain, and is capable of binding to a bulk-serum protein, such as a half-life extending protein. The binding moiety of the first embodiment, in certain instances, further comprises a cleavable linker attached to it. The cleavable linker, for example, comprises a protease cleavage site or a pH dependent cleavage site. The cleavable linker, in certain instances, is cleaved only in a tumor micro-environment. Thus, in some examples, the binding moiety of the first embodiment, bound to the half-life extending protein, connected to the cleavable linker, and further bound to the target antigen binding domain, maintains the target antigen binding domain in an inert state in circulation until the cleavable linker is cleaved off in a tumor microenvironment. The half-life of the target antigen binding domain, such as an antibody or an antigen binding fragment thereof, is thus extended in systemic circulation by using the binding moiety of the first embodiment which acts as a safety switch that keeps the target antigen binding moiety in an inert state until it reaches the tumor microenvironment where it is conditionally activated by cleavage of the linker and is able to bind its target antigen.

In a second embodiment is provided a binding moiety that masks the interaction between a non-immunoglobulin binding molecule and its target. The binding moiety of the second embodiment, in certain instances, is capable of binding to a bulk serum protein. In some instances, the binding moiety of the second embodiment further comprises a cleavable linker attached to it. The cleavable linker, for example, comprises a protease cleavage site or a pH dependent cleavage site. The cleavable linker, in certain instances, is cleaved only in a tumor micro-environment. The non-immunoglobulin binding molecule is, in some cases, maintained in an inert state by the binding moiety of the second embodiment and activated by cleavage of the linker, for example in a target environment. In some instances, the cleavable linker is cleaved off in a tumor microenvironment and in such cases the tumor microenvironment is the target environment. The half-life of the non-immunoglobulin binding molecule is thus extended in systemic circulation by using the binding moiety of the second embodiment which acts as a safety switch that keeps the non-immunoglobulin binding molecule in an inert state until it reaches the target environment where it is conditionally activated by cleavage of the linker. In some examples of the second embodiment where the non-immunoglobulin binding molecule is an imaging agent, said agent is activated in a target environment upon cleavage of the cleavable linker. The target environment, in such cases, is a tissue or a cell or any biological environment that is to be imaged using the imaging agent.

The safety switch described above provides several advantages; some examples including (i) expanding the therapeutic window of the immunoglobulin molecule, such as a target antigen binding domain, a non-immunoglobulin binding molecule; (ii) reducing target-mediated drug disposition by maintaining the immunoglobulin molecule, such as a target antigen binding domain, the non-immunoglobulin binding molecule, in an inert state when a conditionally active protein comprising a binding moiety according to the first or second embodiments is in systemic circulation; (iii) reducing the concentration of undesirable activated proteins in systemic circulation, thereby minimizing the spread of chemistry, manufacturing, and controls related impurities, e.g., pre-activated drug product, endogenous viruses, host-cell proteins, DNA, leachables, anti-foam, antibiotics, toxins, solvents, heavy metals; (iv) reducing the concentration of undesirable activated proteins in systemic circulation, thereby minimizing the spread of product related impurities, aggregates, breakdown products, product variants due to: oxidation, deamidation, denaturation, loss of C-term Lys in MAbs; (v) preventing aberrant activation of the immunoglobulin molecule, such as a target antigen binding domain, or the non-immunoglobulin binding molecule in circulation; (vi) reducing the toxicities associated with the leakage of activated species from diseased tissue or other pathophysiological conditions, e.g., tumors, autoimmune diseases, inflammations, viral infections, tissue remodeling events (such as myocardial infarction, skin wound healing), or external injury (such as X-ray, CT scan, UV exposure); and (vii) reducing non-specific binding of the immunoglobulin molecule, such as a target antigen binding domain, or the non-immunoglobulin binding molecule. Furthermore, post-activation, or in other words post breaking of the safety switch, the immunoglobulin molecule, such as a target antigen binding domain, the non-immunoglobulin binding molecule is separated from the safety switch which provided extended half-life, and thus is cleared from circulation.

In addition, the binding moieties of the first, second, and the third embodiments, in some cases, are used to generate a “biobetter” version of a biologic. Generally, preparing a biobetter form of a molecule, e.g., an antibody or an antigen binding fragment thereof, involves taking the originator molecule and making specific alterations in it to improve its parameters and thereby make it a more efficacious, less frequently dosed, better targeted, and/or a better tolerated drug. Thus, a target antigen binding domain masked by the binding moiety of the first embodiment which is bound to a half-life extending protein, and conditionally activated in a tumor microenvironment by cleavage of the cleavable linker, gives the target antigen binding domain a significantly longer serum half-life and reduces the likelihood of its undesirable activation in circulation, thereby producing a “biobetter” version of the target antigen binding domain. Similarly, the binding moieties of the second embodiment are, in some cases, utilized to generate biobetter versions of the non-immunoglobulin binding molecules. Accordingly, in various embodiments, biobetter versions of immunoglobulin molecules, non-immunoglobulin binding molecules are provided, wherein the biobetter function is attributed to a binding moiety, respectively, according to the first or second embodiments.

The binding moieties described herein comprise at least one non-CDR loop. In some embodiments, a non-CDR loop provides a binding site for binding of the binding moiety of the first embodiment to a target antigen binding domain. In some examples of the first embodiment, a non-CDR loop provides a binding site for binding of the binding moiety of the first embodiment to an immunoglobulin molecules, such as a target antigen binding domain. In some examples of the second embodiment, a non-CDR loop provides a binding site for binding of the binding moiety of the second embodiment to a non-immunoglobulin binding molecule. In some cases, the binding moiety of the first embodiment masks binding of the target binding domain to the target antigen, e.g. via steric occlusion, via specific intermolecular interactions, or a combination of both. The binding moieties of the second embodiment also, in some cases, mask binding of a non-immunoglobulin binding molecule to their targets, via steric occlusion, via specific intermolecular interactions, or a combination of both.

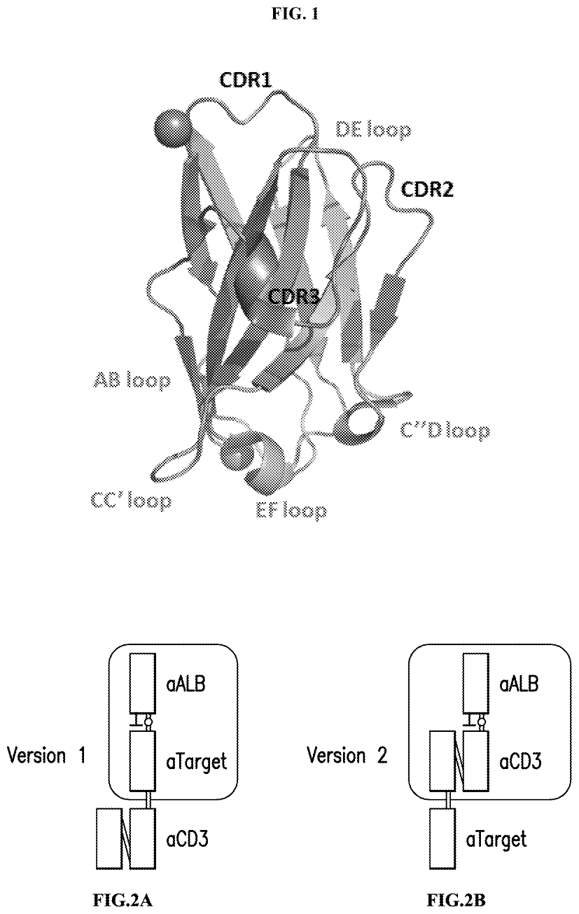

In some embodiments, the binding moieties described herein further comprise complementarity determining regions (CDRs). In some instances, the binding moieties are domains derived from an immunoglobulin molecule (Ig molecule). The Ig may be of any class or subclass (IgG1, IgG2, IgG3, IgG4, IgA, IgE, IgM etc). A polypeptide chain of an Ig molecule folds into a series of parallel beta strands linked by loops. In the variable region, three of the loops constitute the “complementarity determining regions” (CDRs) which determine the antigen binding specificity of the molecule. An IgG molecule comprises at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs) with are hypervariable in sequence and/or involved in antigen recognition and/or usually form structurally defined loops, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments of this disclosure, at least some or all of the amino acid sequences of FR1, FR2, FR3, and FR4 are part of the “non-CDR loop” of the binding moieties described herein. As shown in FIG. 1, a variable domain of an immunoglobulin molecule has several beta strands that are arranged in two sheets. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands B-C, C′-C″, and F-G of the immunoglobulin fold, whereas the bottom loops that connect beta strands AB, CC′, C″-D and E-F of the immunoglobulin fold, and the top loop that connects the D-E strands of the immunoglobulin fold are the non-CDR loops. In some embodiments of this disclosure, at least some amino acid residues of a constant domain, CH1, CH2, or CH3, are part of the “non-CDR loop” of the binding moieties described herein. Non-CDR loops comprise, in some embodiments, one or more of AB, CD, EF, and DE loops of a C1-set domain of an Ig or an Ig-like molecule; AB, CC′, EF, FG, BC, and EC′ loops of a C2-set domain of an Ig or an Ig-like molecule; DE, BD, GF, A(A1A2)B, and EF loops of I(Intermediate)-set domain of an Ig or Ig-like molecule.

Within the variable domain, the CDRs are believed to be responsible for antigen recognition and binding, while the FR residues are considered a scaffold for the CDRs. However, in certain cases, some of the FR residues play an important role in antigen recognition and binding. Framework region residues that affect Ag binding are divided into two categories. The first are FR residues that contact the antigen, thus are part of the binding-site, and some of these residues are close in sequence to the CDRs. Other residues are those that are far from the CDRs in sequence, but are in close proximity to it in the 3-D structure of the molecule, e.g., a loop in heavy chain.

In some embodiments, the non-CDR loop is modified to generate an antigen binding site specific for a bulk serum protein, such as albumin. It is contemplated that various techniques can be used for modifying the non-CDR loop, e.g., site-directed mutagenesis, random mutagenesis, insertion of at least one amino acid that is foreign to the non-CDR loop amino acid sequence, amino acid substitution. An antigen peptide is inserted into a non-CDR loop, in some examples. In some examples, an antigenic peptide is substituted for the non-CDR loop. The modification, to generate an antigen binding site, is in some cases in only one non-CDR loop. In other instances, more than one non-CDR loop are modified. For instance, the modification is in any one of the non-CDR loops shown in FIG. 1, i.e., AB, CC′, C″ D, EF, and D-E. In some cases, the modification is in the DE loop. In other cases the modifications are in all four of AB, CC′, C″-D, E-F loops. In certain examples, the binding moieties described herein are bound to the immunoglobulin molecules, e.g., a target antigen binding domain, the non-immunoglobulin binding molecules via their AB, CC′, C″ D, or EF loop and are bound to a bulk-serum protein, such as albumin, via their B-C, C′-C″, or F-G loop. In certain examples, the binding moiety of the first embodiment is bound to the target antigen binding domain via its AB, CC′, C″ D, and EF loop and is bound to a bulk-serum protein, such as albumin, via its BC, C′C″, and FG loop.

In certain examples, the binding moiety of the first embodiment is bound to a target antigen binding domain via one or more of AB, CC′, C″ D, and E-F loop and is bound to a bulk-serum protein, such as albumin, via one or more of BC, C′C″, and FG loop. In certain examples, the binding moiety of the first embodiment is bound to a bulk serum protein, such as albumin, via its AB, CC′, C″ D, or EF loop and is bound to a target antigen binding domain via its BC, C′C″, or FG loop. In certain examples, the binding moiety of the first embodiment is bound to a bulk serum protein, such as albumin, via its AB, CC′, C″ D, and EF loop and is bound to a target antigen binding domain via its BC, C′C″, and FG loop. In certain examples, the binding moiety of the first embodiment is bound to a bulk serum protein, such as albumin, via one or more of AB, CC′, C″ D, and E-F loop and is bound to the target antigen binding protein, via one or more of BC, C′C″, and FG loop.

In certain examples, the binding moiety of the second embodiment is bound to a non-immunoglobulin molecule via one or more of AB, CC′, C″ D, and E-F loop and is bound to a bulk-serum protein, such as albumin, via one or more of BC, C′C″, and FG loop. In certain examples, the binding moiety of the second embodiment is bound to a bulk serum protein, such as albumin, via its AB, CC′, C″ D, or EF loop and is bound to a non-immunoglobulin molecule via its BC, C′C″, or FG loop. In certain examples, the binding moiety of the second embodiment is bound to a bulk serum protein, such as albumin, via its AB, CC′, C″ D, and EF loop and is bound to a non-immunoglobulin molecule via its BC, C′C″, and FG loop. In certain examples, the binding moiety of the second embodiment is bound to a bulk serum protein, such as albumin, via one or more of AB, CC′, C″ D, and E-F loop and is bound to a non-immunoglobulin molecule, via one or more of BC, C′C″, and FG loop.

The binding moieties are any kinds of polypeptides. For example, in certain instances the binding moieties are natural peptides, synthetic peptides, or fibronectin scaffolds, or engineered bulk serum proteins. The bulk serum protein comprises, for example, albumin, fibrinogen, or a globulin. In some embodiments, the binding moieties are engineered scaffolds. Engineered scaffolds comprise, for example, sdAb, a scFv, a Fab, a VHH, a fibronectin type III domain, immunoglobulin-like scaffold (as suggested in Halaby et al., 1999. Prot Eng 12(7):563-571), DARPin, cystine knot peptide, lipocalin, three-helix bundle scaffold, protein G-related albumin-binding module, or a DNA or RNA aptamer scaffold.

In some cases, the binding moiety of the first embodiment binds to at least one target antigen binding domain. In further embodiments, the non-CDR loops within the binding moiety of the first embodiment provide a binding site for the at least one target antigen binding domain. The target antigen binding domain, in some cases, binds to target antigens expressed on the surface of a diseased cell or tissue, for example a tumor or a cancer cell. Target antigens include but are not limited to EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

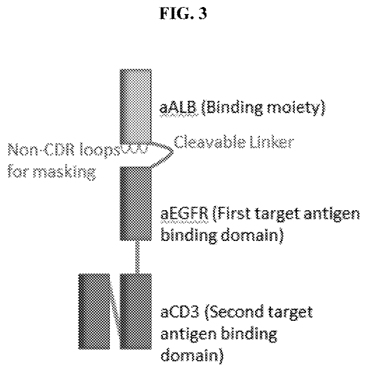

In some cases, the binding moiety of the first embodiment is bound to a first target antigen binding domain via its non-CDR loops and the first target antigen binding domain is further connected to a second target antigen binding domain. Examples of first and second target antigen binding domains include, but are not limited to, a T cell engager, a bispecific T cell engager, a dual-affinity re-targeting antibody, a variable heavy domain (VH), a variable light domain (VL), a scFv comprising a VH and a VL domain, a soluble TCR fragment comprising a Valpha and Vbeta domain, a single domain antibody (sdAb), or a variable domain of camelid derived nanobody (VHH), a non-Ig binding domain, i.e., antibody mimetic, such as anticalins, affilins, affibody molecules, affimers, affitins, alphabodies, avimers, DARPins, fynomers, kunitz domain peptides, and monobodies, a ligand or peptide. In some examples, the first or the second target antigen binding domain is a VHH domain. In some examples, the first or the second target antigen binding domain is a sdAb. In some instances, the first target antigen binding domain is specific for a tumor antigen, such as EGFR, and the second target antigen binding domain is specific for CD3. The binding of the first target antigen binding domain to its target, e.g., a tumor antigen such as EGFR, is masked by the binding moiety of the first embodiment, via its non-CDR loops. One exemplary conditionally active protein, comprising a binding moiety according to the first embodiment, is shown in FIG. 3.

In some cases, the non-CDR loops within the binding moiety of the second embodiment provide a binding site for a non-immunoglobulin binding molecule.

In some cases, the binding moieties comprise a binding site for a bulk serum protein. In some embodiments, the CDRs within the binding moieties provide a binding site for the bulk serum protein. The bulk serum protein is, in some examples, a globulin, albumin, transferrin, IgG1, IgG2, IgG4, IgG3, IgA monomer, Factor XIII, Fibrinogen, IgE, or pentameric IgM. In some embodiments, the binding moieties comprise a binding site for an immunoglobulin light chain. In some embodiments, the CDRs provide a binding site for the immunoglobulin light chain. The immunoglobulin light chain is, in some examples, an Igκ free light chain or an Igλ free light chain.

In some examples, the binding moieties comprise any type of binding domain, including but not limited to, domains from a monoclonal antibody, a polyclonal antibody, a recombinant antibody, a human antibody, a humanized antibody. In some embodiments, the binding moiety is a single chain variable fragment (scFv), a soluble TCR fragment, a single-domain antibody such as a heavy chain variable domain (VH), a light chain variable domain (VL) and a variable domain (VHH) of camelid derived nanobody. In other embodiments, the binding moieties are non-Ig binding domains, i.e., antibody mimetic, such as anticalins, affilins, affibody molecules, affimers, affitins, alphabodies, avimers, DARPins, fynomers, kunitz domain peptides, and monobodies.

TABLE 1
Exemplary Sequences for Masking Sequences within
the Binding Moieties of this Disclosure are
Provided In SEQ ID Nos. 50, 259-301 And 795.

| | | Alt. | |
|---|---|---|---|
| MaskID | Sequence | Names | SEQ ID No. |
| MASK001 | APGKG | WT | SEQ ID No. 795 |
| MASK002 | GGQDGNEEMGGG | CC12 | SEQ ID No. 259 |
| MASK004 | GGQDGNEEGG | CC10 | SEQ ID No. 260 |
| MASK006 | GGGGQDGNEEMGGGGG | CC16 | SEQ ID No. 261 |
| MASK007 | APFGSEM | | SEQ ID No. 262 |
| MASK008 | AWNGPYE | | SEQ ID No. 263 |
| MASK009 | AQDNGDTKTG | | SEQ ID No. 264 |
| MASK010 | AEAKETQG | | SEQ ID No. 265 |
| MASK011 | ATRREQVEG | | SEQ ID No. 266 |
| MASK012 | AQAPSQP | | SEQ ID No. 267 |
| MASK013 | ATRSRTRNDG | | SEQ ID No. 268 |
| MASK014 | ADVDAPDGLG | | SEQ ID No. 269 |
| MASK015 | AADISDPGG | | SEQ ID No. 270 |
| MASK016 | ALSVDPSG | | SEQ ID No. 271 |
| MASK017 | ARLSVDPG | | SEQ ID No. 272 |
| MASK018 | AVEAADRG | | SEQ ID No. 273 |
| MASK020 | GGPDGNEEMGGG | CC12-Q1P | SEQ ID No. 274 |
| MASK021 | GGFDGNEEMGGG | CC12-Q1F | SEQ ID No. 275 |
| MASK022 | GGGDGNEEMGGG | CC12-Q1G | SEQ ID No. 276 |
| MASK023 | GGEMDGEGQNGG | CC12- | SEQ ID No. 277 |
| | | scramble | |
| MASK024 | GGGGGPDGNEEPGG | | SEQ ID No. 278 |
| MASK025 | GGGGSLDGNEEPGG | | SEQ ID No. 279 |
| MASK026 | GGGGALDGNEEPGG | | SEQ ID No. 280 |
| MASK027 | GGGGGLDGNEEPGG | | SEQ ID No. 281 |
| MASK028 | GGGALDGNEEPGG | | SEQ ID No. 282 |
| MASK029 | GGGGGPDGNEEPGGG | | SEQ ID No. 283 |
| MASK030 | GGSGALDGNEEPGG | | SEQ ID No. 284 |
| MASK031 | GGSGSLDGNEEPGG | | SEQ ID No. 285 |
| MASK038 | GGSGGPDGNEEPGG | | SEQ ID No. 286 |
| MASK039 | GGVRDGPDGNEEPGG | | SEQ ID No. 287 |
| MASK040 | GGSGGPDGNEEPGGGG | | SEQ ID No. 288 |
| MASK041 | GGGRGPDGNEEPGG | | SEQ ID No. 289 |
| MASK042 | GGSGGLDGNEEPGG | | SEQ ID No. 290 |
| MASK043 | GGGVGPDGNEEPGG | | SEQ ID No. 291 |
| MASK044 | GGGEGPDGNEEPGG | | SEQ ID No. 292 |
| MASK046 | GGGVALDGNEEPGG | | SEQ ID No. 293 |
| MASK047 | GGGRALDGNEEPGG | | SEQ ID No. 294 |
| MASK048 | GGYAGLDGNEEPGG | | SEQ ID No. 295 |
| MASK049 | GGAGGPDGNEEPGG | | SEQ ID No. 296 |
| MASK051 | GGRGGPDGNEEPGG | | SEQ ID No. 297 |
| MASK052 | GGGGPDGNEEPGGGG | | SEQ ID No. 298 |
| MASK053 | GGGEALDGNEEPGG | | SEQ ID No. 299 |
| MASK054 | GGDASLDGNEEPGG | | SEQ ID No. 300 |
| MASK055 | GGRDAPDGNEEGG | | SEQ ID No. 301 |

It is contemplated herein that in some embodiments of this disclosure the binding moieties described herein comprise at least one cleavable linker. In one aspect, the cleavable linker comprises a polypeptide having a sequence recognized and cleaved in a sequence-specific manner. The cleavage, in certain examples, is enzymatic, based on pH sensitivity of the cleavable linker, or by chemical degradation. The conditionally active binding proteins contemplated herein, in some cases, comprise a protease cleavable linker recognized in a sequence-specific manner by a matrix metalloprotease (MMP), for example a MMP9. In some cases, the protease cleavable linker is recognized by a MMP9 comprises a polypeptide having an amino acid sequence PR(S/T)(L/I)(S/T). In some cases, the protease cleavable linker recognized by a MMP9 comprises a polypeptide having an amino acid sequence LEATA (SEQ ID NO: 4).

In some cases, the protease cleavable linker is recognized in a sequence-specific manner by a MMP11. In some cases, the protease cleavable linker recognized by a MMP11 comprises a polypeptide having an amino acid sequence GGAANLVRGG (SEQ ID NO: 5). In some cases, the protease cleavable linker is recognized by a protease disclosed in Table 3. In some cases, the protease cleavable linker is recognized by a protease disclosed in Table 3 comprises a polypeptide having an amino acid sequence selected from a sequence disclosed in Table 3 (SEQ ID NOS: 1-42, 53, and 58-62). In some cases, the cleavable linker has an amino acid sequence as set forth in SEQ ID No. 59. In some cases, the cleavable linker is recognized by MMP9, matriptase, Urokinase plasminogen activator (uPA) and has an amino acid sequence as set forth in SEQ ID No. 59.

In some embodiments of this disclosure the binding moieties described herein comprise at least one non-cleavable linker. The non-cleavable linker comprises, in some examples, a sequence as set forth in SEQ ID No. 51, SEQ ID No. 302, SEQ ID No. 303, SEQ ID No. 304, or SEQ ID No. 305.

TABLE 2
Exemplary Non-cleavable Linker Sequences

| Non- | | |
|---|---|---|
| cleavable | | |
| Linker ID | Sequence | SEQ ID No. |
| L002 | SGGGGSGGVV | SEQ ID No. 302 |
| L016 | SGGGGSGGGGSGGGGS | SEQ ID No. 303 |
| L017 | SGGGGSGGGGSGGGGGS | SEQ ID No. 304 |
| L046 | SGGGGSGGGS | SEQ ID No. 305 |

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin F, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP7, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, type 11 transmembrane serine proteases (TTSP), neutrophil serine protease, cathepsin G, proteinase 3, neutrophil serine protease 4, mast cell chymase, and mast cell tryptases.

TABLE 3
Exemplary Proteases and Protease Recognition
Sequences

| | | SEQ |
|---|---|---|
| | Cleavage | ID |
| Protease | Domain Sequence | NO: |
| MMP7 | KRALGLPG | 1 |
| MMP7 | (DE)₈RPLALWRS(DR)₈ | 2 |
| MMP9 | PR(S/T)(L/I)(S/T) | 3 |
| MMP9 | LEATA | 4 |
| MMP11 | GGAANLVRGG | 5 |
| MMP14 | SGRIGFLRTA | 6 |
| MMP | PLGLAG | 7 |
| MMP | PLGLAX | 8 |
| MMP | PLGC(me)AG | 9 |
| MMP | ESPAYYTA | 10 |
| MMP | RLQLKL | 11 |
| MMP | RLQLKAC | 12 |
| MMP2, MMP9, MMP14 | EP(Cit)G(Hof)YL | 13 |
| Urokinase plasminogen | SGRSA | 14 |
| activator (uPA) | | |
| Urokinase plasminogen | DAFK | 15 |
| activator (uPA) | | |
| Urokinase plasminogen | GGGRR | 16 |
| activator (uPA) | | |
| Lysosomal Enzyme | GFLG | 17 |
| Lysosomal Enzyme | ALAL | 18 |
| Lysosomal Enzyme | FK | 19 |
| Cathepsin B | NLL | 20 |
| Cathepsin D | PIC(Et)FF | 21 |
| Cathepsin K | GGPRGLPG | 22 |
| Prostate Specific Antigen | HSSKLQ | 23 |
| Prostate Specific Antigen | HSSKLQL | 24 |
| Prostate Specific Antigen | HSSKLQEDA | 25 |
| Herpes Simplex Virus | LVLASSSFGY | 26 |
| Protease | | |
| HIV Protease | GVSQNYPIVG | 27 |
| CMV Protease | GVVQASCRLA | 28 |
| Thrombin | F(Pip)RS | 29 |
| Thrombin | DPRSFL | 30 |
| Thrombin | PPRSFL | 31 |
| Caspase-3 | DEVD | 32 |
| Caspase-3 | DEVDP | 33 |
| Caspase-3 | KGSGDVEG | 34 |
| Interleukin 1β converting | GWEHDG | 35 |
| enzyme | | |
| Enterokinase | EDDDDKA | 36 |
| FAP | KQEQNPGST | 37 |
| Kallikrein 2 | GKAFRR | 38 |
| Plasmin | DAFK | 39 |
| Plasmin | DVLK | 40 |
| Plasmin | DAFK | 41 |
| TOP | ALLLALL | 42 |
| MMP9 + matriptase | KPLGLQARVV | 58 |
| MMP9 + matriptase + uPA | PQASTGRSGG | 59 |
| MMP9 + matriptase + uPA | PQGSTGRAAG | 60 |
| Matriptase + uPA | PPASSGRAGG | 61 |
| MMP9 + matriptase | PIPVQGRAH | 62 |
| MMP9 + matriptase | PQGSTARSAG | 909 |

Proteases are known to be secreted by some diseased cells and tissues, for example tumor or cancer cells, creating a microenvironment that is rich in proteases or a protease-rich microenvironment. In some case, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen binding proteins to have additional specificity because T cells will not be bound by the antigen binding protein except in the protease rich microenvironment of the targeted cells or tissue.

The binding moiety comprising the cleavable linker thus masks the binding of a first or a second target antigen binding domain to their respective targets. In some embodiments, the binding moiety is bound to a first target antigen binding domain, which is further bound to a second target antigen binding domain, in the following order: binding moiety (M): cleavable linker (L): first target antigen binding domain (T1): second antigen binding domain (T2). In other examples, the domains are organized in any one of the following orders: M:L:T2:T1; T2:T1:L:M, T1:T2:L:M. The binding moiety is further bound to a half-life extending protein, such as albumin or any other of its targets as described below. In some instances, the binding moiety is albumin or comprises a binding site for albumin. In some instances the binding moiety comprises a binding site for IgE. In some embodiments, the binding moiety comprises a binding site for Igκ free light chain.

TABLE 4
Exemplary sequences for the binding moieties
comprising a cleavable linker are provided in
SEQ ID Nos. 796-800.
Sequence

SEQ ID No. 796
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSSGGGGKPLGLQARVVGGGGT
SEQ ID No. 797
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSSGGGGPQASTGRSGGGGGGT
SEQ ID No. 798
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSSGGGGPQGSTGRAAGGGGGT
SEQ ID No. 799
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSSGGGGPPASSGRAGGGGGT
SEQ ID No. 800
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSSGGGGPIPVQGRAHGGGGT

### Targets of Conditionally Active Binding Proteins

The conditionally active binding proteins described herein are activated by cleavage of the at least one cleavable linker attached to the binding moieties within said conditionally active proteins. It is contemplated that in some cases the activated binding protein binds to a target antigen involved in and/or associated with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated binding proteins disclosed herein.

In some embodiments, the target antigen is a tumor antigen expressed on a tumor cell. Tumor antigens are well known in the art and include, for example, EpCAM, EGFR, HER-2, HER-3, c-Met, FoIR, PSMA, CD38, BCMA, and CEA. 5T4, AFP, B37-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

In some embodiments, the target antigen is an immune checkpoint protein. Examples of immune checkpoint proteins include but are not limited to CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, inflamed or fibrotic tissue cell.

In some embodiments, the target antigen comprises an immune response modulator that is not a cytokine. Examples of immune response modulator include but are not limited to B7-1 (CD80), B7-2 (CD86), CD3, or GITR.

In some embodiments, the first target antigen binding domain or the second target antigen binding domain comprises an anti-EGFR domain, an anti-EpCAM domain, an anti-DLL3 domain, an anti-MSLN domain, an anti-PSMA domain, an anti-BDMA domain, or any combinations thereof. In some embodiments, the first target antigen binding domain or the second target antigen binding domain comprises an anti-EGFR sdAb, an anti-EpCAM sdAb, an anti-DLL3 sdAb, an anti-MSLN sdAb, an anti-PSMA sdAb, an anti-BDMA sdAb, or any combinations thereof.

In some embodiments, an anti-EGFR domain of this disclosure comprises an amino acid selected from the group consisting of SEQ ID Nos. 55, and 737-785. In some embodiments, an anti-PSMA domain of this disclosure comprises an amino acid selected from the group consisting of SEQ ID Nos. 57-73. In some embodiments, an anti-BCMA domain of this disclosure comprises an amino acid selected from the group consisting of SEQ ID Nos. 91-214. In some embodiments, an anti-MSLN domain of this disclosure comprises an amino acid selected from the group consisting of SEQ ID Nos. 215-258. In some embodiments, an anti-DLL3 domain of this disclosure comprises an amino acid selected from the group consisting of SEQ ID Nos. 306-736. In some embodiments, an anti-EpCAM domain of this disclosure comprises an amino acid selected from the group consisting of SEQ ID Nos. 804-841.

In some embodiments, the first target antigen binding domain or the second target antigen binding domain comprises an anti-CD3 domain. In some embodiments, the anti-CD3 domain comprises an anti-CD3 scFV. In some embodiments, the anti-CD3 scFv comprises an amino acid sequence selected from the group consisting of: SEQ ID Nos. 74-90, and 794.

### Binding Protein Variants

As used herein, the term “binding protein variants” refers to variants and derivatives of the conditionally active target-binding proteins described herein, containing a binding moiety as described above, comprising non-CDR loops that bind to an immunoglobulin binding molecule, such as a first or a second target antigen binding domain, a non-immunoglobulin binding molecule. In certain embodiments, amino acid sequence variants of the conditionally active target-binding proteins described herein are contemplated. For example, in certain embodiments amino acid sequence variants of the conditionally active target-binding proteins described herein are contemplated to improve the binding affinity and/or other biological properties of the binding proteins. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to the various domains to arrive at the final construct, provided that the final construct possesses the desired characteristics, e.g., antigen-binding. In certain embodiments, binding protein variants having one or more amino acid substitutions are provided. Sites of interest for substitution mutagenesis include the CDRs and framework regions. Amino acid substitutions may be introduced into the variable domains of a conditionally active protein of interest and the products screened for a desired activity, e.g., retained/improved antigen binding, decreased immunogenicity, or improved antibody-dependent cell mediated cytotoxicity (ADCC) or complement dependent cytotoxicity (CDC). Both conservative and non-conservative amino acid substitutions are contemplated for preparing the antibody variants.

In another example of a substitution to create a variant conditionally active antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding. For example, an affinity matured variant antibody can be generated, e.g., using phage display-based affinity maturation techniques such as those described herein and known in the field.

In another example, substitutions are made in hypervariable regions (HVR) of a parent conditionally active antibody to generate variants and variants are then selected based on binding affinity, i.e., by affinity maturation. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. Substitutions can be in one, two, three, four, or more sites within a parent antibody sequence.

In some embodiments, a conditionally active binding protein, as described herein comprises a VL domain, or a VH domain, or both, with amino acid sequences corresponding to the amino acid sequence of a naturally occurring VL or VH domain, respectively, but that has been “humanized”, i.e., by replacing one or more amino acid residues in the amino acid sequence of said naturally occurring VL or VH domains (and in particular in the framework sequences) by one or more of the amino acid residues that occur at the corresponding position(s) in a VL or VH domain from a conventional 4-chain antibody from a human being (e.g., as indicated above). This can be performed in a manner known in the field, which will be clear to the skilled person, for example on the basis of the further description herein. Again, it should be noted that such humanized conditionally active target-binding antibodies of the disclosure are obtained in any suitable manner known per se and thus are not strictly limited to polypeptides that have been obtained using a polypeptide that comprises a naturally occurring VL and/or VH domain as a starting material. In some additional embodiments, an conditionally active target-binding antibody, as described herein, comprises a VL and a VH domain with amino acid sequences corresponding to the amino acid sequence of a naturally occurring VL or VH domain, respectively, but that has been “camelized”, i.e., by replacing one or more amino acid residues in the amino acid sequence of a naturally occurring VL or VH domain from a conventional 4-chain antibody by one or more of the amino acid residues that occur at the corresponding position(s) in a VL or a VH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized conditionally active antibodies of the disclosure, in certain embodiments, are obtained in any suitable manner known in the field and thus are not strictly limited to polypeptides that have been obtained using a polypeptide that comprises a naturally occurring VL and/or VH domain as a starting material. For example, both “humanization” and “camelization” is performed by providing a nucleotide sequence that encodes a naturally occurring VL and/or VH domain, respectively, and then changing, one or more codons in said nucleotide sequence in such a way that the new nucleotide sequence encodes a “humanized” or “camelized” conditionally active antibody, respectively. This nucleic acid can then be expressed, so as to provide the desired target-antigen binding capability. Alternatively, in other embodiments, a “humanized” or “camelized” conditionally active antibody is synthesized de novo using known peptide synthesis technique from the amino acid sequence of a naturally occurring antibody comprising a VL and/or VH domain. In some embodiments, a “humanized” or “camelized” conditionally active antibody is synthesized de novo using known peptide synthesis technique from the amino acid sequence or nucleotide sequence of a naturally occurring antibody comprising a VL and/or VH domain, respectively, a nucleotide sequence encoding the desired humanized or camelized conditionally active domain antibody of the disclosure, respectively, is designed and then synthesized de novo using known techniques for nucleic acid synthesis, after which the nucleic acid thus obtained is expressed in using known expression techniques, so as to provide the desired conditionally active antibody of the disclosure.

Other suitable methods and techniques for obtaining the conditionally active binding protein of the disclosure and/or nucleic acids encoding the same, starting from naturally occurring sequences for VL or VH domains for example comprises combining one or more parts of one or more naturally occurring VL or VH sequences (such as one or more framework (FR) sequences and/or complementarity determining region (CDR) sequences), and/or one or more synthetic or semi-synthetic sequences, and/or a naturally occurring sequence for a CH2 domain, and a naturally occurring sequence for a CH3 domain comprising amino acid substitutions that favor formation of heterodimer over homodimer, in a suitable manner, so as to provide a conditionally active binding protein of the disclosure or a nucleotide sequence or nucleic acid encoding the same.

### Affinity Maturation

In designing conditionally active binding proteins for therapeutic applications, it is desirable to create proteins that, for example, modulate a functional activity of a target, and/or improved binding proteins such as binding proteins with higher specificity and/or affinity and/or and binding proteins that are more bioavailable, or stable or soluble in particular cellular or tissue environments.

The conditionally active binding proteins described in the present disclosure exhibit improved the binding affinities towards the target, for example a tumor antigen expressed on a cell surface. In some embodiments, the conditionally active binding protein of the present disclosure is affinity matured to increase its binding affinity to the target, using any known technique for affinity-maturation (e.g., mutagenesis, chain shuffling, CDR amino acid substitution). Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the conditionally active target-binding proteins are isolated by screening combinatorial libraries, for example, by generating phage display libraries and screening such libraries for antibodies possessing the desired binding characteristics towards a target antigen, such as a tumor antigen expressed on a cell surface.

### Conditionally Active Binding Protein Modifications

The conditionally active binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the conditionally active binding proteins described herein, including the peptide backbone, the amino acid side-chains, and the amino or carboxyl termini. Certain common peptide modifications that are useful for modification of the conditionally active binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, the conditionally active binding proteins of the disclosure are conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc.

### Polynucleotides Encoding the Binding Moieties or the Conditionally Active Binding Proteins

Also provided, in some embodiments, are polynucleotide molecules encoding the binding moieties as described herein. In some embodiments, the polynucleotide molecules are provided as a DNA construct. In other embodiments, the polynucleotide molecules are provided as a messenger RNA transcript.

Also provided, in some embodiments, are polynucleotide molecules encoding the conditionally active binding proteins as described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the various domains (e.g. binding moiety, target antigen binding domain, etc.) either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and conditionally active promoters, may be used. The promoter is selected such that it drives the expression of the polynucleotide in the respective host cell.

In some embodiments, the polynucleotides described herein are inserted into vectors, such as expression vectors, which represent further embodiments. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described conditionally active binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the binding moieties or the conditionally active binding proteins comprising the binding moieties as described herein, in some embodiments, are produced by introducing vectors encoding the binding moieties or the binding proteins as described above into host cells and culturing said host cells under conditions whereby the binding moieties or the binding proteins, or domains thereof are expressed.

### Pharmaceutical Compositions

Also provided, in some embodiments, are pharmaceutical compositions comprising a therapeutically effective amount of a conditionally active binding protein of the present disclosure, and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms is, in some cases, ensured by the inclusion of various antibacterial and antifungal agents.

The conditionally active binding proteins described herein are contemplated for use as medicaments. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a conditionally active binding protein as described herein. In some instances, administration induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue. In some embodiments, the target antigen is an immune checkpoint protein.

Also provided herein are methods and uses for a treatment of a disease, disorder or condition associated with a target antigen comprising administering to an individual in need thereof a conditionally active binding protein as described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a melanoma. In a further instance, the cancer is non-small cell lung cancer. In yet further instance, the cancer is breast cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the conditionally active binding proteins described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (y-rays, X-rays, and/or the directed delivery of radioisotopes, microwaves, UV radiation and the like), gene therapies (e.g., antisense, retroviral therapy and the like) and other immunotherapies. In some embodiments, the conditionally active binding proteins described herein are administered in combination with anti-diarrheal agents, anti-emetic agents, analgesics, opioids and/or non-steroidal anti-inflammatory agents. In some embodiments, the conditionally active binding proteins described herein is administered before, during, or after surgery.

### EXAMPLES

The examples below further illustrate the described embodiments without limiting the scope of the disclosure.

### Example 1: Construction of an Exemplary Binding Moiety which Binds to Albumin and a Target Antigen Binding Domain Whose Target is EGFR

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for EGFR. A clone with high specificity for EGFR is selected. Affinity maturation is performed by re-randomizing residues in the non-CDR loop regions as before.

Sequence alignment of non-CDR loop regions of the resultant proteins is performed to determine sequence conservation between proteins with high affinity for the EGFR binding target antigen binding domain. Site directed mutagenesis of one or more amino acids within these regions of sequence conservation is performed to generate additional proteins. Binding of the resultant proteins to an immobilized target antigen binding domain whose target is EGFR is measured in an ELISA. A protein with the highest affinity for the target antigen binding domain is selected.

The sequence of this binding moiety is cloned into a vector comprising a sequence for a cleavable linker, and sequences for a second target antigen binding domain that binds to a second target antigen, e.g., CD3. The resultant vector is expressed in a heterologous expression system to obtain a conditionally active target binding protein that comprises a binding moiety comprising a cleavable linker and non-CDR loops which provide a binding site specific for the target antigen binding domain whose target is EGFR, and CDR loops which are specific for albumin.

### Example 2: Construction of an Exemplary Binding Moiety which Binds to Albumin and a Target Antigen Binding Domain Whose Target is CD3

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for CD3. A clone with high specificity for CD3 is selected. Affinity maturation is performed by re-randomizing residues in the non-CDR loop regions as before.

Sequence alignment of non-CDR loop regions of the resultant proteins is performed to determine sequence conservation between proteins with high affinity for the EGFR binding target antigen binding domain. Site directed mutagenesis of one or more amino acids within these regions of sequence conservation is performed to generate additional proteins. Binding of the resultant proteins to an immobilized target antigen binding domain whose target is CD3 is measured in an ELISA. A protein with the highest affinity for the target antigen binding domain is selected.

The sequence of this binding moiety is cloned into a vector comprising a sequence for a cleavable linker, and sequences for a second target antigen binding domain that binds to a second target antigen, e.g., EGFR. The resultant vector is expressed in a heterologous expression system to obtain a conditionally active target binding protein that comprises a binding moiety comprising a cleavable linker and non-CDR loops which provide a binding site specific for the target antigen binding domain whose target is CD3, and CDR loops which are specific for albumin.

### Example 3: A Conditionally Active Binding Protein of this Disclosure Exhibit Reduced Specificity Toward Cell Line which Overexpresses EGFR but is Protease Deficient

Cells overexpressing EGFR and exhibiting low expression of a matrix metalloprotease are separately incubated with an exemplary conditionally active binding protein according to the present disclosure and a non-conditionally active control binding protein. Cells expressing normal levels of EGFR and proteases are also incubated with a conditionally active binding protein according to the present disclosure and a non-conditionally active control binding protein. Both proteins comprise a target antigen binding domain with specificity toward PSMA.

Results indicate that in the absence of protease secretion, the conditionally active binding protein of the present disclosure interacts with the protease expressing cells but does not interact with the EGFR expressed on the surface of the protease deficient cells. In contrast, the non-conditionally active control binding protein lacks the ability to selectively bind the protease expressing cells over the protease deficient ones. Thus, the exemplary conditionally active binding protein receptor of the present disclosure is advantageous, for example, in terms of reducing off-tumor toxicity.

### Example 4: Treatment with an Exemplary Conditionally Active Binding Protein of the Present Disclosure Inhibits In Vivo Tumor Growth

Murine tumor line CT26 is implanted subcutaneously in Balb/c mice and on day 7 post-implantation the average size of the tumor is measured. Test mice are treated with an exemplary conditionally active binding protein which has a target antigen binding domain specific for CTLA4 and another target antigen binding domain specific for CD3, wherein either the CTLA4 or the CD3 specific domain is bound to a binding moiety via its non-CDR loops, the binding moiety comprises a cleavable linker, and is bound to albumin. Control mice are treated with binding protein that contains CD3/CTLA4 specific domains but do not contain the binding moiety or the cleavable linker, and are not conditionally active. Results show that treatment with the exemplary conditionally active binding protein of the present disclosure inhibits tumor more efficiently than the comparator binding protein which does not contain the moiety with non-CDR loops.

### Example 5: Exemplary Conditionally Active Binding Protein Exhibits Reduced Specificity Toward Cell Line which Overexpresses Antigen but is Protease Deficient

Cells overexpressing CTLA-4 and exhibiting low expression of a matrix metalloprotease are separately incubated with an exemplary CTLA4 specific conditionally active binding protein of this disclosure, containing a binding moiety that binds to a CTLA4 binding domain via its non-CDR loops and albumin via its CDRs; or a control CTLA-4 binding antibody which does not contain the binding moiety which binds to a CTLA4 binding domain via its non-CDR loops and to albumin via its CDRs. Cells expressing normal levels of antigens and proteases are also incubated with the exemplary CTLA4 specific conditionally active binding protein, or the control CTLA4 binding antibody.

Results indicate that in the absence of protease secretion, the conditionally active binding protein of the present disclosure binds the protease expressing cells but does not bind the protease-deficient antigen expressing cells. In contrast, the control antibody lacks the ability to selectively bind the protease expressing cells over the protease deficient ones. Thus, the exemplary conditionally active binding protein of the present disclosure is advantageous, for example, in terms of reducing off-tumor toxicity.

### Example 6: Purification, Pharmacokinetic Analysis, and Potency Assays for Exemplary ProTriTAC Molecules

### A) Expression, Purification and Stability of Exemplary ProTriTAC Molecules

### Protein Production

Sequences of exemplary ProTriTAC (also referred to as Pro-trispecific) molecules were cloned into mammalian expression vector pcDNA 3.4 (Invitrogen) preceded by a leader sequence and followed by a 6× Histidine Tag (SEQ ID NO: 927). Expi293F cells (Life Technologies A14527) were maintained in suspension in Optimum Growth Flasks (Thomson) between 0.2 to 8×1e6 cells/ml in Expi 293 media. Purified plasmid DNA was transfected into Expi293 cells in accordance with Expi293 Expression System Kit (Life Technologies, A14635) protocols, and maintained for 4-6 days post transfection. Alternatively, sequences of trispecific molecules were cloned into mammalian expression vector pDEF38 (CMC ICOS) transfected into CHO-DG44 dhfr-cells, stable pools generated, and cultured in production media for up to 12 days prior to purification. The amount of the exemplary trispecific proteins in conditioned media was quantitated using an Octet RED 96 instrument with Protein A tips (ForteBio/Pall) using a control trispecific protein for a standard curve. Conditioned media from either host cell was filtered and partially purified by affinity and desalting chromatography. Trispecific proteins were subsequently polished by ion exchange and upon fraction pooling formulated in a neutral buffer containing excipients. Final purity was assessed by SDS-PAGE and analytical SEC using an Acquity BEH SEC 200 1.7u 4.6×150 mm column (Waters Corporation) resolved in an aqueous/organic mobile phase with excipients at neutral pH on a 1290 LC system and peaks integrated with Chemstation CDS software (Agilent). Trispecific proteins purified from CHO host cells were analyzed by running an SDS-PAGE, as shown in FIG. 5.

### Stability Assessment

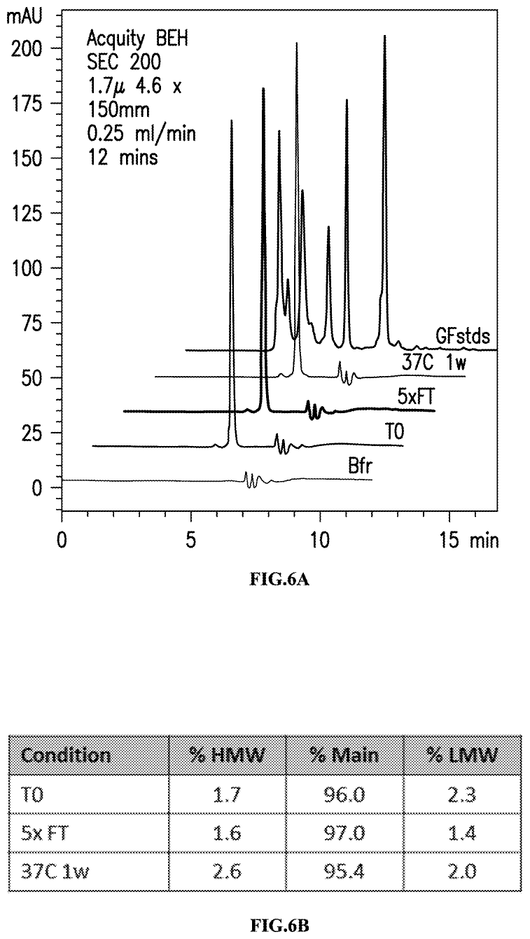

Purified Protrispecific proteins in two formulations were sub-aliquoted into sterile tubes and stressed by five freeze-thaw cycles each comprising greater than 1 hour at −80 °C and room temperature or by incubation at 37 °C for 1 week. Stressed samples were evaluated for concentration and turbidity by UV spectrometry using UV transparent 96 well plates (Corning 3635) with a SpectraMax M2 and SoftMaxPro Software (Molecular Devices), SDS-PAGE, and analytical SEC and compared to the same analysis of control non-stressed samples. An overlay of chromatograms from analytical SEC of control and stressed samples for a single exemplary trispecific ProTriTAC molecule purified from 293 host cells is depicted in FIG. 6.

### B) ProTriTAC Exhibits Potent, Protease-Dependent, Anti-Tumor Activity in a Rodent Tumor Xenograft Model

An exemplary ProTriTAC molecule (SEQ ID NO: 46) containing an EGFR binding domain as the target binding domain, a CD3 binding domain and an albumin binding domain comprising a masking moiety (SEQ ID NO: 50) and a cleavable linker (SEQ ID NO: 53) was evaluated for anti-tumor activity in vivo in an HCT116 subcutaneous xenograft tumor admixed with expanded human T cells in immunocompromised NCG mice. A non-cleavable EGFR targeting ProTriTAC molecule (SEQ ID NO: 47) and a GFP targeting ProTriTAC molecule (SEQ ID NO: 49) were also used in the study. Specifically, 5×10⁶ HCT116 cells were admixed with 2.5×10⁶ expanded T cells per mouse on day 0. Dosing of the test molecules (EGFR targeting ProTriTAC, non-cleavable EGFR targeting Pro-TriTAC, and GFP targeting ProTriTAC) were performed starting on the following day with a q.d.×10 (single daily dose for 10 days) schedule via intraperitoneal injection, at a dose of 0.03 mg/kg. Tumor volumes were determined using caliper measurements and calculated using the formula V=(length×width×width)/2, at the indicated times. Results shown in FIG. 7 indicate that following final dose of test molecules, on day 10, tumor growth was arrested in mice administered the activatable EGFR targeting ProTriTAC molecule. Whereas, administering the GFP targeting ProTriTAC molecule was not effective in inhibiting tumor growth and the administration of the EGFR targeting non-cleavable ProTriTAC molecule was not as potent as the activatable ProTriTAC, in arresting tumor growth.

### C) Demonstration of Functional Masking and Stability of ProTriTAC In Vivo in a Three-Week Cynomolgus Monkey Pharmacokinetic Study

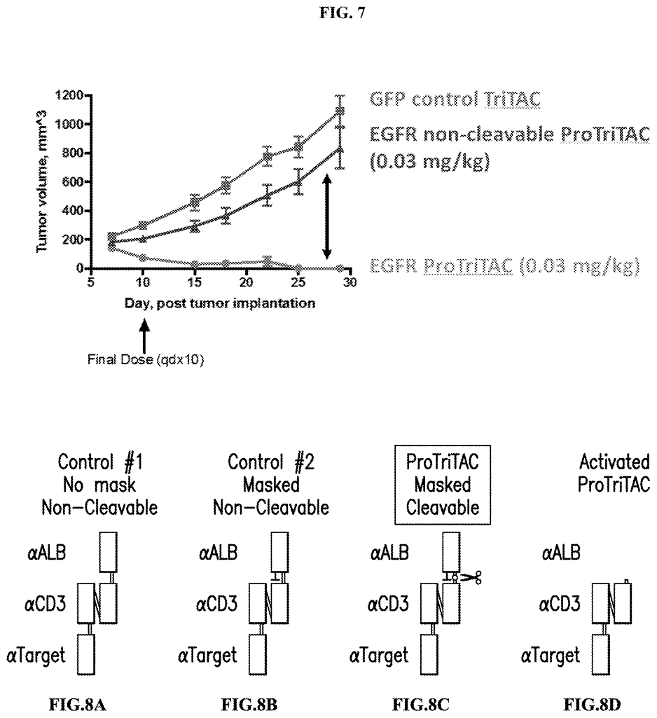

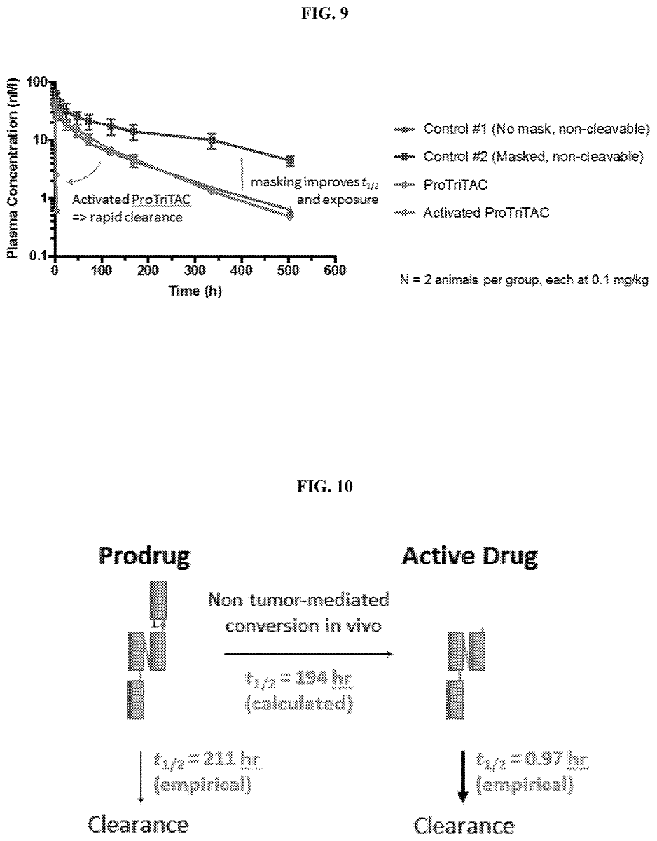

Single doses of PSMA targeting ProTriTAC (SEQ ID NO: 43) containing a PSMA binding domain as the target binding domain, a CD3 binding domain, and an albumin binding domain comprising a masking moiety (SEQ ID NO: 50) and a cleavable linker (SEQ ID NO: 53), non-cleavable PSMA targeting ProTriTAC (SEQ ID NO: 44), non-masked/non-cleavable TriTAC (SEQ ID NO: 52), and active drug mimicking protease-activated PSMA targeting ProTriTAC (SEQ ID NO: 45) were dosed into cynomolgus monkeys at 0.1 mg/kg via intravenous injection. Plasma samples were collected at the time points indicated in FIG. 9. The designs of the above described test molecules are shown in FIG. 8. Concentrations of the various test molecules, as described above, were determined using ligand binding assays with biotinylated recombinant human PSMA (R&D systems) and sulfo-tagged anti-CD3 idiotype antibody cloned 11D3 in a MSD assay (Meso Scale Diagnostic, LLC). Pharmacokinetic parameters were estimated using Phoenix WinNonlin pharmacokinetic software using a non-compartmental approach consistent with the intravenous bolus route of administration.

To calculate the rate of in vivo conversion of the test molecules (i.e., conversion of PSMA targeting ProTriTAC, non-cleavable PSMA targeting ProTriTAC, non-masked/non-cleavable PSMA targeting ProTriTAC) the concentration of active drug in circulation was estimated by solving the following system of differential equations where P is the concentration of prodrug, A is the concentration of active drug, kₐ is the rate of prodrug activation in circulation, k_{c,P} is the clearance rate of the prodrug, and k_{c,A} is the clearance rate of the active drug.

$$\frac{dP}{dt} = - {k}_{c , P} P$$

$$\frac{dA}{dt} = {k}_{a} P - {k}_{c , A} A$$

The clearance rates of the prodrug, active drug, non-masked non-cleavable prodrug control, and a non-cleavable prodrug control (k_{c,NCLV}) were determined empirically in cynomolgus monkeys. To estimate the rate of prodrug activation in circulation, it was assumed that the difference between the clearance rate of cleavable prodrug and the non-cleavable prodrug arose solely from non-specific activation in circulation. Therefore, the rate of prodrug conversion to active drug in circulation was estimated by subtracting the clearance rate of the cleavable prodrug from the non-cleavable prodrug.

kₐ=k_{c,NCLV}−k_{c,P}

The initial concentration of prodrug in circulation was determined empirically and the initial concentration of active drug was assumed to be zero. Further calculations showed that the ProTriTAC comprising the protease cleavable linker was sufficiently stable in circulation, with 50% non-tumor mediated conversion every 194 hours and the t_{1/2} of the molecule was determined, empirically, to be around 211 hours. This indicated that ProTriTAC molecules are sufficiently stable and protected against off-tumor effects. In contrast, the t_{1/2} of the active drug fragment mimicking the activated ProTRITAC molecule was determined, empirically, to be 0.97 hours. Thus, active drug was rapidly cleared from circulation. Results are shown in FIG. 10.

FIG. 11 shows that because the active drug mimicking the activated ProTriTAC molecule was rapidly cleared from circulation and that the ProTriTAC and the control non-masked non-cleavable ProTriTAC had longer half-lives, there is a significant >200-fold differential between the ProTriTAC and the activated ProTriTAC circulating exposure. It was also observed that the masked but non-cleavable ProTriTAC control molecule was present in circulation for a longer time than the non-masked non-cleavable ProTriTAC control, thereby indicating that masking plays a role in increased circulation half-life (t_{1/2}) and limited peripheral T cell binding. The combination of the functional masking that renders ProTriTAC inert outside the tumor environment and the half-life differential that renders any aberrantly activated ProTriTAC in circulation to be rapidly cleared in vivo works together to ensure on-target, off-tumor activity of ProTriTAC is minimized. Supporting pharmacokinetic parameters are shown in Table 5.

TABLE 5
Pharmacokinetics of ProTriTAC, Activated
ProTriTAC and Control Molecules

| | Terminal t_{1/2} | Cₘₐₓ | AUC, 0-last | Clearance |
|---|---|---|---|---|
| Test Article | (hr) | (nM) | (hr*nM) | (mL/hr/kg) |
| Control #1 (No mask, | 118 | 48.2 | 2490 | 0.735 |
| non-cleavable) | | | | |
| Control #2 (Masked, | 211 | 58.0 | 7000 | 0.238 |
| non-cleavable) | | | | |
| ProTriTAC | 101 | 42.7 | 2670 | 0.686 |
| Activated ProTriTAC | 0.969 | 66.6 | 41.5 | 58.5 |

### D) Protease Activation of ProTriTAC Molecule Leads to Significantly Enhanced Activity In Vitro

The aim of this study was to assess the relative potency of protease activatable ProTriTAC molecules, non-cleavable ProTriTAC molecules and recombinant active drug fragment mimicking the protease-activated ProTriTAC molecule, in CD3 binding and T cell mediated cell killing. The active drug fragment mimicking the protease activated ProTriTAC molecule contained the CD3 binding domain and the target antigen binding domain but lacked the albumin binding domain. Whereas the protease activatable ProTriTAC molecule contained the albumin binding domain comprising a masking domain and a protease cleavable site, the CD3 binding domain, and the target antigen binding domain. The non-cleavable ProTriTAC molecule lacked the protease cleavable site but was otherwise identical to the protease activatable ProTriTAC molecule.

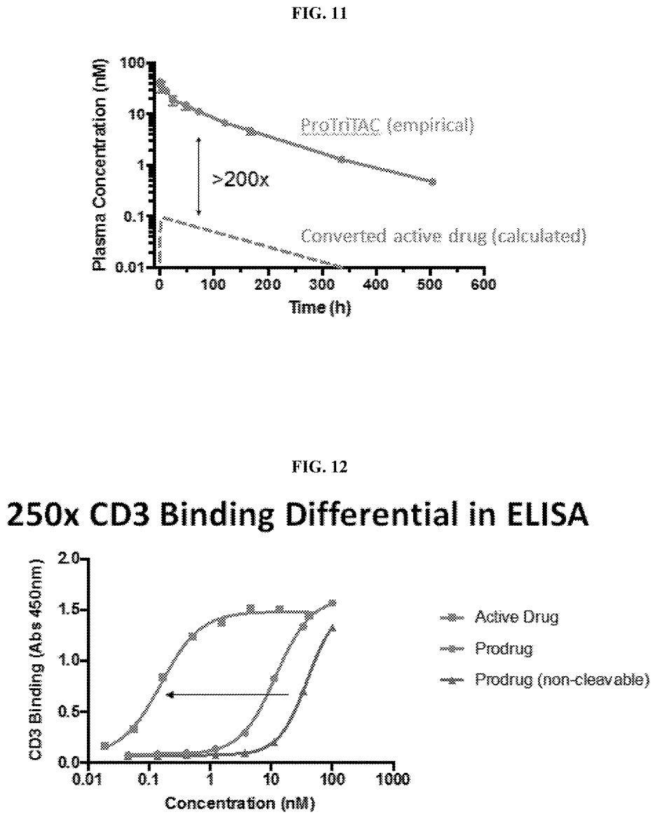

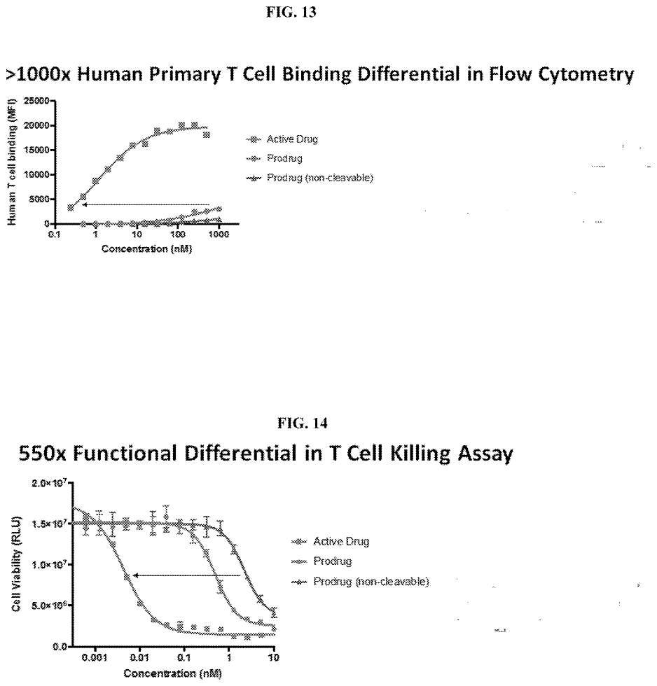

Purified ProTriTAC (labeled as prodrug in FIGS. 12-14), non-cleavable ProTriTAC (labeled as prodrug (non-cleavable) in FIGS. 12-14), and recombinant active drug fragment mimicking the protease-activated ProTriTAC (labeled as active drug in FIGS. 12-14) were tested for binding to recombinant human CD3 in an ELISA assay (FIG. 12), binding to purified human primary T cells in a flow cytometry assay (FIG. 13), and functional potency in a T cell-dependent cellular cytotoxicity assay (FIG. 14).

For ELISA, soluble test molecules (i.e., active drug, prodrug, and prodrug (non-cleavable) at the indicated concentrations were incubated, in multi-well plates, with immobilized recombinant human CD3ε (R&D Systems) for 1 hour at room temperature in PBS supplemented with 15 mg/mL human serum albumin. Plates were blocked using SuperBlock (Thermo Fisher), washed using PBS with 0.05% Tween-20, and detected using a non-competitive anti-CD3 idiotype monoclonal antibody 11D3 followed by peroxidase-labeled secondary antibody and TMB-ELISA substrate solution (Thermo Fisher). Results shown in FIG. 12 demonstrate that the active drug fragment mimicking the protease-activated ProTriTAC molecule was about 250 times more potent in binding CD3 as compared to prodrug non-cleavable. The EC₅₀ values are provided in Table 6. The masking ratio is the ratio between the prodrug EC₅₀ over the active drug EC₅₀: the higher the number, the higher the fold-shift between prodrug and active drug and thus greater functional masking.

TABLE 6
CD3 binding potential

| | EC₅₀ (nM) | Masking Ratio |
|---|---|---|
| Active Drug | 0.16 | — |
| Prodrug | 11.91 | 74 |
| Prodrug (non-cleavable) | 39.44 | 247 |

For binding to human primary T cells, determined by flow cytometry, soluble test molecules (i.e., active drug, prodrug, and prodrug (non-cleavable)) at the indicated concentrations (shown in FIG. 13) were incubated, in multi-well plates, with purified human primary T cells for 1 h at 4 °C in the presence of PBS with 2% fetal bovine serum and 15 mg/ml human serum albumin. Plates were washed with PBS with 2% fetal bovine serum, detected using AlexaFluor 647-labeled non-competitive anti-CD3 idiotype monoclonal antibody 11D3, and data was analyzed using FlowJo 10 (FlowJo, LLC). Results shown in FIG. 13 demonstrate that the active drug fragment mimicking the protease-activated ProTriTAC molecule was greater than 1000 times more potent in binding human primary T cells as compared to prodrug non-cleavable. The EC₅₀ values are provided in Table 7.

TABLE 7
Human primary T cell binding potential

| | EC₅₀ (nM) | Masking Ratio |
|---|---|---|
| Active Drug | 1.19 | — |
| Prodrug | >1000 | n/a |
| Prodrug (non-cleavable) | >1000 | n/a |

For functional potency in a T cell-dependent cellular cytotoxicity assays, soluble test molecules (i.e., active drug, prodrug, and prodrug (non-cleavable)) at the indicated concentrations, shown in FIG. 14, were incubated, in multi-well plates, with purified resting human T cells (effector cell) and HCT116 cancer cell (target cell) at 10:1 effector: target cell ratio for 48 h at 37 °C. The HCT116 target cell line had been stably transfected with a luciferase reporter gene to allow specific T cell-mediated cell killing measurement by ONE-Glo (Promega). Results shown in FIG. 14 demonstrate that the active drug fragment mimicking the protease-activated ProTriTAC molecule was about 500 times more potent in T cell mediated killing of cancer cells, as compared to prodrug non-cleavable. The EC₅₀ values are provided in Table 8.

TABLE 8
T cell mediated cell killing potential

| | EC₅₀ (nM) | Masking Ratio |
|---|---|---|
| Active Drug | 0.004 | — |
| Prodrug | 0.485 | 121 |
| Prodrug (non-cleavable) | 2.197 | 549 |

### Example 7: Anti-Tumor Activity of Exemplary ProTriTAC Molecules Containing Various Exemplary Linkers, in an Admixed Mouse Tumor Model

The aim of this study was to explore the anti-tumor activity of ProTriTAC molecules containing different linkers. NSG female mice, 7 weeks old, were used for this study. At the commencement of the study, on day 0, the NSG female mice were injected with 2.5×10⁶ expanded human T cells, and 5×10⁶ HCT116 (human colorectal carcinoma) tumor cells. The following day, on day 1, the mice were divided into groups and each group was treated with at least one of the ProTriTAC molecules listed in Table 9 (SEQ ID Nos. 786-790), or with a control GFP TriTAC molecule (SEQ ID No. 792), or with a ProTriTAC molecule that contains a non cleavable linker (NCLV) (SEQ ID No. 791).

The ProTriTAC molecules and the ProTriTAC NCLV molecule used in the following examples were targeted to EGFR and had the following orientation of the individual domains: (anti-albumin binding domain (sdAb): anti-CD3 domain (scFV): anti-EGFR domain (sdAb)). The only differences between the ProTriTAC molecules listed in Table 6 were in the linker sequences. The ProTriTAC molecules, ProTriTAC NCLV molecule, or the GFP TriTAC molecule (the GFP TriTAC molecule had the following orientation of individual domains: anti-GFP sdAb: anti-Alb sdAb: anti-CD3 scFv) were administered daily for a period of 10 days (i.e., final dose was administered on day 10 following injection of tumor cells and expanded cells to the animals) and tumor volumes were measured at regular intervals, beginning a few days prior to the administration of the final dose at day 10.

TABLE 9
ProTriTAC sequences and linkers

| | | | Cleavability/ |
|---|---|---|---|
| | Linker | | Recognition |
| ProTriTAC | in the | | of the linker |
| molecule | ProTriTAC | Linker | sequence by |
| sequence | molecule | Sequence | enzymes |
| SEQ ID | L001 (SEQ | KPLGLQARVV | MMP9 + matriptase |
| No. 786 | ID NO: 58) | | |
| SEQ ID | L040 (SEQ | PQASTGRSGG | MMP9 + |
| No. 787 | ID NO: 59) | | matriptase + uPA |
| SEQ ID | L041 (SEQ | PQGSTGRAAG | MMP9 + |
| No. 788 | ID NO: 60) | | matriptase + uPA |
| SEQ ID | L042 (SEQ | PPASSGRAGG | matriptase + uPA |
| No. 789 | ID NO: 61) | | |
| SEQ ID | L045 (SEQ | PIPVQGRAH | MMP9 + matriptase |
| No. 790 | ID NO: 62) | | |
| | L043 (SEQ | PQGSTARSAG | MMP9 + matriptase |
| | ID NO: 909) | | |

As shown in FIG. 18, the ProTriTAC molecules containing linker sequences L001, L045, L040, and L041 demonstrated more potent anti-tumor activity as compared to the GFP TriTAC control of the ProTriTAC NCLV molecule. The statistical significance of the data was determined by repeated measures one-way ANOVA-Dunnett post test. The mean tumor volume of each group of mice were compared to the mean tumor volume of the mice group that received the NCLV molecule.

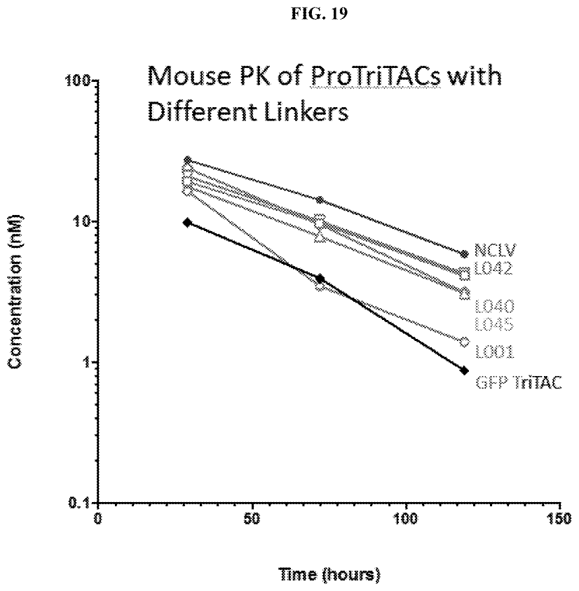

The pharmacokinetics following administration of the various molecules, as described above, were also assessed and the data is shown in FIG. 19. The control GFP TriTAC molecule was rapidly cleared from circulation, following administration, whereas the NCLV molecule remained in circulation for the longest time. The pharmacokinetic clearance profile of the test ProTriTAC molecules were in between the GFP TriTAC and NCLV, except for the ProTriTAC containing the linker sequence L001, which was cleared almost as rapidly as the control GFP TriTAC.

### Example 8: Individual Tumor Volumes of Admixed Xenograft Tumors, Following Treatment with Exemplary ProTriTAC Molecules Containing Various Exemplary Linkers

The ProTriTAC molecules listed in Table 9, the control GFP TriTAC molecule, and the ProTriTAC NCLV molecule were evaluated in an admixed xenograft model, in order to determine the efficacy of the ProTriTAC molecules containing different linkers, in vivo. As described in previous example (Example 7), the xenograft tumor model was generated by injecting 7 week old NSG mice with 2.5×10⁶ expanded human T cells, and 5×10⁶ HCT116 (human colorectal carcinoma) tumor cells. The mice were divided into groups and each group was treated with at least one of the ProTriTAC molecules listed in Table 9, with the control GFP TriTAC molecule, or with the ProTriTAC NCLV molecule. Tumor volumes were measured at regular intervals, starting from day 10 post injection of tumor cells and expanded T cells.

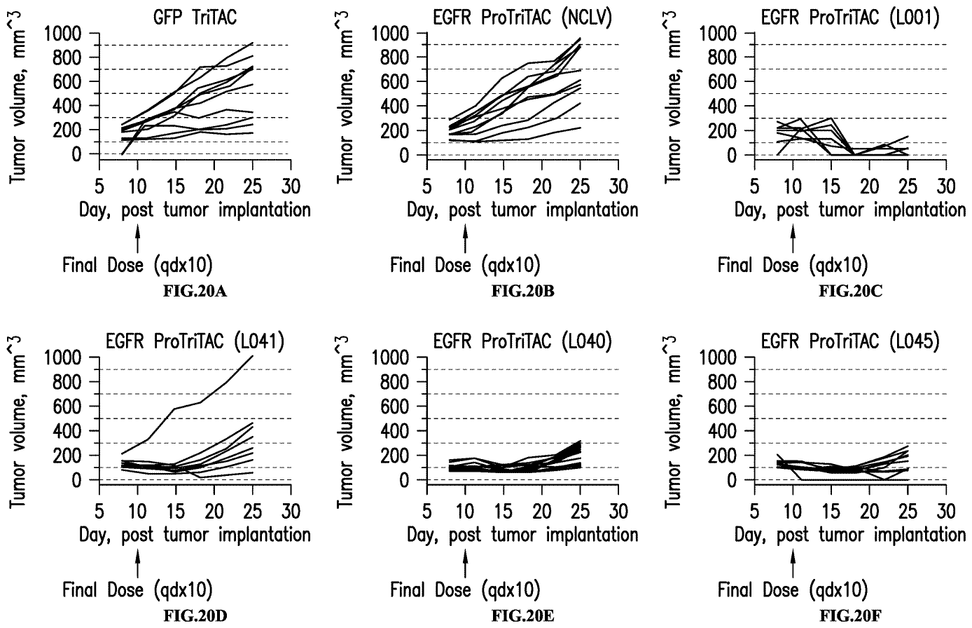

It was observed that in animals treated with the exemplary ProTriTAC molecules containing linker L040 there was a statistically significant delay in tumor growth as compared to the mice group which was treated with the control GFP TriTAC molecule, or the mice group that was treated with the ProTriTAC NCLV molecule. Similar observation was also made for the ProTriTAC molecules containing linker sequences L001, L041, and L045. The data is shown in FIG. 20.

It is also possible to carry out a similar study with xenograft models using other cell lines, such as A549 (non-small cell lung carcinoma) cells, DU-145 (prostate) cells, MCF-7 (breast) cells, Colo 205 (colon) cells, 3T3/]GF-IR (mouse fibroblast) cells, NCI H441 cells, HEP G2 (hepatoma) cells, MDA MB 231 (breast) cells, HT-29 (colon) cells, MDA-MB-435s (breast) cells, U266 cells, SH-SYSY cells, Sk-Mel-2 cells, NCI-H929, RPM18226, and A431 cells.

### Example 9: Demonstration of Reduced Cytokine Levels in Cynomolgus Monkeys, Correlated with Masking of TriTAC Molecules

In this study, cynomolgus monkeys were treated with three different concentrations (30 μg/kg; 300 μg/kg; and 1000 μg/kg) of an exemplary EGFR targeting ProTriTAC molecule containing a non-cleavable linker (ProTriTAC (NCLV),or with three different concentrations (10 μg/kg; 30 μg/kg; and 100 μg/kg) of an exemplary EGFR targeting TriTAC molecule (SEQ ID No. 793).

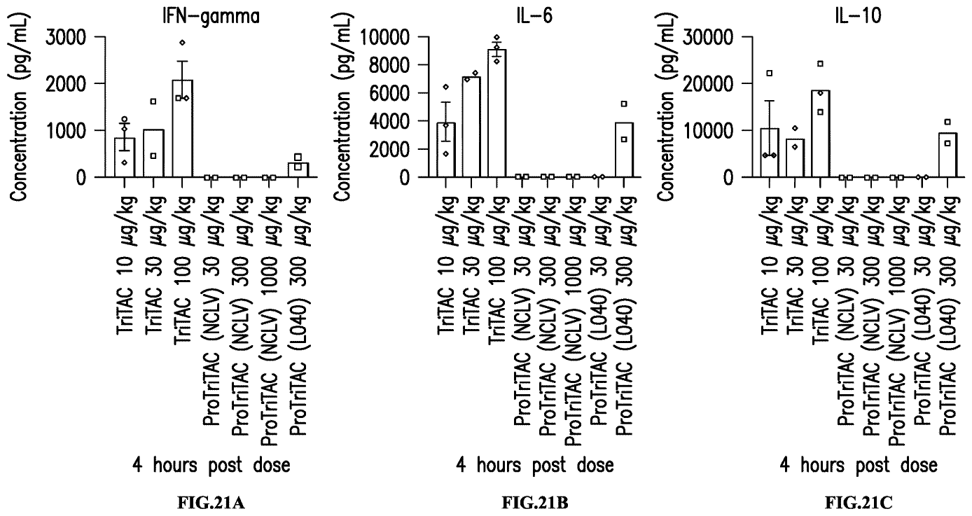

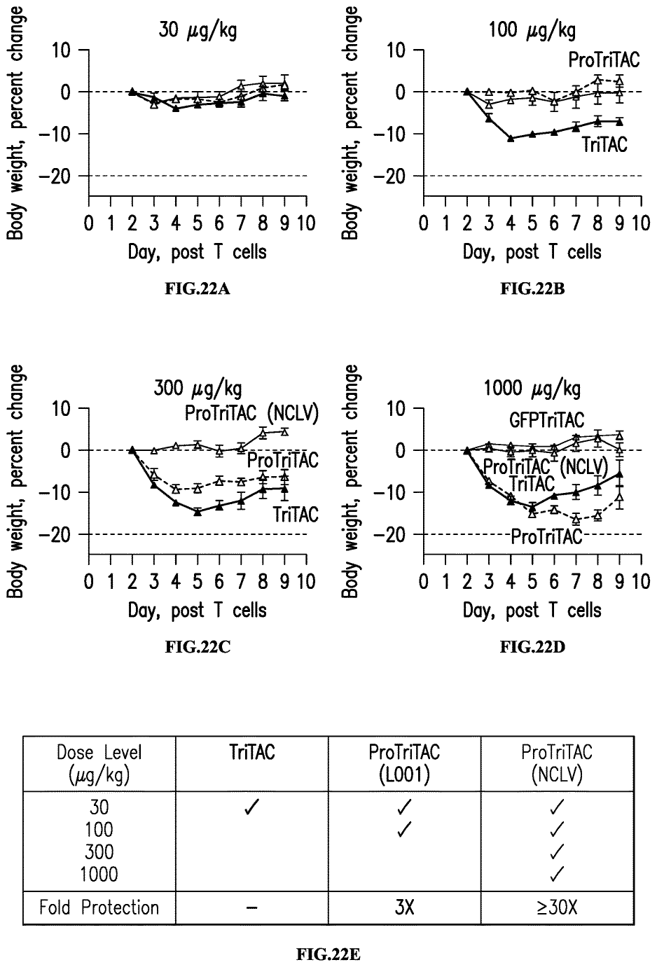

As shown in FIG. 21, after 4 hours following administering the ProTriTAC (NCLV) molecule, IFN-gamma IL-6 and IL-10 levels were significantly lower in comparison with administering the EGFR targeting TriTAC molecule.

### Example 10: Demonstration of Improved Tolerability in Mouse, Conferred by an Exemplary EGFR Targeting ProTriTAC Molecule

In this study, the tolerability of an exemplary EGFR targeting ProTriTAC molecule was assessed. Seven weeks old NSG female tumor free mice were intraperitoneally injected with 2×10⁷ expanded human T cells at the commencement of the study, i.e., at day 0. On day 2, treatment was started by dividing the mice into various groups and administering to them varying concentrations of the exemplary EGFR targeting ProTriTAC molecule, containing the linker sequence L001,an EGFR targeting TriTAC molecule, and an EGFR targeting ProTriTAC molecule containing a non-cleavable linker (ProTriTAC (NCLV). The molecules were administered once daily for 10 days, at the following dosages: 30 μg/kg, 100 μg/kg, 300 μg/kg. Starting from day 2, body weight of the animals were recorded daily.

As shown in FIG. 22, the EGFR targeting ProTriTAC molecule containing a non-cleavable linker (ProTriTAC (NCLV)) and a GFP TriTAC (used as a negative control) were very well tolerated in mice even at the highest dose of 1000 μg/kg. The EGFR targeted ProTriTAC molecule containing the linker sequence of L001 was well tolerated at the dosage of 100 μg/kg, whereas the EGFR targeted TriTAC was well tolerated at 30 μg/kg. It was thus observed that the ProTriTAC containing the L001 linker sequence conferred about 3 fold increase in tolerability and the ProTriTAC (NCLV) conferred about a 30 fold increase in tolerability, in mouse. The mouse maximum tolerated dose for the ProTriTAC (NCLV) and TriTAC molecules was consistent with what was observed in cynomolgus monkeys.

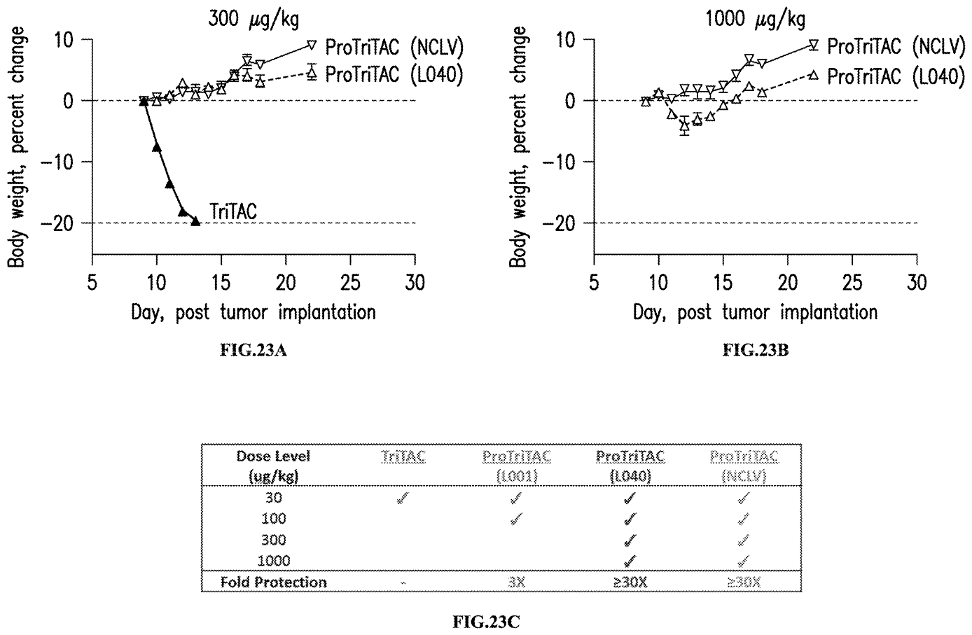

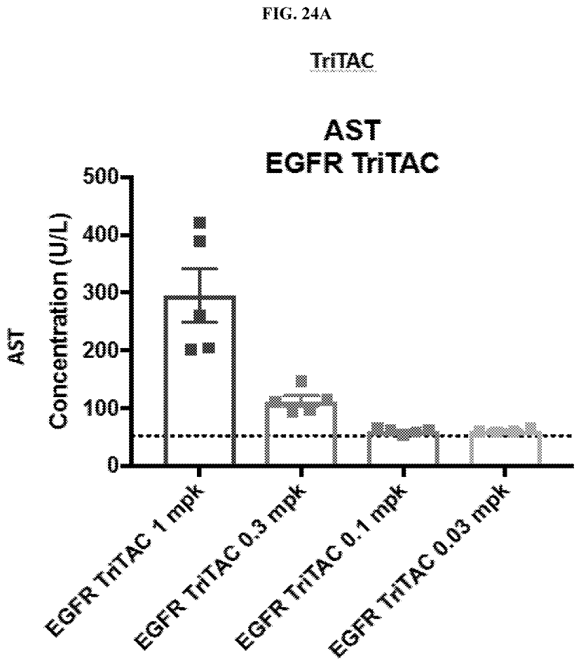

To further explore the role of the linker in tolerability of the EGFR targeting ProTriTAC molecule in mouse, the linker sequence was changed from L001 to that of L040. In this experiment, seven weeks old NSG female tumor free mice were subcutaneously injected with 5×10⁶ HCT116 tumor cells, at the commencement of the study, i.e., at day 0. At day 7 following the tumor cell injection, when the tumor volumes were about 180-200 mm³ (e.g., 183 mm³), the mice were injected intraperitoneally with 2×10⁷ expanded human T cells. Treatment was started on day 9, by dividing the mice into various groups and each group was administered an EGFR targeting TriTAC molecule, an EGFR targeting ProTriTAC molecule with linker sequence L040 (ProTriTAC(L040), and a ProTriTAC molecule containing a non-cleavable linker (ProTriTAC(NCLV). The molecules were administered once daily for 10 days, at the following dosages: 300 μg/kg and 1000 μg/kg. Starting from day 2, body weight of the animals were recorded daily. The results shown in FIG. 23 provide that the EGFR targeting ProTriTAC molecule with the linker sequence L040 conferred better tolerability than when the linker sequence L001 was used. The ProTriTAC (L040) was well-tolerated at 300 μg/kg and at 1000 μg/kg, with body weight percentage change comparable to that with the ProTriTAC (NCLV) molecule. It was thus observed that compared to the TriTAC molecule, the ProTriTAC containing the L040 linker sequence conferred about a 30 fold increase in tolerability, in mouse, similar to the 30 fold increase in tolerability observed with the ProTriTAC (NCLV) molecule.

### Example 11: Clinical Pathology Studies in Mice and Cynomolgus Monkeys

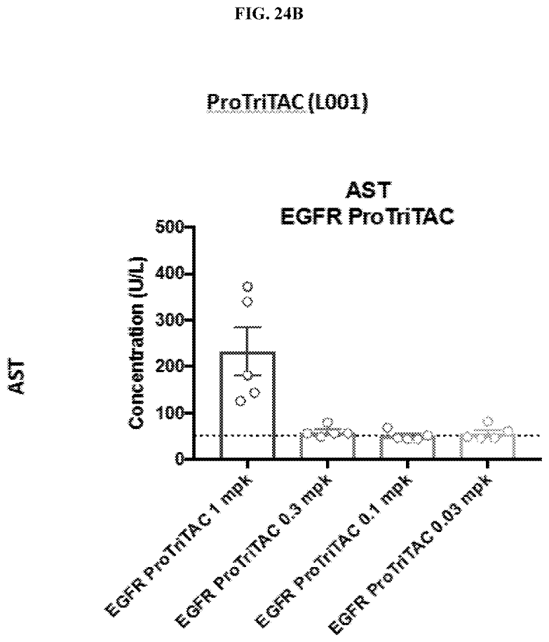

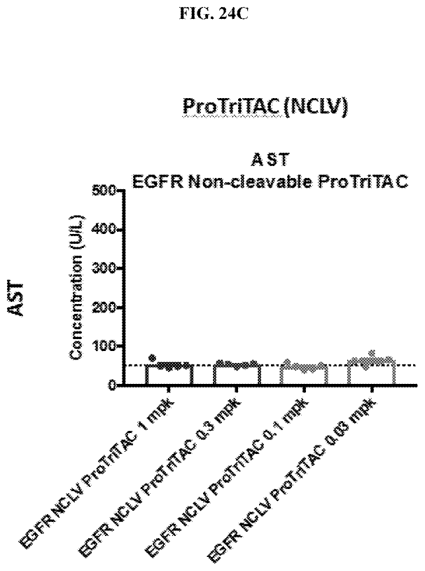

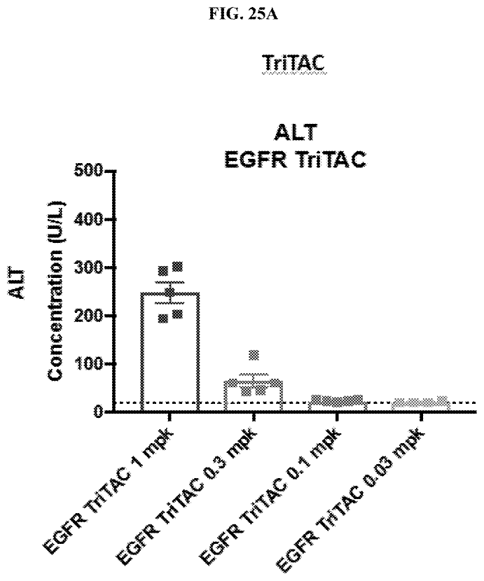

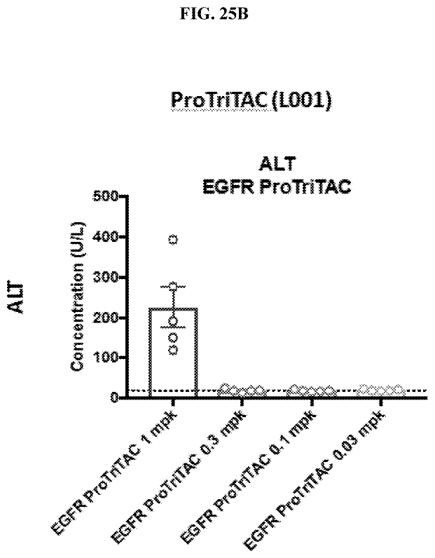

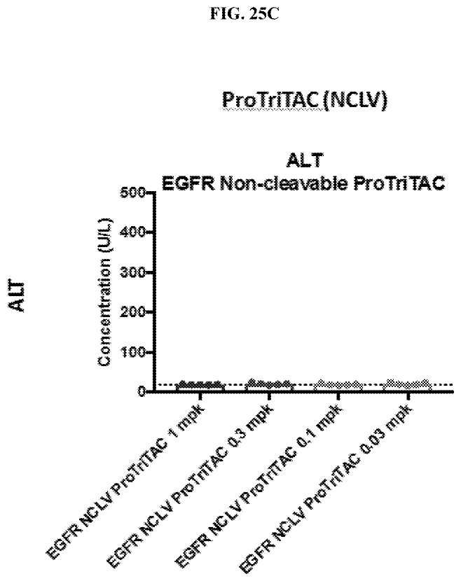

In this study, mice were treated with various concentrations of an EFGR targeting TriTAC molecule, an EGFR targeting ProTriTAC molecule containing the linker sequence L001 (ProTriTAC (L001), and an EGFR targeting ProTriTAC molecule containing a non-cleavable linker (ProTriTAC(NCLV)). Tolerability was assessed by measuring serum concentration of ALT (alanine aminotransferase) and AST (aspartate aminotransferase). Results are shown in FIGS. 24A, 24B, and 24C and FIGS. 25A, 25B, and 25C. It was observed that serum concentrations of AST and ALT were not elevated following administering the ProTriTAC (L001) at dosages up to 0.3 mg/kg, and that the serum concentrations of AST and ALT were not elevated following administering the ProTriTAC(NCLV) at dosages up to 1 mg/kg. In contrast, serum concentration of AST and ALT were not elevated following administering the TriTAC molecule at a dosage of 0.3 mg/kg.

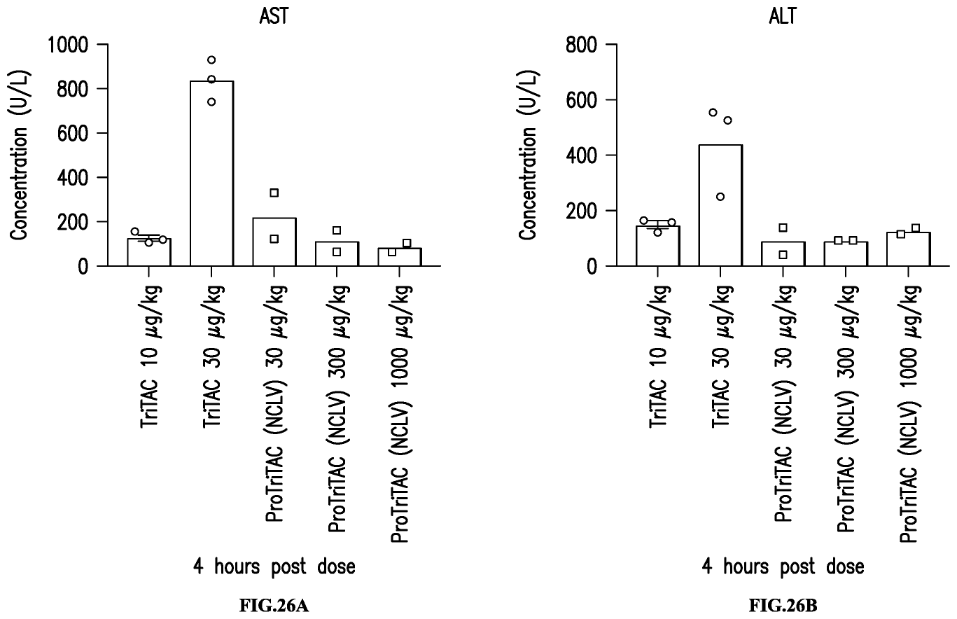

In another study, cynomolgus monkeys were treated with various concentrations of an EFGR targeting TriTAC molecule, and an EGFR targeting ProTriTAC molecule containing a non-cleavable linker (ProTriTAC(NCLV)). Tolerability was assessed by measuring serum concentration of ALT (alanine aminotransferase) and AST (aspartate aminotransferase). Results are shown in FIG. 26. It was observed that serum concentrations of AST and ALT were not elevated following administering the ProTriTAC (NCLV) at dosages up to 1000 μg/kg. In contrast, serum concentration of AST and ALT were not elevated following administering the TriTAC molecule at a dosage of 10 μg/kg.

### Example 12: Demonstration of Therapeutic Window Expansion with an Exemplary ProTriTAC Molecule of this Disclosure in a Tumor-Bearing Mouse Model

The aim of this study was to evaluate the expansion of therapeutic window by measuring anti-tumor activity and observable on-target toxicity in the same tumor-bearing mice. NSG female mice, 7 weeks old, were used for this study.

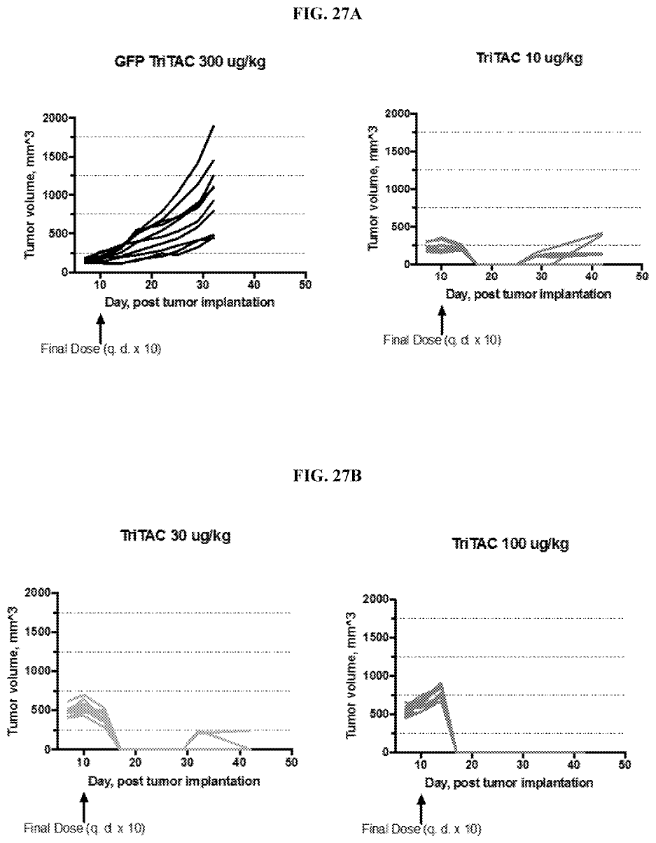

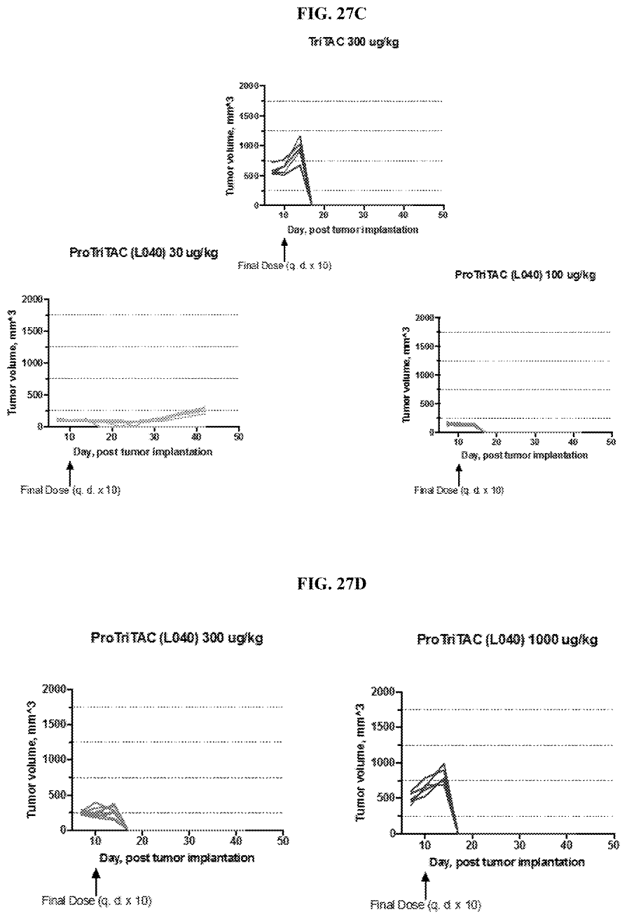

At the commencement of the study, on day 0, the NSG female mice were injected with 2.5×10⁶ expanded human T cells, and 5×10⁶ HCT116 (human colorectal carcinoma) tumor cells. The following day, on day 1, the mice were divided into groups and each group was treated with either GFP TriTAC molecule (SEQ ID No. 792), EGFR TriTAC molecule (SEQ ID No. 793), or an EGFR targeting ProTriTAC molecule containing linker L040, (SEQ ID No. 787) at the indicated dose levels in FIGS. 27A-297D (for GFP TriTAC the dosage was 300 μg/kg; for EGFR TriTAC dosages were 10 μg/kg, 30 μg/kg, 100 μg/kg, and 300 μg/kg; for EGFR ProTriTAC dosages were 30 μg/kg, 100 μg/kg, 300 μg/kg, and 1000 μg/kg) and administered daily for a period of 10 days (i.e., final dose was administered on day 10 following injection of tumor cells and expanded cells to the animals) and tumor volumes were measured at regular intervals, beginning a few days prior to the administration of the final dose at day 10. Results shown in FIGS. 27A-27D.

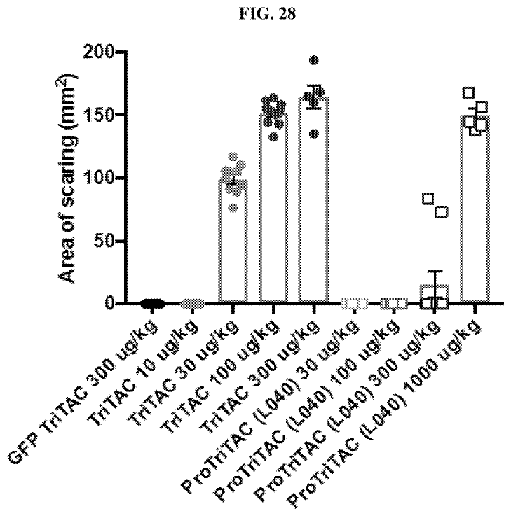

On-target EGFR-related toxicity was determined by measuring the radius of the red scarring skin lesion above the original tumor implantation site with a caliper and applying the equation Area=π*(radius of lesion)² on day 14. Results provided in FIG. 28 show that an exemplary EGFR ProTriTAC has 30× better tolerability compared to an EGFR TriTAC in the same HCT116 tumor-bearing mice as measured by the onset of red scarring skin lesions above the original tumor implantation site. Therapeutic window is defined as the difference between the minimal dose level required for anti-tumor activity and the highest skin lesion-free dose level.

Results (from FIGS. 27 and 28) show that the exemplary protease-cleavable EGFR ProTriTAC is 3× less potent but 30× more tolerated (i.e., has a 10× improved therapeutic window) than the EGFR TriTAC when efficacy and toxicity are measured on the same tumor-bearing mice, as summarized in below Table. This would make it possible to dose the ProTriTAC at a dose that is about 3× higher than the TriTAC, to get at least the same efficiency and better tolerability.

| | Min | Max | Therapeutic |
|---|---|---|---|
| | efficacious dose | scar-free dose | Window |
| TriTAC | 10 μg/kg | 10 μg/kg | 1 |
| ProTriTAC | 30 μg/kg | 300 μg/kg | 10 |

### Example 13: An Exemplary ProTriTAC Molecule Containing a Binding Moiety with Extended Non-CDR Loop into which a Human CD3E Epitope is Grafted

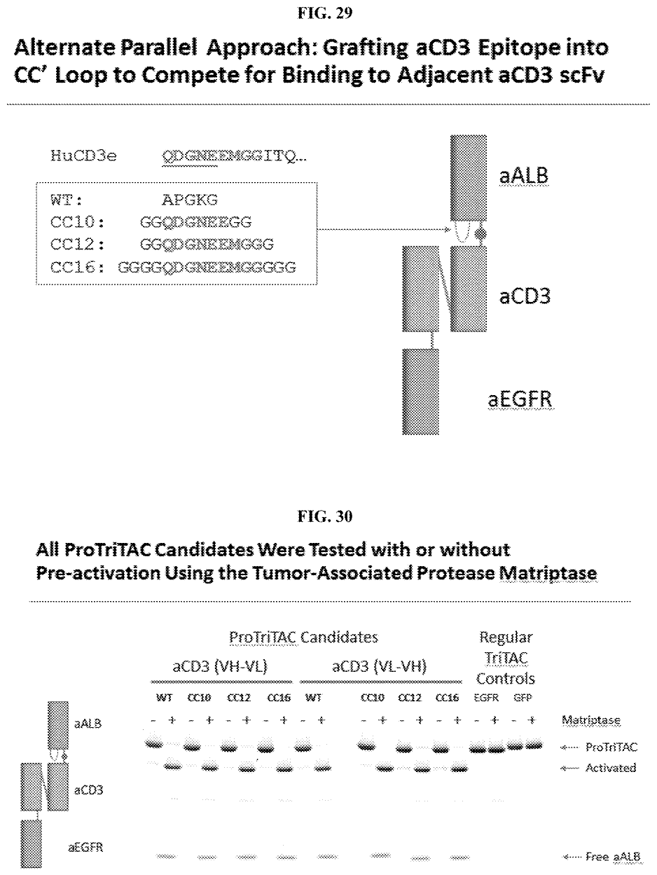

The sequence of a binding moiety comprising non-CDR loops (AB, EF, C″D, and CC′) was obtained. A portion of the human CD3ε sequence was grafted into the CC′ loop of the non-CDR loops within the binding moiety, along with glycine residues to further extend the CC′ loop. FIG. 29 illustrates three different variants comprising 10, 12, or 16 amino acid extensions to the CC′ loop. In case of the variant CC10, the portion of human CD3ε sequence grafted into the CC′ loop, to replace the wild type sequence of APGKG (SEQ ID NO: 795) was QDGNEE (SEQ ID NO. 801), and in addition 4 glycine residues were inserted to extend the CC′ loop. In case of the variant CC12, the portion of human CD3ε sequence grafted into the CC′ loop, to replace the wild type sequence of APGKG (SEQ ID NO: 795) was QDGNEEMGG (SEQ ID No. 802), and in addition 3 glycine residues were inserted to extend the CC′ loop. In case of the variant CC12, the portion of human CD3ε sequence grafted into the CC′ loop, to replace the wild type sequence of APGKG (SEQ ID NO: 795) was QDGNEEMGG (SEQ ID No. 803), and in addition 7 glycine residues were inserted to extend the CC′ loop. The binding moiety comprising extended non-CDR loops comprising the CD3ε sequences, as described above, were cloned into a vector further comprising coding sequences for a protease cleavable linker, a scFv containing a CD3 binding domain, and an EGFR binding domain, to express a ProTriTAC molecule. The ProTriTAC molecule contained an exemplary binding moiety of this disclosure, a CD3 binding scFv, and an EGFR binding domain. The ProTriTAC molecule was subsequently exposed to a tumor associated protease, matriptase, to assay activation of the molecule upon cleavage of the protease cleavable linker, which separates the binding moiety (depicted as aALB in FIGS. 29 and 30) comprising the cleavable linker from the rest of the molecule, i.e., the scFv containing the CD3 binding domain (depicted as aCD3 in FIGS. 29 and 30) and the EGFR binding domain (aEGFR). FIG. 30 shows the activation of the ProTriTAC molecules, containing the CC10, CC12, or CC16 variants of CC′ non-CDR loop, CD3 binding scFv in a VH-VL (left panel) or a VL-VH (right panel) format, upon treatment with matriptase. A ProTriTAC molecule containing a wild-type CC′ loop in the binding moiety was used as a control for the protease activation assay. In addition, a TriTAC molecule that is not in the “pro” form, i.e., a molecule that includes the same domains as the ProTriTAC molecule, except that it has a half-life extension domain, such as albumin, instead of a binding moiety, was also treated with matriptase and used as a control. Results indicated that the ProTriTAC molecules were activated upon cleavage, to generate a free albumin binding domain (depicted as Free aALB in FIG. 30) whereas the albumin domain did not separate from the TriTAC molecules. Thus, the ProTriTAC molecules containing a binding moiety of this disclosure was able to readily dissociate from half-life extending domain upon cleavage in a tumor microenvironment, unlike the TriTAC versions, and thereby were amenable to rapid clearance from the systemic circulation upon activation.

Further studies were carried out to assay the binding of the binding moiety containing the human CD3ε to CD3. It was observed that, in the presence of human serum albumin, the activated forms of ProTriTAC molecules that contained the binding moiety containing the human CD3ε were about 20 times potent in binding CD3 than their activated forms which did not contain the binding moiety. Results are shown in FIG. 31.

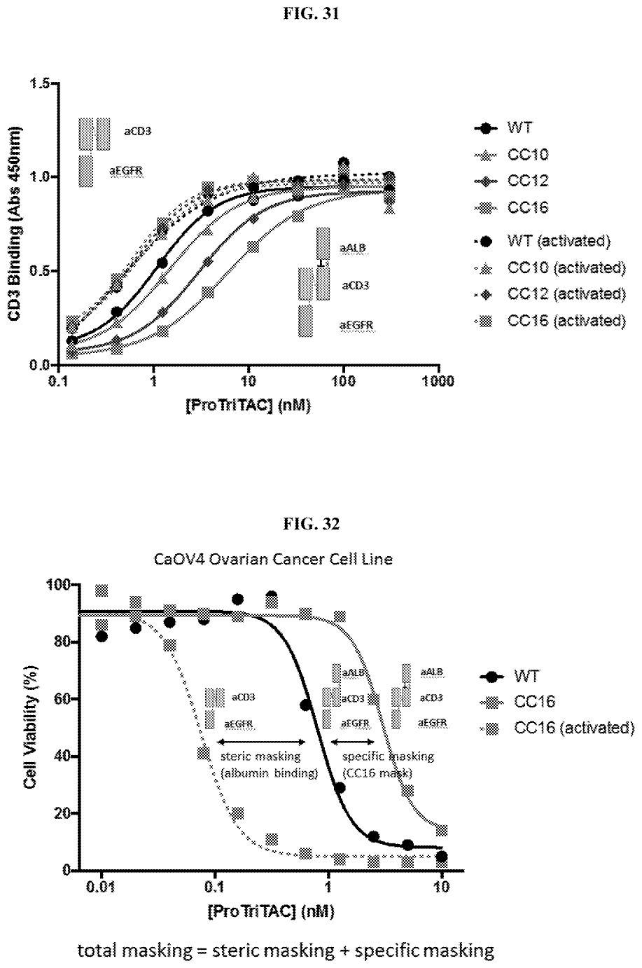

Cell killing potential of a ProTriTAC molecule that contained a binding moiety as described herein was also assayed in a study where CaOV4 cell line was treated with the ProTriTAC molecule or its activated form in the presence of human serum albumin. As shown in FIG. 32, the ProTriTAC molecule containing the CC16 variant of CC′ non-CDR loop was about 50 times more potent in killing cancer cells compared to its activated form, which was separated from the albumin binding domain. The total observed masking is a combination of steric masking due to binding to human serum albumin and specific masking from the CC16 mask in the CC′ non-CDR loop.

### Example 14: Soft Library Mutagenesis Identified CC′ Loop as Most Amenable to Modification

To identify locations within the non-CDR loops (AB, CC′, C″D, and EF) that were most amenable to modification, for creating a masking capability, libraries were assembled and generated using four groups of overlapping DNA oligos containing randomized degenerate “NNK” codons and with different loop lengths, as indicated in the schematic below:

AB loop oligos:
WT: LVQPGN (20%) (SEQ ID NO: 903)
AB0: XXXXXX (20%)
AB1: XXXXXXX (20%)
AB2: XXXXXXXX (20%)
AB3: XXXXXXXXX (20%)

CC′ loop oligos:
WT: APGKG (20%) (SEQ ID NO: 795)
CC0: XXXXX (20%)
CC1: XXXXXX (20%)
CC2: XXXXXXX (20%)
CC3: XXXXXXXX (20%)

C″D loop oligos:
WT: DSVKGR (20%) (SEQ ID NO: 904)
CD0: XXXXXX (20%)
CD1: XXXXXXX (20%)
CD2: XXXXXXXX (20%)
CD3: XXXXXXXXX (20%)

EF loop oligos:
WT: SLRPED (20%) (SEQ ID NO: 905)
EF0: XXXXXX (20%)
EF1: XXXXXXX (20%)
EF2: XXXXXXXX (20%)
EF3: XXXXXXXXX (20%)

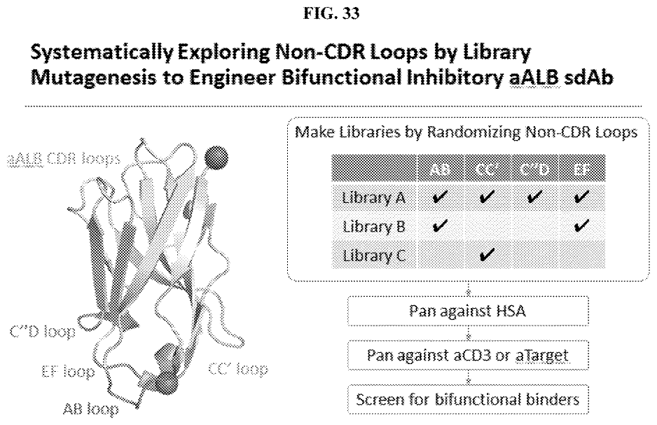

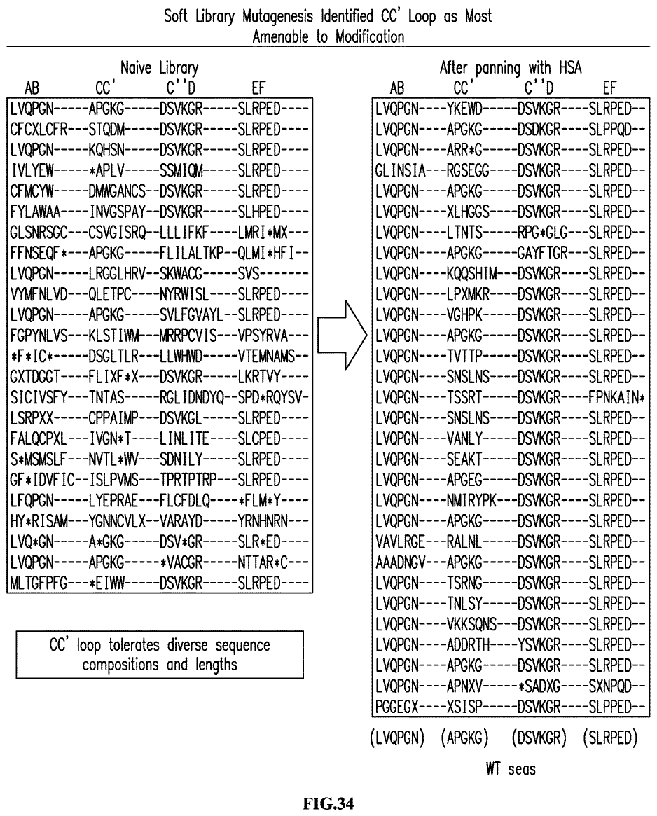

Note: “X” denotes a randomized residue (“NNK” codon) that could be any of the 20 natural amino acids as well as stop codon. The goal was to have approximately 20% of each non-CDR loop be wild-type. These wild-type oligos served as internal benchmarks to gauge the tolerance of each loop to modification (sequence composition and/or length changes). A loop that was less tolerable to change could easily revert to wild-type; in contrast, a loop that was highly amenable to change would maintain the diverse sequence repertoire. To this end, 24 clones were sequenced from the naive library to verify the randomization of non-CDR loops prior to panning with HAS, as shown in FIG. 33. After two rounds of phage panning against HSA, 30 clones were sequenced to examine the non-CDR loop composition. The results showed that 3 out of 4 non-CDR loops (AB, C″D, and EF) mostly utilized the 20% wild-type oligo and reverted back to the wild-type, suggesting that they may be less preferred than the wild-type sequence. However, the CC′ loop kept the diverse sequence repertoire (both sequence and length), suggesting that the CC′ loop may be the loop most tolerable to randomization that we could exploit for specific masking of adjacent domains, as shown in FIG. 34.

### Example 15: Screening of Phage Display Library for Identification of EpCAM Binding Domains

Llamas were immunized with purified EpCAM protein expressed in Expi293 cells. A phage display library for expression of heavy variable antibody domains was constructed from circulating B cells. See van der Linden, de Geus, Stok, Bos, van Wassenaar, Verrips, and Frenken. 2000. J Immunol Methods 240:185-195. Phage clones were screened for binding to EpCAM by expressing anti-EpCAM proteins in E coli, preparing periplasmic extracts, and proteins were screened for human and cynomolgus EpCAM binding activity using a colorimetric ELISA. Thirty-eight unique heavy chain only sequences were identified (SEQ ID Nos. 804-841) that produced a signal in the ELISA screening relative to the control with human and/or cynomolgus EpCAM proteins (as shown in Table 10).

TABLE 10
Binding of Llama anti-Human EpCAM
heavy chain only single domain
antibodies to Human and Cynomolgus
EpCAM, as demonstrated by signal
in an ELISA Assay (absorbance readings
in a colorimetric ELISA assay), relative to
control heavy chain only single domain antibodies

| | ELISA | ELISA | | Human | Cynomolgus |
|---|---|---|---|---|---|
| Sequence | Human | Cynomolgus | ELISA | EpCAM/ | EpCAM/ |
| name | EpCAM | EpCAM | Control | Control | Control |
| EPL90 | 1.6 | 1.7 | 0.2 | 10 | 10 |
| EPL118 | 3.4 | 2.9 | 0.7 | 5 | 4 |
| EPL138 | 3.1 | 2.7 | 0.9 | 3 | 3 |
| EPL145 | 0.6 | 0.4 | 0.2 | 3 | 2 |
| EPL164 | 0.6 | 2.8 | 0.1 | 7 | 34 |
| EPL31 | 0.6 | 0.5 | 0.1 | 7 | 6 |
| EPL55 | 0.4 | 0.6 | 0.1 | 5 | 7 |
| EPL57 | 1.7 | 3.4 | 0.1 | 14 | 27 |
| EPL136 | 0.9 | 1.3 | 0.1 | 9 | 13 |
| EPL15 | 0.7 | 3.2 | 0.1 | 8 | 35 |
| EPL34 | 0.9 | 3.1 | 0.1 | 10 | 36 |
| EPL86 | 0.8 | 3.0 | 0.1 | 8 | 31 |
| EPL153 | 3.1 | 2.2 | 0.1 | 31 | 21 |
| EPL20 | 2.9 | 2.2 | 0.5 | 6 | 4 |
| EPL70 | 3.2 | 2.2 | 0.1 | 24 | 16 |
| EPL125 | 3.5 | 3.5 | 0.5 | 8 | 8 |
| EPL13 | 2.9 | 4.0 | 0.2 | 17 | 23 |
| EPL129 | 2.0 | 3.1 | 0.1 | 20 | 29 |
| EPL159 | 0.4 | 0.2 | 0.1 | 3 | 2 |
| EPL120 | 3.2 | 2.3 | 0.6 | 5 | 4 |
| EPL126 | 3.4 | 2.8 | 0.7 | 5 | 4 |
| EPL60 | 1.1 | 3.5 | 0.1 | 10 | 33 |
| EPL156 | 3.6 | 4.0 | 0.2 | 16 | 17 |
| EPL2 | 2.2 | 3.6 | 0.1 | 15 | 24 |
| EPL43 | 1.8 | 3.8 | 0.1 | 13 | 27 |
| EPL10 | 2.7 | 2.0 | 0.2 | 12 | 9 |
| EPL49 | 1.1 | 0.6 | 0.1 | 8 | 4 |
| EPL58 | 1.1 | 0.5 | 0.1 | 8 | 4 |
| EPL74 | 1.4 | 0.7 | 0.1 | 10 | 5 |
| EPL78 | 3.1 | 2.0 | 0.2 | 17 | 11 |
| EPL82 | 0.9 | 1.1 | 0.2 | 4 | 5 |
| EPL83 | 2.0 | 1.1 | 0.6 | 3 | 2 |
| EPL97 | 2.5 | 1.7 | 0.3 | 8 | 5 |
| EPL109 | 0.4 | 0.1 | 0.1 | 5 | 2 |
| EPL117 | 0.9 | 0.6 | 0.2 | 4 | 3 |
| EPL127 | 0.4 | 0.9 | 0.1 | 5 | 11 |
| EPL152 | 3.4 | 2.8 | 0.8 | 4 | 4 |
| EPL189 | 1.3 | 0.9 | 0.1 | 12 | 8 |

### Example 16: Incorporation of EpCAM Binding Heavy Chain Only Single Domain Antibodies into Fusion Proteins and T Cell Dependent Cellular Cytotoxicity Assays

Selected anti-EpCAM heavy chain only single domain antibodies from Example 15 were cloned into DNA constructs for expression of recombinant proteins. These expression constructs all encoded a signal peptide. One set of anti-EpCAM constructs (SEQ ID Nos. 842 to 868) was designed to express a fusion protein with a humanized anti-CD3 scFv domain on the N-terminus of the mature secreted fusion protein followed by a llama anti-EpCAM domain, with the two domains linked by the sequence GGGGSGGGS (SEQ ID NO: 928), and with a HHHHHH (SEQ ID NO: 927) on the C-terminus. One second of anti-EpCAM constructs (SEQ ID Nos. 869 to 895) was designed to express a fusion protein with a llama anti-EpCAM domain on the N-terminus of the mature secreted fusion protein followed a humanized anti-CD3-scFv domain, with the two domains linked by the sequence GGGGSGGGS (SEQ ID NO: 928), and with a HHHHHH (SEQ ID NO: 927) on the C-terminus.

These anti-EpCAM/anti-CD3 (from N-terminus to C-terminus) or anti-CD3/anti-EpCAM (from N terminus to C terminus) fusion protein constructs were transfected into Expi293 cells. The amount of anti-EpCAM/anti-CD3 fusion protein in the conditioned media from the transfected Expi293 cells was quantitated using by using an Octet instrument with streptavidin and loaded with biotinylated CD3-Fc fusion protein using an anti-CD3 fusion protein of similar molecular weight to the anti-EPCAM/anti-CD3 proteins as a standard.

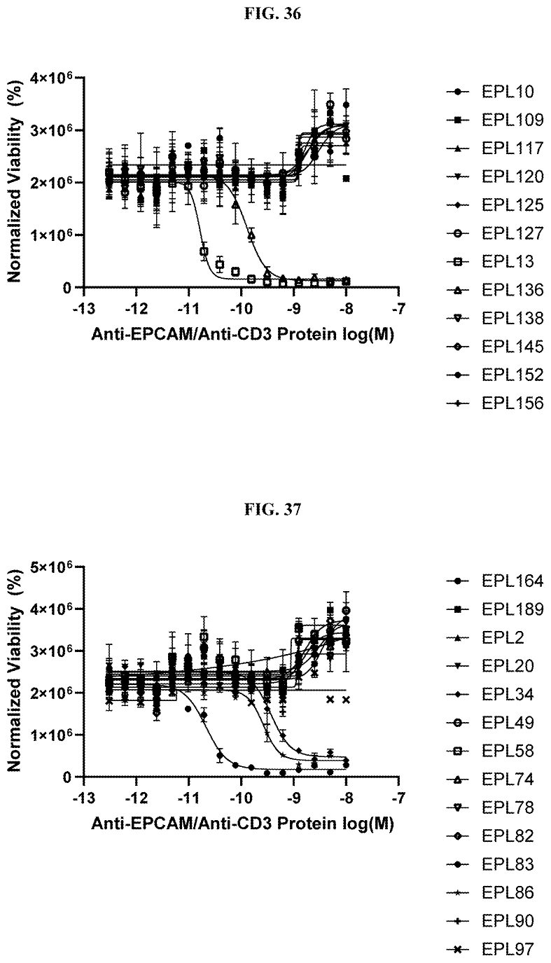

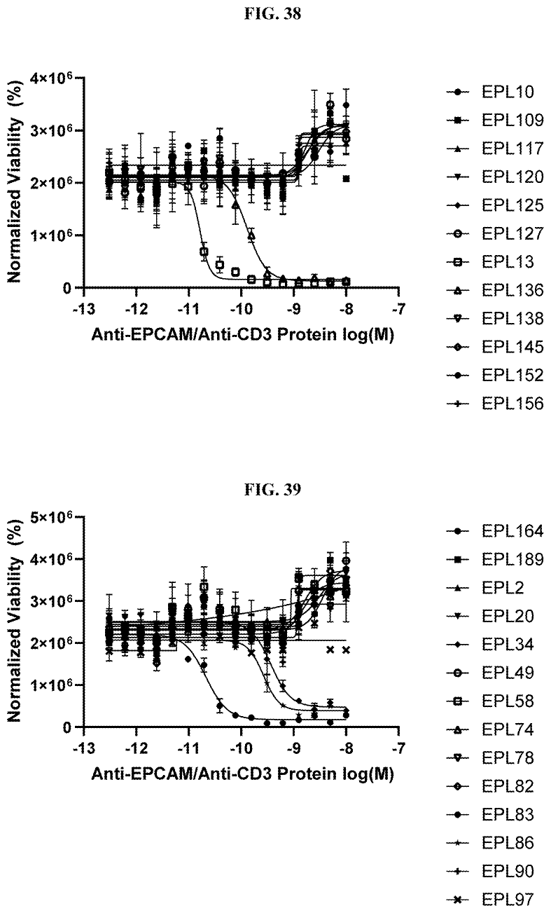

The conditioned media were tested in a T-cell dependent cellular cytotoxicity assay. See Nazarian AA, Archibeque IL, Nguyen YH, Wang P, Sinclair AM, Powers DA. 2015. J Biomol Screen. 20:519-27. In this assay, luciferase labelled NCI-H508 cells, which express EpCAM, were combined with purified human T cells and a titration of the anti-EpCAM/anti-CD3 fusion protein or the anti-CD3/anti-EpCAM. It was hypothesized that if the fusion protein directs T cells to kill the NCI-H508 cells, the signal in a luciferase assay performed at 48 hours after starting the experiment should decrease. FIGS. 36-39 provide the TDCC data in graphical format. EC₅₀ values from the TDCC assays are listed in Table 11 (lists EC₅₀ data for SEQ ID Nos. 842 to 868) and Table 12 (lists EC₅₀ data for SEQ ID Nos. 869-895). The most potent molecule (EPL13) had an EC₅₀ value of about 1.6 pM. Some of the anti-pCAM binding proteins were only active when present in an anti-CD3/anti-EpCAM configuration. One anti-EpCAM sequence, EPL34, was only active in the anti-EpCAM/anti-CD3 configuration. A negative control for the TDCC assays was anti-GFP/anti-CD3 protein, and this protein did not direct the T cells to kill the NCI-H508 cells (data not shown).

TABLE 11
EC₅₀ Values for Redirected T Cell Killing of NCI-H508
Cells by Anti-CD3/Anti-EPCAM Proteins Containing
Llama Anti-EPCAM Sequences (n/a = insufficient
activity to calculate an EC₅₀ using the
protein concentrations tested)

| Anti-EpCAM Sequence | NCI-H508 Cell Killing EC₅₀ (M) |
|---|---|
| EPL10 | n/a |
| EPL109 | 2.2E−09 |
| EPL117 | n/a |
| EPL120 | 5.8e−010 |
| EPL125 | 1.3E−09 |
| EPL127 | n/a |
| EPL13 | 1.6E−12 |
| EPL136 | 6.1E−12 |
| EPL138 | 7.9E−12 |
| EPL145 | n/a |
| EPL152 | n/a |
| EPL153 | 1.4E−10 |
| EPL156 | n/a |
| EPL164 | 3.6E−10 |
| EPL189 | n/a |
| EPL2 | n/a |
| EPL20 | n/a |
| EPL34 | n/a |
| EPL49 | n/a |
| EPL58 | n/a |
| EPL74 | 2.6E−09 |
| EPL78 | n/a |
| EPL82 | n/a |
| EPL83 | 3.1E−10 |
| EPL86 | 4.7E−10 |
| EPL90 | 9.2E−12 |
| EPL97 | n/a |

TABLE 12
EC₅₀ Values for Redirected T Cell Killing of NCI-H508
Cells by Anti-EpCAM/Anti-CD3 Proteins Containing Llama
Anti-EPpCAM Sequences (n/a = insufficient activity to
calculate an EC₅₀ using the protein concentrations tested)

| Anti-EPCAM Sequence | NCI-H508 Cell Killing EC50 (M) |
|---|---|
| EPL10 | n/a |
| EPL109 | n/a |
| EPL117 | n/a |
| EPL120 | n/a |
| EPL125 | n/a |
| EPL127 | n/a |
| EPL13 | 1.6E−11 |
| EPL136 | 1.3E−10 |
| EPL138 | n/a |
| EPL145 | n/a |
| EPL152 | n/a |
| EPL153 | not expressed |
| EPL156 | n/a |
| EPL164 | n/a |
| EPL189 | n/a |
| EPL2 | n/a |
| EPL20 | n/a |
| EPL34 | 3.7E−10 |
| EPL49 | n/a |
| EPL58 | n/a |
| EPL74 | n/a |
| EPL78 | n/a |
| EPL82 | n/a |
| EPL83 | 2.1E−11 |
| EPL86 | 2.7E−10 |
| EPL90 | n/a |
| EPL97 | n/a |

Using conditioned media with known concentrations of anti-EpCAM/anti-CD3 or anti-CD3/anti-EpCAM fusion proteins, the binding affinities of the fusion proteins for human and cynomolgus monkey EpCAM proteins were measured. An Octet instrument with streptavidin tips were loaded with biotinylated human or cynomolgus EpCAM protein, and K_{D} values were calculated by measuring the on rate and off rate of binding of the anti-EPCAM/anti-CD3 fusion or anti-CD3/anti-EpCAM fusion proteins to the biotinylated EpCAM proteins. The K_{D} measurements were made using a single 50 nM concentration of the anti-EPCAM/anti-CD3 or anti-CD3/anti-EpCAM fusion proteins, which allowed for rank ordering potency. The measured relative affinities are listed in Table 13. All of the fusion proteins bound to cynomolgus EpCAM, with K_{D} values ranging from 1.6 to 56 nM. Most, but not all of the fusion proteins were measured binding to human EpCAM with K_{D} values ranging from 0.8 to 74 nM.

TABLE 13
Binding Affinities to Human and Cyno EpCAM
of Anti-EPCAM/Anti-CD3 or Anti-CD3/Anti-EpCAM
Fusion Proteins Containing Llama Anti-EpCAM Sequences

| | Anti-CD3/ | | Anti-EpCAM/ | |
|---|---|---|---|---|
| | Anti-EpCAM | | Anti-CD3 | |
| Humanized anti- | hu KD | cy KD | hu KD | cy KD |
| EpCAM Binder | (nM) | (nM) | (nM) | (nM) |
| EPL13 | 29 | 13 | 14 | 7.6 |
| EPL136 | 74 | 56 | 32 | 31 |
| EPL138 | 2.2 | 2.2 | 0.8 | 1.3 |
| EPL153 | n/q | 3.5 | No expression | |
| EPL164 | 3.1 | 3.6 | 2.1 | 2.9 |
| EPL34 | n/q | 14 | n/q | 6.4 |
| EPL83 | 1.1 | 3 | 1.6 | 3 |
| EPL86 | n/q | 8.3 | n/q | 7 |
| EPL90 | 1.9 | 1.6 | 0.8 | 1.2 |

### Example 17: Humanization of EpCAM Binding Heavy Chain Only Single Domain Antibodies and T Cell Dependent Cellular Cytotoxicity Assays

Three of the llama anti-EpCAM antibodies sequences identified in Example 15 were humanized by grafting their CDR sequences onto human germline sequences, while retaining some llama framework sequences to ensure the antibodies did not lose activity (SEQ ID Nos. 896 to 898).

These sequences were cloned into expression constructs for expression of anti-EpCAM/anti-CD3 fusion proteins (SEQ ID Nos. 899 to 901) in Expi293 cells, as described in Example 16.

The amount of anti-EpCAM/anti-CD3 fusion proteins present in the conditioned medium was quantitated as described in Example 16. The affinities of these humanized proteins for human, cynomolgus, and mouse EpCAM were measured as described in Example 16. The relative K_{D} values calculated from these measurements are listed in Table 14. All three sequences bound to human and cynomolgus EpCAM, with relative K_{D} values ranging from about 0.3 to about 18 nM. Two of the sequences also bound to mouse EpCAM, with K_{D} values ranging from about 1.4 to about 1.8 nM.

TABLE 14
Binding Affinities to Human, Cynomolgus, and Mouse EpCAM of Anti-
EpCAM/Anti-CD3 Fusion Proteins Containing Llama Anti-EpCAM
Sequences (n/q = not quantifiable under the experimental conditions used)

| Humanized Anti- | Human EpCAM | Cynomolgus | Mouse EPCAM |
|---|---|---|---|
| EpCAM Sequence | (nM) | EpCAM (nM) | (nM) |
| H13 | 17 | 18 | n/q |
| H90 | 0.3 | 1.3 | 1.4 |
| H138 | 0.3 | 1.8 | 1.8 |

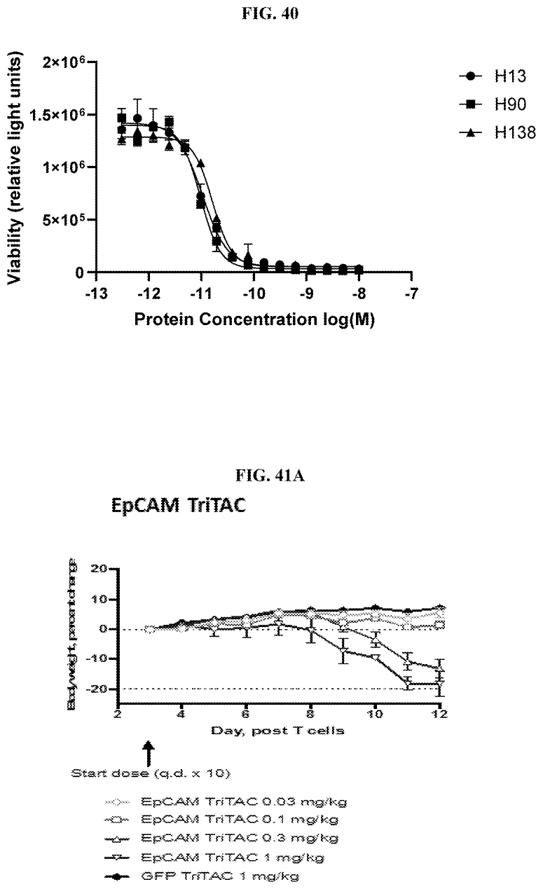

T cell killing potential of the anti-EpCAM/anti-CD3 fusion proteins present in the conditioned medium was assessed as described in Example 16. Results are provided in Table 15 and in FIG. 40.

TABLE 15
EC₅₀ Values for Redirected T Cell Killing of
NCI-H508 Cells by Purified Anti-CD3/Anti-EpCAM
Proteins Containing Humanized Anti-EpCAM Sequences

| Humanized anti-EpCAM Binder Sequence | EC₅₀ (pM) |
|---|---|
| H13 | 10 |
| H90 | 10 |
| H138 | 16 |

### Example 18: Demonstration of Improved Tolerability in Mouse, Conferred by an Exemplary EpCAM Targeting ProTriTAC Molecule

In this study, the tolerability of an exemplary EpCAM targeting ProTriTAC molecule was assessed. Seven weeks old NSG female tumor free mice were intraperitoneally injected with 2×10⁷ expanded human T cells at the commencement of the study, i.e., at day 0. On day 2, treatment was started by dividing the mice into various groups and administering to them varying concentrations of the exemplary EpCAM targeting ProTriTAC molecule, containing the linker sequence L040, an EpCAMR targeting TriTAC molecule, an EpCAM targeting ProTriTAC molecule containing a non-cleavable linker (EpCAM ProTriTAC (NCLV), and a GFP TriTAC molecule (SEQ ID No. 792) as a control. The molecules were administered once daily for 10 days, at the following dosages: 0.03 mg/kg, 0.1 mg/kg, 0.3 mg/kg, and 1 mg/kg. Starting from day 2, body weight of the animals were recorded daily.

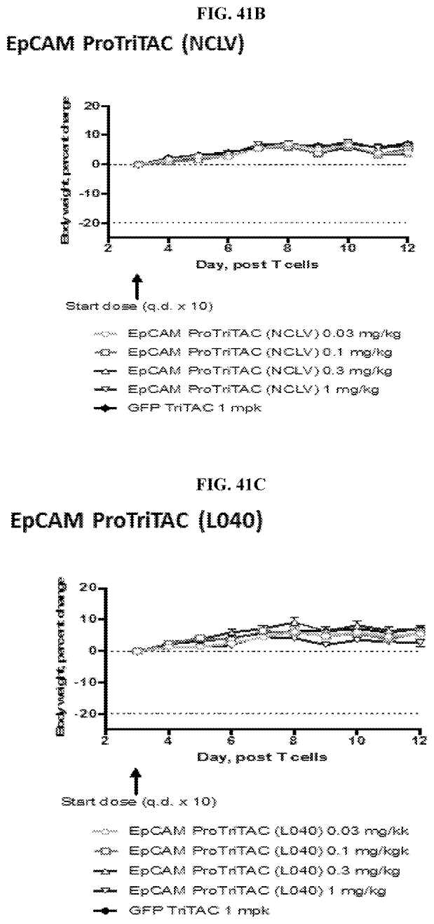

As shown in FIGS. 41A-41C, the EpCAM targeting ProTriTAC molecule containing a non-cleavable linker (ProTriTAC (NCLV)) (SEQ ID No. 908) and a GFP TriTAC (used as a negative control) were very well tolerated in mice even at the highest dose of 1 mg/kg. The EpCAM targeted ProTriTAC molecule containing the linker sequence of L040 (SEQ ID No. 907) was well tolerated at the highest tested dosage of 1 mg/kg, whereas the EpCAM targeted TriTAC (SEQ ID No. 906) was well tolerated 0.1 mg/kg. It was thus observed that the EpCAM targeting ProTriTAC containing the L040 linker sequence conferred at least about 10 times improved tolerability, in mouse, compared to the EpCAM targeting TriTAC.

### Example 19: Xenograft Tumor Model

An EpCAM targeting fusion protein of this disclosure (e.g., a fusion protein which is a trispecific protein comprising an anti-EpCAM heavy chain only single domain antibody, an anti-CD3 scFv, and an anti-Albumin domain) is evaluated in a xenograft model. In order to determine efficacy of the exemplary EpCAM targeting fusion protein in vivo, multiple xenograft tumor models are used. Examples of common tumor cell lines for use in xenograft tumor studies include A549 (non-small cell lung carcinoma) cells, DU-145 (prostate) cells, MCF-7 (breast) cells, Colo 205 (colon) cells, 3T3 (mouse fibroblast) cells, NCI H441 cells, HEP G2 (hepatoma) cells, MDA MB 231 (breast) cells, HT-29 (colon) cells, MDA-MB-435s (breast) cells, U266 cells, SH-SYSY cells, Sk-Mel-2 cells, NCI-H929, RPM18226, and A431 cells. Immune-deficient NOD/scid mice are sub-lethally irradiated (2 Gy) and subcutaneously inoculated with 1×10⁶ tumor cells (e.g., NCI H441 cells) into their right dorsal flank. When tumors reach 100 to 200 mm³, animals are allocated into 3 treatment groups. Groups 2 and 3 are intraperitoneally injected with 1.5×10⁷ activated human T-cells. Three days later, animals from Group 3 are subsequently treated with the exemplary EPCAM targeting trispecific antigen-binding protein of Groups 1 and 2 are only treated with vehicle. Body weight and tumor volume are determined for 30 days, beginning at least 5 days post treatment with the exemplary EPCAM targeting trispecific protein.

It is expected that animals treated with the exemplary EpCAM targeting trispecific protein have a statistically significant delay in tumor growth in comparison to the respective vehicle-treated control group.

### Example 20: Proof-of-Concept Clinical Trial Protocol for Administration of the EpCAM Targeting Trispecific Antigen-Binding Protein of Example 19 to Ovarian Cancer Patients

This is a Phase I/II clinical trial for studying an exemplary EpCAM targeting trispecific antigen-binding protein of this disclosure as a treatment for an epithelial ovarian cancer.

1. Study Outcomes:

2. Primary: Maximum tolerated dose of the exemplary EpCAM targeting trispecific protein.

3. Secondary: To determine whether in vitro response of the exemplary EpCAM targeting trispecific protein is associated with clinical response

### Phase I

4. The maximum tolerated dose (MTD) will be determined in the phase I section of the trial.
1.1 The maximum tolerated dose (MTD) will be determined in the phase I section of the trial.
1.2 Patients who fulfill eligibility criteria will be entered into the trial to EPCAM targeting trispecific proteins of the previous examples.
1.3 The goal is to identify the highest dose of EpCAM targeting trispecific proteins of the previous examples that can be administered safely without severe or unmanageable side effects in participants. The dose given will depend on the number of participants who have been enrolled in the study prior and how well the dose was tolerated. Not all participants will receive the same dose.

### Phase II

2.1 A subsequent phase II section will be treated at the MTD with a goal of determining if therapy with therapy of the exemplary EpCAM targeting trispecific protein results in at least a 20% response rate.
   Primary Outcome for the Phase II—To determine if therapy of EPCAM targeting trispecific protein results in at least 20% of patients achieving a clinical response (blast response, minor response, partial response, or complete response)

### Eligibility:

Histologically or cytologically confirmed epithelial ovarian cancer. May have Recurrent epithelial ovarian carcinoma or disease progression following failure of first-line, platinum-based chemotherapy with no more than one prior platinum based regimen therapy
Adequate laboratory values of bone marrow function, renal function, liver function, and echocardiogram tests

### Phase III

5.3.1A subsequent phase III section will carried out with the exemplary EpCAM targeting trispecific protein, wherein secondary endpoints such as response rate (RR), patient recorded outcomes (PRO), progression-free survival (PFS), duration of progression free survival, time to progression (TIP), overall survival, health-related quality of life assessment, number of participants with overall survival, duration of response, time to response, number of participants with response, and time to tumor growth etc. will be assessed.

SEQUENCE TABLE

| DESCRIPTION | SEQUENCE |
|---|---|
| PSMA Prodrug C1872 | EVQLVESGGGLVQPGNSLRLSCAASGFTFSKFGMSWVRQGGG |
| (SEQ ID NO: 43) | GGLDGNEEPGGLEWVSSISGSGRDTLYADSVKGRFTISRDNAK |
| | TTLYLQMNSLRPEDTAVYYCTIGGSLSVSSQGTLVTVSSGGGGK |
| | PLGLQARVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPEDEA |
| | EYYCTLWYSNRWVFGGGTKLTVLGGGGSGGGGSGGGGSEVQ |
| | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLVESGGGLVQPGGSLTLSCAASRFMISEYHMHWVR |
| | QAPGKGLEWVSTINPAGTTDYAESVKGRFTISRDNAKNTLYLQ |
| | MNSLKPEDTAVYYCDSYGYRGQGTQVTVSSHHHHHH |
| PSMA Non-cleavable Prodrug | EVQLVESGGGLVQPGNSLRLSCAASGFTFSKFGMSWVRQGGG |
| C1873 | GGLDGNEEPGGLEWVSSISGSGRDTLYADSVKGRFTISRDNAK |
| (SEQ ID NO: 44) | TTLYLQMNSLRPEDTAVYYCTIGGSLSVSSQGTLVTVSSGGGGS |
| | GGGGSGGVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPEDEA |
| | EYYCTLWYSNRWVFGGGTKLTVLGGGGSGGGGSGGGGSEVQ |
| | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLVESGGGLVQPGGSLTLSCAASRFMISEYHMHWVR |
| | QAPGKGLEWVSTINPAGTTDYAESVKGRFTISRDNAKNTLYLQ |
| | MNSLKPEDTAVYYCDSYGYRGQGTQVTVSSHHHHHH |
| PSMA Active Drug C1875 (SEQ | VVGGGGTQTVVTQEPSLTVSPGGTVTLTCASSTGAVTSGNYP |
| ID NO: 45) | NWVQQKPGQAPRGLIGGTKFLVPGTPARFSGSLLGGKAALTLS |
| | GVQPEDEAEYYCTLWYSNRWVFGGGTKLTVLGGGGSGGGGS |
| | GGGGSEVQLVESGGGLVQPGGSLKLSCAASGFTFNKYAINWV |
| | RQAPGKGLEWVARIRSKYNNYATYYADQVKDRFTISRDDSKNT |
| | AYLQMNNLKTEDTAVYYCVRHANFGNSYISYWAYWGQGTLV |
| | TVSSGGGGSGGGSEVQLVESGGGLVQPGGSLTLSCAASRFMIS |
| | EYHMHWVRQAPGKGLEWVSTINPAGTTDYAESVKGRFTISRD |
| | NAKNTLYLQMNSLKPEDTAVYYCDSYGYRGQGTQVTVSSHHH |
| | HHH |
| EGFR (G8) Prodrug C1486 (SEQ | EVQLVESGGGLVQPGNSLRLSCAASGFTFSKFGMSWVRQGGG |
| ID NO: 46) | GGLDGNEEPGGLEWVSSISGSGRDTLYADSVKGRFTISRDNAK |
| | TTLYLQMNSLRPEDTAVYYCTIGGSLSVSSQGTLVTVSSGGGGK |
| | PLGLQARVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPEDEA |
| | EYYCTLWYSNRWVFGGGTKLTVLGGGGSGGGGSGGGGSEVQ |
| | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLVESGGGLVQPGGSLTLSCAASGRTFSSYAMGWFR |
| | QAPGKEREFVVAINWASGSTYYADSVKGRFTISRDNSKNTLYL |
| | QMNSLRAEDTAVYYCAAGYQINSGNYNFKDYEYDYWGQGTL |
| | VTVSSHHHHHH |
| EGFR (G8) Non-cleavable | EVQLVESGGGLVQPGNSLRLSCAASGFTFSKFGMSWVRQGGG |
| Prodrug C1756 (SEQ ID NO: 47) | GGLDGNEEPGGLEWVSSISGSGRDTLYADSVKGRFTISRDNAK |
| | TTLYLQMNSLRPEDTAVYYCTIGGSLSVSSQGTLVTVSSGGGGS |
| | GGGGSGGVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPEDEA |
| | EYYCTLWYSNRWVFGGGTKLTVLGGGGSGGGGSGGGGSEVQ |
| | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLVESGGGLVQPGGSLTLSCAASGRTFSSYAMGWFR |
| | QAPGKEREFVVAINWASGSTYYADSVKGRFTISRDNSKNTLYL |
| | QMNSLRAEDTAVYYCAAGYQINSGNYNFKDYEYDYWGQGTL |
| | VTVSSHHHHHH |
| EGFR (G8) Active Drug C1300 | VVGGGGTQTVVTQEPSLTVSPGGTVTLTCASSTGAVTSGNYP |
| (SEQ ID NO: 48) | NWVQQKPGQAPRGLIGGTKFLVPGTPARFSGSLLGGKAALTLS |
| | GVQPEDEAEYYCTLWYSNRWVFGGGTKLTVLGGGGSGGGGS |
| | GGGGSEVQLVESGGGLVQPGGSLKLSCAASGFTFNKYAINWV |
| | RQAPGKGLEWVARIRSKYNNYATYYADQVKDRFTISRDDSKNT |
| | AYLQMNNLKTEDTAVYYCVRHANFGNSYISYWAYWGQGTLV |
| | TVSSGGGGSGGGSEVQLVESGGGLVQPGGSLTLSCAASGRTFS |
| | SYAMGWFRQAPGKEREFVVAINWASGSTYYADSVKGRFTISR |
| | DNSKNTLYLQMNSLRAEDTAVYYCAAGYQINSGNYNFKDYEY |
| | DYWGQGTLVTVSSHHHHHH |
| GFP TriTAC C646 | QVQLVESGGALVQPGGSLRLSCAASGFPVNRYSMRWYRQAP |
| (SEQ ID NO: 49) | GKEREWVAGMSSAGDRSSYEDSVKGRFTISRDDARNTVYLQM |
| | NSLKPEDTAVYYCNVNVGFEYWGQGTQVTVSSGGGGSGGGS |
| | EVQLVESGGGLVQPGNSLRLSCAASGFTFSKFGMSWVRQAPG |
| | KGLEWVSSISGSGRDTLYADSVKGRFTISRDNAKTTLYLQMNSL |
| | RPEDTAVYYCTIGGSLSVSSQGTLVTVSSGGGGSGGGSEVQLVE |
| | SGGGLVQPGGSLKLSCAASGFTFNKYAINWVRQAPGKGLEWV |
| | ARIRSKYNNYATYYADQVKDRFTISRDDSKNTAYLQMNNLKTE |
| | DTAVYYCVRHANFGNSYISYWAYWGQGTLVTVSSGGGGSGG |
| | GGSGGGGSQTVVTQEPSLTVSPGGTVTLTCASSTGAVTSGNYP |
| | NWVQQKPGQAPRGLIGGTKFLVPGTPARFSGSLLGGKAALTLS |
| | GVQPEDEAEYYCTLWYSNRWVFGGGTKLTVLHHHHHH |
| Exemplary masking sequence | GGGGGLDGNEEPGG |
| (SEQ ID NO: 50) | |
| Exemplary non-cleavable linker | GGGGSGGGGSGGVVGGGGT |
| sequence comprising protease | |
| cleavage site | |
| (SEQ ID NO: 51) | |
| PSMA Non-masked Non- | EVQLVESGGGLVQPGNSLRLSCAASGFTFSKFGMSWVRQAPG |
| cleavable Prodrug C1874 | KGLEWVSSISGSGRDTLYADSVKGRFTISRDNAKTTLYLQMNSL |
| (SEQ ID NO: 52) | RPEDTAVYYCTIGGSLSVSSQGTLVTVSSGGGGSGGGGSGGVV |
| | GGGGTQTVVTQEPSLTVSPGGTVTLTCASSTGAVTSGNYPNW |
| | VQQKPGQAPRGLIGGTKFLVPGTPARFSGSLLGGKAALTLSGV |
| | QPEDEAEYYCTLWYSNRWVFGGGTKLTVLGGGGSGGGGSGG |
| | GGSEVQLVESGGGLVQPGGSLKLSCAASGFTFNKYAINWVRQ |
| | APGKGLEWVARIRSKYNNYATYYADQVKDRFTISRDDSKNTAY |
| | LQMNNLKTEDTAVYYCVRHANFGNSYISYWAYWGQGTLVTV |
| | SSGGGGSGGGSEVQLVESGGGLVQPGGSLTLSCAASRFMISEY |
| | HMHWVRQAPGKGLEWVSTINPAGTTDYAESVKGRFTISRDN |
| | AKNTLYLQMNSLKPEDTAVYYCDSYGYRGQGTQVTVSSHHHH |
| | HH |
| Exemplary cleavable linker | GGGGKPLGLQARVVGGGGT |
| (SEQ ID NO: 53) | |
| Exemplary anti-albumin sdAb | EVQLVESGGGLVQPGNSLRLSCAASGFTFSKFGMSWVRQGGG |
| (SEQ ID NO: 54) | GGLDGNEEPGGLEWVSSISGSGRDTLYADSVKGRFTISRDNAK |
| | TTLYLQMNSLRPEDTAVYYCTIGGSLSVSSQGTLVTVSSGGGGK |
| | PLGLQARVVGGGGT |
| Exemplary anti-target (anti- | EVQLVESGGGLVQPGGSLTLSCAASGRTFSSYAMGWFRQAPG |
| EGFR) sdAb | KEREFVVAINWASGSTYYADSVKGRFTISRDNSKNTLYLQMNSL |
| (SEQ ID NO: 55) | RAEDTAVYYCAAGYQINSGNYNFKDYEYDYWGQGTLVTVSS |
| Exemplary anti-CD3 scFv | QTVVTQEPSLTVSPGGTVTLTCASSTGAVTSGNYPNWVQQKP |
| (SEQ ID NO: 56) | GQAPRGLIGGTKFLVPGTPARFSGSLLGGKAALTLSGVQPEDEA |
| | EYYCTLWYSNRWVFGGGTKLTVLGGGGSGGGGSGGGGSEVQ |
| | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSS |
| Exemplary anti-target (anti- | EVQLVESGGGLVQPGGSLTLSCAASRFMISEYHMHWVRQAPG |
| PSMA) sdAb | KGLEWVSTINPAGTTDYAESVKGRFTISRDNAKNTLYLQMNSL |
| (SEQ ID NO: 57) | KPEDTAVYYCDSYGYRGQGTQVTVSS |
| Exemplary anti-target (anti- | EVQLVESGGGLVQPGGSLRLSCAASRFMISEYHMHWVRQAP |
| PSMA) sdAb | GKGLEWVSDINPAGTTDYAESVKGRFTISRDNAKNTLYLQMNS |
| (SEQ ID NO: 63) | LRAEDTAVYYCDSYGYRGQGTLVTVSS |
| Exemplary anti-target (anti- | EVQLVESGGGLVQPGGSLRLSCAASRFMISEYHMHWVRQAP |
| PSMA) sdAb | GKGLEWVSTINPAGTTDYAESVKGRFTISRDNAKNTLYLQMNS |
| (SEQ ID NO: 64) | LRAEDTAVYYCDSYGYRGQGTLVTVSS |
| Exemplary anti-target (anti- | EVQLVESGGGLVQPGGSLRLSCAASRFMISEYSMHWVRQAPG |
| PSMA) sdAb | KGLEWVSTINPAKTTDYAESVKGRFTISRDNAKNTLYLQMNSLR |
| (SEQ ID NO: 65) | AEDTAVYYCDSYGYRGQGTLVTVSS |
| Exemplary anti-target (anti- | EVQLVESGGGLVQPGGSLRLSCAASRFMISPYSMHWVRQAPG |
| PSMA) sdAb | KGLEWVSTINPAGTTDYAESVKGRFTISRDNAKNTLYLQMNSL |
| (SEQ ID NO: 66) | RAEDTAVYYCDGYGYRGQGTLVTVSS |
| Exemplary anti-target (anti- | EVQLVESGGGLVQPGGSLRLSCAASRFMISEYSMHWVRQAPG |
| PSMA) sdAb | KGLEWVSTINPAGQTDYAESVKGRFTISRDNAKNTLYLQMNSL |
| (SEQ ID NO: 67) | RAEDTAVYYCDGYGYRGQGTLVTVSS |
| Exemplary anti-target (anti- | EVQLVESGGGLVQPGGSLRLSCAASRFMISEYSMHWVRQAPG |
| PSMA) sdAb | KGLEWVSTINPAGTTDYAEYVKGRFTISRDNAKNTLYLQMNSL |
| (SEQ ID NO: 68) | RAEDTAVYYCDGYGYRGQGTLVTVSS |
| Exemplary anti-target (anti- | EVQLVESGGGLVQPGGSLRLSCAASRFMISEYHMHWVRQAP |
| PSMA) sdAb | GKGLEWVSDINPAKTTDYAESVKGRFTISRDNAKNTLYLQMNS |
| (SEQ ID NO: 69) | LRAEDTAVYYCDSYGYRGQGTLVTVSS |
| Exemplary anti-target (anti- | EVQLVESGGGLVQPGGSLRLSCAASRFMISPYHMHWVRQAP |
| PSMA) sdAb | GKGLEWVSDINPAGTTDYAESVKGRFTISRDNAKNTLYLQMNS |
| (SEQ ID NO: 70) | LRAEDTAVYYCDSYGYRGQGTLVTVSS |
| Exemplary anti-target (anti- | EVQLVESGGGLVQPGGSLRLSCAASRFMISEYHMHWVRQAP |
| PSMA) sdAb | GKGLEWVSDINPAGQTDYAESVKGRFTISRDNAKNTLYLQMN |
| (SEQ ID NO: 71) | SLRAEDTAVYYCDSYGYRGQGTLVTVSS |
| Exemplary anti-target (anti- | EVQLVESGGGLVQPGGSLRLSCAASRFMISEYHMHWVRQAP |
| PSMA) sdAb | GKGLEWVSDINPAGTTDYAEYVKGRFTISRDNAKNTLYLQMNS |
| (SEQ ID NO: 72) | LRAEDTAVYYCDSYGYRGQGTLVTVSS |
| Exemplary anti-target (anti- | EVQLVESGGGLVQPGGSLTLSCAASRFMISEYHMHWVRQAPG |
| PSMA) sdAb | KGLEWVSDINPAGTTDYAESVKGRFTISRDNAKNTLYLQMNSL |
| (SEQ ID NO: 73) | KPEDTAVYYCDSYGYRGQGTQVTVSS |
| Exemplary anti-CD3 scFv | EVQLVESGGGLVQPGGSLKLSCAASGFTFNKYAINWVRQAPG |
| (SEQ ID NO: 74) | KGLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVL |
| Exemplary anti-CD3 scFv | EVQLVESGGGLVQPGGSLKLSCAASGFEFNKYAMNWVRQAP |
| (SEQ ID NO: 75) | GKGLEWVARIRSKYNKYATYYADSVKDRFTISRDDSKNTAYLQ |
| | MNNLKTEDTAVYYCVRHGNFGNSYISYWAYWGQGTLVTVSS |
| | GGGGSGGGGSGGGGSQTVVTQEPSLTVSPGGTVTLTCGSSFG |
| | AVTSGNYPNWVQQKPGQAPRGLIGGTKFLAPGTPARFSGSLL |
| | GGKAALTLSGVQPEDEAEYYCVLWYDNRWVFGGGTKLTVL |
| Exemplary anti-CD3 scFv | EVQLVESGGGLVQPGGSLKLSCAASGFTFNKYAMNWVRQAP |
| (SEQ ID NO: 76) | GKGLEWVARIRSKYNNYATYYADSVKDRFTISRDDSKNTAYLQ |
| | MNNLKTEDTAVYYCVRHGNFGNSHISYWAYWGQGTLVTVSS |
| | GGGGSGGGGSGGGGSQTVVTQEPSLTVSPGGTVTLTCGSSTG |
| | YVTSGNYPNWVQQKPGQAPRGLIGGTSFLAPGTPARFSGSLLG |
| | GKAALTLSGVQPEDEAEYYCVLWYSNRWIFGGGTKLTVL |
| Exemplary anti-CD3 scFv | EVQLVESGGGLVQPGGSLKLSCAASGFMFNKYAMNWVRQAP |
| (SEQ ID NO: 77) | GKGLEWVARIRSKSNNYATYYADSVKDRFTISRDDSKNTAYLQ |
| | MNNLKTEDTAVYYCVRHGNFGNSYISYWATWGQGTLVTVSS |
| | GGGGSGGGGSGGGGSQTVVTQEPSLTVSPGGTVTLTCGSSFG |
| | AVTSGNYPNWVQQKPGQAPRGLIGGTKLLAPGTPARFSGSLL |
| | GGKAALTLSGVQPEDEAEYYCVLWYSNSWVFGGGTKLTVL |
| Exemplary anti-CD3 scFv | EVQLVESGGGLVQPGGSLKLSCAASGFTFNTYAMNWVRQAP |
| (SEQ ID NO: 78) | GKGLEWVARIRSKYNNYATYYKDSVKDRFTISRDDSKNTAYLQ |
| | MNNLKTEDTAVYYCVRHGNFGNSPISYWAYWGQGTLVTVSS |
| | GGGGSGGGGSGGGGSQTVVTQEPSLTVSPGGTVTLTCGSSTG |
| | AVVSGNYPNWVQQKPGQAPRGLIGGTEFLAPGTPARFSGSLL |
| | GGKAALTLSGVQPEDEAEYYCVLWYSNRWVFGGGTKLTVL |
| Exemplary anti-CD3 scFv | EVQLVESGGGLVQPGGSLKLSCAASGFTYNKYAMNWVRQAP |
| (SEQ ID NO: 79) | GKGLEWVARIRSKYNNYATYYADEVKDRFTISRDDSKNTAYLQ |
| | MNNLKTEDTAVYYCVRHGNFGNSPISYWAYWGQGTLVTVSS |
| | GGGGGGGGSGGGGSQTVVTQEPSLTVSPGGTVTLTCGSSKG |
| | AVTSGNYPNWVQQKPGQAPRGLIGGTKELAPGTPARFSGSLL |
| | GGKAALTLSGVQPEDEAEYYCTLWYSNRWVFGGGTKLTVL |
| Exemplary anti-CD3 scFv | EVQLVESGGGLVQPGGSLKLSCAASGNTFNKYAMNWVRQAP |
| (SEQ ID NO: 80) | GKGLEWVARIRSKYNNYETYYADSVKDRFTISRDDSKNTAYLQ |
| | MNNLKTEDTAVYYCVRHTNFGNSYISYWAYWGQGTLVTVSSG |
| | GGGSGGGGSGGGGSQTVVTQEPSLTVSPGGTVTLTCGSSTGA |
| | VTSGYYPNWVQQKPGQAPRGLIGGTYFLAPGTPARFSGSLLGG |
| | KAALTLSGVQPEDEAEYYCVLWYSNRWVFGGGTKLTVL |
| Exemplary anti-CD3 scFv | EVQLVESGGGLVQPGGSLKLSCAASGFTFNNYAMNWVRQAP |
| (SEQ ID NO: 81) | GKGLEWVARIRSKYNNYATYYADAVKDRFTISRDDSKNTAYLQ |
| | MNNLKTEDTAVYYCVRHGNFGNSQISYWAYWGQGTLVTVSS |
| | GGGGSGGGGSGGGGSQTVVTQEPSLTVSPGGTVTLTCGSSTG |
| | AVTDGNYPNWVQQKPGQAPRGLIGGIKFLAPGTPARFSGSLL |
| | GGKAALTLSGVQPEDEAEYYCVLWYSNRWVFGGGTKLTVL |
| Exemplary anti-CD3 scFv | EVQLVESGGGLVQPGGSLKLSCAASGFTFNKYAVNWVRQAPG |
| (SEQ ID NO: 82) | KGLEWVARIRSKYNNYATYYADSVKDRFTISRDDSKNTAYLQM |
| | NNLKTEDTAVYYCVRHGNFGNSYISYWAYWGQGTLVTVSSGG |
| | GGSGGGGSGGGGSQTVVTQEPSLTVSPGGTVTLTCGESTGAV |
| | TSGNYPNWVQQKPGQAPRGLIGGTKILAPGTPARFSGSLLGGK |
| | AALTLSGVQPEDEAEYYCVLWYSNRWVFGGGTKLTVL |
| Exemplary anti-CD3 scFv | EVQLVESGGGLVQPGGSLKLSCAASGFTFNKYPMNWVRQAP |
| (SEQ ID NO: 83) | GKGLEWVARIRSKYNNYATYYADSVKDRFTISRDDSKNTAYLQ |
| | MNNLKNEDTAVYYCVRHGNFNNSYISYWAYWGQGTLVTVSS |
| | GGGGSGGGGSGGGGSQTVVTQEPSLTVSPGGTVTLTCGSSTG |
| | AVTKGNYPNWVQQKPGQAPRGLIGGTKMLAPGTPARFSGSLL |
| | GGKAALTLSGVQPEDEAEYYCALWYSNRWVFGGGTKLTVL |
| Exemplary anti-CD3 scFv | EVQLVESGGGLVQPGGSLKLSCAASGFTFNGYAMNWVRQAP |
| (SEQ ID NO: 84) | GKGLEWVARIRSKYNNYATYYADEVKDRFTISRDDSKNTAYLQ |
| | MNNLKTEDTAVYYCVRHGNFGNSPISYWAYWGQGTLVTVSS |
| | GGGGSGGGGSGGGGSQTVVTQEPSLTVSPGGTVTLTCGSSTG |
| | AVVSGNYPNWVQQKPGQAPRGLIGGTEFLAPGTPARFSGSLL |
| | GGKAALTLSGVQPEDEAEYYCVLWYSNRWVFGGGTKLTVL |
| Exemplary anti-CD3 scFv | EVQLVESGGGLVQPGGSLKLSCAASGNTFNKYAMNWVRQAP |
| (SEQ ID NO: 85) | GKGLEWVARIRSKYNNYATYYADSVKDRFTISRDDSKNTAYLQ |
| | MNNLKTEDTAVYYCVRHGNFGDSYISYWAYWGQGTLVTVSS |
| | GGGGSGGGGSGGGGSQTVVTQEPSLTVSPGGTVTLTCGSSTG |
| | AVTHGNYPNWVQQKPGQAPRGLIGGTKVLAPGTPARFSGSLL |
| | GGKAALTLSGVQPEDEAEYYCVLWYSNRWVFGGGTKLTVL |
| Exemplary anti-CD3 scFv | EVQLVESGGGLVQPGGSLKLSCAASGFTFNNYAMNWVRQAP |
| (SEQ ID NO: 86) | GKGLEWVARIRSGYNNYATYYADSVKDRFTISRDDSKNTAYLQ |
| | MNNLKTEDTAVYYCVRHGNFGNSYISYWAYWGQGTLVTVSS |
| | GGGGSGGGGSGGGGSQTVVTQEPSLTVSPGGTVTLTCGSYTG |
| | AVTSGNYPNWVQQKPGQAPRGLIGGTKFNAPGTPARFSGSLL |
| | GGKAALTLSGVQPEDEAEYYCVLWYANRWVFGGGTKLTVL |
| Exemplary anti-CD3 scFv | EVQLVESGGGLVQPGGSLKLSCAASGFEFNKYAMNWVRQAP |
| (SEQ ID NO: 87) | GKGLEWVARIRSKYNNYETYYADSVKDRFTISRDDSKNTAYLQ |
| | MNNLKTEDTAVYYCVRHGNFGNSLISYWAYWGQGTLVTVSS |
| | GGGGSGGGGSGGGGSQTVVTQEPSLTVSPGGTVTLTCGSSSG |
| | AVTSGNYPNWVQQKPGQAPRGLIGGTKFGAPGTPARFSGSLL |
| | GGKAALTLSGVQPEDEAEYYCVLWYSNRWVFGGGTKLTVL |
| Exemplary anti-CD3 scFv | EVQLVESGGGLVQPGGSLKLSCAASGFTFNKYAMNWVRQAP |
| (SEQ ID NO: 88) | GKGLEWVARIRSKYNNYATYYADSVKDRFTISRDDSKNTAYLQ |
| | MNNLKTEDTAVYYCVRHGNFGNSYISYWAYWGQGTLVTVSS |
| | GGGGSGGGGSGGGGSQTVVTQEPSLTVSPGGTVTLTCGSSTG |
| | AVTSGNYPNWVQQKPGQAPRGLIGGTKFLAPGTPARFSGSLL |
| | GGKAALTLSGVQPEDEAEYYCVLWYSNRWVFGGGTKLTVL |
| Exemplary anti-CD3 scFv | EVQLVESGGGLVQPGGSLKLSCAASGFTFNKYALNWVRQAPG |
| (SEQ ID NO: 89) | KGLEWVARIRSKYNNYATEYADSVKDRFTISRDDSKNTAYLQM |
| | NNLKTEDTAVYYCVRHGNFGNSPISYWAYWGQGTLVTVSSGG |
| | GGSGGGGSGGGGSQTVVTQEPSLTVSPGGTVTLTCGSSTGAV |
| | TSGNYPNWVQQKPGQAPRGLIGGTNFLAPGTPERFSGSLLGG |
| | KAALTLSGVQPEDEAEYYCVLWYSNRWAFGGGTKLTVL |
| Exemplary anti-CD3 scFv | EVQLVESGGGLVQPGGSLKLSCAASGFTFNEYAMNWVRQAP |
| (SEQ ID NO: 90) | GKGLEWVARIRSKYNNYATYYADDVKDRFTISRDDSKNTAYLQ |
| | MNNLKTEDTAVYYCVRHGNFGNSGISYWAYWGQGTLVTVSS |
| | GGGGSGGGGSGGGGSQTVVTQEPSLTVSPGGTVTLTCGSSTG |
| | AVTVGNYPNWVQQKPGQAPRGLIGGTEFLAPGTPARFSGSLL |
| | GGKAALTLSGVQPEDEAEYYCVLWYSNRWVFGGGTKLTVL |
| Exemplary anti-target (BCMA) | EVQLVESGGGLVQPGRSLTLSCAASTNIFSISPMGWYRQAPGK |
| (SEQ ID NO: 91) | QRELVAAIHGFSTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTDIFSISPMGWYRQAPGK |
| (SEQ ID NO: 92) | QRELVAAIHGGSTLYADSVKGRFTISRDNAKNSIYLQMNSLRPE |
| | DTALYYCNKVPWGDYHPRNVAWGQGTQVTVSS |
| Exemplary anti-target (BCMA) | EVQLVESGGGLVQPGRSLTLSCAASTNDFSISPMGWYRQAPG |
| (SEQ ID NO: 93) | KQRELVAAIHGGSTLYADSVKGRFTISRDNAKNSIYLQMNSLRP |
| | EDTALYYCNKVPWGDYHPRNVAWGQGTQVTVSS |
| Exemplary anti-target (BCMA) | EVQLVESGGGLVQPGRSLTLSCAASTNIFSKSPMGWYRQAPGK |
| (SEQ ID NO: 94) | QRELVAAIHGKSTLYADSVKGRFTISRDNAKNSIYLQMNSLRPE |
| | DTALYYCNKVPWGDYHPRNVVWGQGTQVTVSS |
| Exemplary anti-target (BCMA) | EVQLVESGGGLVQPGRSLTLSCAASTNDFSISPMGWYRQAPG |
| (SEQ ID NO: 95) | KQRELVAAIHGKSTLYADSVKGRFTISRDNAKNSIYLQMNSLRP |
| | EDTALYYCNKVPWGDYHPRNVKWGQGTQVTVSS |
| Exemplary anti-target (BCMA) | EVQLVESGGGLVQPGRSLTLSCAASTNQFSISPMGWYRQAPG |
| (SEQ ID NO: 96) | KQRELVAAIHGKSTLYADSVKGRFTISRDNAKNSIYLQMNSLRP |
| | EDTALYYCNKVPWGDYHPRNVVWGQGTQVTVSS |
| Exemplary anti-target (BCMA) | EVQLVESGGGLVQPGRSLTLSCAASTNIFSSSPMGWYRQAPGK |
| (SEQ ID NO: 97) | QRELVAAINGFSTLYADSVKGRFTISRDNAKNSIYLQMNSLRPE |
| | DTALYYCNKVPWGDYHPRNVHWGQGTQVTVSS |
| Exemplary anti-target (BCMA) | EVQLVESGGGLVQPGRSLTLSCAASTNIFSSSPMGWYRQAPGK |
| (SEQ ID NO: 98) | QRELVAAIHGFSTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNIFSTSPMGWYRQAPGK |
| (SEQ ID NO: 99) | QRELVAAIHGFQTLYADSVKGRFTISRDNAKNSIYLQMNSLRPE |
| | DTALYYCNKVPWGDYHPRNVVWGQGTQVTVSS |
| Exemplary anti-target (BCMA) | EVQLVESGGGLVQPGRSLTLSCAASTNIFSRSPMGWYRQAPGK |
| (SEQ ID NO: 100) | QRELVAAIHGFETLYADSVKGRFTISRDNAKNSIYLQMNSLRPE |
| | DTALYYCNKVPWGDYHPRNVLWGQGTQVTVSS |
| Exemplary anti-target (BCMA) | EVQLVESGGGLVQPGRSLTLSCAASTNIFSESPMGWYRQAPGK |
| (SEQ ID NO: 101) | QRELVAAIHGFTTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIFSDSPMGWYRQAPG |
| (SEQ ID NO: 102) | KQRELVAAIHGFSTLYADSVKGRFTISRDNAKNSIYLQMNSLRP |
| | EDTALYYCNKVPWGDYHPRNVAWGQGTQVTVSS |
| Exemplary anti-target (BCMA) | EVQLVESGGGLVQPGRSLTLSCAASTNIFSNSPMGWYRQAPG |
| (SEQ ID NO: 103) | KQRELVAAIHGGSTLYADSVKGRFTISRDNAKNSIYLQMNSLRP |
| | EDTALYYCNKVPWGDYHPRNVHWGQGTQVTVSS |
| Exemplary anti-target (BCMA) | EVQLVESGGGLVQPGRSLTLSCAASTNIFSNSPMGWYRQAPG |
| (SEQ ID NO: 104) | KQRELVAAIHGRSTLYADSVKGRFTISRDNAKNSIYLQMNSLRP |
| | EDTALYYCNKVPWGDYHPRNVMWGQGTQVTVSS |
| Exemplary anti-target (BCMA) | EVQLVESGGGLVQPGRSLTLSCAASTNIFSNSPMGWYRQAPG |
| (SEQ ID NO: 105) | KQRELVAAIHGPSTLYADSVKGRFTISRDNAKNSIYLQMNSLRP |
| | EDTALYYCNKVPWGDYHPRNVTWGQGTQVTVSS |
| Exemplary anti-target (BCMA) | EVQLVESGGGLVQPGRSLTLSCAASTNIFSNSPMGWYRQAPG |
| (SEQ ID NO: 106) | KQRELVAAIHGDSTLYADSVKGRFTISRDNAKNSIYLQMNSLRP |
| | EDTALYYCNKVPWGDYHPRNVRWGQGTQVTVSS |
| Exemplary anti-target (BCMA) | EVQLVESGGGLVQPGRSLTLSCAASTNIFSRSPMGWYRQAPGK |
| (SEQ ID NO: 107) | QRELVAAIHGDSTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIFSKSPMGWYRQAPGK |
| (SEQ ID NO: 108) | QRELVAAIHGQSTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIFSSSPMGWYRQAPGK |
| (SEQ ID NO: 109) | QRELVAAIHGHSTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIFSSSPMGWYRQAPGK |
| (SEQ ID NO: 110) | QRELVAAIHGESTLYADSVKGRFTISRDNAKNSIYLQMNSLRPE |
| | DTALYYCNKVPWGDYHPRKVYWGQGTQVTVSS |
| Exemplary anti-target (BCMA) | EVQLVESGGGLVQPGRSLTLSCAASTNIFSRSPMGWYRQAPGK |
| (SEQ ID NO: 111) | QRELVAAIHGNSTLYADSVKGRFTISRDNAKNSIYLQMNSLRPE |
| | DTALYYCNKVPWGIYHPRNVYWGQGTQVTVSS |
| Exemplary anti-target (BCMA) | EVQLVESGGGLVQPGRSLTLSCAASTNIFSESPMGWYRQAPGK |
| (SEQ ID NO: 112) | QRELVAAIHGNSTLYADSVKGRFTISRDNAKNSIYLQMNSLRPE |
| | DTALYYCNKVPWGTYHPRNVYWGQGTQVTVSS |
| Exemplary anti-target (BCMA) | EVQLVESGGGLVQPGRSLTLSCAASTNIFSVSPMGWYRQAPG |
| (SEQ ID NO: 113) | KQRELVAAIHGNSTLYADSVKGRFTISRDNAKNSIYLQMNSLRP |
| | EDTALYYCNKVPWGKYHPRNVYWGQGTQVTVSS |
| Exemplary anti-target (BCMA) | EVQLVESGGGLVQPGRSLTLSCAASTNIFSVSPMGWYRQAPG |
| (SEQ ID NO: 114) | KQRELVAAIHGNSTLYADSVKGRFTISRDNAKNSIYLQMNSLRP |
| | EDTALYYCNKVPWGDYHPRNVYWGQGTQVTVSS |
| Exemplary anti-target (BCMA) | EVQLVESGGGLVQPGRSLTLSCAASTNIFSKSPMGWYRQAPGK |
| (SEQ ID NO: 115) | QRELVAAIHGNSTLYADSVKGRFTISRDNAKNSIYLQMNSLRPE |
| | DTALYYCNKVPWGDYHPREVYWGQGTQVTVSS |
| Exemplary anti-target (BCMA) | EVQLVESGGGLVQPGRSLTLSCAASTNIFSDSPMGWYRQAPG |
| (SEQ ID NO: 116) | KQRELVAAIHGTSTLYADSVKGRFTISRDNAKNSIYLQMNSLRP |
| | EDTALYYCNKVPWGDYHPRNVYWGQGTQVTVSS |
| Exemplary anti-target (BCMA) | EVQLVESGGGLVQPGRSLTLSCAASTNIFSRSPMGWYRQAPGK |
| (SEQ ID NO: 117) | QRELVAAIHGTSTLYADSVKGRFTISRDNAKNSIYLQMNSLRPE |
| | DTALYYCNKVPWGKYHPRNVYWGQGTQVTVSS |
| Exemplary anti-target (BCMA) | EVQLVESGGGLVQPGRSLTLSCAASTNIFSHSPMGWYRQAPG |
| (SEQ ID NO: 118) | KQRELVAAIHGTSTLYADSVKGRFTISRDNAKNSIYLQMNSLRP |
| | EDTALYYCNKVPWGRYHPRNVYWGQGTQVTVSS |
| Exemplary anti-target (BCMA) | EVQLVESGGGLVQPGRSLTLSCAASTNIFSTSPMGWYRQAPGK |
| (SEQ ID NO: 119) | QRELVAAIHGTSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSTSPMGWYRQAPGK |
| (SEQ ID NO: 120) | QRELVAAIHGTSTLYADSVKGRFTISRDNAKNSIYLQMNSLRPE |
| | DTALYYCNKVPWGDYHPRNVQWGQGTQVTVSS |
| Exemplary anti-target (BCMA) | EVQLVESGGGLVQPGRSLTLSCAASTNIFSLSPMGWYRQAPGK |
| (SEQ ID NO: 121) | QRELVAAIHGDSTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNIFSISPGGWYRQAPGK |
| (SEQ ID NO: 122) | QRELVAAIHGSSTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNHFSISPMGWYRQAPG |
| (SEQ ID NO: 123) | KQRELVAAIHGSSTLYADSVKGRFTISRDNAKNSIYLQMNSLRP |
| | EDTALYYCNKVPWGDYHPRVVYWGQGTQVTVSS |
| Exemplary anti-target (BCMA) | EVQLVESGGGLVQPGRSLTLSCAASTNIFSASPMGWYRQAPG |
| (SEQ ID NO: 124) | KQRELVAAIHGSSTLYADSVKGRFTISRDNAKNSIYLQMNSLRP |
| | EDTALYYCNKVPWGDYHPRNVNWGQGTQVTVSS |
| Exemplary anti-target (BCMA) | EVQLVESGGGLVQPGRSLTLSCAASTNIFSASPMGWYRQAPG |
| (SEQ ID NO: 125) | KQRELVAAIHGSSTLYADSVKGRFTISRDNAKNSIYLQMNSLRP |
| | EDTALYYCNKVPWGRYHPRNVYWGQGTQVTVSS |
| Exemplary anti-target (BCMA) | EVQLVESGGGLVQPGRSLTLSCAASTNISSISPMGWYRQAPGK |
| (SEQ ID NO: 126) | QRELVAAIHGTSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIESISPMGWYRQAPGK |
| (SEQ ID NO: 127) | QRELVAAIHGTSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSISPYGWYRQAPGKQ |
| (SEQ ID NO: 128) | RELVAAIHGTSTLYADSVKGRFTISRDNAKNSIYLQMNSLRPEDT |
| | ALYYCNKVPWGDYHPGNVYWGQGTQVTVSS |
| Exemplary anti-target (BCMA) | EVQLVESGGGLVQPGRSLTLSCAASTNIASISPMGWYRQAPGK |
| (SEQ ID NO: 129) | QRELVAAIHGTSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIASISPMGWYRQAPGK |
| (SEQ ID NO: 130) | QRELVAAIHGKSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIASISPMGWYRQAPGK |
| (SEQ ID NO: 131) | QRELVAAIHGSSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNITSISPMGWYRQAPGK |
| (SEQ ID NO: 132) | QRELVAAIHGDSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIMSISPMGWYRQAPG |
| (SEQ ID NO: 133) | KQRELVAAIHGNSTLYADSVKGRFTISRDNAKNSIYLQMNSLRP |
| | EDTALYYCNKVPWGDYHPGNVYWGQGTQVTVSS |
| Exemplary anti-target (BCMA) | EVQLVESGGGLVQPGRSLTLSCAASTNITSISPMGWYRQAPGK |
| (SEQ ID NO: 134) | QRELVAAIHGNSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIVSISPMGWYRQAPGK |
| (SEQ ID NO: 135) | QRELVAAIHGHSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIVSISPMGWYRQAPGK |
| (SEQ ID NO: 136) | QRELVAAIHGKSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNVVSISPMGWYRQAPG |
| (SEQ ID NO: 137) | KQRELVAAIHGKSTLYADSVKGRFTISRDNAKNSIYLQMNSLRP |
| | EDTALYYCNKVPWGDYHPNNVYWGQGTQVTVSS |
| Exemplary anti-target (BCMA) | EVQLVESGGGLVQPGRSLTLSCAASTNIISISPMGWYRQAPGK |
| (SEQ ID NO: 138) | QRELVAAIHGASTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSITPMGWYRQAPGK |
| (SEQ ID NO: 139) | QRELVAAIHGASTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNITSISPMGWYRQAPGK |
| (SEQ ID NO: 140) | QRELVAAIHGFETLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIQSISPMGWYRQAPGK |
| (SEQ ID NO: 141) | QRELVAAIHGFETLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTSDFSISPMGWYRQAPGK |
| (SEQ ID NO: 142) | QRELVAAIHGFETLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIDSISPMGWYRQAPGK |
| (SEQ ID NO: 143) | QRELVAAIHGFQTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIMSISPMGWYRQAPG |
| (SEQ ID NO: 144) | KQRELVAAIHGFSTVYADSVKGRFTISRDNAKNSIYLQMNSLRP |
| | EDTALYYCNKVPWGDYHPGNVYWGQGTQVTVSS |
| Exemplary anti-target (BCMA) | EVQLVESGGGLVQPGRSLTLSCAASTNIESISPMGWYRQAPGK |
| (SEQ ID NO: 145) | QRELVAAIHGFSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSSSPMGWYRQAPGK |
| (SEQ ID NO: 146) | QRELVAAIHGFKTLYADSVKGRFTISRDNAKNSIYLQMNSLRPE |
| | DTARYYCNKVPWGDYHPGNVYWGQGTQVTVSS |
| Exemplary anti-target (BCMA) | EVQLVESGGGLVQPGRSLTLSCAASTNIFSSSPMGWYRQAPGK |
| (SEQ ID NO: 147) | QRELVAAIHGFSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSNSPMGWYRQAPG |
| (SEQ ID NO: 148) | KQRELVAAIHGFSTLYADSVKGRFTISRDNAKNSIYLQMNSLRP |
| | EDTALYYCNKVPWGDYHPGNVYWGQGTQVTVSS |
| Exemplary anti-target (BCMA) | EVQLVESGGGLVQPGRSLTLSCAASTNIFSTSPMGWYRQAPGK |
| (SEQ ID NO: 149) | QRELVAAIHGFSTIYADSVKGRFTISRDNAKNSIYLQMNSLRPED |
| | TALYYCNKVPWGDYHPGNVYWGQGTQVTVSS |
| Exemplary anti-target (BCMA) | EVQLVESGGGLVQPGRSLTLSCAASTNIFSTSPMGWYRQAPGK |
| (SEQ ID NO: 150) | QRELVAAIHGFSTIYADSVKGRFTISRDNAKNSIYLQMNSLRPED |
| | TALYYCNKVPWGDYHPLNVYWGQGTQVTVSS |
| Exemplary anti-target (BCMA) | EVQLVESGGGLVQPGRSLTLSCVASTNIFSTSPMGWYRQAPGK |
| (SEQ ID NO: 151) | QRELVAAIHGFSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSDSPMGWYRQAPG |
| (SEQ ID NO: 152) | KQRELVAAIHGFSTFYADSVKGRFTISRDNAKNSIYLQMNSLRP |
| | EDTALYYCNKVPWGDYHPGNVYWGQGTQVTVSS |
| Exemplary anti-target (BCMA) | EVQLVESGGGLVQPGRSLTLSCAASTNIFSQSPMGWYRQAPG |
| (SEQ ID NO: 153) | KQRELVAAIHGDSTLYADSVKGRFTISRDNAKNSIYLQMNSLRP |
| | EDTALYYCNKVPWGDYHPGNVCWGQGTQVTVSS |
| Exemplary anti-target (BCMA) | EVQLVESGGGLVQPGRSLTLSCAASTNIFSQSPMGWYRQAPG |
| (SEQ ID NO: 154) | KQRELVAAIHGKSTLYADSVKGRFTISRDNAKNSIYLQMNSLRP |
| | EDTALYYCNKVPWGDYHPSNVYWGKGTQVTVSS |
| Exemplary anti-target (BCMA) | EVQLVESGGGLVQPGRSLTLSCAASTNIFSRSPMGWYRQAPGK |
| (SEQ ID NO: 155) | QRELVAAIHGESTLYADSVKGRFTISRDNAKNSIYLQMNSLRPE |
| | DTALYYCNKVPWGRYHPGNVYWGQGTQVTVSS |
| Exemplary anti-target (BCMA) | EVQLVESGGGLVQPGRSLTLSCAASTNIFSRSPMGWYRQAPGK |
| (SEQ ID NO: 156) | QRELVAAIHGISTLYADSVKGRFTISRDNAKNSIYLQMNSLRPED |
| | TALYYCNKVPWGDYHPGNVYWGQGTQVTVSS |
| Exemplary anti-target (BCMA) | EVQLVESGGGLVQPGRSLTLSCAASTNIFSRSPMGWYRQAPGK |
| (SEQ ID NO: 157) | QRELVAAIHGSSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSGSPMGWYRQAPG |
| (SEQ ID NO: 158) | KQRELVAAIHGNSTLYADSVKGRFTISRDNAKNSIYLQMNSLRP |
| | EDTALYYCNKVPWGDYHPGNVYWGQGTQVTVSS |
| Exemplary anti-target (BCMA) | EVQLVESGGGLVQPGRSLTLSCAASSNIFSISPMGWYRQAPGK |
| (SEQ ID NO: 159) | QRELVAAIHGSSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSIYPMGWYRQAPGK |
| (SEQ ID NO: 160) | QRELVAAIHGSSTLYADSVKGRFTISRDNAKNSIYLQMNSLRPE |
| | DTALYYCNKVPWGDYHPKNVYWGQGTQVTVSS |
| Exemplary anti-target (BCMA) | EVQLVESGGGLVQPGRSLTLSCAASTNIFSKSPMGWYRQAPGK |
| (SEQ ID NO: 161) | QRELVAAIHGSSTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSKSPMGWYRQAPGK |
| (SEQ ID NO: 162) | QRELVAAIHGSSTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNEFSISPMGWYRQAPGK |
| (SEQ ID NO: 163) | QRELVAAIHGLSTLYADSVKGRFTISRDNAKNSIYLQMNSLRPE |
| | DTALYYCNKVPWGAYHPRNVYWGQGTQVTVSS |
| Exemplary anti-target (BCMA) | EVQLVESGGGLVQPGRSLTLSCAASTNEFSISPMGWYRQAPGK |
| (SEQ ID NO: 164) | QRELVAAIHGESTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIPSISPMGWYRQAPGK |
| (SEQ ID NO: 165) | QRELVAAIHGESTLYADSVKGRFTISRDNAKNSIYLQMNSLRPE |
| | DTALYYCNKVPWGDYHPRNVAWGQGTQVTVSS |
| Exemplary anti-target (BCMA) | EVQLVESGGGLVQPGRSLTLSCAASTNIPSISPMGWYRQAPGK |
| (SEQ ID NO: 166) | QRELVAAIHGASTLYADSVKGRFTISRDNAKNSIYLQMNSLRPE |
| | DTALYYCNKVPWGDYHPRNVAWGQGTQVTVSS |
| Exemplary anti-target (BCMA) | EVQLVESGGGLVQPGRSLTLSCAASTNIPSISPMGWYRQAPGK |
| (SEQ ID NO: 167) | QRELVAAIHGESTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNIPSISPMGWYRQAPGK |
| (SEQ ID NO: 168) | QRELVAAIHGDSTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNITSISPMGWYRQAPGK |
| (SEQ ID NO: 169) | QRELVAAIHGVSTLYADSVKGRFTISRDNAKNSIYLQMNSLRPE |
| | DTALYYCNKVPWGDYHPRNVQWGQGTQVTVSS |
| Exemplary anti-target (BCMA) | EVQLVESGGGLVQPGRSLTLSCAASTNIPSISPMGWYRQAPGK |
| (SEQ ID NO: 170) | QRELVAAIHGQSTLYADSVKGRFTISRDNAKNSIYLQMNSLRPE |
| | DTALYYCNKVPWGDYHPRNVQWGQGTQVTVSS |
| Exemplary anti-target (BCMA) | EVQLVESGGGLVQPGRSLTLSCAASTNIVSISPMGWYRQAPGK |
| (SEQ ID NO: 171) | QRELVAAIHGDSTLYADSVKGRFTISRDNAKNSIYLQMNSLRPE |
| | DTALYYCNKVPWGDYHPRNVSWGQGTQVTVSS |
| Exemplary anti-target (BCMA) | EVQLVESGGGLVQPGRSLTLSCAASSNIFSISPMGWYRQAPGK |
| (SEQ ID NO: 172) | QRELVAAIHGESTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIDSISPMGWYRQAPGK |
| (SEQ ID NO: 173) | QRELVAAIHGESTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIDSISPMGWYRQAPGK |
| (SEQ ID NO: 174) | QRELVAAIHGSSTLYADSVKGRFTISRDNAKNSIYLQMNSLRPE |
| | DTALYYCNKVPWGDYHPRNVTWGQGTQVTVSS |
| Exemplary anti-target (BCMA) | EVQLVESGGGLVQPGRSLTLSCAASTNIRSISPMGWYRQAPGK |
| (SEQ ID NO: 175) | QRELVAAIHGSSTLYADSVKGRFTISRDNAKNSIYLQMNSLRPE |
| | DTALYYCNKVPWGDYHPRNVVWGQGTQVTVSS |
| Exemplary anti-target (BCMA) | EVQLVESGGGLVQPGRSLTLSCAASTNITSISPMGWYRQAPGK |
| (SEQ ID NO: 176) | QRELVAAISGFSTLYADSVKGRFTISRDNAKNSIYLQMNSLRPED |
| | TALYYCNEVPWGDYHPRNVYWGQGTQVTVSS |
| Exemplary anti-target (BCMA) | EVQLVESGGGLVQPGRSLTLSCAASTNITSISPMGWYRQAPGK |
| (SEQ ID NO: 177) | QRELVAAIHGESTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNITSVSPMGWYRQAPG |
| (SEQ ID NO: 178) | KQRELVAAIHGPSTLYADSVKGRFTISRDNAKNSIYLQMNSLRP |
| | EDTALYYCNKVPWGDYHPTNVYWGQGTQVTVSS |
| Exemplary anti-target (BCMA) | EVQLVESGGGLVQPGRSLTLSCAASTNIGSISPMGWYRQAPGK |
| (SEQ ID NO: 179) | QRELVAAIHGQSTLYADSVKGRFTISRDNAKNSIYLQMNSLRPE |
| | DTALYYCNKVPWGDYHPQNVYWGQGTQVTVSS |
| Exemplary anti-target (BCMA) | EVQLVESGGGLVQPGRSLTLSCAASTNIESISPMGWYRQAPGK |
| (SEQ ID NO: 180) | QRELVAAIHGKSTLYADSVKGRFTISRDNAKNSIYLQMNSLRPE |
| | DTALYYCNKVPWGDYHPRRVYWGQGTQVTVSS |
| Exemplary anti-target (BCMA) | EVQLVESGGGLVQPGRSLTLSCAASTNIVSISPMGWYRQAPGK |
| (SEQ ID NO: 189) | QRELVAAIHGDSTLYADSVKGRFTISRDNAKNSIYLQMNSLRPE |
| | DTALYYCNKVPWGDYHPRRVYWGQGTQVTVSS |
| Exemplary anti-target (BCMA) | EVQLVESGGGLVQPGRSLTLSCAASTNIDSISPMGWYRQAPGK |
| (SEQ ID NO: 190) | QRELVAAIHGNSTLYADSVKGRFTISRDNAKNSIYLQMNSLRPE |
| | DTALYYCNKVPWGDYHPRMVYWGQGTQVTVSS |
| Exemplary anti-target (BCMA) | EVQLVESGGGLVQPGRSLTLSCAASTNIFMISPMGWYRQAPG |
| (SEQ ID NO: 191) | KQRELVAAIHGDSTLYADSVKGRFTISRDNAKNSIYLQMNSLRP |
| | EDTALYYCNKVPWGRYHPRNVYWGQGTQVTVSS |
| Exemplary anti-target (BCMA) | EVQLVESGGGLVQPGRSLTLSCAASTNIFRISPMGWYRQAPGK |
| (SEQ ID NO: 192) | QRELVAAIHGDSTLYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNIFSISPMGWYRQAPGK |
| (SEQ ID NO: 193) | QRELVAAIHGDSTLYADSVKGRFTISRDNAKNSIYLQMNSLRPE |
| | DTALYYCNKVPWGEYHPRNVYWGQGTQVTVSS |
| Exemplary anti-target (BCMA) | EVQLVESGGGLVQPGRSLTLSCAASTNIFSISPMGWYRQAPGK |
| (SEQ ID NO: 194) | QRELVAAIHGDSTLYADSVKGRFTISRDNAKNSIYLQMNSLRPE |
| | DTALYYCNKVPWGKYHPRNVYWGQGTQVTVSS |
| Exemplary anti-target (BCMA) | EVQLVESGGGLVQPGRSLTLSCAASTNIESISPMGWYRQAPGK |
| (SEQ ID NO: 195) | QRELVAAIHGSSTLYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNIESISPMGWYRQAPGK |
| (SEQ ID NO: 196) | QRELVAAIHGNSTLYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNISSISPMGWYRQAPGK |
| (SEQ ID NO: 197) | QRELVAAIHGFSTLYADSVKGRFTISRDNAKNSIYLQMNSLRPE |
| | DTALYYCNKVPWGYYHPRNVYWGQGTQVTVSS |
| Exemplary anti-target (BCMA) | EVQLVESGGGLVQPGRSLTLSCAASTNISSISPMGWYRQAPGK |
| (SEQ ID NO: 198) | QRELVAAIHGHSTLYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNISSISPMGWYRQAPGK |
| (SEQ ID NO: 199) | QRELVAAIHGFSTVYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNIFSIRPMGWYRQAPGK |
| (SEQ ID NO: 200) | QRELVAAIHGFSTVYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNIFSIYPMGWYRQAPGK |
| (SEQ ID NO: 201) | QRELVAAIHGFSTYYADSVKGRFTISRDNAKNSIYLQMNSLRPE |
| | DTALYYCNKVPWGSYHPRNVYWGQGTQVTVSS |
| Exemplary anti-target (BCMA) | EVQLVESGGGLVQPGRSLTLSCAASTNIFNISPMGWYRQAPGK |
| (SEQ ID NO: 202) | QRELVAAIHGFSTYYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNIFSSSPMGWYRQAPGK |
| (SEQ ID NO: 203) | QRELVAAIHGFSTWYADSVKGRFTISRDNAKNSIYLQMNSLRP |
| | EDTALYYCNKVPWGRYHPRNVYWGQGTQVTVSS |
| Exemplary anti-target (BCMA) | EVQLVESGGGLVQPGRSLTLSCAASTNISSISPMGWYRQAPGK |
| (SEQ ID NO: 204) | QRELVAAIHGFDTLYADSVKGRFTISRDNAKNSIYLQMNSLRPE |
| | DTALYYCNKVPWGDYHPRNVYWGQGTQVTVSS |
| Exemplary anti-target (BCMA) | EVQLVESGGGLVQPGRSLTLSCAASTNIFSINPMGWYRQAPGK |
| (SEQ ID NO: 205) | QRELVAAIHGFDTLYADSVKGRFTISRDNAKNSIYLQMNSLRPE |
| | DTALYYCNKVPWGDYHPRNVSWGQGTQVTVSS |
| Exemplary anti-target (BCMA) | EVQLVESGGGLVQPGRSLTLSCAASTNIFSITPMGWYRQAPGK |
| (SEQ ID NO: 206) | QRELVAAIHGRSTLYADSVKGRFTISRDNAKNSIYLQMNSLRPE |
| | DTALYYCNKVPWGSYHPRNVYWGQGTQVTVSS |
| Exemplary anti-target (BCMA) | EVQLVESGGGLVQPGRSLTLSCAASTNIFSITPMGWYRQAPGK |
| (SEQ ID NO: 207) | QRELVAAIHGTSTLYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNIFSITPMGWYRQAPGK |
| (SEQ ID NO: 208) | QRELVAAIHGESTLYADSVKGRFTISRDNAKNSIYLQMNSLRPE |
| | DTALYYCNKVPWGRYHPRNVYWGQGTQVTVSS |
| Exemplary anti-target (BCMA) | EVQLVESGGGLVQPGRSLTLSCAASTNIFSITPMGWYRQAPGK |
| (SEQ ID NO: 209) | QRELVAAIHGESTLYADSVKGRFTISRDNAKNSIYLQMNSLRPE |
| | DTALYYCNKVPWGDYHPRDVYWGQGTQVTVSS |
| Exemplary anti-target (BCMA) | EVQLVESGGGLVQPGRSLTLSCAASTNIFSTSPYGWYRQAPGK |
| (SEQ ID NO: 210) | QRELVAAIHGFSTIYADSVKGRFTISRDNAKNSIYLQMNSLRPED |
| | TALYYCNKVPWGDYHPGNVYWGQGTQVTVSS |
| Exemplary anti-target (BCMA) | EVQLVESGGGLVQPGRSLTLSCAASTNIFSTSPGGWYRQAPGK |
| (SEQ ID NO: 211) | QRELVAAIHGFSTIYADSVKGRFTISRDNAKNSIYLQMNSLRPED |
| | TALYYCNKVPWGDYHPGNVYWGQGTQVTVSS |
| Exemplary anti-target (BCMA) | EVQLVESGGGLVQPGRSLTLSCAASTNIFSITPYGWYRQAPGK |
| (SEQ ID NO: 212) | QRELVAAIHGASTLYADSVKGRFTISRDNAKNSIYLQMNSLRPE |
| | DTALYYCNKVPWGDYHPGNVYWGQGTQVTVSS |
| Exemplary anti-target (BCMA) | EVQLVESGGGLVQPGRSLTLSCAASTNIFSITPGGWYRQAPGK |
| (SEQ ID NO: 213) | QRELVAAIHGASTLYADSVKGRFTISRDNAKNSIYLQMNSLRPE |
| | DTALYYCNKVPWGDYHPGNVYWGQGTQVTVSS |
| Exemplary anti-target (BCMA) | QVQLVESGGGLVQPGESLRLSCAASTNIFSISPMGWYRQAPGK |
| (SEQ ID NO: 214) | QRELVAAIHGFSTLYADSVKGRFTISRDNAKNTIYLQMNSLKPE |
| | DTAVYYCNKVPWGDYHPRNVYWGQGTQVTVSS |
| Exemplary anti-target (MSLN) | QVQLVESGGGLVQPGGSLRLSCAASGRTFSVRGMAWYRQAG |
| (SEQ ID NO: 215) | NNRALVATMNPDGFPNYADAVKGRFTISWDIAENTVYLQMN |
| | SLNSEDTTVYYCNSGPYWGQGTQVTVSS |
| Exemplary anti-target (MSLN) | QVQLVESGGGLVQAGGSLRLSCAASGSIPSIEQMGWYRQAPG |
| (SEQ ID NO: 216) | KQRELVAALTSGGRANYADSVKGRFTISGDNVRNMVYLQMNS |
| | LKPEDTAIYYCSAGRFKGDYAQRSGMDYWGKGTLVTVSS |
| Exemplary anti-target (MSLN) | QVQLVESGGGLVQAGGSLRLSCAFSGTTYTFDLMSWYRQAPG |
| (SEQ ID NO: 217) | KQRTVVASISSDGRTSYADSVRGRFTISGENGKNTVYLQMNSL |
| | KLEDTAVYYCLGQRSGVRAFWGQGTQVTVSS |
| Exemplary anti-target (MSLN) | QVQLVESGGGLVQAGGSLRLSCVASGSTSNINNMRWYRQAP |
| (SEQ ID NO: 218) | GKERELVAVITRGGYAIYLDAVKGRFTISRDNANNAIYLEMNSL |
| | KPEDTAVYVCNADRVEGTSGGPQLRDYFGQGTQVTVSS |
| Exemplary anti-target (MSLN) | QVQLVESGGGLVQAGGSLRLSCAASGSTFGINAMGWYRQAP |
| (SEQ ID NO: 219) | GKQRELVAVISRGGSTNYADSVKGRFTISRDNAENTVSLQMNT |
| | LKPEDTAVYFCNARTYTRHDYWGQGTQVTVSS |
| Exemplary anti-target (MSLN) | QVRLVESGGGLVQAGGSLRLSCAASISAFRLMSVRWYRQDPSK |
| (SEQ ID NO: 220) | QREWVATIDQLGRTNYADSVKGRFAISKDSTRNTVYLQMNML |
| | RPEDTAVYYCNAGGGPLGSRWLRGRHWGQGTQVTVSS |
| Exemplary anti-target (MSLN) | QVRLVESGGGLVQAGESLRLSCAASGRPFSINTMGWYRQAPG |
| (SEQ ID NO: 221) | KQRELVASISSSGDFTYTDSVKGRFTISRDNAKNTVYLQMNSLK |
| | PEDTAVYYCNARRTYLPRRFGSWGQGTQVTVSS |
| Exemplary anti-target (MSLN) | QVQPVESGGGLVQPGGSLRLSCVVSGSDFTEDAMAWYRQAS |
| (SEQ ID NO: 222) | GKERESVAFVSKDGKRILYLDSVRGRFTISRDIDKKTVYLQMDNL |
| | KPEDTGVYYCNSAPGAARNYWGQGTQVTVSS |
| Exemplary anti-target (MSLN) | QVQPVESGGGLVQPGGSLRLSCVVSGSDFTEDAMAWYRQAS |
| (SEQ ID NO: 223) | GKERESVAFVSKDGKRILYLDSVRGRFTISRDIYKKTVYLQMDNL |
| | KPEDTGVYYCNSAPGAARNVWGQGTQVTVSS |
| Exemplary anti-target (MSLN) | EVQLVESGGGLVQPGNSLRLSCAASGFTFSSFGMSWVRQAPG |
| (SEQ ID NO: 224) | KGLEWVSSISGSGSDTLYADSVKGRFTISRDNAKTTLYLQMNSL |
| | RPEDTAVYYCTIGGSLSRSSQGTLVTVSS |
| Exemplary anti-target (MSLN) | QVQIVESGGGLVQAGGSLRLSCVASGLTYSIVAVGWYRQAPGK |
| (SEQ ID NO: 225) | EREMVADISPVGNTNYADSVKGRFTISKENAKNTVYLQMNSLK |
| | PEDTAVYYCHIVRGWLDERPGPGPIVYWGQGTQVTVSS |
| Exemplary anti-target (MSLN) | QVQLVESGGGLVQTGGSLRLSCAASGLTFGVYGMEWFRQAP |
| (SEQ ID NO: 226) | GKQREWVASHTSTGYVYYRDSVKGRFTISRDNAKSTVYLQMN |
| | SLKPEDTAIYYCKANRGSYEYWGQGTQVTVSS |
| Exemplary anti-target (MSLN) | QVQLVESGGGLVQAGGSLRLSCAASTTSSINSMSWYRQAQGK |
| (SEQ ID NO: 227) | QREPVAVITDRGSTSYADSVKGRFTISRDNAKNTVYLQMNSLK |
| | PEDTAIYTCHVIADWRGYWGQGTQVTVSS |
| Exemplary anti-target (MSLN) | QVQLVESGGGLVQAGGSLRLSCAASGRTLSRYAMGWFRQAP |
| (SEQ ID NO: 228) | GKERQFVAAISRSGGTTRYSDSVKGRFTISRDNAANTFYLQMN |
| | NLRPDDTAVYYCNVRRRGWGRTLEYWGQGTQVTVSS |
| Exemplary anti-target (MSLN) | QVQLGESGGGLVQAGGSLRLSCAASGSIFSPNAMIWHRQAPG |
| (SEQ ID NO: 229) | KQREPVASINSSGSTNYGDSVKGRFTVSRDIVKNTMYLQMNSL |
| | KPEDTAVYYCSYSDFRRGTQYWGQGTQVTVSS |
| Exemplary anti-target (MSLN) | QVQLVESGGGLVPSGGSLRLSCAASGATSAITNLGWYRRAPGQ |
| (SEQ ID NO: 230) | VREMVARISVREDKEDYEDSVKGRFTISRDNTQNLVYLQMNNL |
| | QPHDTAIYYCGAQRWGRGPGTTWGQGTQVTVSS |
| Exemplary anti-target (MSLN) | QVQLVESGGGLVQAGGSLRLSCAASGSTFRIRVMRWYRQAPG |
| (SEQ ID NO: 231) | TERDLVAVISGSSTYYADSVKGRFTISRDNAKNTLYLQMNNLKP |
| | EDTAVYYCNADDSGIARDYWGQGTQVTVSS |
| Exemplary anti-target (MSLN) | QVQLVESGGGLVQAGESRRLSCAVSGDTSKFKAVGWYRQAPG |
| (SEQ ID NO: 232) | AQRELLAWINNSGVGNTAESVKGRFTISRDNAKNTVYLQMNR |
| | LTPEDTDVYYCRFYRRFGINKNYWGQGTQVTVSS |
| Exemplary anti-target (MSLN) | QVQLVESGGGLVQAGGSLRLSCAASGSTFGNKPMGWYRQAP |
| (SEQ ID NO: 233) | GKQRELVAVISSDGGSTRYAALVKGRFTISRDNAKNTVYLQME |
| | SLVAEDTAVYYCNALRTYYLNDPVVFSWGQGTQVTVSS |
| Exemplary anti-target (MSLN) | QVQLVESGGGLVQAGGSLRLSCAASGSTSSINTMYWYRQAPG |
| (SEQ ID NO: 234) | KERELVAFISSGGSTNVRDSVKGRFSVSRDSAKNIVYLQMNSLT |
| | PEDTAVYYCNTYIPLRGTLHDYWGQGTQVTVSS |
| Exemplary anti-target (MSLN) | QVQLVESGGGLVQAGGSLRLSCVASGRTDRITTMGWYRQAP |
| (SEQ ID NO: 235) | GKQRELVATISNRGTSNYANSVKGRFTISRDNAKNTVYLQMNS |
| | LKPEDTAVYYCNARKWGRNYWGQGTQVTVSS |
| Exemplary anti-target (MSLN) | QVQLVESGGGLVQARGSLRLSCTASGRTIGINDMAWYRQAPG |
| (SEQ ID NO: 236) | NQRELVATITKGGTTDYADSVDGRFTISRDNAKNTVYLQMNSL |
| | KPEDTAVYYCNTKRREWAKDFEYWGQGTQVTVSS |
| Exemplary anti-target (MSLN) | QVQLVESGGGLVQAGGSLRLSCAASAIGSINSMSWYRQAPGK |
| (SEQ ID NO: 237) | QREPVAVITDRGSTSYADSVKGRFTISRDNAKNTVYLQMNSLK |
| | PEDTAIYTCHVIADWRGYWGQGTQVTVSS |
| Exemplary anti-target (MSLN) | QVQLVESGGGLVQAGGSLRLSCAASGSTSSINTMYWFRQAPG |
| (SEQ ID NO: 238) | EERELVATINRGGSTNVRDSVKGRFSVSRDSAKNIVYLQMNRL |
| | KPEDTAVYYCNTYIPYGGTLHDFWGQGTQVTVSS |
| Exemplary anti-target (MSLN) | QVQLVESGGGLVQAGGSLRLSCTTSTTFSINSMSWYRQAPGN |
| (SEQ ID NO: 239) | QREPVAVITNRGTTSYADSVKGRFTISRDNARNTVYLQMDSLK |
| | PEDTAIYTCHVIADWRGYWGQGTQVTVSS |
| Exemplary anti-target (MSLN) | QVQLVESGGGLVQAGGSLTLSCAASGSTFSIRAMRWYRQAPG |
| (SEQ ID NO: 240) | TERDLVAVIYGSSTYYADAVKGRFTISRDNAKNTLYLQMNNLKP |
| | EDTAVYYCNADTIGTARDYWGQGTQVTVSS |
| Exemplary anti-target (MSLN) | QVQLVESGGGLVQAGGSLRLSCVASGRTSTIDTMYWHRQAPG |
| (SEQ ID NO: 241) | NERELVAYVTSRGTSNVADSVKGRFTISRDNAKNTAYLQMNSL |
| | KPEDTAVYYCSVRTTSYPVDFWGQGTQVTVSS |
| Exemplary anti-target (MSLN) | QVQLVESGGGLVQAGGSLRLSCAASGSTSSINTMYWYRQAPG |
| (SEQ ID NO: 242) | KERELVAFISSGGSTNVRDSVKGRFSVSRDSAKNIVYLQMNSLK |
| | PEDTAVYYCNTYIPYGGTLHDFWGQGTQVTVSS |
| Exemplary anti-target (MSLN) | QVQLVESGGGLVQPGGSLRLSCAASGGDWSANFMYWYRQA |
| (SEQ ID NO: 243) | PGKQRELVARISGRGVVDYVESVKGRFTISRDNAKNTVYLQMN |
| | SLKPEDTAVYYCAVASYWGQGTQVTVSS |
| Exemplary anti-target (MSLN) | EVQLVESGGGLVQPGGSLRLSCAASGGDWSANFMYWYRQAP |
| (SEQ ID NO: 244) | GKQRELVARISGRGVVDYVESVKGRFTISRDNSKNTLYLQMNSL |
| | RAEDTAVYYCAVASYWGQGTLVTVSS |
| Exemplary anti-target (MSLN) | EVQLVESGGGLVQPGGSLRLSCAASGGDWSANFMYWVRQAP |
| (SEQ ID NO: 245) | GKGLEWVSRISGRGVVDYVESVKGRFTISRDNSKNTLYLQMNS |
| | LRAEDTAVYYCAVASYWGQGTLVTVSS |
| Exemplary anti-target (MSLN) | EVQLVESGGGLVQAGGSLRLSCAASGSTSSINTMYWYRQAPG |
| (SEQ ID NO: 246) | KERELVAFISSGGSTNVRDSVKGRFTISRDNSKNTLYLQMNSLR |
| | AEDTAVYYCNTYIPYGGTLHDFWGQGTLVTVSS |
| Exemplary anti-target (MSLN) | EVQLVESGGGLVQPGGSLRLSCAASGSTSSINTMYWYRQAPG |
| (SEQ ID NO: 247) | KERELVAFISSGGSTNVRDSVKGRFTISRDNSKNTLYLQMNSLR |
| | AEDTAVYYCNTYIPYGGTLHDFWGQGTLVTVSS |
| Exemplary anti-target (MSLN) | EVQLVESGGGLVQPGGSLRLSCAASGSTSSINTMYWVRQAPG |
| (SEQ ID NO: 248) | KGLEWVSFISSGGSTNVRDSVKGRFTISRDNSKNTLYLQMNSLR |
| | AEDTAVYYCNTYIPYGGTLHDFWGQGTLVTVSS |
| Exemplary anti-target (MSLN) | QVQLVESGGGVVQAGGSLRLSCAASGSTFSIRAMRWYRQAPG |
| (SEQ ID NO: 249) | TERDLVAVIYGSSTYYADAVKGRFTISRDNSKNTLYLQMNSLRA |
| | EDTAVYYCNADTIGTARDYWGQGTLVTVSS |
| Exemplary anti-target (MSLN) | QVQLVESGGGVVQPGGSLRLSCAASGSTFSIRAMRWYRQAPG |
| (SEQ ID NO: 250) | KERELVAVIYGSSTYYADAVKGRFTISRDNSKNTLYLQMNSLRA |
| | EDTAVYYCNADTIGTARDYWGQGTLVTVSSGG |
| Exemplary anti-target (MSLN) | QVQLVESGGGVVQPGGSLRLSCAASGSTFSIRAMRWVRQAPG |
| (SEQ ID NO: 251) | KGLEWVSVIYGSSTYYADAVKGRFTISRDNSKNTLYLQMNSLRA |
| | EDTAVYYCNADTIGTARDYWGQGTLVTVSSGG |
| Exemplary anti-target (MSLN) | EVQLVESGGGLVQAGGSLRLSCVASGRTSTIDTMYWHRQAPG |
| (SEQ ID NO: 252) | NERELVAYVTSRGTSNVADSVKGRFTISRDNSKNTLYLQMNSL |
| | RAEDTAVYYCSVRTTSYPVDFWGQGTLVTVSGG |
| Exemplary anti-target (MSLN) | EVQLVESGGGLVQPGGSLRLSCAASGRTSTIDTMYWHRQAPG |
| (SEQ ID NO: 253) | KERELVAYVTSRGTSNVADSVKGRFTISRDNSKNTLYLQMNSLR |
| | AEDTAVYYCSVRTTSYPVDFWGQGTLVTVSS |
| Exemplary anti-target (MSLN) | EVQLVESGGGLVQPGGSLRLSCAASGRTSTIDTMYWVRQAPG |
| (SEQ ID NO: 254) | KGLEWVSYVTSRGTSNVADSVKGRFTISRDNSKNTLYLQMNSL |
| | RAEDTAVYYCSVRTTSYPVDFWGQGTLVTVSS |
| Exemplary anti-target (MSLN) | QVQLVESGGGVVQAGGSLTLSCAASGSTFSIRAMRWYRQAPG |
| (SEQ ID NO: 255) | TERDLVAVIYGSSTYYADAVKGRFTISRDNSKNTLYLQMNSLRA |
| | EDTAVYYCNADTIGTARDYWGQGTLVTVSS |
| Exemplary anti-target (MSLN) | QVQLVESGGGVVQAGGSLRLSCAASGSTFSIRAMRWYRQAPG |
| (SEQ ID NO: 256) | TERDLVAVIYGSSTYYADAVKGRFTISRDNSKNTLYLQMNSLRA |
| | EDTAVYYCNADTIGTARDYWGQGTLVTVSS |
| Exemplary anti-target (MSLN) | QVQLVESGGGVVQPGGSLRLSCAASGSTFSIRAMRWYRQAPG |
| (SEQ ID NO: 257) | KERELVAVIYGSSTYYADAVKGRFTISRDNSKNTLYLQMNSLRA |
| | EDTAVYYCNADTIGTARDYWGQGTLVTVSS |
| Exemplary anti-target (MSLN) | QVQLVESGGGVVQPGGSLRLSCAASGSTFSIRAMRWVRQAPG |
| (SEQ ID NO: 258) | KGLEWVSVIYGSSTYYADAVKGRFTISRDNSKNTLYLQMNSLRA |
| | EDTAVYYCNADTIGTARDYWGQGTLVTVSS |
| Exemplary anti-target (DLL3) | QVQLQESGGGLVQAGGSLRLSCAASGSIFSIASMGWYRQAPG |
| (SEQ ID NO: 306) | KQRELVAVITSFSSTNYADSVKGRFTISRDNAKNTVYLQMNSLK |
| | PEDTGVYYCNARYFERTDWGQGTQVTVSS |
| Exemplary anti-target (DLL3) | QVQLQESGGGLVQAGGSLRLSCAAPGSIFSIASMGWYRQAPG |
| (SEQ ID NO: 307) | KQRELVAVITSFSSTNYADSVKGRFTISRDNAKNTVYLQMNSLK |
| | PEDTGVYYCNARYFERTDWGQGTQVTVSS |
| Exemplary anti-target (DLL3) | QVQLQESGGGLVQAGGSLRLSCAASGSIFSIASMAWYRQAPG |
| (SEQ ID NO: 308) | KQRELVAAITSFSSTNYADSVKGRFTISRDNAKNTVYLQMNSLK |
| | PEDTGVYYCNARYFERTDWGQGTQVTVSS |
| Exemplary anti-target (DLL3) | QVQLQESGGGLVQAGGSLRLSCAASESIFSINVMAWHRQAPG |
| (SEQ ID NO: 309) | KQRELVARITSGGSTNYADSVKGRFTISRDNAKNTVYLQMNSL |
| | KPEDTGVYYCGAYQGLYAYWGQGTQVTVSS |
| Exemplary anti-target (DLL3) | QVQLQESGGGLVQAGGSLRLSCVASGSSFSITSMAWYRQAPG |
| (SEQ ID NO: 310) | KQRDLVAAITSFGSTNYADSVKDRFTISRDNAKNTVYLQMNSL |
| | KPEDTAVYYCNGRVFDHVYWGQGTQVTVSS |
| Exemplary anti-target (DLL3) | QVQLQESGGGLVQAGGSLKLSCAASSSIFSISSMSWYRQAPGK |
| (SEQ ID NO: 311) | QRELVAAITTFDYTNYADSVKGRFTISRDNAKNMMYLQMNSL |
| | KPEDTAVYLCNARAFGRDYWGQGTQVTVSS |
| Exemplary anti-target (DLL3) | QVQLQESGGGLVQAGGSLKLSCAASSSIFSISSMSWYRQAPGK |
| (SEQ ID NO: 312) | QRELVAAITSFGSTNYADSVKGRFTISRDNAKNMMYLQMNSL |
| | KPEDTAVYRCNARTMGRDYWGQGTQVTVSS |
| Exemplary anti-target (DLL3) | QVQLQESGGGLVQPGGSLRLSCAASGSTLNIKIMAWHRQAPG |
| (SEQ ID NO: 313) | KQRELVATLTSGGNTNYADSVKGRFTISRDNAKNTVYLQMNSL |
| | QPEDTAVYYCGLWDGVGGAYWGRGTQVTVSS |
| Exemplary anti-target (DLL3) | QVQLQESGGGLVQPGGSLRISCAASGSTLNIKIMAWHRQAPG |
| (SEQ ID NO: 314) | KQRELVATLTSGGNTNYADSVKGRFTISRDNAKNTVYLQMNSL |
| | QPEDTAVYYCGLWDGVGGAYWGRGTQVTVSS |
| Exemplary anti-target (DLL3) | QVQLQESGGGLVQAGGSLRLSCAASGSTFNIKTMAWHRQAP |
| (SEQ ID NO: 315) | GNQRELVATLTSGGNTNYADSVKGRFTISRDNAKNTVYLQMN |
| | SLKPEDTAVYYCGLWNGVGGAYWGRGTQVTVSS |
| Exemplary anti-target (DLL3) | QVQLQDGGGLVQPGGSLRLSCAASGSTFNIKLMAWHRQAPG |
| (SEQ ID NO: 316) | NQRELVATLTSGGNTNYADSVKGRFTISRDNASNIVYLQMNSL |
| | KPEDTAVYYCGLWDGVGGAYWGRGTQVTVSS |
| Exemplary anti-target (DLL3) | QVQLQESGGGLVQAGGSLRLSCAASGSTFNFKIMAWHRQAP |
| (SEQ ID NO: 317) | GKQRELVASLTSEGLTNYRDSVKGRFTISRDNAKNTVYLQMNN |
| | LKPEDTAVYYCGLWDGVGGAYWGRGTQVTVSS |
| Exemplary anti-target (DLL3) | QVQLQESGGGLVQPGGSLRLSCAASGFMFSSYSMSWYRQAP |
| (SEQ ID NO: 318) | GKQRELVAAITTWGSTNYADSVKGRFTISRDNAKNTVWLQM |
| | NSLEPEDTAVYFCNARSWNNYWGQGTQVTVSS |
| Exemplary anti-target (DLL3) | QVQLQESGGGLVQVGGSLRLSCAASGFMFSSYSMSWYRQAP |
| (SEQ ID NO: 319) | GKQRELVAAITSYGSTNYADSVKGRFTISRDNAKNTVWLQMN |
| | SLKPEDTAVYFCNARSWNNYWGQGTQVTVSS |
| Exemplary anti-target (DLL3) | QVQLQESGGGLVQPGGSLRLSCAASGFTFSSHSMSWYRQAPG |
| (SEQ ID NO: 320) | KQRELVAAITTYGSTNYIDSVKGRFTISRDNTKNTVYLQMNSLK |
| | PEDTAVYFCNARSWNNYWGQGTQVTVSS |
| Exemplary anti-target (DLL3) | QVQLQESGGGLVQAGGSLRLSCVASGSSFSHNTMGWYRQAP |
| (SEQ ID NO: 321) | GKQRDLVARITTFGTTNYADSVKGRFTISRDNAKNTVYLQMNS |
| | LKPEDTAVYYCNGESFGRIWYNWGQGTQVTVSS |
| Exemplary anti-target (DLL3) | QVQLQESGGGLVQAGASLRLTCTASGGRFSYATMGWSRQAP |
| (SEQ ID NO: 322) | GKQREMVARITSSGFSTNYADSVKGRFTISRDNAKNAVYLQM |
| | DSLKPEDTAVYYCNAQHFGTDSWGQGTQVTVSS |
| Exemplary anti-target (DLL3) | QVQLQESGGGLVQAGASLRLTCTASGSRFSYATMGWSRQAP |
| (SEQ ID NO: 323) | GKQRELVARITSSGFSTNYADSVKGRFTISRDNAKNAVYLQMD |
| | SLKPEDTAVYYCNAQQFGTDSWGQGTQVTVSS |
| Exemplary anti-target (DLL3) | QVQLQESGGGLVQAGGSLRLSCAASGSTFTSNVMGWHRQAP |
| (SEQ ID NO: 324) | GKQRELVANMHSGGSTNYADSVKGRFTISRDNAKNIVYLQMN |
| | NLKIEDTAVYYCRWYGIQRAEGYWGQGTQVTVSS |
| Exemplary anti-target (DLL3) | QVQLQESGGGLVVAGGSLRLSCAASGSSVSFLSMAWYRQAPG |
| (SEQ ID NO: 325) | KKRELVAGISVDGSTNYADSVKGRFTVSRDNAKNTVYLQMNSL |
| | QPEDTAVYYCYAYRWVGRDTYWGQGTQVTVSS |
| Exemplary anti-target (DLL3) | QVQLQESGGGLVVAGGSLRLSCAASGSSVSFLSMAWYRQAPG |
| (SEQ ID NO: 326) | KKRELVAGISVDGSTNYADSVKGRFTISRDNAKNTVYLQMNSL |
| | QPEDTAVYYCYAYRWVGRDTYWGQGTQVTVSS |
| Exemplary anti-target (DLL3) | QVQLQESGGGLVVSGGSLRLSCAASGSSVSFLSMAWYRQAPG |
| (SEQ ID NO: 327) | KKRELVAGISVDGSTNYADSVKGRFTISRDNAKNTVYLQMNSL |
| | QPEDTAAYYCYAYRWVGRDTYWGQGTQVTVSS |
| Exemplary anti-target (DLL3) | QVQLQESGGGLVVAGGSLRLSCAASGSSVSFLSMAWYRQAPG |
| (SEQ ID NO: 328) | KKRELVAGISVDGSTNYADSVKGRFTISRDNAENTVYLQMNSL |
| | QPEDTAVYYCYAYRWEGRDTYWGQGTQVTVSS |
| Exemplary anti-target (DLL3) | QVQLQESGGGLVVAGGSLRLSCAASGSSVSFLSMAWYRQAPG |
| (SEQ ID NO: 329) | KKRELVAGISVDGSTNYADSVKGRFTISRDNAENTVYLQMNSL |
| | QPEDTAVYYCYAYRWEGRNTYWGQGTQVTVSS |
| Exemplary anti-target (DLL3) | QVQLQESGGGLVQPGGSLRLSCAASGSSVSFLSMAWYRQAPG |
| (SEQ ID NO: 330) | KKRELVAGISTDGSTNYVDSVKGRFTISRDNAKNTVYLQMNSL |
| | QPEDTAVYYCYAYRWVGRYTYWGQGTQVTVSS |
| Exemplary anti-target (DLL3) | QVQLQESGGGLVVAGGSLRLSCAASGSSVSFLSMAWYRQAPG |
| (SEQ ID NO: 331) | KKRELVAGISTDGTTNYVDSVKDRFTISRDNAKNTVYLQMNSL |
| | QPEDTAAYYCYAYRWVGRDTYWGQGTQVTVSS |
| Exemplary anti-target (DLL3) | QVQLQESGGGLVVAGGSLRLSCAASGSSVSFLSIAWYRQAPGK |
| (SEQ ID NO: 332) | KRELVAGISTDGTTNYVDSVKDRFTISRDNAKNTVYLQMNSLQ |
| | PEDTAAYYCYAYRWVGRDTYWGQGTQVTVSS |
| Exemplary anti-target (DLL3) | QVQLQESGGGLVQAGGSLTLSCAASGSSVSFLSMAWYRQAPG |
| (SEQ ID NO: 333) | KKRELVAGISTDGTTNYVDSVKDRFTISRDNAKNTVYLQMNSL |
| | QPEDTAAYYCYAYRWVGRDTYWGQGTQVTVSS |
| Exemplary anti-target (DLL3) | QVQLQESGGGLVQAGGSLRLSCAASGSSVSFLSMAWYRQAPG |
| (SEQ ID NO: 334) | KKRELVAGISTDGSTNYADSVKGRFTISEGNAKNTVDLQMNSL |
| | QPEDTAVYYCYAYRWVDRYTYWGQGTQVTVSS |
| Exemplary anti-target (DLL3) | QVQLQESGGGLVVAGGSLRLSCAASGSSVSFLSMAWYRQAPG |
| (SEQ ID NO: 335) | KKRELVAGISTDGSTNYADSVKGRFTISEDNAKNTVDLQMNSL |
| | QPEDTAVYYCYAYRWIDRYTYWGQGTQVTVSS |
| Exemplary anti-target (DLL3) | QVQLQESGGGLVQPGGSLRLSCAASGSSVSFLSMAWYRQAPG |
| (SEQ ID NO: 336) | KKRELVAGISTDGSTNYADSVKGRFTISEDNAKNTVDLQMNSL |
| | QPEDTAVYYCYAYRWVDRYTYWGQGTQVTVSS |
| Exemplary anti-target (DLL3) | QVQLQESGGGLVVAGGSLRLSCAASGSSVSFLSMAWYRQAPG |
| (SEQ ID NO: 337) | KKRELVAGISSDGSTNYVDSVKGRFTISRDNAKNIVFLQMNSLQ |
| | PQDTAVYYCYAYRWVGRDTYWGQGTQVTVSS |
| Exemplary anti-target (DLL3) | QVQLQESGGGLVVAGGSLRLSCAASGSSVSFLSMAWYRQAPG |
| (SEQ ID NO: 338) | KKRELVAGISADGSTDYIDSVKGRFTISRDSANNTMYLQMNSL |
| | QPEDTAVYYCYAYRWTTRYTYWGQGTQVTVSS |
| Exemplary anti-target (DLL3) | QVQLQESGGGLVQPGGSLRLSCAASGFTLDYYAIGWYRQAPG |
| (SEQ ID NO: 339) | KKRELVAGISSDGSTHYVDSVKGRFAISRDNAENTVYLQMNDL |
| | QPDDTAVYYCYAYRWVGGYTYWGQGTQVTVSS |
| Exemplary anti-target (DLL3) | QVQLQESGGGLVQAGGSLRLSCVASGSTSSINAMGWYRRAPG |
| (SEQ ID NO: 340) | KQRELVAGISSDGSKNYADSVKGRFTISRDNAKNTVYLQMNSL |
| | KPEDTAVYYCYYFRTVAASSMQYWGQGTQVTVSS |
| Exemplary anti-target (DLL3) | QVQLQESGGGLVQAGGSLRLSCVASGSTSSINAMGWYRRAPG |
| (SEQ ID NO: 341) | KQRELVAGISSDGSKVYADSVKGRFTISRDNAKNTVYLQMNSL |
| | KPEDTAVFYCYYFRTVSGSSMRYWGQGTQVTVSS |
| Exemplary anti-target (DLL3) | QVQLQESGGGLVQAGGSLRLSCAASGITSSVYSMGWYRQAPG |
| (SEQ ID NO: 342) | KQRELVAGSSSDGSTHYVDSVRGRFTISRDNAKNTVYLQMSSL |
| | KPEDTAVYYCYANRGFAGAPSYWGQGTQVTVSS |
| Exemplary anti-target (DLL3) | QVQLQESGGGLVQAGGSLRLSCAASGRTSMFNSMGWHRQA |
| (SEQ ID NO: 343) | PGKQRELVAIIRSGGSSNYADTVKGRFTISRDNTKNTVYLQMN |
| | DLKPEDTAVYYCFYYFQSSYWGQGTQVTVSS |
| Exemplary anti-target (DLL3) | QVQLQESGGGLVQAGGSLRLSCAASGRTSMVNSMGWHRQA |
| (SEQ ID NO: 344) | PGKQRELVALITSGGSSNYADTVKGRFTISRDNTKNTVYLQMN |
| | DLKPEDTAVYYCFYYFQSSYWGQGTQVTVSS |
| Exemplary anti-target (DLL3) | QVQLQESGGGLVQAGGSLRLSCAASGSVSMFNSMGWHRQP |
| (SEQ ID NO: 910) | PGKQRELVAIITSGGSSNYADTVKGRFTISRDNTKNTVYLQMN |
| | DLKPEDTAVYYCFYYFQSSYWGQGTQVTVSS |
| Exemplary anti-target (DLL3) | QVQLQESGGGLVQAGGSLRLSCTASGSIFSIAVMGWYRQVPG |
| (SEQ ID NO: 345) | KRREWVATIFDGSYTNYADSVKGRFTISRDNARNKVYLQMNN |
| | LKPEDTAVYYCQTHWTQGSVPKESWGQGTQVTVSS |
| Exemplary anti-target (DLL3) | QVQLQESGGGLVQAGGSLRLSCVASSGIFSDMSMVWYRQAP |
| (SEQ ID NO: 346) | GKQRELVASITTFGSTNYADPVKGRFTISRDNAKNTVYLQMNSL |
| | KPEDTAVYYCSGRSYSSDYWGRGTQVTVSS |
| Exemplary anti-target (DLL3) | QVQLQESGGGLVQAGGSLRLSCVASGSISSIIVMGWSRQAPGK |
| (SEQ ID NO: 347) | QRESVATITRDGTRNYADSLKGRFTISRDNAKNTSYLQINSLKPE |
| | DTAVYSCYARYGDINYWGKGTQVTVSS |
| Exemplary anti-target (DLL3) | QVQLQESGGGLVQAGGSLRLSCVASGSISSIIVMGWSRQAPGK |
| (SEQ ID NO: 348) | QRESLATISRGGTRTYADSVKGRFTISRDNAKNTSYLQMNSLKP |
| | EDTAVYSCYARYGDINYWGKGTQVTVSS |
| Exemplary anti-target (DLL3) | QVQLQESGGGLVQAGGSLRLSCVASGSIFTTNSMGWHRQGP |
| (SEQ ID NO: 349) | GKQRELVALIGSAGSTKYADSVKGRFTISRDNAKNTVSLQMDSL |
| | KPEDTAVYYCFYYDSRSYWGQGTQVTVSS |
| Exemplary anti-target (DLL3) | QVQLQESGGGMVQPGGSLRLSCAASGSREISTMGWHRQAPG |
| (SEQ ID NO: 350) | KQRELAARITSGGITKYADSVKGRFTISRDNAKKTVYLQMNSLK |
| | SEDTAVYYCFAYDNINAYWGQGTQVTVSS |
| Exemplary anti-target (DLL3) | QVQLQESGGGWVQAGGSLRLSCAASGSREISTMGWHRQAP |
| (SEQ ID NO: 351) | GKQRELAARITSGGITKYADSVKGRFTISRDNAKKTVYLQMNSL |
| | KSEDTAVYYCFAYDNINAYWGQGTQVTVSS |
| Exemplary anti-target (DLL3) | QVQLQESGGGWVQAGGSLRLSCTASGSREISTMGWHRQAPG |
| (SEQ ID NO: 352) | KQRELAARITSGGITKYADSVKGRFTISRDNAKKTVYLQMDSLK |
| | SEDTAVYYCFAYDNINAYWGQGTQVTVSS |
| Exemplary anti-target (DLL3) | QVQLQESGGGSVQAGRSLGLSCAASGSREISTMGWHRQAPG |
| (SEQ ID NO: 353) | KQRELAARITSGGITKYADSVKGRFTISRDNAKKTVYLQMNSLK |
| | SEDTAVYYCFAYDNINAYWGQGTQVTVSS |
| Exemplary anti-target (DLL3) | QVQLQESGGGLVQAGGSLRLSCTASGSIFRGAAMYWHRQAP |
| (SEQ ID NO: 354) | GKQRELVAAITTSGNTSYADSVKGRFTISRDNAKNTMYLQIISLK |
| | PEDTAVYYCAFWIAGKAYWGQGTQVTVSS |
| Exemplary anti-target (DLL3) | QVQLQESGGGLVQPGGSLRLSCAASGSISSFNFMSWHRQAPG |
| (SEQ ID NO: 355) | KERELAGVITRGGATNYADSVKGRFTISRDNVKNTVYLQMNGL |
| | KPEDTAVYYCHGRSQLGSTWGQGTQVTVSS |
| Exemplary anti-target (DLL3) | QVQLQESGGGLVQAGGSLRLSCLASGTIFTASTMGWHRQPPG |
| (SEQ ID NO: 356) | KQRELVASIAGDGRTNYAESTEGRFTISRDDAKNTMYLQMNSL |
| | KPEDTAVYYCYAYYLDTYAYWGQGTQVTVSS |
| Exemplary anti-target (DLL3) | EVQLVESGGGLVQPGGSLTLSCAASGSIFSIASMGWYRQAPGK |
| (SEQ ID NO: 357) | QRELVAVITSFSSTNYADSVKGRFTISRDNAKNSVYLQMNSLRA |
| | EDTAVYYCNARYFERTDWGQGTLVTVSS |
| Exemplary anti-target (DLL3) | EVQLVESGGGLVQPGGSLTLSCAASGRTSMFNSMGWHRQAP |
| (SEQ ID NO: 358) | GKQRELVAIIRSGGSSNYADTVKGRFTISRDNAKNSVYLQMNSL |
| | RAEDTAVYYCFYYFQSSYWGQGTLVTVSS |
| Exemplary anti-target (DLL3) | EVQLVESGGGLVQPGGSLTLSCAASGSSVSFLSMAWYRQAPG |
| (SEQ ID NO: 359) | KKRELVAGISVDGSTNYADSVKGRFTISRDNAKNSVYLQMNSL |
| | RAEDTAVYYCYAYRWVGRDTYWGQGTLVTVSS |
| Exemplary anti-target (DLL3) | EVQLVESGGGLVQPGGSLTLSCTASGSIFRGAAMYWHRQAPG |
| (SEQ ID NO: 360) | KQRELVAAITTSGNTSYADSVKGRFTISRDNAKNSMYLQMNSL |
| | RAEDTAVYYCAFWIAGKAYWGQGTLVTVSS |
| Exemplary anti-target (DLL3) | EVQLVESGGGLVQPGGSLTLSCAASGSTFNIKTMAWHRQAPG |
| (SEQ ID NO: 361) | NQRELVATLTSGGNTNYADSVKGRFTISRDNAKNSVYLQMNSL |
| | RAEDTAVYYCGLWNGVGGAYWGQGTLVTVSS |
| Exemplary anti-target (DLL3) | EVQLVESGGGLVQPGGSLTLSCAASGSTLNIKIMAWHRQAPGK |
| (SEQ ID NO: 362) | QRELVATLTSGGNTNYADSVKGRFTISRDNAKNSVYLQMNSLR |
| | AEDTAVYYCGLWDGVGGAYWGQGTLVTVSS |
| Exemplary anti-target (DLL3) | EVQLVESGGGLVQPGGSLTLSCAASSSIFSISSMSWYRQAPGK |
| (SEQ ID NO: 363) | QRELVAAITTFDYTNYADSVKGRFTISRDNAKNSMYLQMNSLR |
| | AEDTAVYYCNARAFGRDYWGQGTLVTVSS |
| Exemplary anti-target (DLL3) | EVQLVESGGGLVQPGGSLTLSCVASGSTSSINAMGWYRRAPG |
| (SEQ ID NO: 364) | KQRELVAGISSDGSKNYADSVKGRFTISRDNAKNSVYLQMNSL |
| | RAEDTAVYYCYYFRTVAASSMQYWGQGTLVTVSS |
| Exemplary anti-target (DLL3) | EVQLVESGGGLVQPGGSLTLSCAASGFMFSSYSMSWYRQAPG |
| (SEQ ID NO: 365) | KQRELVAAITSYGSTNYADSVKGRFTISRDNAKNSVYLQMNSLR |
| | AEDTAVYYCNARSWNNYWGQGTLVTVSS |
| Exemplary anti-target (DLL3) | EVQLVESGGGLVQPGGSLTLSCAASGSVSMFNSMGWHRQPP |
| (SEQ ID NO: 366) | GKQRELVAIITSGGSSNYADTVKGRFTISRDNAKNSVYLQMNSL |
| | RAEDTAVYYCFYYFQSSYWGQGTLVTVSS |
| Exemplary anti-target (DLL3) | EVQLVESGGGLVQPGGSLTLSCTASGGRFSYATMGWSRQAPG |
| (SEQ ID NO: 367) | KQREMVARITSSGFSTNYADSVKGRFTISRDNAKNSVYLQMNS |
| | LRAEDTAVYYCNAQHFGTDSWGQGTLVTVSS |
| Exemplary anti-target (DLL3) | EVQLVESGGGLVQPGGSLTLSCAASGSISSFNFMSWHRQAPG |
| (SEQ ID NO: 368) | KERELAGVITRGGATNYADSVKGRFTISRDNAKNSVYLQMNSL |
| | RAEDTAVYYCHGRSQLGSTWGQGTLVTVSS |
| Exemplary anti-target (DLL3) | EVQLVESGGGLVQPGGSLTLSCAASESIFSINVMAWHRQAPGK |
| (SEQ ID NO: 369) | QRELVARITSGGSTNYADSVKGRFTISRDNAKNSVYLQMNSLR |
| | AEDTAVYYCGAYQGLYAYWGQGTLVTVSS |
| Exemplary anti-target (DLL3) | EVQLVESGGGLVQPGGSLTLSCAASGSREISTMGWHRQAPGK |
| (SEQ ID NO: 370) | QRELAARITSGGITKYADSVKGRFTISRDNAKNSVYLQMNSLRA |
| | EDTAVYYCFAYDNINAYWGQGTLVTVSS |
| Exemplary anti-target (DLL3) | EVQLVESGGGLVQPGGSLTLSCTASGSIFSIAVMGWYRQVPGK |
| (SEQ ID NO: 371) | RREWVATIFDGSYTNYADSVKGRFTISRDNAKNSVYLQMNSLR |
| | AEDTAVYYCQTHWTQGSVPKESWGQGTLVTVSS |
| Exemplary anti-target (DLL3) | EVQLVESGGGLVQPGGSLTLSCVASGSTSSINAMGWYRRAPG |
| (SEQ ID NO: 372) | KQRELVAGISSDGSKVYADSVKGRFTISRDNAKNSVYLQMNSL |
| | RAEDTAVYYCYYFRTVSGSSMRYWGQGTLVTVSS |
| Exemplary anti-target (DLL3) | EVQLVESGGGLVQPGGSLTLSCVASSGIFSDMSMVWYRQAPG |
| (SEQ ID NO: 373) | KQRELVASITTFGSTNYADPVKGRFTISRDNAKNSVYLQMNSLR |
| | AEDTAVYYCSGRSYSSDYWGQGTLVTVSS |
| Exemplary anti-target (DLL3) | EVQLVESGGGLVQPGGSLTLSCAASGFMFSSYSMSWYRQAPG |
| (SEQ ID NO: 374) | KQRELVAAITTWGSTNYADSVKGRFTISRDNAKNSVYLQMNSL |
| | RAEDTAVYYCNARSWNNYWGQGTLVTVSS |
| Exemplary anti-target (DLL3) | EVQLVESGGGLVQPGGSLTLSCTASGSRFSYATMGWSRQAPG |
| (SEQ ID NO: 375) | KQRELVARITSSGFSTNYADSVKGRFTISRDNAKNSVYLQMNSL |
| | RAEDTAVYYCNAQQFGTDSWGQGTLVTVSS |
| Exemplary anti-target (DLL3) | EVQLVESGGGLVQPGGSLTLSCAASGSTFTSNVMGWHRQAP |
| (SEQ ID NO: 376) | GKQRELVANMHSGGSTNYADSVKGRFTISRDNAKNSVYLQM |
| | NSLRAEDTAVYYCRWYGIQRAEGYWGQGTLVTVSS |
| Exemplary anti-target (DLL3) | EVQLVESGGGLVQPGGSLTLSCAASGSSVSFLSMAWYRQAPG |
| (SEQ ID NO: 377) | KKRELVAGISTDGSTNYVDSVKGRFTISRDNAKNSVYLQMNSLR |
| | AEDTAVYYCYAYRWVGRYTYWGQGTLVTVSS |
| Exemplary anti-target (DLL3) | EVQLVESGGGLVQPGGSLTLSCVASGSISSIIVMGWSRQAPGK |
| (SEQ ID NO: 378) | QRESVATITRDGTRNYADSLKGRFTISRDNAKNSSYLQMNSLRA |
| | EDTAVYYCYARYGDINYWGQGTLVTVSS |
| Exemplary anti-target (DLL3) | EVQLVESGGGLVQPGGSLTLSCAASGSSVSFLSMAWYRQAPG |
| (SEQ ID NO: 379) | KKRELVAGISADGSTDYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCLASGTIFTASTMGWHRQPPGK |
| (SEQ ID NO: 380) | QRELVASIAGDGRTNYAESTEGRFTISRDNAKNSMYLQMNSLR |
| | AEDTAVYYCYAYYLDTYAYWGQGTLVTVSS |
| Exemplary anti-target (DLL3) | EVQLVESGGGLVQPGGSLTLSCAASGSSVSFLSMAWYRQAPG |
| (SEQ ID NO: 381) | KKRELVAGISVDGSTNYADSVKGRFTISRDNAKNSVYLQMNSL |
| | RAEDTAVYYCYAYRWEGRNTYWGQGTLVTVSS |
| Exemplary anti-target (DLL3) | EVQLVESGGGLVQPGGSLTLSCVASGSSFSHNTMGWYRQAPG |
| (SEQ ID NO: 382) | KQRDLVARITTFGTTNYADSVKGRFTISRDNAKNSVYLQMNSL |
| | RAEDTAVYYCNGESFGRIWYNWGQGTLVTVSS |
| Exemplary anti-target (DLL3) | EVQLVESGGGLVQPGGSLTLSCVASGSISSIIVMGWSRQAPGK |
| (SEQ ID NO: 383) | QRESLATISRGGTRTYADSVKGRFTISRDNAKNSSYLQMNSLRA |
| | EDTAVYYCYARYGDINYWGQGTLVTVSS |
| Exemplary anti-target (DLL3) | EVQLVESGGGLVQPGGSLTLSCVASGSSFSITSMAWYRQAPGK |
| (SEQ ID NO: 384) | QRDLVAAITSFGSTNYADSVKDRFTISRDNAKNSVYLQMNSLR |
| | AEDTAVYYCNGRVFDHVYWGQGTLVTVSS |
| Exemplary anti-target (DLL3) | EVQLVESGGGLVQPGGSLTLSCAASGRTSMVNSMGWHRQAP |
| (SEQ ID NO: 385) | GKQRELVALITSGGSSNYADTVKGRFTISRDNAKNSVYLQMNS |
| | LRAEDTAVYYCFYYFQSSYWGQGTLVTVSS |
| Exemplary anti-target (DLL3) | EVQLVESGGGLVQPGGSLTLSCAASGSTFNFKIMAWHRQAPG |
| (SEQ ID NO: 386) | KQRELVASLTSEGLTNYRDSVKGRFTISRDNAKNSVYLQMNSLR |
| | AEDTAVYYCGLWDGVGGAYWGQGTLVTVSS |
| Exemplary anti-target (DLL3) | EVQLVESGGGLVQPGGSLTLSCAASGFTLDYYAIGWYRQAPGK |
| (SEQ ID NO: 387) | KRELVAGISSDGSTHYVDSVKGRFTISRDNAKNSVYLQMNSLRA |
| | EDTAVYYCYAYRWVGGYTYWGQGTLVTVSS |
| Exemplary anti-target (DLL3) | EVQLVESGGGLVQPGGSLTLSCVASGSIFTTNSMGWHRQGPG |
| (SEQ ID NO: 388) | KQRELVALIGSAGSTKYADSVKGRFTISRDNAKNSVYLQMNSLR |
| | AEDTAVYYCFYYDSRSYWGQGTLVTVSS |
| Exemplary anti-target (DLL3) | EVQLVESGGGLVQPGGSLTLSCAASGITSSVYSMGWYRQAPG |
| (SEQ ID NO: 389) | KQRELVAGSSSDGSTHYVDSVRGRFTISRDNAKNSVYLQMNSL |
| | RAEDTAVYYCYANRGFAGAPSYWGQGTLVTVSS |
| Exemplary anti-target (DLL3) | EVQLVESGGGLVQPGGSLTLSCAASSSIFSISSMSWYRQAPGK |
| (SEQ ID NO: 390) | QRELVAAITSFGSTNYADSVKGRFTISRDNAKNSMYLQMNSLR |
| | AEDTAVYYCNARTMGRDYWGQGTLVTVSS |
| Exemplary anti-target (DLL3) | EVQLVESGGGLVQPGGSLTLSCVASGFTSSINAMGWYRRAPG |
| (SEQ ID NO: 391) | KQRELVAGISSDGSFVYADSVKGRFTISRDNAKNSVYLQMNSLR |
| | AEDTAVYYCYYFRHVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 392) | KQRELVAGISSDGSKVYADSVKGRFTISRDNAKNSVYLQMNSL |
| | RAEDTAVYYCYYFRTVSGSSSRYWGQGTLVTVSS |
| Exemplary anti-target (DLL3) | EVQLVESGGGLVQPGGSLTLSCVASGSTSSINAMGWYRRAPG |
| (SEQ ID NO: 393) | KQRELVAGISSDGSKVYEDSVKGRFTISRDNAKNSVYLQMNSLR |
| | AEDTAVYYCYYFRTVSGSSMRYWGQGTLVTVSS |
| Exemplary anti-target (DLL3) | EVQLVESGGGLVQPGGSLTLSCVASGSPSSINAMGWYRRAPG |
| (SEQ ID NO: 394) | KQRELSAGISSDGSKVYADSVKGRFTISRDNAKNSVYLQMNSLR |
| | AEDTAVYYCYYFRTVRGSSMSYWGQGTLVTVSS |
| Exemplary anti-target (DLL3) | EVQLVESGGGLVQPGGSLTLSCVASGSTSSINAMGWYRRAPG |
| (SEQ ID NO: 395) | KQRELAAGISSDGSSVYADSVKGRFTISRDNAKNSVYLQMNSLR |
| | AEDTAVYYCYYFRTVSGSSKRYWGQGTLVTVSS |
| Exemplary anti-target (DLL3) | EVQLVESGGGLVQPGGSLTLSCVASGSISSINAMGWYRRAPGK |
| (SEQ ID NO: 396) | QRELVAGISSDGSKVYADSVKGRFTISRDNAKNSVYLQMNSLR |
| | AEDTAVYYCYYFRMVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 397) | KQRELVAGISSDGSKLYADSVKGRFTISRDNAKNSVYLQMNSLR |
| | AEDTAVYYCYYFRTVQGSSMRYWGQGTLVTVSS |
| Exemplary anti-target (DLL3) | EVQLVESGGGLVQPGGSLTLSCVASGSTSSINAYGWYRRAPGK |
| (SEQ ID NO: 398) | QRELVAGISSDGSKVYADSVKGRFTISRDNAKNSVYLQMNSLR |
| | AEDTAVYYCYYFRTVYGSSMRYWGQGTLVTVSS |
| Exemplary anti-target (DLL3) | EVQLVESGGGLVQPGGSLTLSCVASGSTSSINAIGWYRRAPGK |
| (SEQ ID NO: 399) | QRELVAGISSDGSKVYIDSVKGRFTISRDNAKNSVYLQMNSLRA |
| | EDTAVYYCYYFRTVSGSSYRYWGQGTLVTVSS |
| Exemplary anti-target (DLL3) | EVQLVESGGGLVQPGGSLTLSCVASGSTSSINAMAWYRRAPG |
| (SEQ ID NO: 400) | KQRELVAGISSDGSKVYSDSVKGRFTISRDNAKNSVYLQMNSLR |
| | AEDTAVYYCYYFRTVLGSSMRYWGQGTLVTVSS |
| Exemplary anti-target (DLL3) | EVQLVESGGGLVQPGGSLTLSCVASGSTSIINAMGWYRRAPGK |
| (SEQ ID NO: 401) | QRELAAGISSDGSKVIADSVKGRFTISRDNAKNSVYLQMNSLRA |
| | EDTAVYYCYYFRRVSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 402) | KQRELVAGISSDGSKIYADSVKGRFTISRDNAKNSVYLQMNSLR |
| | AEDTAVYYCYYFRTVSGSSMRYWGQGTLVTVSS |
| Exemplary anti-target (DLL3) | EVQLVESGGGLVQPGGSLTLSCVASGKTSSINAMAWYRRAPG |
| (SEQ ID NO: 403) | KQRELVAGISSDGSKVYTDSVKGRFTISRDNAKNSVYLQMNSLR |
| | AEDTAVYYCYYFRTVSGSSARYWGQGTLVTVSS |
| Exemplary anti-target (DLL3) | EVQLVESGGGLVQPGGSLTLSCVASGTTSSINAMGWYRRAPG |
| (SEQ ID NO: 404) | KQRELVAGISSDGSLVYADSVKGRFTISRDNAKNSVYLQMNSLR |
| | AEDTAVYYCYYFRIVRGSSMRYWGQGTLVTVSS |
| Exemplary anti-target (DLL3) | EVQLVESGGGLVQPGGSLTLSCVASGSTSSINAMAWYRRAPG |
| (SEQ ID NO: 405) | KQRELVAGISSDGSKVYADSVKGRFTISRDNAKNSVYLQMNSL |
| | RAEDTAVYYCYYYRTVSGSSMRYWGQGTLVTVSS |
| Exemplary anti-target (DLL3) | EVQLVESGGGLVQPGGSLTLSCVASGSGSSINAMGWYRRAPG |
| (SEQ ID NO: 406) | KQRELVAGISSDGSKVYSDSVKGRFTISRDNAKNSVYLQMNSLR |
| | AEDTAVYYCYYFRHVSGSSMRYWGQGTLVTVSS |
| Exemplary anti-target (DLL3) | EVQLVESGGGLVQPGGSLTLSCVASGTTSSINAMGWYRRAPG |
| (SEQ ID NO: 407) | KQRELVAGISSDGSKVYVDSVKGRFTISRDNAKNSVYLQMNSL |
| | RAEDTAVYYCYYFRFVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 408) | KQRELVAGISSDGSKVYVDSVKGRFTISRDNAKNSVYLQMNSL |
| | RAEDTAVYYCYYFRTVSGSSMRYWGQGTLVTVSS |
| Exemplary anti-target (DLL3) | EVQLVESGGGLVQPGGSLTLSCVASGSTSRINAMGWYRRAPG |
| (SEQ ID NO: 409) | KQRELVAGISSDGSKVYADSVKGRFTISRDNAKNSVYLQMNSL |
| | RAEDTAVYYCYYFRTKSGSSMRYWGQGTLVTVSS |
| Exemplary anti-target (DLL3) | EVQLVESGGGLVQPGGSLTLSCVASGSTSRINAMGWYRRAPG |
| (SEQ ID NO: 410) | KQRELVAGISSDGSKVYADSVKGRFTISRDNAKNSVYLQMNSL |
| | RAEDTAVYYCYYFRTVYGSSMRYWGQGTLVTVSS |
| Exemplary anti-target (DLL3) | EVQLVESGGGLVQPGGSLTLSCVASGSTSSINAMGWYRRAPG |
| (SEQ ID NO: 411) | KQRELVAGISSDGSKVYRDSVKGRFTISRDNAKNSVYLQMNSL |
| | RAEDTAVYYCYYFRTVSGSSMGYWGQGTLVTVSS |
| Exemplary anti-target (DLL3) | EVQLVESGGGLVQPGGSLTLSCVASGSTSSINAMAWYRRAPG |
| (SEQ ID NO: 412) | KQRELVAGISSDGSKVYSDSVKGRFTISRDNAKNSVYLQMNSLR |
| | AEDTAVYYCYYFRTVSGSSMRSWGQGTLVTVSS |
| Exemplary anti-target (DLL3) | EVQLVESGGGLVQPGGSLTLSCVASGSTSSINAMAWYRRAPG |
| (SEQ ID NO: 413) | KQRELVAGISSDNSKVYADSVKGRFTISRDNAKNSVYLQMNSL |
| | RAEDTAVYYCYYFRTVGGSSMRYWGQGTLVTVSS |
| Exemplary anti-target (DLL3) | EVQLVESGGGLVQPGGSLTLSCVASGNTSSINAMAWYRRAPG |
| (SEQ ID NO: 414) | KQRELVAGISSDGSKVYADSVKGRFTISRDNAKNSVYLQMNSL |
| | RAEDTAVYYCYYFRTVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 415) | KQRELVAGISSDGSKVYADSVKGRFTISRDNAKNSVYLQMNSL |
| | RAEDTAVYYCYYFRTVSGSHMRYWGQGTLVTVSS |
| Exemplary anti-target (DLL3) | EVQLVESGGGLVQPGGSLTLSCVASGSTSIINAMGWYRRAPGK |
| (SEQ ID NO: 416) | QRELVAGISSDGSKVYEDSVKGRFTISRDNAKNSVYLQMNSLR |
| | AEDTAVYYCYYFRAVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 417) | KQRELVAGISSDGSKVYADSVKGRFTISRDNAKNSVYLQMNSL |
| | RAEDTAVYYCYYFRTVSGSSMRYWGQGTLVTVSS |
| Exemplary anti-target (DLL3) | EVQLVESGGGLVQPGGSLTLSCVASGSTSRINAMGWYRRAPG |
| (SEQ ID NO: 418) | KQRELPAGISSDGSKVYAVSVKGRFTISRDNAKNSVYLQMNSLR |
| | AEDTAVYYCYYFRTVSGSPMRYWGQGTLVTVSS |
| Exemplary anti-target (DLL3) | EVQLVESGGGLVQPGGSLTLSCVASGSTSRINAMGWYRRAPG |
| (SEQ ID NO: 419) | KQRELVAGVSSDGSKVYADSVKGRFTISRDNAKNSVYLQMNSL |
| | RAEDTAVYYCYYFRTVSGSSMSYWGQGTLVTVSS |
| Exemplary anti-target (DLL3) | EVQLVESGGGLVQPGGSLTLSCVASGTTSSINAMGWYRRAPG |
| (SEQ ID NO: 420) | KQRELVAGISSDGSKVYEDSVKGRFTISRDNAKNSVYLQMNSLR |
| | AEDTAVYYCYYFRTVSGSSMRYWGQGTLVTVSS |
| Exemplary anti-target (DLL3) | EVQLVESGGGLVQPGGSLTLSCVASGITSSINAMGWYRRAPGK |
| (SEQ ID NO: 421) | QRELVAGISSDGSKVYAGSVKGRFTISRDNAKNSVYLQMNSLR |
| | AEDTAVYYCYYFRTVRGSSMRYWGQGTLVTVSS |
| Exemplary anti-target (DLL3) | EVQLVESGGGLVQPGGSLTLSCVASGSTSDINAMGWYRRAPG |
| (SEQ ID NO: 422) | KQRELVAGISSDKSKVYADSVKGRFTISRDNAKNSVYLQMNSLR |
| | AEDTAVYYCYYFRTVRGSSMRYWGQGTLVTVSS |
| Exemplary anti-target (DLL3) | EVQLVESGGGLVQPGGSLTLSCVASGSTSSINAMAWYRRAPG |
| (SEQ ID NO: 423) | KQRELVAGISSNGSKVYADSVKGRFTISRDNAKNSVYLQMNSL |
| | RAEDTAVYYCYYFRQVSGSSMRYWGQGTLVTVSS |
| Exemplary anti-target (DLL3) | EVQLVESGGGLVQPGGSLTLSCVASGSTSSINAIGWYRRAPGK |
| (SEQ ID NO: 424) | QRELVAGISSDGSKVLADSVKGRFTISRDNAKNSVYLQMNSLR |
| | AEDTAVYYCYYFRIVSGSSMGYWGQGTLVTVSS |
| Exemplary anti-target (DLL3) | EVQLVESGGGLVQPGGSLTLSCVASGSTSSKNAMGWYRRAPG |
| (SEQ ID NO: 425) | KQRELVAGISSDGSKVYADSVKGRFTISRDNAKNSVYLQMNSL |
| | RAEDTAVYYCYYFRTVSGASMRYWGQGTLVTVSS |
| Exemplary anti-target (DLL3) | EVQLVESGGGLVQPGGSLTLSCVASGSTSSINAMGWYRRAPG |
| (SEQ ID NO: 426) | KQRELVAGISSDNSKVYADSVKGRFTISRDNAKNSVYLQMNSL |
| | RAEDTAVYYCYYFRTVHGSSMRYWGQGTLVTVSS |
| Exemplary anti-target (DLL3) | EVQLVESGGGLVQPGGSLTLSCVASGLTSSINAMGWYRRAPG |
| (SEQ ID NO: 427) | KQRELVAGISSDGSKVYADSVKGRFTISRDNAKNSVYLQMNSL |
| | RAEDTAVYYCYYFRMVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 428) | KQRELVAGISSDGSKVYTDSVKGRFTISRDNAKNSVYLQMNSLR |
| | AEDTAVYYCYYFRTISGSSMRYWGQGTLVTVSS |
| Exemplary anti-target (DLL3) | EVQLVESGGGLVQPGGSLTLSCVASGSTSSNNAMAWYRRAPG |
| (SEQ ID NO: 429) | KQRELVAGISSDGSKVYTDSVKGRFTISRDNAKNSVYLQMNSLR |
| | AEDTAVYYCYYFRTRSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 430) | KQRELVAGISSDNSKVYADSVKGRFTISRDNAKNSVYLQMNSL |
| | RAEDTAVYYCYYFRTVSGHSMRYWGQGTLVTVSS |
| Exemplary anti-target (DLL3) | EVQLVESGGGLVQPGGSLTLSCVASGSTSHINAMGWYRRAPG |
| (SEQ ID NO: 431) | KQRELVAGISSDGSRVYADSVKGRFTISRDNAKNSVYLQMNSL |
| | RAEDTAVYYCYYFRTVSGGSMRYWGQGTLVTVSS |
| Exemplary anti-target (DLL3) | EVQLVESGGGLVQPGGSLTLSCVASGQTSSINAMGWYRRAPG |
| (SEQ ID NO: 432) | KQRELVAGISSDGSQVYADSVKGRFTISRDNAKNSVYLQMNSL |
| | RAEDTAVYYCYYFRTKSGSSMRYWGQGTLVTVSS |
| Exemplary anti-target (DLL3) | EVQLVESGGGLVQPGGSLTLSCVASGSTSRINGMGWYRRAPG |
| (SEQ ID NO: 433) | KQRELPAGISSDGSKAYADSVKGRFTISRDNAKNSVYLQMNSL |
| | RAEDTAVYYCYYFRTASGTSMRYWGQGTLVTVSS |
| Exemplary anti-target (DLL3) | EVQLVESGGGLVQPGGSLTLSCVASGSTSVINAMAWYRRAPG |
| (SEQ ID NO: 434) | KQRELAAGISSDGSKVYAKSAKGRFTISRDNAKNSVYLQMNSLR |
| | AEDTAVYYCYYFNTVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 435) | KQRELVAGISSDGSKVYNDSVKGRFTISRDNAKNSVYLQMNSL |
| | RAEDTAVYYCYYFRTVRGSSQRYWGQGTLVTVSS |
| Exemplary anti-target (DLL3) | EVQLVESGGGLVQPGGSLTLSCVASGKTSSINAMGWYRRAPG |
| (SEQ ID NO: 436) | KQRELVAGISSDGSKVIADSVKGRFTISRDNAKNSVYLQMNSLR |
| | AEDTAVYYCYYFRTVLGSSMRYWGQGTLVTVSS |
| Exemplary anti-target (DLL3) | EVQLVESGGGLVQPGGSLTLSCVASGSTSSINAMAWYRRAPG |
| (SEQ ID NO: 437) | KQRELVAGISSDGSKVYTDSVKGRFTISRDNAKNSVYLQMNSLR |
| | AEDTAVYYCYYFRTRSGSSMRYWGQGTLVTVSS |
| Exemplary anti-target (DLL3) | EVQLVESGGGLVQPGGSLTLSCVASGSVSSINAMGWYRRAPG |
| (SEQ ID NO: 438) | KQRELVAGISSDGSKVYIDSVKGRFTISRDNAKNSVYLQMNSLR |
| | AEDTAVYYCYYFRTVSGLSMRYWGQGTLVTVSS |
| Exemplary anti-target (DLL3) | EVQLVESGGGLVQPGGSLTLSCVASGNTSSINAMGWYRRAPG |
| (SEQ ID NO: 439) | KQRELVAGISSDGSKVYYDSVKGRFTISRDNAKNSVYLQMNSLR |
| | AEDTAVYYCYYFRTVRGSSQRYWGQGTLVTVSS |
| Exemplary anti-target (DLL3) | EVQLVESGGGLVQPGGSLTLSCVASGSTSSTNAMGWYRRAPG |
| (SEQ ID NO: 440) | KQRELVAGISSDGSKVYVDSVKGRFTISRDNAKNSVYLQMNSL |
| | RAEDTAVYYCYYFRTVSGSSMVYWGQGTLVTVSS |
| Exemplary anti-target (DLL3) | EVQLVESGGGLVQPGGSLTLSCVASGSTSSINAMGWYRRAPG |
| (SEQ ID NO: 441) | KQRELVAGISSDGSKVYGDSVKGRFTISRDNAKNSVYLQMNSL |
| | RAEDTAVYYCYYFRTVSRSSMRYWGQGTLVTVSS |
| Exemplary anti-target (DLL3) | EVQLVESGGGLVQPGGSLTLSCVASGSTSSINAMAWYRRAPG |
| (SEQ ID NO: 442) | KQRELAAGISSDQSKVYADSAKGRFTISRDNAKNSVYLQMNSL |
| | RAEDTAVYYCYYFRTVSGSSMSYWGQGTLVTVSS |
| Exemplary anti-target (DLL3) | EVQLVESGGGLVQPGGSLTLSCVASGGTSSINAMGWYRRAPG |
| (SEQ ID NO: 443) | KQRELVAGISSDGSKVYSDSVKGRFTISRDNAKNSVYLQMNSLR |
| | AEDTAVYYCYYFRTVSGSSARYWGQGTLVTVSS |
| Exemplary anti-target (DLL3) | EVQLVESGGGLVQPGGSLTLSCVASGSTRSINAMGWYRRAPG |
| (SEQ ID NO: 444) | KQRELVAGISSDGSKVYADSVKGRFTISRDNAKNSVYLQMNSL |
| | RAEDTAVYYCYYFHTVSGSSMRYWGQGTLVTVSS |
| Exemplary anti-target (DLL3) | EVQLVESGGGLVQPGGSLTLSCVASGTTSSINAMGWYRRAPG |
| (SEQ ID NO: 445) | KQRELVAGISSDGSKVIADSVKGRFTISRDNAKNSVYLQMNSLR |
| | AEDTAVYYCYYFRTVLGSSMRYWGQGTLVTVSS |
| Exemplary anti-target (DLL3) | EVQLVESGGGLVQPGGSLTLSCVASGSTSSINAMGWYRRAPG |
| (SEQ ID NO: 446) | KQRELVAGISSDGSKVDADSVKGRFTISRDNAKNSVYLQMNSL |
| | RAEDTAVYYCYYFRTVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 447) | KQRELVAGISSDGSKVYKDSVKGRFTISRDNAKNSVYLQMNSLR |
| | AEDTAVYYCYYFRNVSGSSMRYWGQGTLVTVSS |
| Exemplary anti-target (DLL3) | EVQLVESGGGLVQPGGSLTLSCVASGNTSSINAMGWYRRAPG |
| (SEQ ID NO: 448) | KQRELVAGISSNGSKVYADSVKGRFTISRDNAKNSVYLQMNSL |
| | RAEDTAVYYCYYFRTVTGSSMRYWGQGTLVTVSS |
| Exemplary anti-target (DLL3) | EVQLVESGGGLVQPGGSLTLSCVASGSTSRINAMGWYRRAPG |
| (SEQ ID NO: 449) | KQRELVAGISSDGSKVYKDSVKGRFTISRDNAKNSVYLQMNSLR |
| | AEDTAVYYCYYFRTVSGSSMRYWGQGTLVTVSS |
| Exemplary anti-target (DLL3) | EVQLVESGGGLVQPGGSLTLSCVASGSTSSINAIGWYRRAPGK |
| (SEQ ID NO: 450) | QRELVAGISSDGSKVYADSVKGRFTISRDNAKNSVYLQMNSLR |
| | AEDTAVYYCYYFRTVKGSSMRYWGQGTLVTVSS |
| Exemplary anti-target (DLL3) | EVQLVESGGGLVQPGGSLTLSCVASGLTSSINAMGWYRRAPG |
| (SEQ ID NO: 451) | KQRELVAGISSDGSKVYQDSVKGRFTISRDNAKNSVYLQMNSL |
| | RAEDTAVYYCYYFRTNSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 452) | KQRELVAGISSDGSKVYAESVKGRFTISRDNAKNSVYLQMNSLR |
| | AEDTAVYYCYYFRTVSGASMRYWGQGTLVTVSS |
| Exemplary anti-target (DLL3) | EVQLVESGGGLVQPGGSLTLSCVASGSTSSTNAMGWYRRAPG |
| (SEQ ID NO: 453) | KQRELVAGISSDGSKVLADSVKGRFTISRDNAKNSVYLQMNSLR |
| | AEDTAVYYCYYFRTVNLSSMRYWGQGTLVTVSS |
| Exemplary anti-target (DLL3) | EVQLVESGGGLVQPGGSLTLSCVASGSTSSINAIGWYRRAPGK |
| (SEQ ID NO: 454) | QRELVAGISSDGSKYYADSVKGRFTISRDNAKNSVYLQMNSLR |
| | AEDTAVYYCYYFRTVTGSSMRYWGQGTLVTVSS |
| Exemplary anti-target (DLL3) | EVQLVESGGGLVQPGGSLTLSCVASGSTSSINAIGWYRRAPGK |
| (SEQ ID NO: 455) | QRELVAGISSDGSKVYAVSVKGRFTISRDNAKNSVYLQMNSLR |
| | AEDTAVYYCYYFRKVSGSSARYWGQGTLVTVSS |
| Exemplary anti-target (DLL3) | EVQLVESGGGLVQPGGSLTLSCVASGSTSSINAMGWYRRAPG |
| (SEQ ID NO: 456) | KQRELVAGISSDGSKVVADSVKGRFTISRDNAKNSVYLQMNSL |
| | RAEDTAVYYCYYFRTYSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 457) | KQRELVAGISSDGSKVYADSVKGRFTISRDNAKNSVYLQMNSL |
| | RAEDTAVYYCYYFRTVSKSSMRYWGQGTLVTVSS |
| Exemplary anti-target (DLL3) | EVQLVESGGGLVQPGGSLTLSCVASGSTSSINAMAWYRRAPG |
| (SEQ ID NO: 458) | KQRELVAGISSDGSKVYADSVKGRFTISRDNAKNSVYLQMNSL |
| | RAEDTAVYYCYYFKTVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 459) | KQRELAAGISSDNSKVYADSVKGRFTISRDNAKNSVYLQMNSL |
| | RAEDTAVYYCYYFRTRSGSSMRYWGQGTLVTVSS |
| Exemplary anti-target (DLL3) | EVQLVESGGGLVQPGGSLTLSCVASGSKSSINAMGWYRRAPG |
| (SEQ ID NO: 460) | KQRELAAGISSDGSKVYAQSVKGRFTISRDNAKNSVYLQMNSL |
| | RAEDTAVYYCYYFRTSSGSSMRYWGQGTLVTVSS |
| Exemplary anti-target (DLL3) | EVQLVESGGGLVQPGGSLTLSCVASGTTSSINAMGWYRRAPG |
| (SEQ ID NO: 461) | KQRELVAGISSDGSKVYVDSVKGRFTISRDNAKNSVYLQMNSL |
| | RAEDTAVYYCYYFRFLSGSSMRYWGQGTLVTVSS |
| Exemplary anti-target (DLL3) | EVQLVESGGGLVQPGGSLTLSCVASGSTSSINAFGWYRRAPGK |
| (SEQ ID NO: 462) | QRELVAGISSDGSKVYSDSVKGRFTISRDNAKNSVYLQMNSLRA |
| | EDTAVYYCYYFRTVSGSSMRYWGQGTLVTVSS |
| Exemplary anti-target (DLL3) | EVQLVESGGGLVQPGGSLTLSCVASGSTFSINAMGWYRRAPG |
| (SEQ ID NO: 463) | KQRELVAGISSDGSKVLADSVKGRFTISRDNAKNSVYLQMNSLR |
| | AEDTAVYYCYYFRLVSGSSMRYWGQGTLVTVSS |
| Exemplary anti-target (DLL3) | EVQLVESGGGLVQPGGSLTLSCVASGSTRSINAMGWYRRAPG |
| (SEQ ID NO: 464) | KQRELVAGISSDGSKVYNDSVKGRFTISRDNAKNSVYLQMNSL |
| | RAEDTAVYYCYYFRTVSGSSMRFWGQGTLVTVSS |
| Exemplary anti-target (DLL3) | EVQLVESGGGLVQPGGSLTLSCVASGSTSSINAIGWYRRAPGK |
| (SEQ ID NO: 465) | QRELVAGISSDGSKVYNDSVKGRFTISRDNAKNSVYLQMNSLR |
| | AEDTAVYYCYYFRTQSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 466) | KQRELVAGISSDGSKVYVDSVKGRFTISRDNAKNSVYLQMNSL |
| | RAEDTAVYYCYYFRTVSGSSMPYWGQGTLVTVSS |
| Exemplary anti-target (DLL3) | EVQLVESGGGLVQPGGSLTLSCVASGSTSSINAMGWYRRAPG |
| (SEQ ID NO: 467) | KQRELVAGISSDGSKVVADSVKGRFTISRDNAKNSVYLQMNSL |
| | RAEDTAVYYCYYFRTLSGSSMRYWGQGTLVTVSS |
| Exemplary anti-target (DLL3) | EVQLVESGGGLVQPGGSLTLSCVASGTTSSINAMGWYRRAPG |
| (SEQ ID NO: 468) | KQRELVAGISSDGSKVYGDSVKGRFTISRDNAKNSVYLQMNSL |
| | RAEDTAVYYCYYFRTVSGSAMRYWGQGTLVTVSS |
| Exemplary anti-target (DLL3) | EVQLVESGGGLVQPGGSLTLSCVASGSTSSINAMAWYRRAPG |
| (SEQ ID NO: 469) | KQRELVAGISSDGSKVYTDSVKGRFTISRDNAKNSVYLQMNSLR |
| | AEDTAVYYCYYFRTTSGSSMRYWGQGTLVTVSS |
| Exemplary anti-target (DLL3) | EVQLVESGGGLVQPGGSLTLSCVASGRTSSINAMGWYRRAPG |
| (SEQ ID NO: 470) | KQRELVAGISSDGSKVYNDSVKGRFTISRDNAKNSVYLQMNSL |
| | RAEDTAVYYCYYFRTVSGTSMRYWGQGTLVTVSS |
| Exemplary anti-target (DLL3) | EVQLVESGGGLVQPGGSLTLSCVASGSTSSRNAMGWYRRAPG |
| (SEQ ID NO: 471) | KQRELVAGISSDGSKVTADSVKGRFTISRDNAKNSVYLQMNSL |
| | RAEDTAVYYCYYFRTRSGSSMRYWGQGTLVTVSS |
| Exemplary anti-target (DLL3) | EVQLVESGGGLVQPGGSLTLSCVASGSTKSINAMGWYRRAPG |
| (SEQ ID NO: 472) | KQRELVAGISSDGSKVYRDSVKGRFTISRDNAKNSVYLQMNSL |
| | RAEDTAVYYCYYFRTSSGSSMRYWGQGTLVTVSS |
| Exemplary anti-target (DLL3) | EVQLVESGGGLVQPGGSLTLSCVASGSTSSRNAMGWYRRAPG |
| (SEQ ID NO: 473) | KQRELVAGISSNGSKVYSDSVKGRFTISRDNAKNSVYLQMNSLR |
| | AEDTAVYYCYYFRTVSGSSMSYWGQGTLVTVSS |
| Exemplary anti-target (DLL3) | EVQLVESGGGLVQPGGSLTLSCVASGSTSSINAIGWYRRAPGK |
| (SEQ ID NO: 474) | QRELVAGISSDGSKVYSDSVKGRFTISRDNAKNSVYLQMNSLRA |
| | EDTAVYYCYYFRPVSGSSMRYWGQGTLVTVSS |
| Exemplary anti-target (DLL3) | EVQLVESGGGLVQPGGSLTLSCVASGSTSLINAMGWYRRAPG |
| (SEQ ID NO: 475) | KQRELVAGISSDGSKVYADSVKGRFTISRDNAKNSVYLQMNSL |
| | RAEDTAVYYCYYFRHVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 476) | KQRELVAGISSDGSKVYADSVKGRFTISRDNAKNSVYLQMNSL |
| | RAEDTAVYYCYYFRTKSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 477) | KQRELVAGISSDGSLVYADSVKGRFTISRDNAKNSVYLQMNSLR |
| | AEDTAVYYCYYFTTVSGSSMRYWGQGTLVTVSS |
| Exemplary anti-target (DLL3) | EVQLVESGGGLVQPGGSLTLSCVASGTTSSINAMGWYRRAPG |
| (SEQ ID NO: 478) | KQRELVAGISSDGTKVYADSVKGRFTISRDNAKNSVYLQMNSL |
| | RAEDTAVYYCYYFHTVSGSSMRYWGQGTLVTVSS |
| Exemplary anti-target (DLL3) | EVQLVESGGGLVQPGGSLTLSCVASGSTSSINAFGWYRRAPGK |
| (SEQ ID NO: 479) | QRELVAGISSDGSKVYADSVKGRFTISRDNAKNSVYLQMNSLR |
| | AEDTAVYYCYYFRTVRGSSMRYWGQGTLVTVSS |
| Exemplary anti-target (DLL3) | EVQLVESGGGLVQPGGSLTLSCVASGSTSSRNAMGWYRRAPG |
| (SEQ ID NO: 480) | KQRELVAGISSDGSKLYLDSVKGRFTISRDNAKNSVYLQMNSLR |
| | AEDTAVYYCYYFRTVLGSSMRYWGQGTLVTVSS |
| Exemplary anti-target (DLL3) | EVQLVESGGGLVQPGGSLTLSCVASGNTSSINAMGWYRRAPG |
| (SEQ ID NO: 481) | KQRELVAGISSDGSRVYADSVKGRFTISRDNAKNSVYLQMNSL |
| | RAEDTAVYYCYYFRTVSGSSMRSWGQGTLVTVSS |
| Exemplary anti-target (DLL3) | EVQLVESGGGLVQPGGSLTLSCVASGTTSSINAMGWYRRAPG |
| (SEQ ID NO: 482) | KQRELVAGISSDGSKVYNDSVKGRFTISRDNAKNSVYLQMNSL |
| | RAEDTAVYYCYYFRTVRGSSMRYWGQGTLVTVSS |
| Exemplary anti-target (DLL3) | EVQLVESGGGLVQPGGSLTLSCVASGSTASINAMGWYRRAPG |
| (SEQ ID NO: 483) | KQRELVAGISSDGSKVYADSVKGRFTISRDNAKNSVYLQMNSL |
| | RAEDTAVYYCYYFRYVSGSSMRYWGQGTLVTVSS |
| Exemplary anti-target (DLL3) | EVQLVESGGGLVQPGGSLTLSCVASGSTSSINAVGWYRRAPGK |
| (SEQ ID NO: 484) | QRELVAGISSDGSKVYVDSVKGRFTISRDNAKNSVYLQMNSLR |
| | AEDTAVYYCYYFRTVYGSSMRYWGQGTLVTVSS |
| Exemplary anti-target (DLL3) | EVQLVESGGGLVQPGGSLTLSCVASGSTSSRNAMGWYRRAPG |
| (SEQ ID NO: 485) | KQRELVAGISSDGSKLYADSVKGRFTISRDNAKNSVYLQMNSLR |
| | AEDTAVYYCYYFRTVLGSSMRYWGQGTLVTVSS |
| Exemplary anti-target (DLL3) | EVQLVESGGGLVQPGGSLTLSCVASGSTNSINAMGWYRRAPG |
| (SEQ ID NO: 486) | KQRELVAGISSDGSKVYKDSVKGRFTISRDNAKNSVYLQMNSLR |
| | AEDTAVYYCYYYRTVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 487) | KQRELVAGISSDGSKVYADSVKGRFTISRDNAKNSVYLQMNSL |
| | RAEDTAVYYCYYFRSVSGSSMRYWGQGTLVTVSS |
| Exemplary anti-target (DLL3) | EVQLVESGGGLVQPGGSLTLSCVASGSTSRINAMGWYRRAPG |
| (SEQ ID NO: 488) | KQRELVAGISSDGSKVYQDSVKGRFTISRDNAKNSVYLQMNSL |
| | RAEDTAVYYCYYFRRVSGSSMRYWGQGTLVTVSS |
| Exemplary anti-target (DLL3) | EVQLVESGGGLVQPGGSLTLSCVPSGSTSNINAMGWYRRAPG |
| (SEQ ID NO: 489) | KQRELPAGISSDGTKIYADSAKVPFTITRDNAKNSVYLQMNSLR |
| | AEDTAVYYCYYFRTVSGTSMRYWGQGTLVTVSS |
| Exemplary anti-target (DLL3) | EVQLVESGGGLVQPGGSLTLSCVASGSTSKINAMGWYRRAPG |
| (SEQ ID NO: 490) | KQRELVAGISSDRSKVYADSVKGRFTISRDNAKNSVYLQMNSLR |
| | AEDTAVYYCYYFRTVAGSSMRYWGQGTLVTVSS |
| Exemplary anti-target (DLL3) | EVQLVESGGGLVQPGGSLTLSCVASGSTSSINALGWYRRAPGK |
| (SEQ ID NO: 491) | QRELVAGISSDGSLVYADSVKGRFTISRDNAKNSVYLQMNSLR |
| | AEDTAVYYCYYFRIVSGSSMRYWGQGTLVTVSS |
| Exemplary anti-target (DLL3) | EVQLVESGGGLVQPGGSLTLSCVASGKTSSINAMGWYRRAPG |
| (SEQ ID NO: 911) | KQRELVAGISSDGSKVYADSVKGRFTISRDNAKNSVYLQMNSL |
| | RAEDTAVYYCYYFRTVSGVSMRYWGQGTLVTVSS |
| Exemplary anti-target (DLL3) | EVQLVESGGGLVQPGGSLTLSCVASGSTSSINAVGWYRRAPGK |
| (SEQ ID NO: 912) | QRELVAGISSDGSKVYRDSVKGRFTISRDNAKNSVYLQMNSLR |
| | AEDTAVYYCYYFRTVQGSSMRYWGQGTLVTVSS |
| Exemplary anti-target (DLL3) | EVQLVESGGGLVQPGGSLTLSCVASGSTSRINAMGWYRRAPG |
| (SEQ ID NO: 492) | KQRELVAGISSDGSKVYADSVKGRFTISRDNAKNSVYLQMNSL |
| | RAEDTAVYYCYYFRTASGSSMRYWGQGTLVTVSS |
| Exemplary anti-target (DLL3) | EVQLVESGGGLVQPGGSLTLSCVASGSTSSINAMGWYRRAPG |
| (SEQ ID NO: 493) | KQRELVAGISSDGSKVYSDSVKGRFTISRDNAKNSVYLQMNSLR |
| | AEDTAVYYCYYFRTVSGSSSRYWGQGTLVTVSS |
| Exemplary anti-target (DLL3) | EVQLVESGGGLVQPGGSLTLSCVASGSTSSINAVGWYRRAPGK |
| (SEQ ID NO: 494) | QRELVAGISSDGTKVYRDSVKGRFTISRDNAKNSVYLQMNSLR |
| | AEDTAVYYCYYFRTVQGSSMRYWGQGTLVTVSS |
| Exemplary anti-target (DLL3) | EVQLVESGGGLVQPGGSLTLSCVASGTTSSINAMGWYRRAPG |
| (SEQ ID NO: 495) | KQRELAAGISSDGSKVYNDSVKGRFTISRDNAKNSVYLQMNSL |
| | RAEDTAVYYCYYFRTVRGSSMRYWGQGTLVTVSS |
| Exemplary anti-target (DLL3) | EVQLVESGGGLVQPGGSLTLSCVASGSTSSINAMGWYRRAPG |
| (SEQ ID NO: 496) | KQRELVAGISSDGSKVYADSVKGRFTISRDNAKNSVYLQMNSL |
| | RAEDTAVYYCYYFRTKSGSSMRYWGQGTLVTVSS |
| Exemplary anti-target (DLL3) | EVQLVESGGGLVQPGGSLTLSCVASGTTSSINAMGWYRRAPG |
| (SEQ ID NO: 497) | KQRELVAGISSDGSKVYADSVKGRFTISRDNAKNSVYLQMNSL |
| | RAEDTAVYYCYYFRTVWGSSMRYWGQGTLVTVSS |
| Exemplary anti-target (DLL3) | EVQLVESGGGLVQPGGSLTLSCVASGKTSSINAMGWYRRAPG |
| (SEQ ID NO: 498) | KQRELVAGISSDGSKVYTDSVKGRFTISRDNAKNSVYLQMNSLR |
| | AEDTAVYYCYYFRTRSGSSMRYWGQGTLVTVSS |
| Exemplary anti-target (DLL3) | EVQLVESGGGLVQPGGSLTLSCVASGSTSSINAMGWYRRAPFK |
| (SEQ ID NO: 499) | QGELPAGISPDGTKAYADSAKVRFTISRDNAKNSVYLQMNSLR |
| | AEDTAVYYCYYFHTVCGTSMGYWGQGTLVTVSS |
| Exemplary anti-target (DLL3) | EVQLVESGGGLVQPGGSLTLSCVASGSTSAINAMGWYRRAPG |
| (SEQ ID NO: 500) | KQRELVAGISSDGSKVYVDSVKGRFTISRDNAKNSVYLQMNSL |
| | RAEDTAVYYCYYFRTVSGSSQRYWGQGTLVTVSS |
| Exemplary anti-target (DLL3) | EVQLVESGGGLVQPGGSLTLSCVASGSPSSINAYGWYRRAPGK |
| (SEQ ID NO: 501) | QRELVAGISSDGSKVYSDSVKGRFTISRDNAKNSVYLQMNSLRA |
| | EDTAVYYCYYFRTVSGSSMSYWGQGTLVTVSS |
| Exemplary anti-target (DLL3) | EVQLVESGGGLVQPGGSLTLSCVASGSTSSINAMGWYRRAPG |
| (SEQ ID NO: 502) | KQRELVAGISSDGSKVYASSVKGRFTISRDNAKNSVYLQMNSLR |
| | AEDTAVYYCYYFRTVRGSSMRYWGQGTLVTVSS |
| Exemplary anti-target (DLL3) | EVQLVESGGGLVQPGGSLTLSCVASGSRSSINAMGWYRRAPG |
| (SEQ ID NO: 503) | KQRELVAGISADGSKVYADSVKGRFTISRDNAKNSVYLQMNSL |
| | RAEDTAVYYCYYFRTQSGSSMRYWGQGTLVTVSS |
| Exemplary anti-target (DLL3) | EVQLVESGGGLVQPGGSLTLSCVASGSVSSINAMGWYRRAPG |
| (SEQ ID NO: 504) | KQRELVAGISSDGSKVYASSAKGRFTISRDNAKNSVYLQMNSLR |
| | AEDTAVYYCYYFRTLSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 505) | KQRELVAGISSDGSKVYADSVKGRFTISRDNAKNSVYLQMNSL |
| | RAEDTAVYYCYYFHTVSGSSMRYWGQGTLVTVSS |
| Exemplary anti-target (DLL3) | EVQLVESGGGLVQPGGSLTLSCVASGSTSSINAIGWYRRAPGK |
| (SEQ ID NO: 506) | QRELVAGISSDGSSVYADSVKGRFTISRDNAKNSVYLQMNSLR |
| | AEDTAVYYCYYFRRVSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 507) | KQRELVAGISSDGSKVYSDSVKGRFTISRDNAKNSVYLQMNSLR |
| | AEDTAVYYCYYFRLVSGSSMRYWGQGTLVTVSS |
| Exemplary anti-target (DLL3) | EVQLVESGGGLVQPGGSLTLSCVASGSTSSINAVGWYRRAPGK |
| (SEQ ID NO: 508) | QRELVAGISSDGSKVYAGSVKGRFTISRDNAKNSVYLQMNSLR |
| | AEDTAVYYCYYFRTVSGSYMRYWGQGTLVTVSS |
| Exemplary anti-target (DLL3) | EVQLVESGGGLVQPGGSLTLSCVASGSTSSINAMAWYRRAPG |
| (SEQ ID NO: 509) | KQRELAAGISSDNSKVYADSVKGRFTISRDNAKNSVYLQMNSL |
| | RAEDTAVYYCYYFRTVGGSSMRYWGQGTLVTVSS |
| Exemplary anti-target (DLL3) | EVQLVESGGGLVQPGGSLTLSCVASGSTSSINAYGWYRRAPGK |
| (SEQ ID NO: 510) | QRELVAGISSDGSAVYADSVKGRFTISRDNAKNSVYLQMNSLR |
| | AEDTAVYYCYYFRTHSGSSMRYWGQGTLVTVSS |
| Exemplary anti-target (DLL3) | EVQLVESGGGLVQPGGSLTLSCVASGSTSSINAVGWYRRAPGK |
| (SEQ ID NO: 511) | QRELVAGISSDGSSVYADSVKGRFTISRDNAKNSVYLQMNSLR |
| | AEDTAVYYCYYFRTVSTSSMRYWGQGTLVTVSS |
| Exemplary anti-target (DLL3) | EVQLVESGGGLVQPGGSLTLSCVASGSKSSINAMGWYRRAPG |
| (SEQ ID NO: 512) | KQRELPAGISSNGTKVYADSAKVRFTISRDNAKNSVYLQMNSL |
| | RAEDTAVYYCYYFRTVLGTSMRYWGQGTLVTVSS |
| Exemplary anti-target (DLL3) | EVQLVESGGGLVQPGGSLTLSCVASGSTSSINAMAWYRRAPG |
| (SEQ ID NO: 513) | KQRELVAGISSDGSKLYADSVKGRFTISRDNAKNSVYLQMNSLR |
| | AEDTAVYYCYYFRTVSGSSMRYWGQGTLVTVSS |
| Exemplary anti-target (DLL3) | EVQLVESGGGLVQPGGSLTLSCVASGSVSSINAMGWYRRAPG |
| (SEQ ID NO: 514) | KQRELVAGISSDGSKVYKDSVKGRFTISRDNAKNSVYLQMNSLR |
| | AEDTAVYYCYYFRTVSGSSMGYWGQGTLVTVSS |
| Exemplary anti-target (DLL3) | EVQLVESGGGLVQPGGSLTLSCVASGSTSSINAMGWYRRAPG |
| (SEQ ID NO: 515) | KQRELVAGISSDGSLVYADSVKGRFTISRDNAKNSVYLQMNSLR |
| | AEDTAVYYCYYFRTVSGSSMRAWGQGTLVTVSS |
| Exemplary anti-target (DLL3) | EVQLVESGGGLVQPGGSLTLSCVASGSTSSINAMGWYRRAPG |
| (SEQ ID NO: 516) | KQRELVAGISSDGSLVYADSVKGRFTISRDNAKNSVYLQMNSLR |
| | AEDTAVYYCYYFRILSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 517) | KQRELVAGISSDGSKVYADSVKGRFTISRDNAKNSVYLQMNSL |
| | RAEDTAVYYCYYFRTVQGSSMRYWGQGTLVTVSS |
| Exemplary anti-target (DLL3) | EVQLVESGGGLVQPGGSLTLSCVASGSTSYINAMGWYRRAPG |
| (SEQ ID NO: 518) | KQRELVAGISSDGSKVYADSVKGRFTISRDNAKNSVYLQMNSL |
| | RAEDTAVYYCYYFRTVSGQSMGYWGQGTLVTVSS |
| Exemplary anti-target (DLL3) | EVQLVESGGGLVQPGGSLTLSCVASGSTSSINAMGWYRRAPG |
| (SEQ ID NO: 913) | KQRELVAGVSSDGSKVYADSVKGRFTISRDNAKNSVYLQMNSL |
| | RAEDTAVYYCYYFRTVSGSSARYWGQGTLVTVSS |
| Exemplary anti-target (DLL3) | EVQLVESGGGLVQPGGSLTLSCVASGSTSSINAMGWYRRAPG |
| (SEQ ID NO: 914) | KQRELPAGISRDGSKVYADSVKGRFTISRDNAKNSVYLQMNSL |
| | RAEDTAVYYCYYFRYVSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 915) | KQRELAAGISSDGSKLYADSVKGRFTISRDNAKNSVYLQMNSLR |
| | AEDTAVYYCYYFRTVSGSSMRYWGQGTLVTVSS |
| Exemplary anti-target (DLL3) | EVQLVESGGGLVQPGGSLTLSCVASGSTSRINAMGWYRRAPG |
| (SEQ ID NO: 916) | KQRELVAGISSDGSKVYADSVKGRFTISRDNAKNSVYLQMNSL |
| | RAEDTAVYYCYYFRRVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 917) | KQRELAAGISSDGSKVYADSVKGRFTISRDNAKNSVYLQMNSL |
| | RAEDTAVYYCYYFHTVSGSSMRYWGQGTLVTVSS |
| Exemplary anti-target (DLL3) | EVQLVESGGGLVQPGGSLTLSCVASGSTSSINAMAWYRRAPG |
| (SEQ ID NO: 918) | KQRELVAGISSDGSKVYADSVKGRFTISRDNAKNSVYLQMNSL |
| | RAEDTAVYYCYYFRQVSGSSMRYWGQGTLVTVSS |
| Exemplary anti-target (DLL3) | EVQLVESGGGLVQPGGSLTLSCVASGSTSSINAMGWYRRAPG |
| (SEQ ID NO: 919) | KQRELVAGISSDTSKVYADSVKGRFTISRDNAKNSVYLQMNSLR |
| | AEDTAVYYCYYFRTVSGSYMRYWGQGTLVTVSS |
| Exemplary anti-target (DLL3) | EVQLVESGGGLVQPGGSLTLSCVASGSTSTINAMGWYRRAPG |
| (SEQ ID NO: 920) | KQRELVAGISSDGSKVYADSVKGRFTISRDNAKNSVYLQMNSL |
| | RAEDTAVYYCYYFRTASGSSMRYWGQGTLVTVSS |
| Exemplary anti-target (DLL3) | EVQLVESGGGLVQPGGSLTLSCVASGSTSSINAVGWYRRAPGK |
| (SEQ ID NO: 519) | QRELVAGISSDGSTVYADSVKGRFTISRDNAKNSVYLQMNSLR |
| | AEDTAVYYCYYFRTVSGHSMRYWGQGTLVTVSS |
| Exemplary anti-target (DLL3) | EVQLVESGGGLVQPGGSLTLSCVASGSTSSINAMGWYRRAPG |
| (SEQ ID NO: 520) | KQRELAAGISKDGSKVYADSAKGRFTISRDNAKNSVYLQMNSL |
| | RAEDTAVYYCYYFRTVSGSSSRYWGQGTLVTVSS |
| Exemplary anti-target (DLL3) | EVQLVESGGGLVQPGGSLTLSCVASGSPSSINAYGWYRRAPGK |
| (SEQ ID NO: 521) | QRELVAGISSDGSKVYSDSVKGRFTISRDNAKNSVYLQMNSLRA |
| | EDTAVYYCYYFRTVSGSSYSYWGQGTLVTVSS |
| Exemplary anti-target (DLL3) | EVQLVESGGGLVQPGGSLTLSCVASGSPSSINAYGWYRRAPGK |
| (SEQ ID NO: 522) | QRELVAGISSDGSKVYSDSVKGRFTISRDNAKNSVYLQMNSLRA |
| | EDTAVYYCYYFRTVSGSSQSYWGQGTLVTVSS |
| Exemplary anti-target (DLL3) | EVQLVESGGGLVQPGGSLTLSCAASGSSVKFLSMAWYRQAPG |
| (SEQ ID NO: 523) | KKRELVAGISADGSTDYIDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVSFLSLAWYRQAPGK |
| (SEQ ID NO: 524) | KRELVAGISADGSTAYIDSVKGRFTISRDNAKNSVYLQMNSLRA |
| | EDTAVYYCYAYRWRTRYTYWGQGTLVTVSS |
| Exemplary anti-target (DLL3) | EVQLVESGGGLVQPGGSLTLSCAASGSRVSFLSMAWYRQAPG |
| (SEQ ID NO: 525) | KKRELVAGISRDGSTDYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSQVSFLSMAWYRQAPG |
| (SEQ ID NO: 526) | KKRELVAGISRDGSTDYIDSVKGRFTISRDNAKNSVYLQMNSLR |
| | AEDTAVYYCYAYRWTTRYIYWGQGTLVTVSS |
| Exemplary anti-target (DLL3) | EVQLVESGGGLVQPGGSLTLSCAASGSSVSFLSMAWYRQAPG |
| (SEQ ID NO: 527) | KKRELVAGISEAGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLRCAASGSKVSFLSMAWYRQAPG |
| (SEQ ID NO: 528) | KKRELVAGISADGSTDYVDSVKGRFTISRDNAKNSVYLQMNSL |
| | RAEDTAVYYCYAYRWTRRYTYWGQGTLVTVSS |
| Exemplary anti-target (DLL3) | EVQLVESGGGLVQPGGSLTLSCAASGSSVGFLSMAWYRQAPG |
| (SEQ ID NO: 529) | KKRELVAGISADGSTDYIRSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVSFLSMAWYRQAPG |
| (SEQ ID NO: 530) | KKRELVAGISADGSVDYIDSVKGRFTISRDNAKNSVYLQMNSLR |
| | AEDTAVYYCYAYRWTTRYIYWGQGTLVTVSS |
| Exemplary anti-target (DLL3) | EVQLVESGGGLVQPGGSLTLSCAASGSRVSFLSMAWYRQAPG |
| (SEQ ID NO: 531) | KKRELVAGISADGSTLYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSSVSFLSLAWYRQAPGK |
| (SEQ ID NO: 532) | KRELVAGISTDGSTDYIDSVKGRFTISRDNAKNSVYLQMNSLRA |
| | EDTAVYYCYAYRWRTRYTYWGQGTLVTVSS |
| Exemplary anti-target (DLL3) | EVQLVESGGGLVQPGGSLTLSCAASGSSVSFLSMAWYRQAPG |
| (SEQ ID NO: 533) | KKRELVAGISGDGSTDYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVQFLSMAWYRQAPG |
| (SEQ ID NO: 534) | KKRELVAGISADGSTDYINSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSNVSFLSMAWYRQAPG |
| (SEQ ID NO: 535) | KKRELVAGISARGSTDYIDSVKGRFTISRDNAKNSVYLQMNSLR |
| | AEDTAVYYCYAYHWTTRYTYWGQGTLVTVSS |
| Exemplary anti-target (DLL3) | EVQLVESGGGLVQPGGSLTLSCVASGSSVKFLSMAWYRQAPG |
| (SEQ ID NO: 536) | KKRELVAGISADGSTTYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGKSVSFLSMAWYRQAPG |
| (SEQ ID NO: 537) | KKRELVAGISKDGSTDYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSRVSFLSMAWYRQAPG |
| (SEQ ID NO: 538) | KKRELVAGISADGSTTYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSHVSFLSMAWYRQAPG |
| (SEQ ID NO: 539) | KKRELVAGISANGSTDYIDSVKGRFTISRDNAKNSVYLQMNSLR |
| | AEDTAVYYCYAYRWTTRYAYWGQGTLVTVSS |
| Exemplary anti-target (DLL3) | EVQLVESGGGLVQPGGSLTLSCAASGSSVSFLSMAWYRQAPG |
| (SEQ ID NO: 540) | KKRELVAGISRDGSTDYIDSVKGRFTISRDNAKNSVYLQMNSLR |
| | AEDTAVYYCYAYRWVTRYTYWGQGTLVTVSS |
| Exemplary anti-target (DLL3) | EVQLVESGGGLVQPGGSLTLSCAASGSSVSFLSMAWYRQAPG |
| (SEQ ID NO: 541) | KKRELVAGISADGSADYIDSVKGRFTISRDNAKNSVYLQMNSLR |
| | AEDTAVYYCYAYRWVTRYTYWGQGTLVTVSS |
| Exemplary anti-target (DLL3) | EVQLVESGGGLVQPGGSLTLSCAASGSSVRFLSMAWYRQAPG |
| (SEQ ID NO: 542) | KKRELVAGISAHGSTDYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSSVRFLSMAWYRQAPG |
| (SEQ ID NO: 543) | KKRELVAGISADGSTIYIDSVKGRFTISRDNAKNSVYLQMNSLRA |
| | EDTAVYYCYAYRWRTRYTYWGQGTLVTVSS |
| Exemplary anti-target (DLL3) | EVQLVESGGGLVQPGGSLTLSCAASGSSVRFLSMAWYRQAPG |
| (SEQ ID NO: 544) | KKRELVAGISRDGSTVYIDSVKGRFTISRDNAKNSVYLQMNSLR |
| | AEDTAVYYCYAYRWTTRGTYWGQGTLVTVSS |
| Exemplary anti-target (DLL3) | EVQLVESGGGLVQPGGSLTLSCAASGSHVSFLSMAWYRQAPG |
| (SEQ ID NO: 545) | KKRELVAGISADGPTDYIDSVKGRFTISRDNAKNSVYLQMNSLR |
| | AEDTAVYYCYAYRWDTRYTYWGQGTLVTVSS |
| Exemplary anti-target (DLL3) | EVQLVESGGGLVQPGGSLTLSCVASGTSVSFLSMAWYRQAPG |
| (SEQ ID NO: 546) | KKRELVAGISADGSTTYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGTSVSFLSIAWYRQAPGK |
| (SEQ ID NO: 547) | KRELVAGISADGSTDYIASVKGRFTISRDNAKNSVYLQMNSLRA |
| | EDTAVYYCYAYRWRTRYTYWGQGTLVTVSS |
| Exemplary anti-target (DLL3) | EVQLVESGGGLVQPGGSLTLSCAASGSSVKFLSMAWYRQAPG |
| (SEQ ID NO: 548) | KKRELVAGISLDGSTDYIDSVKGRFTISRDNAKNSVYLQMNSLR |
| | AEDTAVYYCYAYRWTGRYTYWGQGTLVTVSS |
| Exemplary anti-target (DLL3) | EVQLVESGGGLVQPGGSLTLSCAASGSSVSFLSMAWYRQAPG |
| (SEQ ID NO: 549) | KKRELVAGISADGSTIYIDSVKGRFTISRDNAKNSVYLQMNSLRA |
| | EDTAVYYCYAYRWRTRYTYWGQGTLVTVSS |
| Exemplary anti-target (DLL3) | EVQLVESGGGLVQPGGSLTLSCAASGSSVRFLSMAWYRQAPG |
| (SEQ ID NO: 550) | KKRELVAGISAHGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVSFLSMAWYRQAPG |
| (SEQ ID NO: 551) | KKRELVAGISRDGSTDYIDSVKGRFTISRDNAKNSVYLQMNSLR |
| | AEDTAVYYCYAYRWITRYTYWGQGTLVTVSS |
| Exemplary anti-target (DLL3) | EVQLVESGGGLVQPGGSLTLSCAASGSSVRFLSMAWYRQAPG |
| (SEQ ID NO: 552) | KKRELVAGISRDGSTDYIDSVKGRFTISRDNAKNSVYLQMNSLR |
| | AEDTAVYYCYAYRWITRYTYWGQGTLVTVSS |
| Exemplary anti-target (DLL3) | EVQLVESGGGLVQPGGSLTLSCAASGSSVVFLSMAWYRQAPG |
| (SEQ ID NO: 553) | KKRELVAGISADGSMDYIDSVKGRFTISRDNAKNSVYLQMNSL |
| | RAEDTAVYYCYAYRWRTRYTYWGQGTLVTVSS |
| Exemplary anti-target (DLL3) | EVQLVESGGGLVQPGGSLTLSCAASGSSVRFLSMAWYRQAPG |
| (SEQ ID NO: 554) | KKRELVAGISADGSTDYIDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVRFLSMAWYRQAPG |
| (SEQ ID NO: 555) | KKRELVAGISADGSTDYIDSVKGRFTISRDNAKNSVYLQMNSLR |
| | AEDTAVYYCYAYSWTTRYTYWGQGTLVTVSS |
| Exemplary anti-target (DLL3) | EVQLVESGGGLVQPGGSLTLSCAASGSSVRFLSMAWYRQAPG |
| (SEQ ID NO: 556) | KKRELVAGISANGSTDYIDSVKGRFTISRDNAKNSVYLQMNSLR |
| | AEDTAVYYCYAYRWTNRYTYWGQGTLVTVSS |
| Exemplary anti-target (DLL3) | EVQLVESGGGLVQPGGSLTLSCAASGSSVSRLSMAWYRQAPG |
| (SEQ ID NO: 557) | KKRELVAGISANGSTTYIDSVKGRFTISRDNAKNSVYLQMNSLR |
| | AEDTAVYYCYAYRWTTRYRYWGQGTLVTVSS |
| Exemplary anti-target (DLL3) | EVQLVESGGGLVQPGGSLTLSCAASGSSKSFLSMAWYRQAPG |
| (SEQ ID NO: 558) | KKRELVAGISADGSTSYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVSRLSMAWYRQAPG |
| (SEQ ID NO: 559) | KKRELVAGISADGSRDYIDSVKGRFTISRDNAKNSVYLQMNSLR |
| | AEDTAVYYCYAYRWTTRYKYWGQGTLVTVSS |
| Exemplary anti-target (DLL3) | EVQLVESGGGLVQPGGSLTLSCAASGSSVKFLSMAWYRQAPG |
| (SEQ ID NO: 560) | KKRELVAGISADGSTMYIDSVKGRFTISRDNAKNSVYLQMNSLR |
| | AEDTAVYYCYAYRWHTRYTYWGQGTLVTVSS |
| Exemplary anti-target (DLL3) | EVQLVESGGGLVQPGGSLTLSCAASGSGVRFLSMAWYRQAPG |
| (SEQ ID NO: 561) | KKRELVAGISPDGSTDYIDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVRFLSMAWYRQAPG |
| (SEQ ID NO: 562) | KKRELVAGISGDGSTDYIDSVKGRFTISRDNAKNSVYLQMNSLR |
| | AEDTAVYYCYAYRWMTRYTYWGQGTLVTVSS |
| Exemplary anti-target (DLL3) | EVQLVESGGGLVQPGGSLTLSCAASGSSVHFLSMAWYRQAPG |
| (SEQ ID NO: 563) | KKRELVAGISRDGSTDYIDSVKGRFTISRDNAKNSVYLQMNSLR |
| | AEDTAVYYCYAYRWTTRYRYWGQGTLVTVSS |
| Exemplary anti-target (DLL3) | EVQLVESGGGLVQPGGSLTLSCAASGSSVRFLSMAWYRQAPG |
| (SEQ ID NO: 564) | KKRELVAGISRDGSTDYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSKVSFLSMAWYRQAPG |
| (SEQ ID NO: 565) | KKRELVAGISRDGSTDYIDSVKGRFTISRDNAKNSVYLQMNSLR |
| | AEDTAVYYCYAYRWTTRYTFWGQGTLVTVSS |
| Exemplary anti-target (DLL3) | EVQLVESGGGLVQPGGSLTLSCAASGSSVSFLSMAWYRQAPG |
| (SEQ ID NO: 566) | KKRELVAGISADGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSKVSFLSMAWYRQAPG |
| (SEQ ID NO: 567) | KKRELVAGISTDGSTDYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSRVSFLSMAWYRQAPG |
| (SEQ ID NO: 568) | KKRELVAGISADGSTSYIDSVKGRFTISRDNAKNSVYLQMNSLR |
| | AEDTAVYYCYAYRWATRYTYWGQGTLVTVSS |
| Exemplary anti-target (DLL3) | EVQLVESGGGLVQPGGSLTLSCAASGSRVSFLSMAWYRQAPG |
| (SEQ ID NO: 569) | KKRELVAGISADGSTLYIDSVKGRFTISRDNAKNSVYLQMNSLR |
| | AEDTAVYYCYAYRWHTRYTYWGQGTLVTVSS |
| Exemplary anti-target (DLL3) | EVQLVESGGGLVQPGGSLTLSCAASGSSVRFLSMAWYRQAPG |
| (SEQ ID NO: 570) | KKRELVAGISRDGSTDYIDSVKGRFTISRDNAKNSVYLQMNSLR |
| | AEDTAVYYCYAYRWGTRYTYWGQGTLVTVSS |
| Exemplary anti-target (DLL3) | EVQLVESGGGLVQPGGSLTLSCAASYSSVSRLSMAWYRQAPG |
| (SEQ ID NO: 571) | KKRELVAGISADGSTVYIDSVKGRFTISRDNAKNSVYLQMNSLR |
| | AEDTAVYYCYAYRWTTRNTYWGQGTLVTVSS |
| Exemplary anti-target (DLL3) | EVQLVESGGGLVQPGGSLTLSCAASGSSVSFLSMAWYRQAPG |
| (SEQ ID NO: 572) | KKRELVAGISTDGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVSFLSMAWYRQAPG |
| (SEQ ID NO: 573) | KKRELVAGISADGSTLYIDSVKGRFTISRDNAKNSVYLQMNSLR |
| | AEDTAVYYCYAYRWTTRYAYWGQGTLVTVSS |
| Exemplary anti-target (DLL3) | EVQLVESGGGLVQPGGSLTLSCAASGSSVSFLSMAWYRQAPG |
| (SEQ ID NO: 574) | KKRELVAGISADGRTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCVASGTSVSFLSMAWYRQAPG |
| (SEQ ID NO: 575) | KKRELVAGISADGSTIYIDSVKGRFTISRDNAKNSVYLQMNSLRA |
| | EDTAVYYCYAYRWTTRRTYWGQGTLVTVSS |
| Exemplary anti-target (DLL3) | EVQLVESGGGLVQPGGSLTLSCAASGSSVKFLSMAWYRQAPG |
| (SEQ ID NO: 576) | KKRELVAGISADGSTLYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSSVSFLSMAWYRQAPG |
| (SEQ ID NO: 577) | KKRELVAGISRDGSTDYIDSVKGRFTISRDNAKNSVYLQMNSLR |
| | AEDTAVYYCYAYRWTSRYTYWGQGTLVTVSS |
| Exemplary anti-target (DLL3) | EVQLVESGGGLVQPGGSLTLSCAASGSRVSFLSMAWYRQAPG |
| (SEQ ID NO: 578) | KKRELVAGISKDGSTDYIDSVKGRFTISRDNAKNSVYLQMNSLR |
| | AEDTAVYYCYAYRWTTRVTYWGQGTLVTVSS |
| Exemplary anti-target (DLL3) | EVQLVESGGGLVQPGGSLTLSCAASGSSVSVLSMAWYRQAPG |
| (SEQ ID NO: 579) | KKRELVAGISADGSTDYIGSVKGRFTISRDNAKNSVYLQMNSLR |
| | AEDTAVYYCYAYRWTTRTTYWGQGTLVTVSS |
| Exemplary anti-target (DLL3) | EVQLVESGGGLVQPGGSLTLSCAASGSSVSFLSMAWYRQAPG |
| (SEQ ID NO: 580) | KKRELVAGISVDGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSRVSFLSMAWYRQAPG |
| (SEQ ID NO: 581) | KKRELVAGISADGSTGYIDSVKGRFTISRDNAKNSVYLQMNSLR |
| | AEDTAVYYCYAYRWATRYTYWGQGTLVTVSS |
| Exemplary anti-target (DLL3) | EVQLVESGGGLVQPGGSLTLSCVASGSSVKFLSMAWYRQAPG |
| (SEQ ID NO: 582) | KKRELVAGISGDGSTTYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSSVRFLSMAWYRQAPG |
| (SEQ ID NO: 583) | KKRELVAGISTDGSTDYIDSVKGRFTISRDNAKNSVYLQMNSLR |
| | AEDTAVYYCYALRWTTRYTYWGQGTLVTVSS |
| Exemplary anti-target (DLL3) | EVQLVESGGGLVQPGGSLTLSCAASGSSVSQLSMAWYRQAPG |
| (SEQ ID NO: 584) | KKRELVAGISADGSTDYFDSVKGRFTISRDNAKNSVYLQMNSLR |
| | AEDTAVYYCYAYRWTTRGTYWGQGTLVTVSS |
| Exemplary anti-target (DLL3) | EVQLVESGGGLVQPGGSLTLSCAASGSRVSFLSMAWYRQAPG |
| (SEQ ID NO: 585) | KKRELVAGISADGSTSYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASKSSVSFLSMAWYRQAPGK |
| (SEQ ID NO: 586) | KRELVAGISADGSTDYIDSVKGRFTISRDNAKNSVYLQMNSLRA |
| | EDTAVYYCYAYRWTTRATYWGQGTLVTVSS |
| Exemplary anti-target (DLL3) | EVQLVESGGGLVQPGGSLTLSCAASGSSVSFLSMAWYRQAPG |
| (SEQ ID NO: 587) | KKRELVAGISADGSTAYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSSVKFLSMAWYRQAPG |
| (SEQ ID NO: 588) | KKRELVAGISADGSTVYIDSVKGRFTISRDNAKNSVYLQMNSLR |
| | AEDTAVYYCYAYRWPTRYTYWGQGTLVTVSS |
| Exemplary anti-target (DLL3) | EVQLVESGGGLVQPGGSLTLSCAASGSSVRFLSMAWYRQAPG |
| (SEQ ID NO: 589) | KKRELVAGISQDGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVRFLSMAWYRQAPG |
| (SEQ ID NO: 590) | KKRELVAGISNDGSTDYIDSVKGRFTISRDNAKNSVYLQMNSLR |
| | AEDTAVYYCYAYRWKTRYTYWGQGTLVTVSS |
| Exemplary anti-target (DLL3) | EVQLVESGGGLVQPGGSLTLSCAASGSRVSFLSMAWYRQAPG |
| (SEQ ID NO: 591) | KKRELVAGISARGSTDYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSSVSFLSLAWYRQAPGK |
| (SEQ ID NO: 592) | KRELVAGISADGSTDYIDSVKGRFTISRDNAKNSVYLQMNSLRA |
| | EDTAVYYCYAYRWKTRRTYWGQGTLVTVSS |
| Exemplary anti-target (DLL3) | EVQLVESGGGLVQPGGSLTLSCAASGSSVRFLSMAWYRQAPG |
| (SEQ ID NO: 593) | KKRELVAGISRDGSTDYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSKVSFLSMAWYRQAPG |
| (SEQ ID NO: 594) | KKRELVAGISADGSTLYIDSVKGRFTISRDNAKNSVYLQMNSLR |
| | AEDTAVYYCYAYRWTTRYRYWGQGTLVTVSS |
| Exemplary anti-target (DLL3) | EVQLVESGGGLVQPGGSLTLSCAASGSSVSFLSMAWYRQAPG |
| (SEQ ID NO: 595) | KKRELVAGISADGSTNYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVRFLSMAWYRQAPG |
| (SEQ ID NO: 596) | KKRELVAGISADGSTVYIDSVKGRFTISRDNAKNSVYLQMNSLR |
| | AEDTAVYYCYAYRWTTRYKYWGQGTLVTVSS |
| Exemplary anti-target (DLL3) | EVQLVESGGGLVQPGGSLTLSCAASGSKVSFLSMAWYRQAPG |
| (SEQ ID NO: 597) | KKRELVAGISADGSTTYIDSVKGRFTISRDNAKNSVYLQMNSLR |
| | AEDTAVYYCYAYRWKTRYTYWGQGTLVTVSS |
| Exemplary anti-target (DLL3) | EVQLVESGGGLVQPGGSLTLSCAASGSSVKFLSMAWYRQAPG |
| (SEQ ID NO: 598) | KKRELVAGISADGSTDYIGSVKGRFTISRDNAKNSVYLQMNSLR |
| | AEDTAVYYCYAYRWTTRVTYWGQGTLVTVSS |
| Exemplary anti-target (DLL3) | EVQLVESGGGLVQPGGSLTLSCAASGSSVKFLSMAWYRQAPG |
| (SEQ ID NO: 599) | KKRELVAGISRDGSTDYIDSVKGRFTISRDNAKNSVYLQMNSLR |
| | AEDTAVYYCYAYRWTTRFTYWGQGTLVTVSS |
| Exemplary anti-target (DLL3) | EVQLVESGGGLVQPGGSLTLSCAASGSRVSFLSMAWYRQAPG |
| (SEQ ID NO: 600) | KKRELVAGISADGSTTYIDSVKGRFTISRDNAKNSVYLQMNSLR |
| | AEDTAVYYCYAYRWTTRFTYWGQGTLVTVSS |
| Exemplary anti-target (DLL3) | EVQLVESGGGLVQPGGSLTLSCAASGSSVLFLSMAWYRQAPG |
| (SEQ ID NO: 601) | KKRELVAGVSSDGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSRVSFLSMAWYRQAPG |
| (SEQ ID NO: 602) | KKRELVAGISADGHTDYIDSVKGRFTISRDNAKNSVYLQMNSLR |
| | AEDTAVYYCYAYRWTTRYTHWGQGTLVTVSS |
| Exemplary anti-target (DLL3) | EVQLVESGGGLVQPGGSLTLSCAASGSRVSFLSMAWYRQAPG |
| (SEQ ID NO: 603) | KKRELVAGISADGSTDYFDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVGFLSMAWYRQAPG |
| (SEQ ID NO: 604) | KKRELVAGISADGSTVYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVSFMSMAWYRQAP |
| (SEQ ID NO: 605) | GKKRELVAGISADGSTDYIASVKGRFTISRDNAKNSVYLQMNSL |
| | RAEDTAVYYCYAYRWTTRSTYWGQGTLVTVSS |
| Exemplary anti-target (DLL3) | EVQLVESGGGLVQPGGSLTLSCAASGSSVSFLSMAWYRQAPG |
| (SEQ ID NO: 606) | KKRELVAGISADGSTDYISSVKGRFTISRDNAKNSVYLQMNSLR |
| | AEDTAVYYCYAYSWTTRYTYWGQGTLVTVSS |
| Exemplary anti-target (DLL3) | EVQLVESGGGLVQPGGSLTLSCAASGSSVTFLSMAWYRQAPG |
| (SEQ ID NO: 607) | KKRELVAGISADGSTVYIDSVKGRFTISRDNAKNSVYLQMNSLR |
| | AEDTAVYYCYAYRWTTRGTYWGQGTLVTVSS |
| Exemplary anti-target (DLL3) | EVQLVESGGGLVQPGGSLTLSCAASGSSVRFLSMAWYRQAPG |
| (SEQ ID NO: 608) | KKRELVAGISADGSTVYIDSVKGRFTISRDNAKNSVYLQMNSLR |
| | AEDTAVYYCYAYRWKTRYTYWGQGTLVTVSS |
| Exemplary anti-target (DLL3) | EVQLVESGGGLVQPGGSLTLSCAASGSKVSFLSMAWYRQAPG |
| (SEQ ID NO: 609) | KKRELVAGISADGSTTYIDSVKGRFTISRDNAKNSVYLQMNSLR |
| | AEDTAVYYCYAYRWTTRFTYWGQGTLVTVSS |
| Exemplary anti-target (DLL3) | EVQLVESGGGLVQPGGSLTLSCAASGSKVSFMSMAWYRQAP |
| (SEQ ID NO: 610) | GKKRELVAGISVDGSTDYIDSVKGRFTISRDNAKNSVYLQMNSL |
| | RAEDTAVYYCYAYRWRTRYTYWGQGTLVTVSS |
| Exemplary anti-target (DLL3) | EVQLVESGGGLVQPGGSLTLSCAASGSSVSNLSMAWYRQAPG |
| (SEQ ID NO: 611) | KKRELVAGISADGSTAYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASNSSVSKLSMAWYRQAPG |
| (SEQ ID NO: 612) | KKRELVAGISADGSTAYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSKVSFLSMAWYRQAPG |
| (SEQ ID NO: 613) | KKRELVAGISADGSKDYIDSVKGRFTISRDNAKNSVYLQMNSLR |
| | AEDTAVYYCYAYRWTTRLTYWGQGTLVTVSS |
| Exemplary anti-target (DLL3) | EVQLVESGGGLVQPGGSLTLSCVASGSQVSFLSMAWYRQAPG |
| (SEQ ID NO: 614) | KKRELVAGISADGSTDYFDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSKVSFMSMAWYRQAP |
| (SEQ ID NO: 615) | GKKRELVAGISADGSTDYIDSVKGRFTISRDNAKNSVYLQMNSL |
| | RAEDTAVYYCYAYRWTTRLTYWGQGTLVTVSS |
| Exemplary anti-target (DLL3) | EVQLVESGGGLVQPGGSLTLSCAASGSRVSFLSMAWYRQAPG |
| (SEQ ID NO: 616) | KKRELVAGISADGSTVYIDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVRFLSMAWYRQAPG |
| (SEQ ID NO: 617) | KKRELVAGISADGSTVYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGSKVSFLSMAWYRQAPG |
| (SEQ ID NO: 618) | KKRELVAGISARGSTDYIDSVKGRFTISRDNAKNSVYLQMNSLR |
| | AEDTAVYYCYAYQWTTRYTYWGQGTLVTVSS |
| Exemplary anti-target (DLL3) | EVQLVESGGGLVQPGGSLTLSCAASGSSVRFLSMAWYRQAPG |
| (SEQ ID NO: 619) | KKRELVAGISATGSTDYIDSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVSFLSIAWYRQAPGK |
| (SEQ ID NO: 620) | KRELVAGISKDGSTDYIDSVKGRFTISRDNAKNSVYLQMNSLRA |
| | EDTAVYYCYAYRWTTRMTYWGQGTLVTVSS |
| Exemplary anti-target (DLL3) | EVQLVESGGGLVQPGGSLTLSCAASGSSSSFLSMAWYRQAPGK |
| (SEQ ID NO: 621) | KRELVAGISADGSTVYIDSVKGRFTISRDNAKNSVYLQMNSLRA |
| | EDTAVYYCYAYRWTTRRTYWGQGTLVTVSS |
| Exemplary anti-target (DLL3) | EVQLVESGGGLVQPGGSLTLSCAASGSSVKFLSMAWYRQAPG |
| (SEQ ID NO: 622) | KKRELVAGISPDGSTDYIDSVKGRFTISRDNAKNSVYLQMNSLR |
| | AEDTAVYYCYAYRWTTRYRYWGQGTLVTVSS |
| Exemplary anti-target (DLL3) | EVQLVESGGGLVQPGGSLTLSCAASGSSVNFLSMAWYRQAPG |
| (SEQ ID NO: 623) | KKRELVAGISADGSTHYIDSVKGRFTISRDNAKNSVYLQMNSLR |
| | AEDTAVYYCYAYRWLTRYTYWGQGTLVTVSS |
| Exemplary anti-target (DLL3) | EVQLVESGGGLVQPGGSLTLSCAASGSSVKFLSMAWYRQAPG |
| (SEQ ID NO: 624) | KKRELVAGISADGSTDYILSVKGRFTISRDNAKNSVYLQMNSLR |
| | AEDTAVYYCYAYEWTTRYTYWGQGTLVTVSS |
| Exemplary anti-target (DLL3) | EVQLVESGGGLVQPGGSLTLSCAASGSSVSFLSMAWYRQAPG |
| (SEQ ID NO: 625) | KKRELVAGISADGSTDYIHSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVRFLSMAWYRQAPG |
| (SEQ ID NO: 626) | KKRELVAGISVDGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVSFLSVAWYRQAPGK |
| (SEQ ID NO: 627) | KRELVAGISRDGSTDYIDSVKGRFTISRDNAKNSVYLQMNSLRA |
| | EDTAVYYCYAYRWRTRYTYWGQGTLVTVSS |
| Exemplary anti-target (DLL3) | EVQLVESGGGLVQPGGSLTLSCAASGSQVSFLSMAWYRQAPG |
| (SEQ ID NO: 628) | KKRELVAGISADGSTVYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGTSVSFLSMAWYRQAPG |
| (SEQ ID NO: 629) | KKRELVAGISADGSTDYIRSVKGRFTISRDNAKNSVYLQMNSLR |
| | AEDTAVYYCYAYRWTTRLTYWGQGTLVTVSS |
| Exemplary anti-target (DLL3) | EVQLVESGGGLVQPGGSLTLSCAASGSRVSFLSMAWYRQAPG |
| (SEQ ID NO: 630) | KKRELVAGISADGSTMYIDSVKGRFTISRDNAKNSVYLQMNSLR |
| | AEDTAVYYCYAYRWTTRLTYWGQGTLVTVSS |
| Exemplary anti-target (DLL3) | EVQLVESGGGLVQPGGSLTLSCAASGSSVKFLSMAWYRQAPG |
| (SEQ ID NO: 631) | KKRELVAGISTDGSTDYIDSVKGRFTISRDNAKNSVYLQMNSLR |
| | AEDTAVYYCYAYKWTTRYTYWGQGTLVTVSS |
| Exemplary anti-target (DLL3) | EVQLVESGGGLVQPGGSLTLSCAASGSSVSFLSSAWYRQAPGK |
| (SEQ ID NO: 632) | KRELVAGISADGSTLYIDSVKGRFTISRDNAKNSVYLQMNSLRA |
| | EDTAVYYCYAYRWTTRSTYWGQGTLVTVSS |
| Exemplary anti-target (DLL3) | EVQLVESGGGLVQPGGSLTLSCAASGSSVKFLSMAWYRQAPG |
| (SEQ ID NO: 633) | KKRELVAGISADGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSSVSFLSMAWYRQAPG |
| (SEQ ID NO: 634) | KKRELVAGISATGSTDYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSTVSFLSMAWYRQAPG |
| (SEQ ID NO: 635) | KKRELVAGISHDGSTDYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVQFLSMAWYRQAPG |
| (SEQ ID NO: 636) | KKRELVAGISYDGSTDYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASRSSVSFLSMAWYRQAPG |
| (SEQ ID NO: 637) | KKRELVAGISTDGSTDYIDSVKGRFTISRDNAKNSVYLQMNSLR |
| | AEDTAVYYCYAYRWLTRYTYWGQGTLVTVSS |
| Exemplary anti-target (DLL3) | EVQLVESGGGLVQPGGSLTLSCAASGSKVSFLSMAWYRQAPG |
| (SEQ ID NO: 638) | KKRELVAGISADGSTAYIDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSRVSFLSMAWYRQAPG |
| (SEQ ID NO: 639) | KKRELVAGISADGSTDYIESVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVSFLSMAWYRQAPG |
| (SEQ ID NO: 640) | KKRELVAGISIDGSTDYIKSVKGRFTISRDNAKNSVYLQMNSLRA |
| | EDTAVYYCYAYRWTTRYRYWGQGTLVTVSS |
| Exemplary anti-target (DLL3) | EVQLVESGGGLVQPGGSLTLSCAASGSKVSFLSMAWYRQAPG |
| (SEQ ID NO: 641) | KKRELVAGISADGSKDYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVRFLSMAWYRQAPG |
| (SEQ ID NO: 642) | KKRELVAGISADGSTVYIDSVKGRFTISRDNAKNSVYLQMNSLR |
| | AEDTAVYYCYAYRWPTRYTYWGQGTLVTVSS |
| Exemplary anti-target (DLL3) | EVQLVESGGGLVQPGGSLTLSCAASGSSVKFLSMAWYRQAPG |
| (SEQ ID NO: 643) | KKRELVAGISRDGSTDYIDSVKGRFTISRDNAKNSVYLQMNSLR |
| | AEDTAVYYCYAYRWTTRHTYWGQGTLVTVSS |
| Exemplary anti-target (DLL3) | EVQLVESGGGLVQPGGSLTLSCAASGSSVKFLSMAWYRQAPG |
| (SEQ ID NO: 644) | KKRELVAGISADGSTDYIHSVKGRFTISRDNAKNSVYLQMNSLR |
| | AEDTAVYYCYAYRWTRRYTYWGQGTLVTVSS |
| Exemplary anti-target (DLL3) | EVQLVESGGGLVQPGGSLTLSCAASGSSVSILSMAWYRQAPGK |
| (SEQ ID NO: 645) | KRELVAGISADGSTIYIDSVKGRFTISRDNAKNSVYLQMNSLRAE |
| | DTAVYYCYAYRWHTRYTYWGQGTLVTVSS |
| Exemplary anti-target (DLL3) | EVQLVESGGGLVQPGGSLTLSCAASGSSVSFLSVAWYRQAPGK |
| (SEQ ID NO: 646) | KRELVAGISANGSTDYIDSVKGRFTISRDNAKNSVYLQMNSLRA |
| | EDTAVYYCYAYRWTNRYTYWGQGTLVTVSS |
| Exemplary anti-target (DLL3) | EVQLVESGGGLVQPGGSLTLSCAASGSSVRFLSMAWYRQAPG |
| (SEQ ID NO: 647) | KKRELVAGISTDGSTDYIDSVKGRFTISRDNAKNSVYLQMNSLR |
| | AEDTAVYYCYAYRWTTRYRYWGQGTLVTVSS |
| Exemplary anti-target (DLL3) | EVQLVESGGGLVQPGGSLTLSCAASGSRVSFLSMAWYRQAPG |
| (SEQ ID NO: 648) | KKRELVAGISYDGSTDYIDSVKGRFTISRDNAKNSVYLQMNSLR |
| | AEDTAVYYCYAYRWTTRRTYWGQGTLVTVSS |
| Exemplary anti-target (DLL3) | EVQLVESGGGLVQPGGSLTLSCAASGHSVSFLSMAWYRQAPG |
| (SEQ ID NO: 649) | KKRELVAGISADGSTDYIASVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSSVRFLSMAWYRQAPG |
| (SEQ ID NO: 650) | KKRELVAGISADGSTDYIGSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSSVSFLSMAWYRQAPG |
| (SEQ ID NO: 651) | KKRELVAGISANGSTDYYDSVKGRFTISRDNAKNSVYLQMNSLR |
| | AEDTAVYYCYAYRWRTRYTYWGQGTLVTVSS |
| Exemplary anti-target (DLL3) | EVQLVESGGGLVQPGGSLTLSCAASGSRVSFLSMAWYRQAPG |
| (SEQ ID NO: 652) | KKRELVAGISADGSTSYIDSVKGRFTISRDNAKNSVYLQMNSLR |
| | AEDTAVYYCYAYRWTTRYTYWGQGTLVTVSS |
| Exemplary anti-target (DLL3) | EVQLVESGGGLVQPGGSLTLSCAASGSSVKFLSMAWYRQAPG |
| (SEQ ID NO: 653) | KKRELVAGVSADGSTDYIDSVKGRFTISRDNAKNSVYLQMNSL |
| | RAEDTAVYYCYAYEWTTRYTYWGQGTLVTVSS |
| Exemplary anti-target (DLL3) | EVQLVESGGGLVQPGGSLTLSCAASGSSVSRLSMAWYRQAPG |
| (SEQ ID NO: 654) | KKRELVAGISARGSTDYIDSVKGRFTISRDNAKNSVYLQMNSLR |
| | AEDTAVYYCYAYRWTTRSTYWGQGTLVTVSS |
| Exemplary anti-target (DLL3) | EVQLVESGGGLVQPGGSLTLSCAASGRSVSFLSMAWYRQAPG |
| (SEQ ID NO: 655) | KKRELVAGISADGSTIYIDSVKGRFTISRDNAKNSVYLQMNSLRA |
| | EDTAVYYCYAYRWTTRYTYWGQGTLVTVSS |
| Exemplary anti-target (DLL3) | EVQLVESGGGLVQPGGSLTLSCAASGRSVSFLSMAWYRQAPG |
| (SEQ ID NO: 656) | KKRELVAGISANGSTDYIDSVKGRFTISRDNAKNSVYLQMNSLR |
| | AEDTAVYYCYAYRWSTRYTYWGQGTLVTVSS |
| Exemplary anti-target (DLL3) | EVQLVESGGGLVQPGGSLTLSCAASGSSVKFLSMAWYRQAPG |
| (SEQ ID NO: 657) | KKRELVAGISADGSTDYVDSVKGRFTISRDNAKNSVYLQMNSL |
| | RAEDTAVYYCYAYRWSTRYTYWGQGTLVTVSS |
| Exemplary anti-target (DLL3) | EVQLVESGGGLVQPGGSLTLSCAASGSSVSKLSMAWYRQAPG |
| (SEQ ID NO: 658) | KKRELVAGISADGSTDYRDSVKGRFTISRDNAKNSVYLQMNSLR |
| | AEDTAVYYCYAYRWTYRYTYWGQGTLVTVSS |
| Exemplary anti-target (DLL3) | EVQLVESGGGLVQPGGSLTLSCAASGSSVSRLSMAWYRQAPG |
| (SEQ ID NO: 659) | KKRELVAGISVDGSTDYIDSVKGRFTISRDNAKNSVYLQMNSLR |
| | AEDTAVYYCYAYRWTTRLTYWGQGTLVTVSS |
| Exemplary anti-target (DLL3) | EVQLVESGGGLVQPGGSLTLSCAASGSSVKFLSLAWYRQAPGK |
| (SEQ ID NO: 660) | KRELVAGISADGSTDYILSVKGRFTISRDNAKNSVYLQMNSLRA |
| | EDTAVYYCYAYEWTTRYTYWGQGTLVTVSS |
| Exemplary anti-target (DLL3) | EVQLVESGGGLVQPGGSLTLSCAASGSSVSRLSLAWYRQAPGK |
| (SEQ ID NO: 661) | KRELVAGISVDGSTDYIDSVKGRFTISRDNAKNSVYLQMNSLRA |
| | EDTAVYYCYAYRWTTRLTYWGQGTLVTVSS |
| Exemplary anti-target (DLL3) | EVQLVESGGGLVQPGGSLTLSCVASGTSSSINAMGWYRRAPG |
| (SEQ ID NO: 662) | KQRELVAGISSDGSKVFNESVKGRFTISRDNAKNSVYLQMNSLR |
| | AEDTAVYYCYYFRPAAGSPMRYWGQGTLVTVSS |
| Exemplary anti-target (DLL3) | EVQLVESGGGLVQPGGSLTLSCVASGTTSSINAIGWYRRAPGK |
| (SEQ ID NO: 663) | QRELVAGISSDGSEVYTDSVKGRFTISRDNAKNSVYLQMNSLRA |
| | EDTAVYYCYYFRTVDGSPLRYWGQGTLVTVSS |
| Exemplary anti-target (DLL3) | EVQLVESGGGLVQPGGSLTLSCVASGSTSSINAMAWYRRAPG |
| (SEQ ID NO: 664) | KQRELVAGISSDDSNVYYESVKGRFTISRDNAKNSVYLQMNSLR |
| | AEDTAVYYCYYFRTVSGSSKRYWGQGTLVTVSS |
| Exemplary anti-target (DLL3) | EVQLVESGGGLVQPGGSLTLSCVASGQTYRVNAFGWYRRAPG |
| (SEQ ID NO: 665) | KQRELVAGISSDGSKVYADSVKGRFTISRDNAKNSVYLQMNSL |
| | RAEDTAVYYCYYFSAGSGTEMSYWGQGTLVTVSS |
| Exemplary anti-target (DLL3) | EVQLVESGGGLVQPGGSLTLSCVASGSTSSINAMAWYRRAPG |
| (SEQ ID NO: 666) | KQRELVAGISSDESTLYVDSVKGRFTISRDNAKNSVYLQMNSLR |
| | AEDTAVYYCYYFGSLSGSSTTYWGQGTLVTVSS |
| Exemplary anti-target (DLL3) | EVQLVESGGGLVQPGGSLTLSCVASGSASLTNATGWYRRAPGK |
| (SEQ ID NO: 667) | QRELVAGISSDDSKVYSDSVKGRFTISRDNAKNSVYLQMNSLRA |
| | EDTAVYYCYYFGSVSGSWTRYWGQGTLVTVSS |
| Exemplary anti-target (DLL3) | EVQLVESGGGLVQPGGSLTLSCVASGYPSLNNAMGWYRRAPG |
| (SEQ ID NO: 668) | KQRELVAGISSDGSQVYGASVKGRFTISRDNAKNSVYLQMNSL |
| | RAEDTAVYYCYYFRLVSGSSMSYWGQGTLVTVSS |
| Exemplary anti-target (DLL3) | EVQLVESGGGLVQPGGSLTLSCVASGSSSTINAIGWYRRAPGK |
| (SEQ ID NO: 669) | QRELVAGISSDGSKVYADSVKGRFTISRDNAKNSVYLQMNSLR |
| | AEDTAVYYCYYFRTGSGTSKSYWGQGTLVTVSS |
| Exemplary anti-target (DLL3) | EVQLVESGGGLVQPGGSLTLSCVASGSTSYINAMGWYRRAPG |
| (SEQ ID NO: 670) | KQRELVAGISSDGSNMYADSVKGRFTISRDNAKNSVYLQMNSL |
| | RAEDTAVYYCYYFSNMSGTTRRYWGQGTLVTVSS |
| Exemplary anti-target (DLL3) | EVQLVESGGGLVQPGGSLTLSCVASGSTSSVNALGWYRRAPGK |
| (SEQ ID NO: 671) | QRELVAGISSDGSKVYTDSVKGRFTISRDNAKNSVYLQMNSLR |
| | AEDTAVYYCYYFRTVPGSAMGYWGQGTLVTVSS |
| Exemplary anti-target (DLL3) | EVQLVESGGGLVQPGGSLTLSCVASGSTSLSNAVGWYRRAPGK |
| (SEQ ID NO: 672) | QRELVAGISSDGSKVSAESVKGRFTISRDNAKNSVYLQMNSLRA |
| | EDTAVYYCYYFRAESGSSMGYWGQGTLVTVSS |
| Exemplary anti-target (DLL3) | EVQLVESGGGLVQPGGSLTLSCVASGSTSSTNAIGWYRRAPGK |
| (SEQ ID NO: 673) | QRELVAGISSDGSKVYDDSVKGRFTISRDNAKNSVYLQMNSLR |
| | AEDTAVYYCYYFRTLYGSSRSYWGQGTLVTVSS |
| Exemplary anti-target (DLL3) | EVQLVESGGGLVQPGGSLTLSCVASGLTSTINAMGWYRRAPG |
| (SEQ ID NO: 674) | KQRELVAGISSDGSKVYDDSVKGRFTISRDNAKNSVYLQMNSL |
| | RAEDTAVYYCYYFSPFSGSDTGYWGQGTLVTVSS |
| Exemplary anti-target (DLL3) | EVQLVESGGGLVQPGGSLTLSCVASGVSPSKNAIGWYRRAPGK |
| (SEQ ID NO: 675) | QRELVAGISSDGSAVYVGSVKGRFTISRDNAKNSVYLQMNSLR |
| | AEDTAVYYCYYFSTFSGSSISYWGQGTLVTVSS |
| Exemplary anti-target (DLL3) | EVQLVESGGGLVQPGGSLTLSCVASGSTSSINAVGWYRRAPGK |
| (SEQ ID NO: 676) | QRELVAGISSDGSYVYSESVKGRFTISRDNAKNSVYLQMNSLRA |
| | EDTAVYYCYYFRTLAGSEMRYWGQGTLVTVSS |
| Exemplary anti-target (DLL3) | EVQLVESGGGLVQPGGSLTLSCVASGSTTMNNAMAWYRRAP |
| (SEQ ID NO: 677) | GKQRELVAGISSDSSHVYADSVKGRFTISRDNAKNSVYLQMNS |
| | LRAEDTAVYYCYYFRTVSGSGVRYWGQGTLVTVSS |
| Exemplary anti-target (DLL3) | EVQLVESGGGLVQPGGSLTLSCVASGSTSKINAIGWYRRAPGK |
| (SEQ ID NO: 678) | QRELVAGISSDSSIVYTDSVKGRFTISRDNAKNSVYLQMNSLRA |
| | EDTAVYYCYYFRPGAGHSNSYWGQGTLVTVSS |
| Exemplary anti-target (DLL3) | EVQLVESGGGLVQPGGSLTLSCVASGQTTALNAMGWYRRAP |
| (SEQ ID NO: 679) | GKQRELVAGISSDGSEVNTDSVKGRFTISRDNAKNSVYLQMNS |
| | LRAEDTAVYYCYYFRRASGTAMSYWGQGTLVTVSS |
| Exemplary anti-target (DLL3) | EVQLVESGGGLVQPGGSLTLSCVASGATSSINAIGWYRRAPGK |
| (SEQ ID NO: 680) | QRELVAGISSDGSKLSSDSVKGRFTISRDNAKNSVYLQMNSLRA |
| | EDTAVYYCYYFTSASGTDLSYWGQGTLVTVSS |
| Exemplary anti-target (DLL3) | EVQLVESGGGLVQPGGSLTLSCVASGSTSTINAMGWYRRAPG |
| (SEQ ID NO: 681) | KQRELVAGISSDNSKVYADSVKGRFTISRDNAKNSVYLQMNSL |
| | RAEDTAVYYCYYFRSANGSSKRYWGQGTLVTVSS |
| Exemplary anti-target (DLL3) | EVQLVESGGGLVQPGGSLTLSCVASGSTSSINAMGWYRRAPG |
| (SEQ ID NO: 682) | KQRELVAGISSDGSRVYFDSVKGRFTISRDNAKNSVYLQMNSLR |
| | AEDTAVYYCYYFKTIAGAGMRYWGQGTLVTVSS |
| Exemplary anti-target (DLL3) | EVQLVESGGGLVQPGGSLTLSCVASGSTSLVNAMGWYRRAPG |
| (SEQ ID NO: 683) | KQRELVAGISSDGSLVYAESVKGRFTISRDNAKNSVYLQMNSLR |
| | AEDTAVYYCYYFRYGSGSSLSYWGQGTLVTVSS |
| Exemplary anti-target (DLL3) | EVQLVESGGGLVQPGGSLTLSCVASGSTSLNNAIGWYRRAPGK |
| (SEQ ID NO: 684) | QRELVAGISSDGSVVYVDSVKGRFTISRDNAKNSVYLQMNSLR |
| | AEDTAVYYCYYFRTVPGASMKYWGQGTLVTVSS |
| Exemplary anti-target (DLL3) | EVQLVESGGGLVQPGGSLTLSCVASGSTSPVNAMAWYRRAPG |
| (SEQ ID NO: 685) | KQRELVAGISSDGSKVYVDSVKGRFTISRDNAKNSVYLQMNSL |
| | RAEDTAVYYCYYFRTVDGSAISYWGQGTLVTVSS |
| Exemplary anti-target (DLL3) | EVQLVESGGGLVQPGGSLTLSCVASGTTSSMNAIGWYRRAPG |
| (SEQ ID NO: 686) | KQRELVAGISSDGSKLYDESVKGRFTISRDNAKNSVYLQMNSLR |
| | AEDTAVYYCYYFRTVKGSGGSYWGQGTLVTVSS |
| Exemplary anti-target (DLL3) | EVQLVESGGGLVQPGGSLTLSCVASGETSSINAMAWYRRAPG |
| (SEQ ID NO: 687) | KQRELVAGISSDYSKLYADSVKGRFTISRDNAKNSVYLQMNSLR |
| | AEDTAVYYCYYFRTVSGSSRGYWGQGTLVTVSS |
| Exemplary anti-target (DLL3) | EVQLVESGGGLVQPGGSLTLSCVASGSTSTINAIGWYRRAPGK |
| (SEQ ID NO: 688) | QRELVAGISSDSSKVYTESVKGRFTISRDNAKNSVYLQMNSLRA |
| | EDTAVYYCYYFRPGPGSQMAYWGQGTLVTVSS |
| Exemplary anti-target (DLL3) | EVQLVESGGGLVQPGGSLTLSCVASGSTYSMNAMGWYRRAP |
| (SEQ ID NO: 689) | GKQRELVAGISSDGSQVYVDSVKGRFTISRDNAKNSVYLQMNS |
| | LRAEDTAVYYCYYFRTVAGSASGYWGQGTLVTVSS |
| Exemplary anti-target (DLL3) | EVQLVESGGGLVQPGGSLTLSCVASGSPSSINAYGWYRRAPGK |
| (SEQ ID NO: 690) | QRELVAGISSDGSKVYSDSVKGRFTISRDNAKNSVYLQMNSLRA |
| | EDTAVYYCYYFRTVSGSSYSYWGQGTLVTVSS |
| Exemplary anti-target (DLL3) | EVQLVESGGGLVQPGGSLTLSCVASGSTSTINAIGWYRRAPGK |
| (SEQ ID NO: 691) | QRELVAGISSDGSKVYVDSVKGRFTISRDNAKNSVYLQMNSLR |
| | AEDTAVYYCYYFINLKGSSMAYWGQGTLVTVSS |
| Exemplary anti-target (DLL3) | EVQLVESGGGLVQPGGSLTLSCVASGSTSSINAIGWYRRAPGK |
| (SEQ ID NO: 692) | QRELVAGISSDGSKVYADSVKGRFTISRDNAKNSVYLQMNSLR |
| | AEDTAVYYCYYFRMVTGSYGGYWGQGTLVTVSS |
| Exemplary anti-target (DLL3) | EVQLVESGGGLVQPGGSLTLSCVASGSISSINAMGWYRRAPGK |
| (SEQ ID NO: 693) | QRELVAGISSDGSSVYADSVKGRFTISRDNAKNSVYLQMNSLR |
| | AEDTAVYYCYYFKSSYGLPMRYWGQGTLVTVSS |
| Exemplary anti-target (DLL3) | EVQLVESGGGLVQPGGSLTLSCVASGSTQVNNAMAWYRRAP |
| (SEQ ID NO: 694) | GKQRELVAGISSDGSQVYYGSVKGRFTISRDNAKNSVYLQMNS |
| | LRAEDTAVYYCYYFKTVSGQSLRYWGQGTLVTVSS |
| Exemplary anti-target (DLL3) | EVQLVESGGGLVQPGGSLTLSCVASGSTASFNAMAWYRRAPG |
| (SEQ ID NO: 695) | KQRELVAGISSDGSKVYTDSVKGRFTISRDNAKNSVYLQMNSLR |
| | AEDTAVYYCYYFRTVTGRAARYWGQGTLVTVSS |
| Exemplary anti-target (DLL3) | EVQLVESGGGLVQPGGSLTLSCVASGSPLSINAIGWYRRAPGK |
| (SEQ ID NO: 696) | QRELVAGISSDGSKVSADSVKGRFTISRDNAKNSVYLQMNSLR |
| | AEDTAVYYCYYFGPAIGASRTYWGQGTLVTVSS |
| Exemplary anti-target (DLL3) | EVQLVESGGGLVQPGGSLTLSCVASGSTTFINAIGWYRRAPGK |
| (SEQ ID NO: 697) | QRELVAGISSDGSKVYEDSVKGRFTISRDNAKNSVYLQMNSLR |
| | AEDTAVYYCYYFRTVSGAPKSYWGQGTLVTVSS |
| Exemplary anti-target (DLL3) | EVQLVESGGGLVQPGGSLTLSCVASGSTSSINAIGWYRRAPGK |
| (SEQ ID NO: 698) | QRELVAGISSDRSKVYADSVKGRFTISRDNAKNSVYLQMNSLR |
| | AEDTAVYYCYYFHTVSGSSMSYWGQGTLVTVSS |
| Exemplary anti-target (DLL3) | EVQLVESGGGLVQPGGSLTLSCVASGETDTINAVGWYRRAPGK |
| (SEQ ID NO: 699) | QRELVAGISSDGSKVYAESVKGRFTISRDNAKNSVYLQMNSLRA |
| | EDTAVYYCYYFRRLEGYSNRYWGQGTLVTVSS |
| Exemplary anti-target (DLL3) | EVQLVESGGGLVQPGGSLTLSCVASGSTSPINAIGWYRRAPGK |
| (SEQ ID NO: 700) | QRELVAGISSDGSVVTTESVKGRFTISRDNAKNSVYLQMNSLRA |
| | EDTAVYYCYYFRTGSGSSMGYWGQGTLVTVSS |
| Exemplary anti-target (DLL3) | EVQLVESGGGLVQPGGSLTLSCVASGSITSSNAMGWYRRAPG |
| (SEQ ID NO: 701) | KQRELVAGISSDGSHVHQESVKGRFTISRDNAKNSVYLQMNSL |
| | RAEDTAVYYCYYFTTVTGSSMSYWGQGTLVTVSS |
| Exemplary anti-target (DLL3) | EVQLVESGGGLVQPGGSLTLSCAASRYSVSNLSMAWYRQAPG |
| (SEQ ID NO: 702) | KKRELVAGISADGSTVYVESVKGRFTISRDNAKNSVYLQMNSLR |
| | AEDTAVYYCYAYYWTERRPYWGQGTLVTVSS |
| Exemplary anti-target (DLL3) | EVQLVESGGGLVQPGDSLTLSCAASMSTVSVLSMAWYRQAPG |
| (SEQ ID NO: 703) | KKRELVAGISSDGSTVYIDSVKGRFTISRDNAKNSVYLQMNSLR |
| | AEDTAIYYCYAYSWDDAHPYWGQGTLVTVSS |
| Exemplary anti-target (DLL3) | EVQLVESGGGLVQPGGSLTLSCAASDSYVSLLSMAWYRQAPG |
| (SEQ ID NO: 704) | KKRELVAGISVDGSTHYVASVKGRFTISRDNAKNSVYLQMNSLR |
| | AEDTAVYYCYAYRWMTRLTYWGQGTLVTVSS |
| Exemplary anti-target (DLL3) | EVQLVESGGGLVQPGGSLTLSCAASDSAVSVLSIAWYRQAPGK |
| (SEQ ID NO: 705) | KRELVAGISTDGSKHYIDSVKGRFTISRDNAKNSVYLQMNSLRA |
| | EDTAVYYCYAYDWADAQPYWGQGTLVTVSS |
| Exemplary anti-target (DLL3) | EVQLVESGGGLVQPGGSLTLSCAASHSSVTSLSLAWYRQAPGK |
| (SEQ ID NO: 706) | KRELVAGISYDGSKYYAESVKGRFTISRDNAKNSVYLQMNSLRA |
| | EDTAVYYCYAYSWTDRLPYWGQGTLVTVSS |
| Exemplary anti-target (DLL3) | EVQLVESGGGLVQPGGSLTLSCAASDSVVKFLSMAWYRQAPG |
| (SEQ ID NO: 707) | KKRELVAGISANGSRTYMESVKGRFTISRDNAKNSVYLQMNSL |
| | RAEDTAVYYCYAYRWATRLPYWGQGTLVTVSS |
| Exemplary anti-target (DLL3) | EVQLVESGGGLVQPGGSLTLSCAASDPSVWNLSMAWYRQAP |
| (SEQ ID NO: 708) | GKKRELVAGISPDGSTDYVDSVKGRFTISRDNAKNSVYLQMNS |
| | LRAEDTAVYYCYAYKWSNRLPYWGQGTLVTVSS |
| Exemplary anti-target (DLL3) | EVQLVESGGGLVQPGGSLTLSCAASGTSVMLLSLAWYRQAPG |
| (SEQ ID NO: 709) | KKRELVAGISPNGSAVYTESVKGRFTISRDNAKNSVYLQMNSLR |
| | AEDTAVYYCYAYGWKTRQPYWGQGTLVTVSS |
| Exemplary anti-target (DLL3) | EVQLVESGGGLVQPGGSLTLSCAASSSPVSNLSLAWYRQAPGK |
| (SEQ ID NO: 710) | KRELVAGISPDGSTAYMESVKGRFTISRDNAKNSVYLQMNSLR |
| | AEDTAVYYCYAYRWPNRRGYWGQGTLVTVSS |
| Exemplary anti-target (DLL3) | EVQLVESGGGLVQPGGSLTLSCAASWRSVLLLSVAWYRQAPG |
| (SEQ ID NO: 711) | KKRELVAGISNDGSTDYIDSVKGRFTISRDNAKNSVYLQMNSLR |
| | AEDTAVYYCYAYDWTTRQRYWGQGTLVTVSS |
| Exemplary anti-target (DLL3) | EVQLVESGGGLVQPGGSLTLSCAASSSSVQYLSMAWYRQAPG |
| (SEQ ID NO: 712) | KKRELVAGISTDGSAVYFDSVKGRFTISRDNAKNSVYLQMNSLR |
| | AEDTAVYYCYAYNWSYAQPYWGQGTLVTVSS |
| Exemplary anti-target (DLL3) | EVQLVESGGGLVQPGGSLTLSCAASGTSVSLLSLAWYRQAPGK |
| (SEQ ID NO: 713) | KRELVAGISTGGSTHYIESVKGRFTISRDNAKNSVYLQMNSLRA |
| | EDTAVYYCYAYNWTDSLQYWGQGTLVTVSS |
| Exemplary anti-target (DLL3) | EVQLVESGGGLVQPGGSLTLSCAASLSSVSNLSIAWYRQAPGKK |
| (SEQ ID NO: 714) | RELVAGISTDGSTVYIDSVKGRFTISRDNAKNSVYLQMNSLRAE |
| | DTAVYYCYAYSWTTSLPYWGQGTLVTVSS |
| Exemplary anti-target (DLL3) | EVQLVESGGGLVQPGGSLTLSCAASMYSVSFLSMAWYRQAPG |
| (SEQ ID NO: 715) | KKRELVAGISNEGSTYYMDSVKGRFTISRDNAKNSVYLQMNSL |
| | RAEDTAVYYCYAYKWRSRSTYWGQGTLVTVSS |
| Exemplary anti-target (DLL3) | EVQLVESGGGLVQPGGSLTLSCAASKSSVSHLSLAWYRQAPGK |
| (SEQ ID NO: 716) | KRELVAGISADGSHVYTNSVKGRFTISRDNAKNSVYLQMNSLR |
| | AEDTAVYYCYAYSQTTRDPYWGQGTLVTVSS |
| Exemplary anti-target (DLL3) | EVQLVESGGGLVQPGGSLTLSCAASYTSVLDLSIAWYRQAPGK |
| (SEQ ID NO: 717) | KRELVAGISDDGSRYYTDSVKGRFTISRDNAKNSVYLQMNSLRA |
| | EDTAVYYCYAYRWTARDTYWGQGTLVTVSS |
| Exemplary anti-target (DLL3) | EVQLVESGGGLVQPGGSLTLSCAASMSDVSFLSMAWYRQAPG |
| (SEQ ID NO: 718) | KKRELVAGISAEGSTLYMESVKGRFTISRDNAKNSVYLQMNSLR |
| | AEDTAVYYCYAYRWTSRLSYWGQGTLVTVSS |
| Exemplary anti-target (DLL3) | EVQLVESGGGLVQPGGSLTLSCAASESSVSFLSSAWYRQAPGK |
| (SEQ ID NO: 719) | KRELVAGISTDGSTVYIDSVKGRFTISRDNAKNSVYLQMNSLRA |
| | EDTAVYYCYAYSWTTRSRYWGQGTLVTVSS |
| Exemplary anti-target (DLL3) | EVQLVESGGGLVQPGGSLTLSCAASGDSVSLLSMAWYRQAPG |
| (SEQ ID NO: 720) | KKRELVAGISANGSTSYIDSVKGRFTISRDNAKNSVYLQMNSLR |
| | AEDTAVYYCYAYNWTSRYRYWGQGTLVTVSS |
| Exemplary anti-target (DLL3) | EVQLVESGGGLVQPGGSLTLSCAASGSDVWYLSLAWYRQAPG |
| (SEQ ID NO: 721) | KKRELVAGISDDGSRHYIESVKGRFTISRDNAKNSVYLQMNSLR |
| | AEDTAVYYCYAYSWKTRFPYWGQGTLVTVSS |
| Exemplary anti-target (DLL3) | EVQLVESGGGLVQPGGSLTLSCAASKSAVAFLSIAWYRQAPGK |
| (SEQ ID NO: 722) | KRELVAGISPDGSTVYIESVKGRFTISRDNAKNSVYLQMNSLRA |
| | EDTAVYYCYAYSWTTRYPYWGQGTLVTVSS |
| Exemplary anti-target (DLL3) | EVQLVESGGGLVQPGGSLTLSCAASFSAVAYLSMAWYRQAPG |
| (SEQ ID NO: 723) | KKRELVAGISDDGSTVYVDSVKGRFTISRDNAKNSVYLQMNSL |
| | RAEDTAVYYCYAYEWTNALPYWGQGTLVTVSS |
| Exemplary anti-target (DLL3) | EVQLVESGGGLVQPGGSLTLSCAASVYSVYDLSTAWYRQAPGK |
| (SEQ ID NO: 724) | KRELVAGISDDGSTVYFDSVKGRFTISRDNAKNSVYLQMNSLRA |
| | EDTAVYYCYAYSWITRSPYWGQGTLVTVSS |
| Exemplary anti-target (DLL3) | EVQLVESGGGLVQPGGSLTLSCAASGDSVSFLSMAWYRQAPG |
| (SEQ ID NO: 725) | KKRELVAGISDEGSTVYIGSVKGRFTISRDNAKNSVYLQMNSLR |
| | AEDTAVYYCYAYSWTTRRQYWGQGTLVTVSS |
| Exemplary anti-target (DLL3) | EVQLVESGGGLVQPGGSLTLSCAASSSSVSLLSLAWYRQAPGKK |
| (SEQ ID NO: 726) | RELVAGISDDGSIVYMDSVKGRFTISRDNAKNSVYLQMNSLRA |
| | EDTAVYYCYAYSWITRSPYWGQGTLVTVSS |
| Exemplary anti-target (DLL3) | EVQLVESGGGLVQPGGSLTLSCAASADSVSFLSIAWYRQAPGK |
| (SEQ ID NO: 727) | KRELVAGISDDGSKHYFDSVKGRFTISRDNAKNSVYLQMNSLR |
| | AEDTAVYYCYAYRWEESRQYWGQGTLVTVSS |
| Exemplary anti-target (DLL3) | EVQLVESGGGLVQPGGSLTLSCAASASSVTLLSIAWYRQAPGKK |
| (SEQ ID NO: 728) | RELVAGISTDGSTDYLHSVKGRFTISRDNAKNSVYLQMNSLRAE |
| | DTAVYYCYAYTWTTRLPYWGQGTLVTVTS |
| Exemplary anti-target (DLL3) | EVQLVESGGGLVQPGGSLTLSCAASADSVSFLSIAWYRQAPGK |
| (SEQ ID NO: 729) | KRELVAGISDDGSKHYFDSVKGRFTISRDNAKNSVYLQMNSLR |
| | AEDTAVYYCYAYRWEESRQYWGQGTLVTVSS |
| Exemplary anti-target (DLL3) | EVQLVESGGGLVQPGGSLTLSCAASGTSVWLLSMAWYRQAP |
| (SEQ ID NO: 730) | GKKRELVAGISYDGSTVYVESVKGRFTISRDNAKNSVYLQMNSL |
| | RAEDTAVYYCYAYSWTTRQPYWGQGTLVTVSS |
| Exemplary anti-target (DLL3) | EVQLVESGGGLVQPGGSLTLSCAASGSSVSILSIAWYRQAPGKK |
| (SEQ ID NO: 731) | RELVAGISDDGSTVYIDSVKGRFTISRDNAKNSVYLQMNSLRAE |
| | DTAVYYCYAYVWGTRLPYWGQGTLVTVSS |
| Exemplary anti-target (DLL3) | EVQLVESGGGLVQPGGSLTLSCAASGTAVSNLSIAWYRQAPGK |
| (SEQ ID NO: 732) | KRELVAGISDDGSTVYVDSVKGRFTISRDNAKNSVYLQMNSLR |
| | AEDTAVYYCYAYEWTNALPYWGQGTLVTVSS |
| Exemplary anti-target (DLL3) | EVQLVESGGGLVQPGGSLTLSCAASGSAVSMLSLAWYRQAPG |
| (SEQ ID NO: 733) | KKRELVAGISDDGSQVYIDSVKGRFTISRDNAKNSVYLQMNSLR |
| | AEDTAVYYCYAYRWEDALTYWGQGTLVTVSS |
| Exemplary anti-target (DLL3) | EVQLVESGGGLVQPGGSLTLSCAASGMTVFFLSMAWYRQAP |
| (SEQ ID NO: 734) | GKKRELVAGISVDGSTVYSDSVKGRFTISRDNAKNSVYLQMNSL |
| | RAEDTAVYYCYAYSWTTRYPYWGQGTLVTVSS |
| Exemplary anti-target (DLL3) | EVQLVESGGGLVQPGGSLTLSCAASQYSVTFLSVAWYRQAPGK |
| (SEQ ID NO: 735) | KRELVAGISDDGSNVYIDSVKGRFTISRDNAKNSVYLQMNSLRA |
| | EDTAVYYCYAYSWIDSLRYWGQGTLVTVSS |
| Exemplary anti-target (DLL3) | EVQLVESGGGLVQPGGSLTLSCAASGETVSFLSLAWYRQAPGK |
| (SEQ ID NO: 736) | KRELVAGISTDGSTVYFVSVKGRFTISRDNAKNSVYLQMNSLRA |
| | EDTAVYYCYAYSWTTPRAYWGQGTLVTVSS |
| Exemplary anti-target (EGFR) | QVQLQESGGGLVQAGGSLRLSCAASGSIAYIYTMDWYRQAPG |
| (SEQ ID NO: 737) | KQRELVATSTRDGNVDYAESVKGRFTISRDNAKNTVYLQMNSL |
| | KPEDTAVYYCNADLRTAVDLIRANYWGQGTQVTVSS |
| Exemplary anti-target (EGFR) | QVQLQESGGGLVQAGDSLRLSCVVSGRTDSWYVMGWFRQA |
| (SEQ ID NO: 738) | PGKDREFVAGVSWSYGNTYYADSVKGRFTASRDNAKNTAYLQ |
| | MNSLNAEDTAVYYCAARVSREVIPTRWDLYNYWGQGTQVTV |
| | SS |
| Exemplary anti-target (EGFR) | QVQLQESGGGSVQPGGSLRVSCVVSRTIISINAMTWYHQAPG |
| (SEQ ID NO: 739) | KRRELVAIITSGGETNYADSVKGRFTISRDNAKNTAYLQMNNLK |
| | PEDTGVYYCNVVPPLGSWGQGTQVTVSS |
| Exemplary anti-target (EGFR) | QVQLQESGGGRVQAGGSLRLSCSASARTLRLYAVGWFRQAPG |
| (SEQ ID NO: 740) | KEREFVAGIGRSERTYYTDSVKGRFTLSRDNAKNTVFLEMNDLE |
| | PEDTAVYFCALTFQTTDMVDVPTTQHEYDYWGRGTQVTVSS |
| Exemplary anti-target (EGFR) | QVQLQESGGGLVQAGGSLRLSCAASGSIAYIYTMDWYRQAPG |
| (SEQ ID NO: 741) | KQRELVATSTHDGNTDYADSVKGRFTISRDNVKNTVYLQMNSL |
| | KPEDTAVYYCNADLRTAVDLIRANYWGQGTQVTVSS |
| Exemplary anti-target (EGFR) | QVQLQESGGGLVQPGGSLRLSCAASGSIAYIYTMDWYRQAPG |
| (SEQ ID NO: 742) | KQRELVATSTRDGNTDYADSVKGRFTISRDNAKDTVYLQMNSL |
| | KPEDTAVYYCNADLRTAVDLIRANYWGQGTQVTVSS |
| Exemplary anti-target (EGFR) | QVQLQESGGGLVQAGGSLTLSCAASGRYQMAWFRQAPEKER |
| (SEQ ID NO: 743) | EFVGTISSGDSTWYTNSVKGRFAISRDSARNTVYLQMNDLKPE |
| | DTAIYYCAAALYYRDSRRAADYPYWGQGTQVTVSS |
| Exemplary anti-target (EGFR) | QVQLQESGGGRVQAGESLRLSCSTSTRTLKLYAVGWFRQAPG |
| (SEQ ID NO: 744) | KERDFVAGIGRSERIYYIDSVKGRFTLSRDNAKNTVFLEMNDLEP |
| | EDTAVYFCAATFQTSDNVGVPTVQHEYDYWGQGTQVTVSS |
| Exemplary anti-target (EGFR) | QVQLQESGGGLVQAGGSLRLSCAASGSIAYIYTMDWYRQAPG |
| (SEQ ID NO: 745) | KQRELVATSTHDGNTDYADSVKGRFTISRDNAKNTVTLQMNSL |
| | KPEDTAVYYCNADLRTAVDLIRANYWGQGTQVTVSS |
| Exemplary anti-target (EGFR) | QVQLQESGGGLVQAGGSLRLSCAASGSIAYIYTMDWYRQAPG |
| (SEQ ID NO: 746) | KQRELVATFTRDGNTDYADSVKGRFTISRDNAKNTVYLQMNSL |
| | KPEDTAVYYCNTDLRTAVDLIRANYWGQGTQVTVSS |
| Exemplary anti-target (EGFR) | QVQLQESGGGLVQAGGSLRLSCAASGSIAYIYTMDWYRQAPG |
| (SEQ ID NO: 747) | KQRELVATSTHDGNTDYADSLKGRFTISRDNAKNTVYLQMNSL |
| | KPEDTAVYYCNADLRTAVDLIRANYWGQGTQVTVSS |
| Exemplary anti-target (EGFR) | QVQLQESGGGSVQAGGSLRLSCAASGRYQMAWFRQAPEKER |
| (SEQ ID NO: 748) | EFVGTISSGDSTWYTNSLKGRFAISRDSARDTVYLQMNDLKPE |
| | DTAVYYCAAALYYRDSRRAADYPYWGQGTQVTVSS |
| Exemplary anti-target (EGFR) | QVQLQESGGGLVQTGGSLRLSCAVSGSIVTINAMTWYRQAPG |
| (SEQ ID NO: 749) | KRRELVAIITSGGETNYADSVKGRFTISRDNAKNTAHLQMNSLN |
| | PEDTGVYYCNVVPPLGSWGQGTQVTVSS |
| Exemplary anti-target (EGFR) | QVQLQESGGGLVQTGGSLRLSCAVSRSIVSIKSMTWYRQAPGK |
| (SEQ ID NO: 750) | RRELVALITSGGETNYSDSVKGRFTISRDNAKNTVYLQMNSLKP |
| | EDTGVYYCNVVPPLGSWGQGTQVTVSS |
| Exemplary anti-target (EGFR) | QVQLQESGGGLVETGGSLRLSCAGSGSTFRHHAMAWFRQTP |
| (SEQ ID NO: 751) | GKEREFVSAINDHGDRTKYLDSVRGRFTISRDNTDNMVYLQM |
| | TDLRPEDTANYSCAAGPLVDYLETTPLVYTYWGHGTQVTVSS |
| Exemplary anti-target (EGFR) | QVQLQESGGGLVQAGGSLRLSCAASGSIAYIYTMDWYRQAPG |
| (SEQ ID NO: 752) | KQRELVATSTHDGNTDYADSVKGRFTISRDNAKNTVYLQMNSL |
| | KPEDTAVYYCNADLRTAVDLIRANYWGQGTQVTVSS |
| Exemplary anti-target (EGFR) | QVQLQESGGGSVQAGGSLTLSCAASGSIAYIYTMDWYRQAPG |
| (SEQ ID NO: 753) | KQRELVATSTHDGNTDYADSVKGRFTISRDNAKNTVYLQMNSL |
| | KPEDTAVYYCNADLRTAVDLIRANYWGQGTQVTVSS |
| Exemplary anti-target (EGFR) | QVQLQESGGGLVQPGGSLRLSCTASVSIFSVNAVDWYRQSPG |
| (SEQ ID NO: 754) | KERELVAIMTSDGSTNYGDSVKGRFTISRDNAKNTVYLQMNNL |
| | KPEDTAVYYCNTVPPRYWGQGTQVTVSS |
| Exemplary anti-target (EGFR) | QVQLQESGGGLVQPGGSLRLSCAASGSIAYIYTMDWYRQAPG |
| (SEQ ID NO: 755) | KQRELVATSTRDGNIDYADSVKGRFTISRDSAKNTVYLQMSSLK |
| | PEDTAVYYCNADLRTAVDLIRANYWGQGTQVTVSS |
| Exemplary anti-target (EGFR) | QVQLQESGGGLVQAGGSLTLSCAASGRYQLAWFRQAPEKVRE |
| (SEQ ID NO: 756) | FVGTISSGDSTWYTNSVKGRFAISRDSARNTVYLQMNDLKPED |
| | TAVYYCAAALYYRDSRRAADYPYWGQGTQVTVSS |
| Exemplary anti-target (EGFR) | QVQLQESGGGSVQAGGSLRLSCAASGRYHMAWFRQAPEKER |
| (SEQ ID NO: 757) | EFVGTISSGDSTWYTNSVKGRFAISRDSARNTAYLQMNDLKPE |
| | DTAVYYCAAALYYGDSRRAADYPYWGQGTQVTVSS |
| Exemplary anti-target (EGFR) | QVQLQESGGGSVQAGGSLRLSCAASGSIAYIYTMDWYRQAPG |
| (SEQ ID NO: 758) | KQRELVATSTHDGNTDYTDSVKGRFTISRDNAKNTVYLQMNSL |
| | KPEDTAVYYCNADLRTAVDLIRANYWGQGTQVTVSS |
| Exemplary anti-target (EGFR) | QVQLQESGGGLVQAGGSLRLSCAASGSIAYIYTMDWYRQTPG |
| (SEQ ID NO: 759) | KQRELVATSTRDANTDYAGSVKGRFTISRDNAKDTVYLQMNSL |
| | KPEDTAVYYCHADLRTAVDLIRANYWGQGTQVTVSS |
| Exemplary anti-target (EGFR) | QVQLQESGGGLVQAGGSLRLSCAASGRYNMAWFRQAPEKER |
| (SEQ ID NO: 760) | EFVGTITSADSTWYTNSVKGRFAITQDSARNTVYLQMNDLKPE |
| | DTAVYYCAAALYYGDSRRAADYPYWGQGTQVTVSS |
| Exemplary anti-target (EGFR) | QVQLQESGGGLVQPGGSLRLSCAASGRYQMAWFRQAPEKER |
| (SEQ ID NO: 761) | EFVGTISSGDSTWYTNSVKGRFAISRDSARTTVYLQMNDLKPE |
| | DTAVYYCAAALYYRDSWRAADYPYWGQGTQVTVSS |
| Exemplary anti-target (EGFR) | QVQLQESGGGLVQPGESLRLSCAATGRYHLAWFRQAPEKERE |
| (SEQ ID NO: 762) | FVGTITSADSTWYTNSVKGRFAITRDSARNTVYLQMNDLKPED |
| | TAVYYCAAALYYGDSRRAADYPYWGQGTQVTVSS |
| Exemplary anti-target (EGFR) | QVQLQESGGGLVQAGGSLKLSCADSGRSFSNYIMGWFRQAP |
| (SEQ ID NO: 763) | GKEREFVAGLGWSPGNTYYADSVKGRFTISRDNAKNMVYLQ |
| | MNSLNPEDTAVYYCAARRGDVIYTTPWNYVYWGQGTQVTVS |
| | S |
| Exemplary anti-target (EGFR) | QVQLQESGGGLVQAGGSLRLSCAASGSIAYIYTMDWYRQAPG |
| (SEQ ID NO: 764) | KQRELVATSTHDGNTDYADSVKGRFTISRDNAKNTVYLQMNSL |
| | KPEDTAVYYCNADLRTPVDLIRANYWGQGTQVTVSS |
| Exemplary anti-target (EGFR) | QVQLQESGGGSVQAGGSLRLSCAAPGRYQMAWFRQAPEKER |
| (SEQ ID NO: 765) | EFVGTISSGDSTWYTNSVKGRFAISRDSARNTVYLQMNDLKSE |
| | DTAVYYCAAALYYRDSRRAIDYPYWGQGTQVTVSS |
| Exemplary anti-target (EGFR) | QVQLQESGGGSVQAGGSLRLSCAASGLTFSSYAMAWFRQAP |
| (SEQ ID NO: 766) | GKQRELVARITSGGTTDYADSVKGRFTISRDNAKNTMYLQMN |
| | SLKPEDTAVYYCAADLTYRNLLLKLPHYWGQGTQVTVSS |
| Exemplary anti-target (EGFR) | QVQLQESGGGLVQAGGSLRLSCAASGNIAYIYTMDWYRQAPG |
| (SEQ ID NO: 767) | KQRELVATSTHDGSTDYADSVKGRFTISRDNAKNTVYLQMNSL |
| | KPEDTAVYYCNADLRTPVDLIRANYWGQGTQVTVSS |
| Exemplary anti-target (EGFR) | QVQLQESGGGLVQPGGSLRLSCAASGSIAYIYTMDWYRQAPG |
| (SEQ ID NO: 768) | KQRELVATSTWDGNTDYADSVKGRFTISRDNAKNTVYLQMNS |
| | LKPEDTAVYYCNADLRTAVDLIRANYWGQGTQVTVSS |
| Exemplary anti-target (EGFR) | QVQLQESGGGLVQAGGSLRLSCAASGSIAYIYTMDWYRQAPG |
| (SEQ ID NO: 769) | KQRELVATSTHDGNTDYADSVKGRFTISRDNAKNTVYLQMSSL |
| | KPDDTAVYYCNADLRTAVDLIRANYWGQGTQVTVSS |
| Exemplary anti-target (EGFR) | QVQLQESGGGLVQAGESLSLSCAASGNDFVITDMHWYRQAP |
| (SEQ ID NO: 770) | GKQREWVATITRFATTNYADSVKGRFTISRDNAKNTWYLQMN |
| | SLKPDDTAVYYCKAIGLRGVPDVNRQFEVWGQGTQVTVSS |
| Exemplary anti-target (EGFR) | QVQLQESGGGLVQAGGSLRLSCAASGAIAYIYGMGWYRQAP |
| (SEQ ID NO: 771) | GNQRELVAAISSGGSTDYADSVKGRFTISRDNAKNTVYLQMSS |
| | LKPEDTAVYYCNADVRTSRNLVRSDYWGQGTQVTVSS |
| Exemplary anti-target (EGFR) | QVQLQESGGGLVQAGGSLRLSCAASGRYHTAWFRQAPEKERE |
| (SEQ ID NO: 772) | FVGTISSGDSTWYTNSVKGRFAISRDSARNTVYLQMNDLKPED |
| | TAVYYCAAALYYGDSRRAGDYPYWGQGTQVTVSS |
| Exemplary anti-target (EGFR) | QVQLQESGGGLVQAGGSLRLSCAASGNIAYIYTMNWYRQAPG |
| (SEQ ID NO: 773) | KQRELVATSTHAGNTDYADSVKGRFTISRDNAKNTVYLQMNSL |
| | KPEDTAVYYCNVDLRTAVDLIRANYWGQGTQVTVSS |
| Exemplary anti-target (EGFR) | QVQLQESGGGLVQPGGSLRLSCAASGNIAYIYTMGWYRQAPG |
| (SEQ ID NO: 774) | KQRELVATSTHDGNSDYADSVKGRFTISRDNAKNTVYLQMNTL |
| | KPDDTAVYYCNADLRTPVDRIRGNFWGQGTQVTVSS |
| Exemplary anti-target (EGFR) | EVQLLESGGGLVQPGGSLTLSCAASGSIAYIYTMDWYRQAPGK |
| (SEQ ID NO: 775) | QRELVATSTRDGNVDYADSVKGRFTISRDNSKNTLYLQMNSLR |
| | AEDTAVYYCNADLRTAVDLIRANYWGLGTQVTVSS |
| Exemplary anti-target (EGFR) | EVQLLESGGGLVQPGGSLTLSCAASGRTDSWYVMGWFRQAP |
| (SEQ ID NO: 776) | GKDREFVAGVSWSYGNTYYADSVKGRFTISRDNSKNTLYLQM |
| | NSLRAEDTAVYYCAARVSREVIPTRWDLYNYWGLGTQVTVSS |
| Exemplary anti-target (EGFR) | EVQLLESGGGLVQPGGSLTLSCAASARTLRLYAVGWFRQAPGK |
| (SEQ ID NO: 777) | EREFVAGIGRSERTYYTDSVKGRFTISRDNSKNTLYLQMNSLRA |
| | EDTAVYYCALTFQTTDMVDVPTTQHEYDYWGLGTQVTVSS |
| Exemplary anti-target (EGFR) | EVQLLESGGGLVQPGGSLTLSCAASGSIVTINAMTWYRQAPGK |
| (SEQ ID NO: 778) | RRELVAIITSGGETNYADSVKGRFTISRDNSKNTLYLQMNSLRAE |
| | DTAVYYCNVVPPLGSWGLGTQVTVSS |
| Exemplary anti-target (EGFR) | EVQLLESGGGLVQPGGSLTLSCAASGRYHMAWFRQAPGKERE |
| (SEQ ID NO: 779) | FVGTISSGDSTWYTNSVKGRFTISRDNSKNTLYLQMNSLRAEDT |
| | AVYYCAAALYYGDSRRAADYPYWGLGTQVTVSS |
| Exemplary anti-target (EGFR) | EVQLLESGGGLVQPGGSLTLSCAASGSTFRHHAMAWFRQTPG |
| (SEQ ID NO: 780) | KEREFVSAINDHGDRTKYLDSVKGRFTISRDNSKNTLYLQMNSL |
| | RAEDTAVYYCAAGPLVDYLETTPLVYTYWGLGTQVTVSS |
| Exemplary anti-target (EGFR) | EVQLLESGGGLVQPGGSLTLSCAASGRSFSNYIMGWFRQAPG |
| (SEQ ID NO: 781) | KEREFVAGLGWSPGNTYYADSVKGRFTISRDNSKNTLYLQMNS |
| | LRAEDTAVYYCAARRGDVIYTTPWNYVYWGLGTQVTVSS |
| Exemplary anti-target (EGFR) | EVQLLESGGGLVQPGGSLTLSCAASGLTFSSYAMAWFRQAPGK |
| (SEQ ID NO: 782) | QRELVARITSGGTTDYADSVKGRFTISRDNSKNTLYLQMNSLRA |
| | EDTAVYYCAADLTYRNLLLKLPHYWGLGTQVTVSS |
| Exemplary anti-target (EGFR) | EVQLLESGGGLVQPGGSLTLSCAASVSIFSVNAVDWYRQSPGK |
| (SEQ ID NO: 783) | ERELVAIMTSDGSTNYDDSVKGRFTISRDNSKNTLYLQMNSLR |
| | AEDTAVYYCNTVPPRYWGLGTQVTVSS |
| Exemplary anti-target (EGFR) | EVQLLESGGGLVQPGGSLTLSCAASGNDFVITDMHWYRQAPG |
| (SEQ ID NO: 784) | KQREWVATITRFATTNYADSVKGRFTISRDNSKNTLYLQMNSL |
| | RAEDTAVYYCKAIGLRGVPDVNRQFEVWGLGTQVTVSS |
| Exemplary anti-target (EGFR) | EVQLLESGGGLVQPGGSLTLSCAASGAIAYIYGMGWYRQAPGK |
| (SEQ ID NO: 785) | QRELVAAISSGGSTDYADSVKGRFTISRDNSKNTLYLQMNSLRA |
| | EDTAVYYCNADVRTSRNLVRSDYWGLGTQVTVSS |
| Exemplary EGFR ProTriTAC | EVQLVESGGGLVQPGNSLRLSCAASGFTFSKFGMSWVRQGGG |
| containing linker sequence | GGLDGNEEPGGLEWVSSISGSGRDTLYADSVKGRFTISRDNAK |
| L001 (identified by residues in | TTLYLQMNSLRPEDTAVYYCTIGGSLSVSSQGTLVTVSS |
| bold and italics) and masking | GGGGKPLGLQARVVGGGGT |
| sequence M027(identified by | QTVVTQEPSLTVSPGGTVTLTCASSTGAVTSGNYPNWVQQKP |
| residues that are bold and | GQAPRGLIGGTKFLVPGTPARFSGSLLGGKAALTLSGVQPEDEA |
| underlined) | EYYCTLWYSNRWVFGGGTKLTVLGGGGSGGGGSGGGGSEVQ |
| (SEQ ID No. 786) | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLLESGGGLVQPGGSLTLSCAASGRTDSWYVMGWF |
| | RQAPGKDREFVAGVSWSYGNTYYADSVKGRFTISRDNSKNTLY |
| | LQMNSLRAEDTAVYYCAARVSREVIPTRWDLYNYWGLGTQVT |
| | VSSHHHHHH |
| Exemplary EGFR ProTriTAC | EVQLVESGGGLVQPGNSLRLSCAASGFTFSKFGMSWVRQGGG |
| containing linker sequence | GGLDGNEEPGGLEWVSSISGSGRDTLYADSVKGRFTISRDNAK |
| L040 (identified by residues in | TTLYLQMNSLRPEDTAVYYCTIGGSLSVSSQGTLVTVSSGGGGP |
| bold and italics) and masking | QASTGRSGGGGGGT |
| sequence M027(identified by | QTVVTQEPSLTVSPGGTVTLTCASSTGAVTSGNYPNWVQQKP |
| residues that are bold and | GQAPRGLIGGTKFLVPGTPARFSGSLLGGKAALTLSGVQPEDEA |
| underlined) | EYYCTLWYSNRWVFGGGTKLTVLGGGGSGGGGSGGGGSEVQ |
| (SEQ ID No. 787) | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLLESGGGLVQPGGSLTLSCAASGRTDSWYVMGWF |
| | RQAPGKDREFVAGVSWSYGNTYYADSVKGRFTISRDNSKNTLY |
| | LQMNSLRAEDTAVYYCAARVSREVIPTRWDLYNYWGLGTQVT |
| | VSSHHHHHH |
| Exemplary EGFR ProTriTAC | EVQLVESGGGLVQPGNSLRLSCAASGFTFSKFGMSWVRQGGG |
| containing linker sequence | GGLDGNEEPGGLEWVSSISGSGRDTLYADSVKGRFTISRDNAK |
| L041 (identified by residues in | TTLYLQMNSLRPEDTAVYYCTIGGSLSVSSQGTLVTVSS |
| bold and italics) and masking | GGGGPQGSTGRAAGGGGGT |
| sequence M027(identified by | QTVVTQEPSLTVSPGGTVTLTCASSTGAVTSGNYPNWVQQKP |
| residues that are bold and | GQAPRGLIGGTKFLVPGTPARFSGSLLGGKAALTLSGVQPEDEA |
| underlined) | EYYCTLWYSNRWVFGGGTKLTVLGGGGSGGGGSGGGGSEVQ |
| (SEQ ID No. 788) | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSS |
| | GGGGSGGGSEVQLLESGGGLVQPGGSLTLSCAASGRTDSWYV |
| | MGWFRQAPGKDREFVAGVSWSYGNTYYADSVKGRFTISRDN |
| | SKNTLYLQMNSLRAEDTAVYYCAARVSREVIPTRWDLYNYWGL |
| | GTQVTVSSHHHHHH |
| Exemplary EGFR ProTriTAC | EVQLVESGGGLVQPGNSLRLSCAASGFTFSKFGMSWVRQGGG |
| containing linker sequence | GGLDGNEEPGGLEWVSSISGSGRDTLYADSVKGRFTISRDNAK |
| L042 (identified by residues in | TTLYLQMNSLRPEDTAVYYCTIGGSLSVSSQGTLVTVSSGGGGP |
| bold and italics) and masking | PASSGRAGGGGGT |
| sequence M027(identified by | QTVVTQEPSLTVSPGGTVTLTCASSTGAVTSGNYPNWVQQKP |
| residues that are bold and | GQAPRGLIGGTKFLVPGTPARFSGSLLGGKAALTLSGVQPEDEA |
| underlined) | EYYCTLWYSNRWVFGGGTKLTVLGGGGSGGGGSGGGGSEVQ |
| (SEQ ID No. 789) | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSS |
| | GGGGSGGGSEVQLLESGGGLVQPGGSLTLSCAASGRTDSWYV |
| | MGWFRQAPGKDREFVAGVSWSYGNTYYADSVKGRFTISRDN |
| | SKNTLYLQMNSLRAEDTAVYYCAARVSREVIPTRWDLYNYWGL |
| | GTQVTVSSHHHHHH |
| Exemplary EGFR ProTriTAC | EVQLVESGGGLVQPGNSLRLSCAASGFTFSKFGMSWVRQGGG |
| containing linker sequence | GGLDGNEEPGGLEWVSSISGSGRDTLYADSVKGRFTISRDNAK |
| L045 (identified by residues in | TTLYLQMNSLRPEDTAVYYCTIGGSLSVSSQGTLVTVSSGGGGP |
| bold and italics) and masking | IPVQGRAHGGGGTQTVVTQEPSLTVSPGGTVTLTCASSTGAVT |
| sequence M027(identified by | SGNYPNWVQQKPGQAPRGLIGGTKFLVPGTPARFSGSLLGGK |
| residues that are bold and | AALTLSGVQPEDEAEYYCTLWYSNRWVFGGGTKLTVLGGGGS |
| underlined) | GGGGSGGGGSEVQLVESGGGLVQPGGSLKLSCAASGFTFNKY |
| (SEQ ID No. 790) | AINWVRQAPGKGLEWVARIRSKYNNYATYYADQVKDRFTISR |
| | DDSKNTAYLQMNNLKTEDTAVYYCVRHANFGNSYISYWAYW |
| | GQGTLVTVSSGGGGSGGGS |
| | EVQLLESGGGLVQPGGSLTLSCAASGRTDSWYVMGWFRQAP |
| | GKDREFVAGVSWSYGNTYYADSVKGRFTISRDNSKNTLYLQM |
| | NSLRAEDTAVYYCAARVSREVIPTRWDLYNYWGLGTQVTVSSH |
| | HHHHH |
| Exemplary EGFR ProTriTAC | EVQLVESGGGLVQPGNSLRLSCAASGFTFSKFGMSWVRQGGG |
| containing a non cleavable | GGLDGNEEPGGLEWVSSISGSGRDTLYADSVKGRFTISRDNAK |
| linker sequence(identified by | TTLYLQMNSLRPEDTAVYYCTIGGSLSVSSQGTLVTVSSGGGGS |
| residues in bold and italics) | GGGGSGGVVGGGGTQTVVTQEPSLTVSPGGTVTLTCASSTGA |
| and masking sequence | VTSGNYPNWVQQKPGQAPRGLIGGTKFLVPGTPARFSGSLLG |
| M027(identified by residues | GKAALTLSGVQPEDEAEYYCTLWYSNRWVFGGGTKLTVLGGG |
| that are bold and underlined) | GSGGGGSGGGGSEVQLVESGGGLVQPGGSLKLSCAASGFTFN |
| (SEQ ID No. 791) | KYAINWVRQAPGKGLEWVARIRSKYNNYATYYADQVKDRFTIS |
| | RDDSKNTAYLQMNNLKTEDTAVYYCVRHANFGNSYISYWAY |
| | WGQGTLVTVSS |
| | GGGGSGGGSEVQLLESGGGLVQPGGSLTLSCAASGRTDSWYV |
| | MGWFRQAPGKDREFVAGVSWSYGNTYYADSVKGRFTISRDN |
| | SKNTLYLQMNSLRAEDTAVYYCAARVSREVIPTRWDLYNYWGL |
| | GTQVTVSSHHHHHH |
| Exemplary GFP TriTAC | QVQLVESGGALVQPGGSLRLSCAASGFPVNRYSMRWYRQAP |
| Sequence | GKEREWVAGMSSAGDRSSYEDSVKGRFTISRDDARNTVYLQM |
| (SEQ ID No. 792) | NSLKPEDTAVYYCNVNVGFEYWGQGTQVTVSSGGGGSGGGS |
| | EVQLVESGGGLVQPGNSLRLSCAASGFTFSKFGMSWVRQAPG |
| | KGLEWVSSISGSGRDTLYADSVKGRFTISRDNAKTTLYLQMNSL |
| | RPEDTAVYYCTIGGSLSVSSQGTLVTVSSGGGGSGGGSEVQLVE |
| | SGGGLVQPGGSLKLSCAASGFTFNKYAINWVRQAPGKGLEWV |
| | ARIRSKYNNYATYYADQVKDRFTISRDDSKNTAYLQMNNLKTE |
| | DTAVYYCVRHANFGNSYISYWAYWGQGTLVTVSSGGGGSGG |
| | GGSGGGGSQTVVTQEPSLTVSPGGTVTLTCASSTGAVTSGNYP |
| | NWVQQKPGQAPRGLIGGTKFLVPGTPARFSGSLLGGKAALTLS |
| | GVQPEDEAEYYCTLWYSNRWVFGGGTKLTVLHHHHHH |
| Exemplary EGFR TriTAC | EVQLLESGGGLVQPGGSLTLSCAASGRTDSWYVMGWFRQAP |
| Sequence | GKDREFVAGVSWSYGNTYYADSVKGRFTISRDNSKNTLYLQM |
| (SEQ ID No. 793) | NSLRAEDTAVYYCAARVSREVIPTRWDLYNYWGLGTQVTVSSG |
| | GGGSGGGSEVQLVESGGGLVQPGNSLRLSCAASGFTFSKFGM |
| | SWVRQAPGKGLEWVSSISGSGRDTLYADSVKGRFTISRDNAKT |
| | TLYLQMNSLRPEDTAVYYCTIGGSLSVSSQGTLVTVSSGGGGSG |
| | GGSEVQLVESGGGLVQPGGSLKLSCAASGFTFNKYAINWVRQ |
| | APGKGLEWVARIRSKYNNYATYYADQVKDRFTISRDDSKNTAY |
| | LQMNNLKTEDTAVYYCVRHANFGNSYISYWAYWGQGTLVTV |
| | SSGGGGSGGGGSGGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLH |
| | HHHHH |
| Exemplary anti-CD3 scFv | QTVVTQEPSLTVSPGGTVTLTCASSTGAVTSGNYPNWVQQKP |
| (SEQ ID NO: 794) | GQAPRGLIGGTKFLVPGTPARFSGSLLGGKAALTLSGVQPEDEA |
| | EYYCTLWYSNRWVFGGGTKLTVLGGGGSGGGGSGGGGSEVQ |
| | LVESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSS |
| Exemplary anti-target (EpCAM) | QVQLQESGGGLVQAGGSLRLSCAASGFIFRAASMAWYRQSPG |
| (SEQ ID NO: 804) EPL90 | NERELVASISSGAFTNYADSVKARFTISRDNAKNTVYLQMNSLK |
| | PEDTAVYFCGATFLRSDGHHTINGQGTQVTVSS |
| Exemplary anti-target (EpCAM) | QVQLQESGGGLVQAGGSLRLSCAASGFIFRAASMGWFRQSPG |
| (SEQ ID NO: 805) EPL118 | NERELVATVSSGDFTNYADSVKGRFTISRDNAKNTVYLQMNSL |
| | KPEDTAVYFCGATFVRSDGHHTIYGQGTQVTVSS |
| Exemplary anti-target (EpCAM) | QVQLQESGGGLVQAGGSLRLSCAASGFIFRAASMDWYRQFPG |
| (SEQ ID NO: 806) EPL138 | NERESIATISSGGFTNYADSVKGRFTISRDNAKNTVYLQMNSLK |
| | PEDTAVYFCGATFLRSDGHHTINGQGTQVTVSS |
| Exemplary anti-target (EpCAM) | QVQLQESGGGLVQAGGSLRLSCAASGFIFRAASMGWFRQSPG |
| (SEQ ID NO: 807) EPL145 | NERELVATVSSGGFTNYADSVKGRFTISRDNAKNTVYLQMNSL |
| | KPEDTAVYFCGATFVRSDGHHTIYGQGTQVTVSS |
| Exemplary anti-target (EpCAM) | QVQLQESGGGLVQAGGSLRLSCAASGFIFRAASMDWYRQSPG |
| (SEQ ID NO: 808) EPL164 | TQPELVATISSTGFTNYANSVKGRFTISRDNAKNTVYLQMNSLK |
| | PEDTAVYFCGATFLRSDGQHSIYGQGTQVTVSS |
| Exemplary anti-target (EpCAM) | QVQLQESGGGLVHTGGSLRLSCAASGDTFLRYAMGWFRQAP |
| (SEQ ID NO: 809) EPL31 | GKEREFVAAITWNGGNTDYAGSLKGRFTISRDNTKNTVYLQM |
| | NSLKPEDTAVYYCAADLTFGLASSHYQYDYWGQGTQVTVSS |
| Exemplary anti-target (EpCAM) | QVQLQESGGGLVQAGGSLRLSCAASGDTFLRYAMGWFRQAP |
| (SEQ ID NO: 810) EPL55 | GKEREFVAAITWNGGNTDYAGSLKGRFTISRDNTKNTVYLQM |
| | NSLKPEDTAVYYCAADLTFGLASSHYQYDYWGQGTQVTVSS |
| Exemplary anti-target (EpCAM) | QVQLQESGGGLVHTGGSLRLSCAFSGDTFLRYAMGWFRQAP |
| (SEQ ID NO: 811) EPL57 | GKEREFVAAITWNGGNTDYADSLKGRFTISRDNTKNTVYLQM |
| | NSLRPEDTAVYYCAADLTFGLASSHYQYDYWGQGTQVTVSS |
| Exemplary anti-target (EpCAM) | QVQLQESGGGLVQPGGSLRLSCAASGDTFLRYAMGWFRQAP |
| (SEQ ID NO: 812) EPL136 | GKEREFVAAITWNGGNTDYAGSLKGRFTISRDNTKNTVYLQM |
| | NSLKPEDTAVYYCAADLTFGLASSHYQYDYWGQGTQVTVSS |
| Exemplary anti-target (EpCAM) | QVQLQESGGGSVLAGGSLRLSCAASGFTFSSYYMSWVRQAPG |
| (SEQ ID NO: 813) EPL15 | KGLEWVSGIHYTGDWTNYADSVKGRFTISRDNAKNELYLEMN |
| | NLKPEDTAVYYCARGSDKGQGTQVTVSS |
| Exemplary anti-target (EpCAM) | QVQLQESGGGSVQAGGSLRLSCAASGFTFSSYYMSWVRQAP |
| (SEQ ID NO: 814) EPL34 | GKGLEWVSGIHYTGDWTNYADSVKGRFTISRDNAKNELYLEM |
| | NNLKPEDTAVYYCARGSDKGQGTQVTVSS |
| Exemplary anti-target (EpCAM) | QVQLQESGGGLVQPGGSLRLSCAASGFTFSDWAMSWVRQAP |
| (SEQ ID NO: 815) EPL86 | GKGLEWVSGIHYGDHTTHYADFVKGRFTISRDDAKNTLYLQM |
| | NSLKPEDTAVYYCARGSTKGQGTQVTVSS |
| Exemplary anti-target (EpCAM) | QVQLQESGGGLVQPGGSLRLSCAASGFTFSDWAMSWVRQAP |
| (SEQ ID NO: 816) EPL153 | GKGLEWVSSIHYGDHTTHYADFVKGRFTISRDDAKNTLYLQMN |
| | SLKPEDTAVYYCEKGTTRGQGTQVTVSS |
| Exemplary anti-target (EpCAM) | QVQLQESGGGLVQAGGSLKLSCAASGNVFRAATMAWYRQAP |
| (SEQ ID NO: 817) EPL20 | EKQREMVATIASGGTTNYADFVKGRFTISRDNAKNTVYLQMN |
| | TLKPEDTAVYYCNAGYLTSLGPKNYWGQGTQVTVSS |
| Exemplary anti-target (EpCAM) | QVQLQESGGGLVQPGGSLRLSCAASGNVFRAATMAWYRQAP |
| (SEQ ID NO: 818) EPL70 | EKXREMVATIASGGTTNYADFVKGRFTISRDNAKNTVYLQMNT |
| | LKPEDTAVYYCNAGYLTSLGPKNYWGQGTQVTVSS |
| Exemplary anti-target (EpCAM) | QVQLQESGGGLVQAGGSLRLSCAASGNVFRAATMAWYRQVP |
| (SEQ ID NO: 819) EPL125 | EKQREMVATIASGGTTNYADFVKGRFTISRDNAKNTVYLQMN |
| | TLKPEDTAVYYCNALYLTSLGPKSYWGQGTQVTVSS |
| Exemplary anti-target (EpCAM) | QVQLQESGGGLVQPGGSLRLSCAASGFAFGNHWMYWYRQA |
| (SEQ ID NO: 820) EPL13 | PGRGRELVASISSGGSTNYVDSVKGRFTISRDNARNTVYLQMYS |
| | LKPEDTAVYYCGTSDNWGQGTQVTVSS |
| Exemplary anti-target (EpCAM) | QVQLQESGGGLVQAGGSLRLSCAASGFAFGNHWMYWYRQA |
| (SEQ ID NO: 821) EPL129 | PGRGRELVASISSGGSTNYVDSVKGRFTISRDNARNTVYLQMYS |
| | LRPEDTAVYYCGTSDNWGQGTQVTVSS |
| Exemplary anti-target (EpCAM) | QVQLQESGGGLVQAGGSLRLSCAASGFAFGNHWMYWYRQA |
| (SEQ ID NO: 822) EPL159 | PGRGRELVASISSGGSTNYVDSVKGRFTISRDNARNTVYLQMYS |
| | LKPEDTAVYYCGTSDNWGQGTQVTVSS |
| Exemplary anti-target (EpCAM) | QVQLQESGGGLVQAGGSLRLSCAASGFIFRAASISWYRQSPGN |
| (SEQ ID NO: 823) EPL120 | ERELVATINSGGFTNYADSVLGRFTISRDNAKNTGYLQMNSLKP |
| | EDTAVYFCAATFLRSDGQPPIWGQGTQVTVSS |
| Exemplary anti-target (EpCAM) | QVQLQESGGGLVQAGGSLRLSCAASGFIFRAASMGWYRQSPG |
| (SEQ ID NO: 824) EPL126 | NERELVATINSGGFTNYADSVKGRFTISRDNAKNTGYLQMNSL |
| | KPEDTAVYFCAATFLRSDGQPPIWGQGTQVTVSS |
| Exemplary anti-target (EpCAM) | QVQLQESGGGLVQAGGSLRLSCAASEYILSMYRMAWYRQAP |
| (SEQ ID NO: 825) EPL60 | GKVRELVADMSSGGTTNYADFVKGRFTISRDNDRNTVYLQMN |
| | RLQPEDTAAYYCNVAGRTGPPSYDAFNNWGQGTQVTVSS |
| Exemplary anti-target (EpCAM) | QVQLQESGGGLVQPGASLRVSCAASEYILSMYRMAWYRQAP |
| (SEQ ID NO: 826) EPL156 | GKVRELVADMSSGGTTNYADFVKGRFTISRDNDRNTVYLQMN |
| | RLQPEDTAAYYCNVAGRTGPPSYDAFNNWGQGTQVTVSS |
| Exemplary anti-target (EpCAM) | QVQLQESGGGLVQPGGSLRLSCAASESISSFIAVGWYRQAPGK |
| (SEQ ID NO: 827) EPL2 | ERELVAGINRSGFTYYTDSVKGRFSISRDNAKNTVLLQMTSLKP |
| | EDTAVYYCNAGGLYFSNAYTQGDYWGQGTQVTVSS |
| Exemplary anti-target (EpCAM) | QVQLQESGGGLVQTGGSLRLSCAASESISSFIAVGWYRQAPGK |
| (SEQ ID NO: 828) EPL43 | ERELVAGINRSGFTYYTDSVKGRFSISRDNAKNTVLLQMTSLKP |
| | EDTAVYYCNAGGLYFSNAYTQGDYWGQGTQVTVSS |
| Exemplary anti-target (EpCAM) | QVQLQESGGGLVQAGGSLRLSCAASGSVFRANVMGWYRQAP |
| (SEQ ID NO: 829) EPL10 | GKQHELVARIDPGGTTTYADPVKGRFTISRDNAKKTVYLQMNS |
| | LKPDDTAVYYCNAIILLSGGPKDYWGQGTQVTVSS |
| Exemplary anti-target (EpCAM) | QVQLQESGGGLVQAGGSLRLSCAPSGRTSSIFGMGWFRQAPG |
| (SEQ ID NO: 830) EPL49 | KEREFVASINWSGGSTSYADSVKGRFTISRDNAKNEMYLQMN |
| | SLKFEDTAVYVCAAAVLTNKPSWNFWGQGTQVTVSS |
| Exemplary anti-target (EpCAM) | QVQLQESGGGLVQAGGSLRLSCAASGPIFSDTIRTMGWYRQA |
| (SEQ ID NO: 831) EPL58 | AGKQRELVATIASFPSRTNYVDSVKGRFTISRDIAKNTVYLQMD |
| | SLKPEDTAVYYCNVDLASIPTKTYWGQGTQVTVSS |
| Exemplary anti-target (EpCAM) | QVQLQESGGGLVQAGGSLRLSCAASGSIFGINAMGWYRQAP |
| (SEQ ID NO: 832) EPL74 | GKQRESVAFITIGGNTNYLDSVKGRFTISRDNAKNTVYLQMNG |
| | LKPEDTAVYYCNTNPPLILTAGGLYWGQGTQVTVSS |
| Exemplary anti-target (EpCAM) | QVQLQESGGGLVQPGGSLRLSCATSANRFNINVMGWYRQAP |
| (SEQ ID NO: 833) EPL78 | GQQRELVATINIGGSTDYADSVKGRFTISRDNAKNTVYLQLSDL |
| | KPEDTAVYYCNVKLRVSGPTGPNVYWGQGTQVTVSS |
| Exemplary anti-target (EpCAM) | QVQLQESGGGLVQAGGSLKLSCTASGTILSTMAWYRQAPGKQ |
| (SEQ ID NO: 834) EPL82 | RELVATISRGGTTNYSDSVKGRFAISRDSTKNTVYLQMNSLKPE |
| | DTAVYYCNTPLTDYGMGYNWGQGTQVTVSS |
| Exemplary anti-target (EpCAM) | QVQLQESGGGLVQAGGSLRLSCAVSGSIFSLNTLAWYRQAPGR |
| (SEQ ID NO: 835) EPL83 | QRDLIARITGGGTTVYADSVKGRFTISRDNAKNTVYLQMNSLKP |
| | EDTAVYYCNLMVRHPSGSTYEYWGQGTQVTVSS |
| Exemplary anti-target (EpCAM) | QVQLQESGGGLVQAGGSLRLSCAASGIIFRGTTMGWFRQAPG |
| (SEQ ID NO: 836) EPL97 | KQRESVASISPLGTTSYSGSVEGRFTVSRDNAKNTLFLQMNSLK |
| | SEDTAVYYCNAIQVTNVGPRVYWGQGTQVTVSS |
| Exemplary anti-target (EpCAM) | QVQLQESGGGLVQPGGSLRLSCASSGFTLDDYTIGWFRQAPG |
| (SEQ ID NO: 837) EPL109 | KEREGVSCISRRDDSTYYADSVKGRFTISRDNAKNTVDLQMISL |
| | RPEDTAVYYCAATPRSYTLRCLGKFDFQGQGTQVTVSS |
| Exemplary anti-target (EpCAM) | QVQLQESGGGLVQAGGSLRLSCAASGNIVRMTNMAWYRQA |
| (SEQ ID NO: 838) EPL117 | PGKQREFVATISAGGSTTYVDSVKDRFTISRDNTKNTVYLQMN |
| | YLKPEDTAVYYCATGSILTNRGAIPGSWGHGTQVTVSS |
| Exemplary anti-target (EpCAM) | QVQLQESGGGLVQAGGSLRLSCAAPGFAFNDHAILWFRQAPG |
| (SEQ ID NO: 839) EPL127 | KEREGVSEICRDGTTYYTDSVKGRFTISSDNAKNTVYLQMNSVK |
| | TDDTAVYYCAVDRRRYYCSGNRAFSSDYYYWGQGTQVTVSS |
| Exemplary anti-target (EpCAM) | QVQLQESGGGLVQAGGSLRLSCVHSGSIFRASTMAWYRQAPG |
| (SEQ ID NO: 840) EPL152 | KQRELVAQIMSGGGTNYAGSVKGRFTISRDNANNTVYLQMNS |
| | LKPEDTAVYYCNAAQITSWGPKVYWGQGTQVTVSS |
| Exemplary anti-target (EpCAM) | QVQLQESGGGLVQPGGSLRLSCAASGRINSINTMGWYRQAPG |
| (SEQ ID NO: 841) EPL189 | NQRELVAEITRGGTTNYADSVQGRYAISRDNAKNLVYLQMNSL |
| | KPEDTDVYYCNAQTFPTFSRPTGLDYWGQGTQVTVSS |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 842) EPL10 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGSVFR |
| | ANVMGWYRQAPGKQHELVARIDPGGTTTYADPVKGRFTISRD |
| | NAKKTVYLQMNSLKPDDTAVYYCNAILLSGGPKDYWGQGTQ |
| | VTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 843) EPL109 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCASSGFTLD |
| | DYTIGWFRQAPGKEREGVSCISRRDDSTYYADSVKGRFTISRDN |
| | AKNTVDLQMISLRPEDTAVYYCAATPRSYTLRCLGKFDFQGQG |
| | TQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 844) EPL117 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGNIVR |
| | MTNMAWYRQAPGKQREFVATISAGGSTTYVDSVKDRFTISRD |
| | NTKNTVYLQMNYLKPEDTAVYYCATGSILTNRGAIPGSWGHGT |
| | QVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 845) EPL120 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGFIFR |
| | AASISWYRQSPGNERELVATINSGGFTNYADSVLGRFTISRDNA |
| | KNTGYLQMNSLKPEDTAVYFCAATFLRSDGQPPIWGQGTQVT |
| | VSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 846) EPL125 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGNVFR |
| | AATMAWYRQVPEKQREMVATIASGGTTNYADFVKGRFTISRD |
| | NAKNTVYLQMNTLKPEDTAVYYCNALYLTSLGPKSYWGQGTQ |
| | VTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 847) EPL127 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAAPGFAFN |
| | DHAILWFRQAPGKEREGVSEICRDGTTYYTDSVKGRFTISSDNA |
| | KNTVYLQMNSVKTDDTAVYYCAVDRRRYYCSGNRAFSSDYYY |
| | WGQGTQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 848) EPL13 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCAASGFAFG |
| | NHWMYWYRQAPGRGRELVASISSGGSTNYVDSVKGRFTISRD |
| | NARNTVYLQMYSLKPEDTAVYYCGTSDNWGQGTQVTVSSHH |
| | HHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 849) EPL136 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCAASGDTFL |
| | RYAMGWFRQAPGKEREFVAAITWNGGNTDYAGSLKGRFTISR |
| | DNTKNTVYLQMNSLKPEDTAVYYCAADLTFGLASSHYQYDYW |
| | GQGTQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 850) EPL138 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGFIFR |
| | AASMDWYRQFPGNERESIATISSGGFTNYADSVKGRFTISRDN |
| | AKNTVYLQMNSLKPEDTAVYFCGATFLRSDGHHTINGQGTQV |
| | TVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 851) EPL145 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGFIFR |
| | AASMGWFRQSPGNERELVATVSSGGFTNYADSVKGRFTISRD |
| | NAKNTVYLQMNSLKPEDTAVYFCGATFVRSDGHHTIYGQGTQ |
| | VTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 852) EPL152 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCVHSGSIFR |
| | ASTMAWYRQAPGKQRELVAQIMSGGGTNYAGSVKGRFTISR |
| | DNANNTVYLQMNSLKPEDTAVYYCNAAQITSWGPKVYWGQ |
| | GTQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 853) EPL153 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCAASGFTFS |
| | DWAMSWVRQAPGKGLEWVSSIHYGDHTTHYADFVKGRFTIS |
| | RDDAKNTLYLQMNSLKPEDTAVYYCEKGTTRGQGTQVTVSSH |
| | HHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 854) EPL156 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGASLRVSCAASEYILS |
| | MYRMAWYRQAPGKVRELVADMSSGGTTNYADFVKGRFTISR |
| | DNDRNTVYLQMNRLQPEDTAAYYCNVAGRTGPPSYDAFNN |
| | WGQGTQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 855) EPL164 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGFIFR |
| | AASMDWYRQSPGTQPELVATISSTGFTNYANSVKGRFTISRDN |
| | AKNTVYLQMNSLKPEDTAVYFCGATFLRSDGQHSIYGQGTQV |
| | TVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 856) EPL189 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCAASGRINSI |
| | NTMGWYRQAPGNQRELVAEITRGGTTNYADSVQGRYAISRD |
| | NAKNLVYLQMNSLKPEDTDVYYCNAQTFPTFSRPTGLDYWGQ |
| | GTQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 857) EPL2 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCAASESISSFI |
| | AVGWYRQAPGKERELVAGINRSGFTYYTDSVKGRFSISRDNAK |
| | NTVLLQMTSLKPEDTAVYYCNAGGLYFSNAYTQGDYWGQGT |
| | QVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 858) EPL20 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLKLSCAASGNVFR |
| | AATMAWYRQAPEKQREMVATIASGGTTNYADFVKGRFTISRD |
| | NAKNTVYLQMNTLKPEDTAVYYCNAGYLTSLGPKNYWGQGT |
| | QVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 859) EPL34 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGSVQAGGSLRLSCAASGFTFS |
| | SYYMSWVRQAPGKGLEWVSGIHYTGDWTNYADSVKGRFTISR |
| | DNAKNELYLEMNNLKPEDTAVYYCARGSDKGQGTQVTVSSHH |
| | HHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 860) EPL49 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAPSGRTSSI |
| | FGMGWFRQAPGKEREFVASINWSGGSTSYADSVKGRFTISRD |
| | NAKNEMYLQMNSLKFEDTAVYVCAAAVLTNKPSWNFWGQG |
| | TQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 861) EPL58 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGPIFS |
| | DTIRTMGWYRQAAGKQRELVATIASFPSRTNYVDSVKGRFTIS |
| | RDIAKNTVYLQMDSLKPEDTAVYYCNVDLASIPTKTYWGQGTQ |
| | VTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 862) EPL74 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGSIFGI |
| | NAMGWYRQAPGKQRESVAFITIGGNTNYLDSVKGRFTISRDN |
| | AKNTVYLQMNGLKPEDTAVYYCNTNPPLILTAGGLYWGQGTQ |
| | VTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 863) EPL78 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCATSANRFN |
| | INVMGWYRQAPGQQRELVATINIGGSTDYADSVKGRFTISRD |
| | NAKNTVYLQLSDLKPEDTAVYYCNVKLRVSGPTGPNVYWGQG |
| | TQVTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 864) EPL82 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLKLSCTASGTILST |
| | MAWYRQAPGKQRELVATISRGGTTNYSDSVKGRFAISRDSTKN |
| | TVYLQMNSLKPEDTAVYYCNTPLTDYGMGYNWGQGTQVTVS |
| | SHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 865) EPL83 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAVSGSIFSL |
| | NTLAWYRQAPGRQRDLIARITGGGTTVYADSVKGRFTISRDNA |
| | KNTVYLQMNSLKPEDTAVYYCNLMVRHPSGSTYEYWGQGTQ |
| | VTVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 866) EPL86 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQPGGSLRLSCAASGFTFS |
| | DWAMSWVRQAPGKGLEWVSGIHYGDHTTHYADFVKGRFTIS |
| | RDDAKNTLYLQMNSLKPEDTAVYYCARGSTKGQGTQVTVSSH |
| | HHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 867) EPL90 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGFIFR |
| | AASMAWYRQSPGNERELVASISSGAFTNYADSVKARFTISRDN |
| | AKNTVYLQMNSLKPEDTAVYFCGATFLRSDGHHTINGQGTQV |
| | TVSSHHHHHH* |
| Exemplary anti-CD3/anti- | VVGGGGTQTVVTQEPSLTVSPGGTVTLTCASSTGAVTSGNYPN |
| EpCAM fusion protein(SEQ ID | WVQQKPGQAPRGLIGGTKFLVPGTPARFSGSLLGGKAALTLSG |
| NO: 868) EPL97 | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSQVQLQESGGGLVQAGGSLRLSCAASGIIFRG |
| | TTMGWFRQAPGKQRESVASISPLGTTSYSGSVEGRFTVSRDNA |
| | KNTLFLQMNSLKSEDTAVYYCNAIQVTNVGPRVYWGQGTQVT |
| | VSSHHHHHH* |
| Exemplary anti-EpCAM/anti- | GGGSQVQLQESGGGLVQAGGSLRLSCAASGSVFRANVMGWY |
| EpCAM fusion protein(SEQ ID | RQAPGKQHELVARIDPGGTTTYADPVKGRFTISRDNAKKTVYL |
| NO: 869) EPL10 | QMNSLKPDDTAVYYCNAIILLSGGPKDYWGQGTQVTVSSGGG |
| | GSGGGSEVQLVESGGGLVQPGGSLKLSCAASGFTFNKYAINW |
| | VRQAPGKGLEWVARIRSKYNNYATYYADQVKDRFTISRDDSKN |
| | TAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKLT |
| | VLHHHHHH* |
| Exemplary anti-EpCAM/anti- | GGGSQVQLQESGGGLVQPGGSLRLSCASSGFTLDDYTIGWFR |
| EpCAM fusion protein(SEQ ID | QAPGKEREGVSCISRRDDSTYYADSVKGRFTISRDNAKNTVDLQ |
| NO: 870) EPL109 | MISLRPEDTAVYYCAATPRSYTLRCLGKFDFQGQGTQVTVSSG |
| | GGGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQAGGSLRLSCAASGNIVRMTNMAW |
| EpCAM fusion protein(SEQ ID | YRQAPGKQREFVATISAGGSTTYVDSVKDRFTISRDNTKNTVYL |
| NO: 871) EPL117 | QMNYLKPEDTAVYYCATGSILTNRGAIPGSWGHGTQVTVSSG |
| | GGGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQAGGSLRLSCAASGFIFRAASISWYRQ |
| EpCAM fusion protein(SEQ ID | SPGNERELVATINSGGFTNYADSVLGRFTISRDNAKNTGYLQM |
| NO: 872) EPL120 | NSLKPEDTAVYFCAATFLRSDGQPPIWGQGTQVTVSSGGGGS |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGGSGGGGSQTVVTQEPSLTVSPGGTVTLTCAS |
| | STGAVTSGNYPNWVQQKPGQAPRGLIGGTKFLVPGTPARFSG |
| | SLLGGKAALTLSGVQPEDEAEYYCTLWYSNRWVFGGGTKLTVL |
| | HHHHHH* |
| Exemplary anti-EpCAM/anti- | GGGSQVQLQESGGGLVQAGGSLRLSCAASGNVFRAATMAWY |
| EpCAM fusion protein(SEQ ID | RQVPEKQREMVATIASGGTTNYADFVKGRFTISRDNAKNTVYL |
| NO: 873) EPL125 | QMNTLKPEDTAVYYCNALYLTSLGPKSYWGQGTQVTVSSGGG |
| | GSGGGSEVQLVESGGGLVQPGGSLKLSCAASGFTFNKYAINW |
| | VRQAPGKGLEWVARIRSKYNNYATYYADQVKDRFTISRDDSKN |
| | TAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKLT |
| | VLHHHHHH* |
| Exemplary anti-EpCAM/anti- | GGGSQVQLQESGGGLVQAGGSLRLSCAAPGFAFNDHAILWFR |
| EpCAM fusion protein(SEQ ID | QAPGKEREGVSEICRDGTTYYTDSVKGRFTISSDNAKNTVYLQ |
| NO: 874) EPL127 | MNSVKTDDTAVYYCAVDRRRYYCSGNRAFSSDYYYWGQGTQ |
| | VTVSSGGGGSGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWAY |
| | WGQGTLVTVSSGGGGSGGGGSGGGGSQTVVTQEPSLTVSPG |
| | GTVTLTCASSTGAVTSGNYPNWVQQKPGQAPRGLIGGTKFLV |
| | PGTPARFSGSLLGGKAALTLSGVQPEDEAEYYCTLWYSNRWVF |
| | GGGTKLTVLHHHHHH* |
| Exemplary anti-EpCAM/anti- | GGGSQVQLQESGGGLVQPGGSLRLSCAASGFAFGNHWMYW |
| EpCAM fusion protein(SEQ ID | YRQAPGRGRELVASISSGGSTNYVDSVKGRFTISRDNARNTVYL |
| NO: 875) EPL13 | QMYSLKPEDTAVYYCGTSDNWGQGTQVTVSSGGGGSGGGSE |
| | VQLVESGGGLVQPGGSLKLSCAASGFTFNKYAINWVRQAPGK |
| | GLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti-EpCAM/anti- | GGGSQVQLQESGGGLVQPGGSLRLSCAASGDTFLRYAMGWF |
| EpCAM fusion protein(SEQ ID | RQAPGKEREFVAAITWNGGNTDYAGSLKGRFTISRDNTKNTVY |
| NO: 876) EPL136 | LQMNSLKPEDTAVYYCAADLTFGLASSHYQYDYWGQGTQVTV |
| | SSGGGGSGGGSEVQLVESGGGLVQPGGSLKLSCAASGFTFNKY |
| | AINWVRQAPGKGLEWVARIRSKYNNYATYYADQVKDRFTISR |
| | DDSKNTAYLQMNNLKTEDTAVYYCVRHANFGNSYISYWAYW |
| | GQGTLVTVSSGGGGSGGGGSGGGGSQTVVTQEPSLTVSPGGT |
| | VTLTCASSTGAVTSGNYPNWVQQKPGQAPRGLIGGTKFLVPG |
| | TPARFSGSLLGGKAALTLSGVQPEDEAEYYCTLWYSNRWVFGG |
| | GTKLTVLHHHHHH* |
| Exemplary anti-EpCAM/anti- | GGGSQVQLQESGGGLVQAGGSLRLSCAASGFIFRAASMDWY |
| EpCAM fusion protein(SEQ ID | RQFPGNERESIATISSGGFTNYADSVKGRFTISRDNAKNTVYLQ |
| NO: 877) EPL138 | MNSLKPEDTAVYFCGATFLRSDGHHTINGQGTQVTVSSGGGG |
| | SGGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGGSGGGGSQTVVTQEPSLTVSPGGTVTLTCAS |
| | STGAVTSGNYPNWVQQKPGQAPRGLIGGTKFLVPGTPARFSG |
| | SLLGGKAALTLSGVQPEDEAEYYCTLWYSNRWVFGGGTKLTVL |
| | HHHHHH* |
| Exemplary anti-EpCAM/anti- | GGGSQVQLQESGGGLVQAGGSLRLSCAASGFIFRAASMGWF |
| EpCAM fusion protein(SEQ ID | RQSPGNERELVATVSSGGFTNYADSVKGRFTISRDNAKNTVYL |
| NO: 878) EPL145 | QMNSLKPEDTAVYFCGATFVRSDGHHTIYGQGTQVTVSSGGG |
| | GSGGGSEVQLVESGGGLVQPGGSLKLSCAASGFTFNKYAINW |
| | VRQAPGKGLEWVARIRSKYNNYATYYADQVKDRFTISRDDSKN |
| | TAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKLT |
| | VLHHHHHH* |
| Exemplary anti-EpCAM/anti- | GGGSQVQLQESGGGLVQAGGSLRLSCVHSGSIFRASTMAWYR |
| EpCAM fusion protein(SEQ ID | QAPGKQRELVAQIMSGGGTNYAGSVKGRFTISRDNANNTVYL |
| NO: 879) EPL152 | QMNSLKPEDTAVYYCNAAQITSWGPKVYWGQGTQVTVSSGG |
| | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQPGGSLRLSCAASGFTFSDWAMSWV |
| EpCAM fusion protein(SEQ ID | RQAPGKGLEWVSSIHYGDHTTHYADFVKGRFTISRDDAKNTLY |
| NO: 880) EPL153 | LQMNSLKPEDTAVYYCEKGTTRGQGTQVTVSSGGGGSGGGSE |
| | VQLVESGGGLVQPGGSLKLSCAASGFTFNKYAINWVRQAPGK |
| | GLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti-EpCAM/anti- | GGGSQVQLQESGGGLVQPGASLRVSCAASEYILSMYRMAWY |
| EpCAM fusion protein(SEQ ID | RQAPGKVRELVADMSSGGTTNYADFVKGRFTISRDNDRNTVY |
| NO: 881) EPL156 | LQMNRLQPEDTAAYYCNVAGRTGPPSYDAFNNWGQGTQVT |
| | VSSGGGGSGGGSEVQLVESGGGLVQPGGSLKLSCAASGFTFNK |
| | YAINWVRQAPGKGLEWVARIRSKYNNYATYYADQVKDRFTISR |
| | DDSKNTAYLQMNNLKTEDTAVYYCVRHANFGNSYISYWAYW |
| | GQGTLVTVSSGGGGSGGGGSGGGGSQTVVTQEPSLTVSPGGT |
| | VTLTCASSTGAVTSGNYPNWVQQKPGQAPRGLIGGTKFLVPG |
| | TPARFSGSLLGGKAALTLSGVQPEDEAEYYCTLWYSNRWVFGG |
| | GTKLTVLHHHHHH* |
| Exemplary anti-EpCAM/anti- | GGGSQVQLQESGGGLVQAGGSLRLSCAASGFIFRAASMDWY |
| EpCAM fusion protein(SEQ ID | RQSPGTQPELVATISSTGFTNYANSVKGRFTISRDNAKNTVYLQ |
| NO: 882) EPL164 | MNSLKPEDTAVYFCGATFLRSDGQHSIYGQGTQVTVSSGGGG |
| | SGGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGGSGGGGSQTVVTQEPSLTVSPGGTVTLTCAS |
| | STGAVTSGNYPNWVQQKPGQAPRGLIGGTKFLVPGTPARFSG |
| | SLLGGKAALTLSGVQPEDEAEYYCTLWYSNRWVFGGGTKLTVL |
| | HHHHHH* |
| Exemplary anti-EpCAM/anti- | GGGSQVQLQESGGGLVQPGGSLRLSCAASGRINSINTMGWYR |
| EpCAM fusion protein(SEQ ID | QAPGNQRELVAEITRGGTTNYADSVQGRYAISRDNAKNLVYLQ |
| NO: 883) EPL189 | MNSLKPEDTDVYYCNAQTFPTFSRPTGLDYWGQGTQVTVSSG |
| | GGGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQPGGSLRLSCAASESISSFIAVGWYRQ |
| EpCAM fusion protein(SEQ ID | APGKERELVAGINRSGFTYYTDSVKGRFSISRDNAKNTVLLQMT |
| NO: 884) EPL2 | SLKPEDTAVYYCNAGGLYFSNAYTQGDYWGQGTQVTVSSGG |
| | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQAGGSLKLSCAASGNVFRAATMAWY |
| EpCAM fusion protein(SEQ ID | RQAPEKQREMVATIASGGTTNYADFVKGRFTISRDNAKNTVYL |
| NO: 885) EPL20 | QMNTLKPEDTAVYYCNAGYLTSLGPKNYWGQGTQVTVSSGG |
| | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGSVQAGGSLRLSCAASGFTFSSYYMSWVR |
| EpCAM fusion protein(SEQ ID | QAPGKGLEWVSGIHYTGDWTNYADSVKGRFTISRDNAKNELY |
| NO: 886) EPL34 | LEMNNLKPEDTAVYYCARGSDKGQGTQVTVSSGGGGSGGGS |
| | EVQLVESGGGLVQPGGSLKLSCAASGFTFNKYAINWVRQAPG |
| | KGLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti-EpCAM/anti- | GGGSQVQLQESGGGLVQAGGSLRLSCAPSGRTSSIFGMGWFR |
| EpCAM fusion protein(SEQ ID | QAPGKEREFVASINWSGGSTSYADSVKGRFTISRDNAKNEMYL |
| NO: 887) EPL49 | QMNSLKFEDTAVYVCAAAVLTNKPSWNFWGQGTQVTVSSG |
| | GGGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQAGGSLRLSCAASGPIFSDTIRTMGW |
| EpCAM fusion protein(SEQ ID | YRQAAGKQRELVATIASFPSRTNYVDSVKGRFTISRDIAKNTVYL |
| NO: 888) EPL58 | QMDSLKPEDTAVYYCNVDLASIPTKTYWGQGTQVTVSSGGGG |
| | SGGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGGGGGSGGGGSQTVVTQEPSLTVSPGGTVTLTCAS |
| | STGAVTSGNYPNWVQQKPGQAPRGLIGGTKFLVPGTPARFSG |
| | SLLGGKAALTLSGVQPEDEAEYYCTLWYSNRWVFGGGTKLTVL |
| | HHHHHH* |
| Exemplary anti-EpCAM/anti- | GGGSQVQLQESGGGLVQAGGSLRLSCAASGSIFGINAMGWYR |
| EpCAM fusion protein(SEQ ID | QAPGKQRESVAFITIGGNTNYLDSVKGRFTISRDNAKNTVYLQ |
| NO: 889) EPL74 | MNGLKPEDTAVYYCNTNPPLILTAGGLYWGQGTQVTVSSGGG |
| | GSGGGSEVQLVESGGGLVQPGGSLKLSCAASGFTFNKYAINW |
| | VRQAPGKGLEWVARIRSKYNNYATYYADQVKDRFTISRDDSKN |
| | TAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKLT |
| | VLHHHHHH* |
| Exemplary anti-EpCAM/anti- | GGGSQVQLQESGGGLVQPGGSLRLSCATSANRFNINVMGWY |
| EpCAM fusion protein(SEQ ID | RQAPGQQRELVATINIGGSTDYADSVKGRFTISRDNAKNTVYL |
| NO: 890) EPL78 | QLSDLKPEDTAVYYCNVKLRVSGPTGPNVYWGQGTQVTVSSG |
| | GGGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQAGGSLKLSCTASGTILSTMAWYRQA |
| EpCAM fusion protein(SEQ ID | PGKQRELVATISRGGTTNYSDSVKGRFAISRDSTKNTVYLQMNS |
| NO: 891) EPL82 | LKPEDTAVYYCNTPLTDYGMGYNWGQGTQVTVSSGGGGSGG |
| | GSEVQLVESGGGLVQPGGSLKLSCAASGFTFNKYAINWVRQAP |
| | GKGLEWVARIRSKYNNYATYYADQVKDRFTISRDDSKNTAYLQ |
| | MNNLKTEDTAVYYCVRHANFGNSYISYWAYWGQGTLVTVSS |
| | GGGGGGGGSGGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLHH |
| | HHHH* |
| Exemplary anti-EpCAM/anti- | GGGSQVQLQESGGGLVQAGGSLRLSCAVSGSIFSLNTLAWYR |
| EpCAM fusion protein(SEQ ID | QAPGRQRDLIARITGGGTTVYADSVKGRFTISRDNAKNTVYLQ |
| NO: 892) EPL83 | MNSLKPEDTAVYYCNLMVRHPSGSTYEYWGQGTQVTVSSGG |
| | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGTK |
| | LTVLHHHHHH* |
| Exemplary anti-EpCAM/anti- | GGGSQVQLQESGGGLVQPGGSLRLSCAASGFTFSDWAMSWV |
| EpCAM fusion protein(SEQ ID | RQAPGKGLEWVSGIHYGDHTTHYADFVKGRFTISRDDAKNTLY |
| NO: 893) EPL86 | LQMNSLKPEDTAVYYCARGSTKGQGTQVTVSSGGGGSGGGSE |
| | VQLVESGGGLVQPGGSLKLSCAASGFTFNKYAINWVRQAPGK |
| | GLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti-EpCAM/anti- | GGGSQVQLQESGGGLVQAGGSLRLSCAASGFIFRAASMAWYR |
| EpCAM fusion protein(SEQ ID | QSPGNERELVASISSGAFTNYADSVKARFTISRDNAKNTVYLQ |
| NO: 894) EPL90 | MNSLKPEDTAVYFCGATFLRSDGHHTINGQGTQVTVSSGGGG |
| | SGGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGGSGGGGSQTVVTQEPSLTVSPGGTVTLTCAS |
| | STGAVTSGNYPNWVQQKPGQAPRGLIGGTKFLVPGTPARFSG |
| | SLLGGKAALTLSGVQPEDEAEYYCTLWYSNRWVFGGGTKLTVL |
| | HHHHHH* |
| Exemplary anti-EpCAM/anti- | GGGSQVQLQESGGGLVQAGGSLRLSCAASGIIFRGTTMGWFR |
| EpCAM fusion protein(SEQ ID | QAPGKQRESVASISPLGTTSYSGSVEGRFTVSRDNAKNTLFLQ |
| NO: 895) EPL97 | MNSLKSEDTAVYYCNAIQVTNVGPRVYWGQGTQVTVSSGGG |
| | GSGGGSEVQLVESGGGLVQPGGSLKLSCAASGFTFNKYAINW |
| | VRQAPGKGLEWVARIRSKYNNYATYYADQVKDRFTISRDDSKN |
| | TAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKLT |
| | VLHHHHHH* |
| Exemplary humanized anti- | EVQLVESGGGLVQPGGSLTLSCAASGFAFGNHWMYWYRQAP |
| EpCAM sequence (SEQ ID No. | GRGRELVASISSGGSTNYVDSVKGRFTISRDNAKNTLYLQMNSL |
| 896) | RAEDTAVYYCGTSDNWGQGTLVTVSS |
| Exemplary humanized anti- | EVQLLESGGGLVQPGGSLTLSCAASGFIFRAASMDWYRQFPG |
| EpCAM sequence (SEQ ID No. | NERESIATISSGGFTNYADSVKGRFTISRDNSKNTLYLQMNSLRA |
| 897) | EDTAVYYCGATFLRSDGHHTINGQGTLVTVSS |
| Exemplary humanized anti- | EVQLLESGGGLVQPGGSLTLSCAASGFIFRAASMAWYRQSPG |
| EpCAM sequence (SEQ ID No. | NERELVASISSGAFTNYADSVKARFTISRDNSKNTLYLQMNSLR |
| 898) | AEDTAVYYCGATFLRSDGHHTINGQGTLVTVSS |
| Exemplary Humanized Anti- | VVGGGGTQTVVTQEPSLTVSPGGTVTLTCASSTGAVTSGNYPN |
| CD3/Anti-EpCAM sequence | WVQQKPGQAPRGLIGGTKFLVPGTPARFSGSLLGGKAALTLSG |
| name H13 (SEQ ID No. 899) | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSEVQLVESGGGLVQPGGSLTLSCAASGFAFG |
| | NHWMYWYRQAPGRGRELVASISSGGSTNYVDSVKGRFTISRD |
| | NAKNTLYLQMNSLRAEDTAVYYCGTSDNWGQGTLVTVSSHH |
| | HHHH* |
| Exemplary Humanized Anti- | VVGGGGTQTVVTQEPSLTVSPGGTVTLTCASSTGAVTSGNYPN |
| CD3/Anti-EpCAM sequence | WVQQKPGQAPRGLIGGTKFLVPGTPARFSGSLLGGKAALTLSG |
| name H138 (SEQ ID No. 900) | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSEVQLLESGGGLVQPGGSLTLSCAASGFIFRA |
| | ASMDWYRQFPGNERESIATISSGGFTNYADSVKGRFTISRDNS |
| | KNTLYLQMNSLRAEDTAVYYCGATFLRSDGHHTINGQGTLVTV |
| | SSHHHHHH* |
| Exemplary Humanized Anti- | VVGGGGTQTVVTQEPSLTVSPGGTVTLTCASSTGAVTSGNYPN |
| CD3/Anti-EpCAM sequence | WVQQKPGQAPRGLIGGTKFLVPGTPARFSGSLLGGKAALTLSG |
| name H90 (SEQ ID No. 901) | VQPEDEAEYYCTLWYSNRWVFGGGTKLTVLGGGGSGGGGSG |
| | GGGSEVQLVESGGGLVQPGGSLKLSCAASGFTFNKYAINWVR |
| | QAPGKGLEWVARIRSKYNNYATYYADQVKDRFTISRDDSKNTA |
| | YLQMNNLKTEDTAVYYCVRHANFGNSYISYWAYWGQGTLVT |
| | VSSGGGGSGGGSEVQLLESGGGLVQPGGSLTLSCAASGFIFRA |
| | ASMAWYRQSPGNERELVASISSGAFTNYADSVKARFTISRDNS |
| | KNTLYLQMNSLRAEDTAVYYCGATFLRSDGHHTINGQGTLVTV |
| | SSHHHHHH* |
| SEQ ID No. 902 | QDGNEEMGGITQ |
| SEQ ID No. 903 | LVQPGN |
| AB loop oligo WT | |
| SEQ ID No. 904 | DSVKGR |
| C″D loop oligo WT | |
| SEQ ID No. 905 | SLRPED |
| EF loop oligo WT | |
| EpCAM H90 TriTAC | EVQLVESGGGLVQPGNSLRLSCAASGFTFSKFGMSWVRQ |
| C2854 | APGKGLEWVSSISGSGRDTLYADSVKGRFTISRDNAKTT |
| (SEQ ID No. 906) | LYLQMNSLRPEDTAVYYCTIGGSLSVSSQGTLVTVSSGG |
| | GGSGGGSQTVVTQEPSLTVSPGGTVTLTCASSTGAVTSG |
| | NYPNWVQQKPGQAPRGLIGGTKFLVPGTPARFSGSLLGG |
| | KAALTLSGVQPEDEAEYYCTLWYSNRWVFGGGTKLTVLG |
| | GGGSGGGGSGGGGSEVQLVESGGGLVQPGGSLKLSCAAS |
| | GFTENKYAINWVRQAPGKGLEWVARIRSKYNNYATYYAD |
| | QVKDRFTISRDDSKNTAYLQMNNLKTEDTAVYYCVRHAN |
| | FGNSYISYWAYWGQGTLVTVSSGGGGSGGGSEVQLLESG |
| | GGLVQPGGSLTLSCAASGFIFRAASMAWYRQSPGNEREL |
| | VASISSGAFTNYADSVKARFTISRDNSKNTLYLQMNSLR |
| | AEDTAVYYCGATFLRSDGHHTINGQGTLVTVSSHHHHHH |
| EpCAM H90 ProTriTAC | QTVVTQEPSLTVSPGGTVTLTCASSTGAVTSGNYPNWVQ |
| (L040) C2704 | QKPGQAPRGLIGGTKFLVPGTPARFSGSLLGGKAALTLS |
| (SEQ ID No. 907) | GVQPEDEAEYYCTLWYSNRWVFGGGTKLTVLGGGGSGGG |
| | GSGGGGSEVQLVESGGGLVQPGGSLKLSCAASGFTENKY |
| | AINWVRQAPGKGLEWVARIRSKYNNYATYYADQVKDRFT |
| | ISRDDSKNTAYLQMNNLKTEDTAVYYCVRHANFGNSYIS |
| | YWAYWGQGTLVTVSSGGGGSGGGSEVQLLESGGGLVQPG |
| | GSLTLSCAASGFIFRAASMAWYRQSPGNERELVASISSG |
| | AFTNYADSVKARFTISRDNSKNTLYLQMNSLRAEDTAVY |
| | YCGATFLRSDGHHTINGQGTLVTVSSHHHHHH |
| EpCAM H90 ProTriTAC | EVQLVESGGGLVQPGNSLRLSCAASGFTFSKFGMSWVRQ |
| (NCLV) C2302 | GGGGGLDGNEEPGGLEWVSSISGSGRDTLYADSVKGRFT |
| (SEQ ID No. 908) | ISRDNAKTTLYLQMNSLRPEDTAVYYCTIGGSLSVSSQG |
| | TLVTVSSGGGGSGGGGSGGVVGGGGTQTVVTQEPSLTVS |
| | PGGTVTLTCASSTGAVTSGNYPNWVQQKPGQAPRGLIGG |
| | TKFLVPGTPARFSGSLLGGKAALTLSGVQPEDEAEYYCT |
| | LWYSNRWVFGGGTKLTVLGGGGSGGGGSGGGGSEVQLVE |
| | SGGGLVQPGGSLKLSCAASGFTENKYAINWVRQAPGKGL |
| | EWVARIRSKYNNYATYYADQVKDRFTISRDDSKNTAYLQ |
| | MNNLKTEDTAVYYCVRHANFGNSYISYWAYWGQGTLVTV |
| | SSGGGGSGGGSEVQLLESGGGLVQPGGSLTLSCAASGFI |
| | FRAASMAWYRQSPGNERELVASISSGAFTNYADSVKARF |
| | TISRDNSKNTLYLQMNSLRAEDTAVYYCGATFLRSDGHH |
| | TINGQGTLVTVSSHHHHHH |

What is claimed is:

1. A binding moiety comprising a non-CDR loop and a cleavable linker, wherein the moiety is capable of masking the binding of a binding molecule to its target, wherein the binding molecule comprises an immunoglobulin molecule or a non-immunoglobulin molecule.

2. The binding moiety of claim 1, wherein the moiety is a natural peptide, a synthetic peptide, an engineered scaffold, or an engineered bulk serum protein.

3. The binding moiety of claim 1 or 2, wherein the engineered scaffold comprises a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold.

4. The binding moiety of any one of claims 1-3, wherein the moiety is capable of binding to a bulk serum protein.

5. The binding moiety of any one of claims 1-4, wherein the non-CDR loop is from a variable domain, a constant domain, a C1-set domain, a C2-set domain, an I-domain, or any combinations thereof.

6. The binding moiety of any one of claims 1-5, wherein the moiety further comprises complementarity determining regions (CDRs).

7. The binding moiety of claim 6, wherein the moiety is capable of binding to a bulk serum protein.

8. The binding moiety of claim 7, wherein the bulk serum protein is a half-life extending protein.

9. The binding moiety of claim 7 or 8, wherein the bulk serum protein is albumin, transferrin, IgG1, IgG2, IgG4, IgG3, IgA monomer, Factor XIII, Fibrinogen, IgE, or pentameric IgM.

10. The binding moiety of any one of claims 1-9, wherein the moiety comprises a binding site specific for an immunoglobulin light chain.

11. The binding moiety of claim 10, wherein the immunoglobulin light chain is an Igκ free light chain.

12. The binding moiety of any one of claims 6-11, wherein the CDRs provide the binding site specific for the bulk serum protein or the immunoglobulin light chain.

13. The binding moiety of any one of claims 1-12, wherein the immunoglobulin molecule is a target antigen binding domain.

14. The binding moiety of claim 13, wherein the moiety is bound to the target antigen binding domain.

15. The binding moiety of claim 13 or 14, wherein the moiety is covalently linked to the target antigen binding domain.

16. The binding moiety of claim 13, 14, or 15, wherein the moiety is capable of masking the binding of the target antigen binding domain to its target via specific intermolecular interactions between the binding moiety and the target antigen binding domain.

17. The binding moiety of any one of claims 13-16, wherein the non-CDR loop provides a binding site specific for binding of the moiety to the target antigen binding domain.

18. The binding moiety of any one of claims 13-17, wherein upon cleavage of the cleavable linker, the binding moiety is separated from the target antigen binding domain and the target antigen binding domain binds to its target.

19. The binding moiety of any one of claims 13-18, wherein the target antigen domain binds to a tumor antigen.

20. The binding moiety of claim 19, wherein the tumor antigen comprises EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

21. The binding moiety of any one of claims 13-18, wherein the target antigen domain binds to an immune checkpoint protein.

22. The binding moiety of claim 21, wherein the immune checkpoint protein is CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

23. The binding moiety of any one of claims 13-18, wherein the target antigen binding domain binds to a T-cell.

24. The binding moiety of any one of claims 13-18, wherein the target antigen binding domain binds to CD3.

25. The binding moiety of any one of claims 1-24, wherein the cleavable linker comprises a cleavage site.

26. The binding moiety of claim 25, wherein the cleavage site is recognized by a protease.

27. The binding moiety of claim 26, wherein the protease cleavage site is recognized by a serine protease, a cysteine protease, an aspartate protease, a threonine protease, a glutamic acid protease, a metalloproteinase, a gelatinase, or a asparagine peptide lyase.

28. The binding moiety of claim 26, wherein the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, or dipeptidyl peptidase IV (DPPIV/CD26), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26).

29. A conditionally active binding protein comprising a binding moiety (M) which comprises a non-CDR loop, a cleavable linker (L), a first target antigen binding domain (T1), and a second target antigen binding domain (T2), wherein the first target antigen binding domain (T1) comprises an immunoglobulin molecule, wherein the non-CDR loop is capable of binding to the first target antigen binding domain, and wherein the binding moiety is capable of masking the binding of the first target antigen binding domain to its target.

30. The conditionally active binding protein of claim 29, wherein the binding moiety is capable of binding to a half-life extending protein.

31. The conditionally active binding protein of claim 29 or 30, wherein the binding moiety is a natural peptide, a synthetic peptide, an engineered scaffold, or an engineered serum bulk protein.

32. The conditionally active binding protein of claim 31, wherein the engineered scaffold comprises a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold.

33. The conditionally active binding protein of any one of claims 29-32, wherein the non-CDR loop is from a variable domain, a constant domain, a C1-set domain, a C2-set domain, an I-domain, or any combinations thereof.

34. The conditionally active binding protein of any one of claims 29-33, wherein the binding moiety further comprises complementarity determining regions (CDRs).

35. The conditionally active binding protein of any one of claims 29-34, wherein the binding moiety comprises a binding site specific for a bulk serum protein.

36. The conditionally active binding protein of claim 35, wherein the bulk serum protein is albumin, transferrin, IgG1, IgG2, IgG4, IgG3, IgA monomer, Factor XIII, Fibrinogen, IgE, or pentameric IgM.

37. The conditionally active binding protein of any one of claims 29-36, wherein the binding moiety further comprises a binding site specific for an immunoglobulin light chain.

38. The conditionally active binding protein of claim 37, wherein the immunoglobulin light chain is an Igκ free light chain.

39. The conditionally active binding protein of any one of claims 34-38, wherein the CDRs provide the binding site specific for the bulk serum protein or the immunoglobulin light chain, or any combinations thereof.

40. The conditionally active binding protein of any one of claims 34-39, wherein the binding moiety is capable of masking the binding of the first target antigen binding domain to its target via specific intermolecular interactions between the binding moiety and the first target antigen binding domain.

41. The conditionally active binding protein of any one of claims 29-40, wherein the non-CDR loop provides a binding site specific for binding of the binding moiety to the first target antigen binding domain.

42. The conditionally active binding protein of any one of claims 29-41, wherein the first or the second target antigen binding domain binds to a tumor antigen.

43. The conditionally active binding protein of claim 42, wherein the tumor antigen comprises EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

44. The conditionally active binding protein of any one of claims 29-41, wherein the first or the second target antigen binding domain binds to an immune checkpoint protein.

45. The conditionally active binding protein of claim 44, wherein the immune checkpoint protein is CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

46. The conditionally active binding protein of any one of claims 29-41, wherein first or the second target antigen binding domain binds to an immune cell.

47. The conditionally active binding protein of any one of claims 29-46, wherein the first or the second target antigen binding domain binds to a T-cell.

48. The conditionally active binding protein of any one of claims 29-47, wherein the first or the second target antigen binding domain binds to CD3.

49. The conditionally active binding protein of any one of claims 29-48, wherein the binding moiety (M), the cleavable linker (L), the first target antigen binding domain (T1), and the second target antigen binding domain (T2) are in one of the following configurations: M:L:T1:T2, and T2:T1:L:M.

50. The conditionally active binding protein of any one of claims 29-49, wherein the cleavable linker comprises a cleavage site.

51. The conditionally active binding protein of claim 50, wherein the cleavage site is recognized by a protease.

52. The conditionally active binding protein of claim 51, wherein the protease cleavage site is recognized by a serine protease, a cysteine protease, an aspartate protease, a threonine protease, a glutamic acid protease, a metalloproteinase, a gelatinase, or a asparagine peptide lyase.

53. The conditionally active binding protein of claim 51, wherein the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP8, a MMP9, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1l converting enzyme, a thrombin, a FAP (FAP-α), a dipeptidyl peptidase, or dipeptidyl peptidase IV (DPPIV/CD26), a type II transmembrane serine protease (TTSP), a neutrophil elastase, a cathepsin G, a proteinase 3, a neutrophil serine protease 4, a mast cell chymase, a mast cell tryptase, a dipeptidyl peptidase, and a dipeptidyl peptidase IV (DPPIV/CD26).

54. The conditionally active binding protein of claim 29, further comprising a half-life extension domain bound to the binding moiety, wherein the half-life extension domain provides the binding protein with a safety switch, and wherein upon cleavage of the linker the binding protein is activated by separation of the binding moiety and the half-life extension domain from the first target antigen binding domain, and the binding protein is thereby separated from the safety switch.

55. The conditionally active binding protein of claim 54, wherein the cleavage of the linker is in a tumor microenvironment.

56. A conditionally active binding protein, comprising a binding moiety linked to a target antigen binding domain via a non-CDR loop within the binding moiety, wherein the binding moiety is further linked to a half-life extension domain and comprises a cleavable linker, wherein the target antigen binding domain comprises an immunoglobulin molecule, wherein the binding protein has an extended half-life prior to its activation by cleavage of the linker, and wherein upon activation the binding moiety and the half-life extension domain are separated from the target antigen binding domain, and wherein the binding protein, in its activated state, does not have an extended half-life.

57. The conditionally active binding protein of claim 56, wherein the cleavage of the linker is in a tumor microenvironment.

58. A method of treatment comprising administering to a subject a conditionally active binding protein according to any one of claims 1-56, or a pharmaceutical composition comprising the same.

59. The conditionally active binding protein of any one of claims 29-56, wherein the non-CDR loop comprises a CC′ loop of at least one of: a camelid VHH domain, a human VH domain, a humanized VH domain, or a single domain antibody.

60. The conditionally active binding protein of any one of claims 29-56, wherein the binding moiety comprises a binding site specific for a CD3e domain, and wherein the binding site for the CD3e domain comprises at least one of the following motifs: QDGNE, QDGNEE, DGNE, and DGNEE.

61. The conditionally active binding protein of any one of claims 29-56, wherein the non-CDR loop comprises a CC′ loop of at least one of: a camelid VHH domain, a human VH domain, a humanized VH domain, or a single domain antibody.

62. The conditionally active binding protein of any one of claims 29-56, wherein the binding moiety comprises a binding site specific for a CD3e domain, and wherein the binding site for the CD3e domain comprises at least one of the following motifs: QDGNE, QDGNEE, DGNE, and DGNEE.

63. The binding moiety any one of claims 1-28, wherein the non-CDR loop comprises a CC′ loop of at least one of: a camelid VHH domain, a human VH domain, a humanized VH domain, or a single domain antibody.

64. The binding moiety of any one of claims 1-28, wherein the binding moiety comprises a binding site specific for a CD3e domain, and wherein the binding site for the CD3e domain comprises at least one of the following motifs: QDGNE, QDGNEE, DGNE, and DGNEE.
